SEQUENCE LISTING (167 sequences)

SEQ ID NO: 1 - protein, 5 aa, synthetic construct
SYSMN

SEQ ID NO: 2 - protein, 17 aa, synthetic construct
SISRSSSYIYYADSVKG

SEQ ID NO: 3 - protein, 12 aa, synthetic construct
EHGYSNSDAFDI

SEQ ID NO: 4 - protein, 11 aa, synthetic construct
RASQGISNYLA

SEQ ID NO: 5 - protein, 7 aa, synthetic construct
AASSLQS

SEQ ID NO: 6 - protein, 9 aa, synthetic construct
LQHNSYPRT

SEQ ID NO: 7 - protein, 12 aa, synthetic construct
IHGYSNSDAFDK

SEQ ID NO: 8 - protein, 12 aa, synthetic construct
IHGYSNSDAFDI

SEQ ID NO: 9 - protein, 11 aa, synthetic construct
RASQGISHYLV

SEQ ID NO: 10 - protein, 17 aa, synthetic construct
SISSSSSYIYYADSVKG

SEQ ID NO: 11 - protein, 12 aa, synthetic construct
RHGYSNSDAFDN

SEQ ID NO: 12 - protein, 9 aa, synthetic construct
LQHNSYPWT

SEQ ID NO: 13 - protein, 5 aa, synthetic construct
TYWMH

SEQ ID NO: 14 - protein, 17 aa, synthetic construct
RINGDGSRTNYADSVKG

SEQ ID NO: 15 - protein, 7 aa, synthetic construct
SSYAFDV

SEQ ID NO: 16 - protein, 17 aa, synthetic construct
RSSQSLLDSDDGSTYLD

SEQ ID NO: 17 - protein, 7 aa, synthetic construct
LLSNRAS

SEQ ID NO: 18 - protein, 9 aa, synthetic construct
MQRIEFPLT

SEQ ID NO: 19 - protein, 17 aa, synthetic construct
RINSDGSRTNYADSVKG

SEQ ID NO: 20 - protein, 7 aa, synthetic construct
SSYAFHV

SEQ ID NO: 21 - protein, 17 aa, synthetic construct
  VARIANT 4: note = X is R or S
SISXSSSYIYYADSVKG

SEQ ID NO: 22 - protein, 12 aa, synthetic construct
  VARIANT 1: note = X is E, I, or R
  VARIANT 12: note = X is I, K, or N
XHGYSNSDAFDX

SEQ ID NO: 23 - protein, 11 aa, synthetic construct
  VARIANT 8: note = X is N or H
  VARIANT 11: note = X is A or V
RASQGISXYLX

SEQ ID NO: 24 - protein, 9 aa, synthetic construct
  VARIANT 8: note = X is R or W
LQHNSYPXT

SEQ ID NO: 25 - protein, 17 aa, synthetic construct
  VARIANT 4: note = X is G or S
RINXDGSRTNYADSVKG

SEQ ID NO: 26 - protein, 7 aa, synthetic construct
  VARIANT 6: note = X is D or H
SSYAFXV

SEQ ID NO: 27 - protein, 121 aa, synthetic construct
EVQLVESGGGLVKPGGSLRLSCVASGFTFSSYSMNWVRQAPGKGLEWVSSISRSSSYIYYADSVKGRFTISRDNAKNSLYLQMNSLRAEDTAVYYCAREHGYSNSDAFDIWGQGTLVTVSS

SEQ ID NO: 28 - protein, 107 aa, synthetic construct
DIQMTQSPSAMSASVGDRVTITCRASQGISNYLAWFQQKPGKVPKRLIYAASSLQSGVPSRFSGSGSGTEFTLTISSLQPEDFATYYCLQHNSYPRTFGQGTKVEIK

SEQ ID NO: 29 - protein, 121 aa, synthetic construct
EVQLVESGGGLVKPGGSLRLSCVASGFTFSSYSMNWVRQAPGKGLEWVSSISRSSSYIYYADSVKGRFTISRDNAKNSLYLQMNSLRAEDTAVYYCARIHGYSNSDAFDKWGQGTLVTVSS

SEQ ID NO: 30 - protein, 121 aa, synthetic construct
EVQLVESGGGLVKPGGSLRLSCVASGFTFSSYSMNWVRQAPGKGLEWVSSISRSSSYIYYADSVKGRFTISRDNAKNSLYLQMNSLRAEDTAVYYCARIHGYSNSDAFDIWGQGTLVTVSS

SEQ ID NO: 31 - protein, 107 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPRTFGQGTKVEIK

SEQ ID NO: 32 - protein, 121 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSS

SEQ ID NO: 33 - protein, 107 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIK

SEQ ID NO: 34 - protein, 116 aa, synthetic construct
EVQLVESGGGLVQPGGSLRLSCAASGFTFRTYWMHWVRQAPGKGLLWVSRINGDGSRTNYADSVKGRFTISRDNAKKTLYLQMNSLRAEDTAVYFCARSSYAFDVWGQGTMVTVSS

SEQ ID NO: 35 - protein, 113 aa, synthetic construct
DVVMTQTPLSLPVTPGEPASISCRSSQSLLDSDDGSTYLDWYLQKPGQSPQLLIYLLSNRASGVPDRFSGSGSGTVFTLKISSVEAADVGVYYCMQRIEFPLTFGGGTKVEIK

SEQ ID NO: 36 - protein, 116 aa, synthetic construct
EVQLVESGGGLVQPGGSLRLSCAASGFTFRTYWMHWVRQAPGKGLVWVSRINSDGSRTNYADSVKGRFTISRDNAKNTLYLQMNSLRAEDTAVYYCARSSYAFDVWGQGTLVTVSS

SEQ ID NO: 37 - protein, 113 aa, synthetic construct
DIVMTQTPLSLPVTPGEPASISCRSSQSLLDSDDGSTYLDWYLQKPGQSPQLLIYLLSNRASGVPDRFSGSGSGTDFTLKISRVEAEDVGVYYCMQRIEFPLTFGGGTKVEIK

SEQ ID NO: 38 - protein, 116 aa, synthetic construct
EVQLVESGGGLVQPGGSLRLSCAASGFTFRTYWMHWVRQAPGKGLVWVSRINSDGSRTNYADSVKGRFTISRDNAKNTLYLQMNSLRAEDTAVYYCARSSYAFHVWGQGTLVTVSS

SEQ ID NO: 39 - protein, 5 aa, synthetic construct
ETAVA

SEQ ID NO: 40 - protein, 17 aa, synthetic construct
GIGGGVDITYYADSVKG

SEQ ID NO: 41 - protein, 17 aa, synthetic construct
RPGRPLITSKVADLYPY

SEQ ID NO: 42 - protein, 128 aa, synthetic construct
EVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 43 - protein, 5 aa, synthetic construct
SYAIE

SEQ ID NO: 44 - protein, 17 aa, synthetic construct
GILPGSGTINYNEKFKG

SEQ ID NO: 45 - protein, 11 aa, synthetic construct
MSSNSDQGFDL

SEQ ID NO: 46 - protein, 11 aa, synthetic construct
KASQGISRFLS

SEQ ID NO: 47 - protein, 7 aa, synthetic construct
AVSSLVD

SEQ ID NO: 48 - protein, 9 aa, synthetic construct
VQYNSYPYG

SEQ ID NO: 49 - protein, 120 aa, synthetic construct
QVQLVQSGAEVKKPGSSVKVSCKASGYTFSSYAIEWVRQAPGQGLEWMGGILPGSGTINYNEKFKGRVTITADKSTSTAYMELSSLRSEDTAVYYCARMSSNSDQGFDLWGQGTLVTVSS

SEQ ID NO: 50 - protein, 107 aa, synthetic construct
DIQMTQSPSSLSASVGDRVTITCKASQGISRFLSWFQQKPGKAPKSLIYAVSSLVDGVPSRFSGSGSGTDFTLTISSLQPEDFATYYCVQYNSYPYGFGGGTKVEIK

SEQ ID NO: 51 - protein, 446 aa, synthetic construct
  VARIANT 127: note = X is S or C
QVQLVQSGAEVKKPGSSVKVSCKASGYTFSSYAIEWVRQAPGQGLEWMGGILPGSGTINYNEKFKGRVTITADKSTSTAYMELSSLRSEDTAVYYCARMSSNSDQGFDLWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLG

SEQ ID NO: 52 - protein, 214 aa, synthetic construct
DIQMTQSPSSLSASVGDRVTITCKASQGISRFLSWFQQKPGKAPKSLIYAVSSLVDGVPSRFSGSGSGTDFTLTISSLQPEDFATYYCVQYNSYPYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 447 aa, synthetic construct
EVQLVESGGGLVKPGGSLRLSCVASGFTFSSYSMNWVRQAPGKGLEWVSSISRSSSYIYYADSVKGRFTISRDNAKNSLYLQMNSLRAEDTAVYYCAREHGYSNSDAFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 54 - protein, 214 aa, synthetic construct
DIQMTQSPSAMSASVGDRVTITCRASQGISNYLAWFQQKPGKVPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 447 aa, synthetic construct
  VARIANT 128: note = X is S or C
EVQLVESGGGLVKPGGSLRLSCVASGFTFSSYSMNWVRQAPGKGLEWVSSISRSSSYIYYADSVKGRFTISRDNAKNSLYLQMNSLRAEDTAVYYCARIHGYSNSDAFDKWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 447 aa, synthetic construct
EVQLVESGGGLVKPGGSLRLSCVASGFTFSSYSMNWVRQAPGKGLEWVSSISRSSSYIYYADSVKGRFTISRDNAKNSLYLQMNSLRAEDTAVYYCARIHGYSNSDAFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 447 aa, synthetic construct
  VARIANT 128: note = X is S or C
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 442 aa, synthetic construct
EVQLVESGGGLVQPGGSLRLSCAASGFTFRTYWMHWVRQAPGKGLLWVSRINGDGSRTNYADSVKGRFTISRDNAKKTLYLQMNSLRAEDTAVYFCARSSYAFDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 220 aa, synthetic construct
DVVMTQTPLSLPVTPGEPASISCRSSQSLLDSDDGSTYLDWYLQKPGQSPQLLIYLLSNRASGVPDRFSGSGSGTVFTLKISSVEAADVGVYYCMQRIEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 442 aa, synthetic construct
EVQLVESGGGLVQPGGSLRLSCAASGFTFRTYWMHWVRQAPGKGLVWVSRINSDGSRTNYADSVKGRFTISRDNAKNTLYLQMNSLRAEDTAVYYCARSSYAFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 63 - protein, 220 aa, synthetic construct
DIVMTQTPLSLPVTPGEPASISCRSSQSLLDSDDGSTYLDWYLQKPGQSPQLLIYLLSNRASGVPDRFSGSGSGTDFTLKISRVEAEDVGVYYCMQRIEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 442 aa, synthetic construct
  VARIANT 123: note = X is S or C
EVQLVESGGGLVQPGGSLRLSCAASGFTFRTYWMHWVRQAPGKGLVWVSRINSDGSRTNYADSVKGRFTISRDNAKNTLYLQMNSLRAEDTAVYYCARSSYAFHVWGQGTLVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 223 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKC

SEQ ID NO: 66 - protein, 381 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKCDKTHTGGGGQGGGGQGGGGQGGGGQGGGGQEVQLLESGGGLVQPGGSLRLSCAASGRYIDETAVAWFRQAPGKGREFVAGIGGGVDITYYADSVKGRFTISRDNSKNTLYLQMNSLRPEDTAVYYCGARPGRPLITSKVADLYPYWGQGTLVTVSSPP

SEQ ID NO: 67 - protein, 214 aa, synthetic construct
DIQMTQSPSAMSASVGDRVTITCRASQGISHYLVWFQQKPGKVPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQCGNSQESVTEQDSKDSTYSLSSTLTLSKADYEKHKVYACEVTHQGLSSPVTKSFNRGEC

SEQ ID NO: 68 - protein, 447 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDICVEWESNGQPENNYKTTPPVLDSDGSFFLYSVLTVDKSRWQEGNVFSCSVMHEALHNHYTQKSLSLSLG

SEQ ID NO: 69 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDICVEWESNGQPENNYKTTPPVLDSDGSFFLASRLTVDKSRWQEGNVFSCSVMHEALHNHYTQKSLSLSLG

SEQ ID NO: 70 - protein, 20 aa, synthetic construct
GGGGQGGGGQGGGGQGGGGQ

SEQ ID NO: 71 - protein, 6 aa, synthetic construct
GSYWIC

SEQ ID NO: 72 - protein, 18 aa, synthetic construct
CIYSTSGGRTYYASWVKG

SEQ ID NO: 73 - protein, 12 aa, synthetic construct
GDDSISDAYFDL

SEQ ID NO: 74 - protein, 13 aa, synthetic construct
QSSQSVYNNNRLA

SEQ ID NO: 75 - protein, 7 aa, synthetic construct
DASTLAS

SEQ ID NO: 76 - protein, 12 aa, synthetic construct
QGTYFSSGWSWA

SEQ ID NO: 77 - protein, 121 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSS

SEQ ID NO: 78 - protein, 112 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVK

SEQ ID NO: 79 - protein, 447 aa, synthetic construct
QSLEESGGDLVKPEGSLTLTCTASGFSFSGSYWICWVRQAPGKGLEWIGCIYSTSGGRTYYASWVKGRFTISKTSSTTVTLQMTSLTAADTATYFCARGDDSISDAYFDLWGPGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 219 aa, synthetic construct
ALDMTQTASPVSAAVGGTVTINCQSSQSVYNNNRLAWYQQKPGQPPKLLIYDASTLASGVPSRFKGSGSGTQFTLTISGVQSDDSATYYCQGTYFSSGWSWAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - RNA, 21 nt, synthetic construct
  source 1..21: mol_type = other RNA
ctgtacaagtgctcagttcca

SEQ ID NO: 82 - RNA, 23 nt, synthetic construct
  source 1..23: mol_type = other RNA
tggaactgagcacttgtacagga

SEQ ID NO: 83 - RNA, 21 nt, synthetic construct
  source 1..21: mol_type = other RNA
tgtacaagtgctcagttccaa

SEQ ID NO: 84 - RNA, 23 nt, synthetic construct
  source 1..23: mol_type = other RNA
ttggaactgagcacttgtacagg

SEQ ID NO: 85 - RNA, 21 nt, synthetic construct
  source 1..21: mol_type = other RNA
gagcaagtgacaaatgttgga

SEQ ID NO: 86 - RNA, 23 nt, synthetic construct
  source 1..23: mol_type = other RNA
tccaacatttgtcacttgctctt

SEQ ID NO: 87 - RNA, 21 nt, synthetic construct
  source 1..21: mol_type = other RNA
ttccaatgtgcccagtcatga

SEQ ID NO: 88 - RNA, 23 nt, synthetic construct
  source 1..23: mol_type = other RNA
tcatgactgggcacattggaact

SEQ ID NO: 89 - RNA, 21 nt, synthetic construct
  source 1..21: mol_type = other RNA
agtgactaccacttatttcta

SEQ ID NO: 90 - RNA, 23 nt, synthetic construct
  source 1..23: mol_type = other RNA
tagaaataagtggtagtcactta

SEQ ID NO: 91 - RNA, 21 nt, synthetic construct
  source 1..21: mol_type = other RNA
gtgactaccacttatttctaa

SEQ ID NO: 92 - RNA, 23 nt, synthetic construct
  source 1..23: mol_type = other RNA
ttagaaataagtggtagtcactt

SEQ ID NO: 93 - RNA, 21 nt, synthetic construct
  misc_feature 1..21: note = /note="Description of Artificial Sequence: Synthetic oligonucleotide"
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
ctgtacaagtgctcagttcca

SEQ ID NO: 94 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluorocytidine
  modified_base 7: mod_base = um
  modified_base 8: mod_base = gm
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
  source 1..23: mol_type = other RNA
tggaactgagcacttgtacagga

SEQ ID NO: 95 - RNA, 21 nt, synthetic construct
  misc_feature 1..21: note = /note="Description of Artificial Sequence: Synthetic oligonucleotide"
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
ctgtacaagtgctcagttcca

SEQ ID NO: 96 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-fluoroadenosine
  modified_base 6: mod_base = cm
  modified_base 7: mod_base = OTHER; note = 2'-fluorouridine
  modified_base 8: mod_base = gm
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
  source 1..23: mol_type = other RNA
tggaactgagcacttgtacagga

SEQ ID NO: 97 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-fluoroadenosine
  modified_base 6: mod_base = cm
  modified_base 7: mod_base = um
  modified_base 8: mod_base = OTHER; note = 2'-fluoroguanosine
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
  source 1..23: mol_type = other RNA
tggaactgagcacttgtacagga

SEQ ID NO: 98 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = OTHER; note = 2'-fluoroguanosine
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = OTHER; note = 2'-fluorouridine
  modified_base 8: mod_base = gm
  modified_base 9: mod_base = OTHER; note = 2'-O-methyladenosine
  modified_base 10: mod_base = gm
  modified_base 11: mod_base = cm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = um
  modified_base 16: mod_base = OTHER; note = 2'-fluoroguanosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = OTHER; note = 2'-O-methylguanosine with a 3'-phosphorothioate linkage
  modified_base 22: mod_base = OTHER; note = 2'-O-methylguanosine with a 3'-phosphorothioate linkage
  modified_base 23: mod_base = OTHER; note = 2'-O-methyladenosine
  source 1..23: mol_type = other RNA
tggaactgagcacttgtacagga

SEQ ID NO: 99 - RNA, 21 nt, synthetic construct
  misc_feature 1..21: note = /note="Description of Artificial Sequence: Synthetic oligonucleotide"
  modified_base 1: mod_base = OTHER; note = 2'-O-methylcytidine with a 3'-phosphorothioate linker and a 5'-iAb substitution
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
ctgtacaagtgctcagttcca

SEQ ID NO: 100 - RNA, 21 nt, synthetic construct
  misc_feature 1..21: note = /note="Description of Artificial Sequence: Synthetic oligonucleotide"
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = gm
  modified_base 16: mod_base = um
  modified_base 17: mod_base = um
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
tgtacaagtgctcagttccaa

SEQ ID NO: 101 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-fluoroadenosine
  modified_base 7: mod_base = cm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-fluorocytidine
  modified_base 15: mod_base = um
  modified_base 16: mod_base = OTHER; note = 2'-fluorouridine
  modified_base 17: mod_base = gm
  modified_base 18: mod_base = um
  modified_base 19: mod_base = OTHER; note = 2'-O-methyladenosine
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylguanosine with a 3'-phosphorothioate linkage
  modified_base 23: mod_base = gm
  source 1..23: mol_type = other RNA
ttggaactgagcacttgtacagg

SEQ ID NO: 102 - RNA, 21 nt, synthetic construct
  misc_feature 1..21: note = /note="Description of Artificial Sequence: Synthetic oligonucleotide"
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = OTHER; note = 2'-O-methyladenosine
  modified_base 5: mod_base = cm
  modified_base 6: mod_base = um
  modified_base 7: mod_base = OTHER; note = 2'-fluoroadenosine
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = um
  modified_base 14: mod_base = OTHER; note = 2'-O-methyladenosine
  modified_base 15: mod_base = um
  modified_base 16: mod_base = um
  modified_base 17: mod_base = um
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
gtgactaccacttatttctaa

SEQ ID NO: 103 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = OTHER; note = 2'-O-methyladenosine
  modified_base 4: mod_base = gm
  modified_base 5: mod_base = OTHER; note = 2'-O-methyladenosine
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = OTHER; note = 2'-O-methyladenosine
  modified_base 10: mod_base = OTHER; note = 2'-O-methyladenosine
  modified_base 11: mod_base = gm
  modified_base 12: mod_base = um
  modified_base 13: mod_base = gm
  modified_base 14: mod_base = OTHER; note = 2'-fluoroguanosine
  modified_base 15: mod_base = um
  modified_base 16: mod_base = OTHER; note = 2'-fluoroadenosine
  modified_base 17: mod_base = gm
  modified_base 18: mod_base = um
  modified_base 19: mod_base = cm
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
  source 1..23: mol_type = other RNA
ttagaaataagtggtagtcactt

SEQ ID NO: 104 - RNA, 21 nt, synthetic construct
  misc_feature 1..21: note = /note="Description of Artificial Sequence: Synthetic oligonucleotide"
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = um
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluorocytidine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-O-methyladenosine
  modified_base 15: mod_base = um
  modified_base 16: mod_base = gm
  modified_base 17: mod_base = um
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
gagcaagtgacaaatgttgga

SEQ ID NO: 105 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorocytidine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluorocytidine
  modified_base 7: mod_base = OTHER; note = 2'-O-methyladenosine
  modified_base 8: mod_base = um
  modified_base 9: mod_base = um
  modified_base 10: mod_base = um
  modified_base 11: mod_base = gm
  modified_base 12: mod_base = um
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluorouridine
  modified_base 17: mod_base = um
  modified_base 18: mod_base = gm
  modified_base 19: mod_base = cm
  modified_base 20: mod_base = um
  modified_base 21: mod_base = OTHER; note = 2'-O-methylcytidine with a 3'-phosphorothioate linkage
  modified_base 22: mod_base = OTHER; note = 2'-O-methyluridine with a 3'-phosphorothioate linkage
  modified_base 23: mod_base = um
  source 1..23: mol_type = other RNA
tccaacatttgtcacttgctctt

SEQ ID NO: 106 - RNA, 21 nt, synthetic construct
  misc_feature 1..21: note = /note="Description of Artificial Sequence: Synthetic oligonucleotide"
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = cm
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = cm
  modified_base 13: mod_base = um
  modified_base 14: mod_base = um
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
agtgactaccacttatttcta

SEQ ID NO: 107 - RNA, 23 nt, synthetic construct
  misc_feature 1..23: note = /note="Description of Artificial Sequence: Synthetic oligonucleotide"
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroadenosine with a 3'-phosphorothioate linkage
  modified_base 3: mod_base = gm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-fluoroadenosine
  modified_base 7: mod_base = um
  modified_base 8: mod_base = OTHER; note = 2'-O-methyladenosine
  modified_base 9: mod_base = OTHER; note = 2'-O-methyladenosine
  modified_base 10: mod_base = gm
  modified_base 11: mod_base = um
  modified_base 12: mod_base = gm
  modified_base 13: mod_base = gm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = cm
  modified_base 19: mod_base = OTHER; note = 2'-O-methyladenosine
  modified_base 20: mod_base = cm
  modified_base 21: mod_base = OTHER; note = 2'-O-methyluridine with a 3'-phosphorothioate linkage
  modified_base 22: mod_base = OTHER; note = 2'-O-methyluridine with a 3'-phosphorothioate linkage
  modified_base 23: mod_base = OTHER; note = 2'-O-methyladenosine
  source 1..23: mol_type = other RNA
tagaaataagtggtagtcactta

SEQ ID NO: 108 - RNA, 21 nt, synthetic construct
  misc_feature 1..21: note = /note="Description of Artificial Sequence: Synthetic oligonucleotide"
  modified_base 1: mod_base = OTHER; note = 2'-O-methyladenosine with a 3'-phosphorothioate linkage and a 5'-iAb substitution
  modified_base 2: mod_base = OTHER; note = 2'-O-methylguanosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = cm
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = cm
  modified_base 13: mod_base = um
  modified_base 14: mod_base = um
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
agtgactaccacttatttcta

SEQ ID NO: 109 - DNA, 3177 bp, Homo sapiens
  source 1..3177: mol_type = genomic DNA
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 110 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 111 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 112 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 113 - protein, 681 aa, Mus musculus
HHHHHHCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 114 - protein, 678 aa, Homo sapiens
HHHHHHCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 115 - protein, 193 aa, Homo sapiens
HHHHHHHHGKPIPNPLLGLDSTGGGGSDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGGGGGGLPNIPVQTISRAAAEKLFGNMEGDCPSDWKTDSTCRMVTSESKNVKLTVS

SEQ ID NO: 116 - RNA, 21 nt, synthetic construct
  source 1..21: mol_type = other RNA
  modified_base 10: mod_base = OTHER; note = abasic nucleotide
ctgtacaagngctcagttcca

SEQ ID NO: 117 - RNA, 21 nt, synthetic construct
  misc_feature 1..21: note = /note="Description of Artificial Sequence: Synthetic oligonucleotide"
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = abasic nucleotide
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
ctgtacaagngctcagttcca

SEQ ID NO: 118 - RNA, 21 nt, synthetic construct
  misc_feature 1..21: note = /note="Description of Artificial Sequence: Synthetic oligonucleotide"
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = abasic nucleotide
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate link to C6-NH2
  source 1..21: mol_type = other RNA
ctgtacaagngctcagttcca

SEQ ID NO: 119 - protein, 19 aa, Homo sapiens
FGNMEGDCPSDWKTDSTCR

SEQ ID NO: 120 - RNA, 21 nt, synthetic construct
  source 1..21: mol_type = other RNA
gtggaagtaaaatctgagaaa

SEQ ID NO: 121 - RNA, 23 nt, synthetic construct
  source 1..23: mol_type = other RNA
tttctcagattttacttccacct

SEQ ID NO: 122 - RNA, 21 nt, synthetic construct
  source 1..21: mol_type = other RNA
ccaagtgtggctcattaggca

SEQ ID NO: 123 - RNA, 23 nt, synthetic construct
  source 1..23: mol_type = other RNA
tgcctaatgagccacacttggag

SEQ ID NO: 124 - RNA, 21 nt, synthetic construct
  source 1..21: mol_type = other RNA
tgcaaatagtctacaaaccaa

SEQ ID NO: 125 - RNA, 23 nt, synthetic construct
  source 1..23: mol_type = other RNA
ttggtttgtagactatttgcacc

SEQ ID NO: 126 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = um
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
gtggaagtaaaatctgagaaa

SEQ ID NO: 127 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = um
  modified_base 4: mod_base = cm
  modified_base 5: mod_base = um
  modified_base 6: mod_base = OTHER; note = 2'-fluorocytidine
  modified_base 7: mod_base = OTHER; note = 2'-O-methyladenosine
  modified_base 8: mod_base = gm
  modified_base 9: mod_base = OTHER; note = 2'-O-methyladenosine
  modified_base 10: mod_base = um
  modified_base 11: mod_base = um
  modified_base 12: mod_base = um
  modified_base 13: mod_base = um
  modified_base 14: mod_base = OTHER; note = 2'-fluoroadenosine
  modified_base 15: mod_base = cm
  modified_base 16: mod_base = OTHER; note = 2'-fluorouridine
  modified_base 17: mod_base = um
  modified_base 18: mod_base = cm
  modified_base 19: mod_base = cm
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylcytidine with a 3'-phosphorothioate linkage
  modified_base 23: mod_base = um
  source 1..23: mol_type = other RNA
tttctcagattttacttccacct

SEQ ID NO: 128 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = OTHER; note = 2'-fluoroguanosine
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
ccaagtgtggctcattaggca

SEQ ID NO: 129 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = um
  modified_base 6: mod_base = OTHER; note = 2'-fluoroadenosine
  modified_base 7: mod_base = OTHER; note = 2'-O-methyladenosine
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = cm
  modified_base 18: mod_base = um
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = gm
  source 1..23: mol_type = other RNA
tgcctaatgagccacacttggag

SEQ ID NO: 130 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
tgcaaatagtctacaaaccaa

SEQ ID NO: 131 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = um
  modified_base 6: mod_base = OTHER; note = 2'-fluorouridine
  modified_base 7: mod_base = um
  modified_base 8: mod_base = gm
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggtttgtagactatttgcacc

SEQ ID NO: 132 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = um
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
gtggaagtaaaatctgagaaa

SEQ ID NO: 133 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = um
  modified_base 4: mod_base = cm
  modified_base 5: mod_base = OTHER; note = 2'-fluorouridine
  modified_base 6: mod_base = cm
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-O-methyladenosine
  modified_base 10: mod_base = um
  modified_base 11: mod_base = um
  modified_base 12: mod_base = um
  modified_base 13: mod_base = um
  modified_base 14: mod_base = OTHER; note = 2'-fluoroadenosine
  modified_base 15: mod_base = cm
  modified_base 16: mod_base = OTHER; note = 2'-fluorouridine
  modified_base 17: mod_base = um
  modified_base 18: mod_base = cm
  modified_base 19: mod_base = cm
  modified_base 20: mod_base = OTHER; note = 2'-O-methyladenosine
  modified_base 21: mod_base = OTHER; note = 2'-O-methylcytidine with a 3'-phosphorothioate linkage
  modified_base 22: mod_base = OTHER; note = 2'-O-methylcytidine with a 3'-phosphorothioate linkage
  modified_base 23: mod_base = um
  source 1..23: mol_type = other RNA
tttctcagattttacttccacct

SEQ ID NO: 134 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
ccaagtgtggctcattaggca

SEQ ID NO: 135 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluoroguanosine with a 3'-phosphorothioate linkage
  modified_base 3: mod_base = cm
  modified_base 4: mod_base = cm
  modified_base 5: mod_base = OTHER; note = 2'-fluorouridine
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluorouridine
  modified_base 9: mod_base = gm
  modified_base 10: mod_base = OTHER; note = 2'-O-methyladenosine
  modified_base 11: mod_base = gm
  modified_base 12: mod_base = cm
  modified_base 13: mod_base = cm
  modified_base 14: mod_base = OTHER; note = 2'-fluoroadenosine
  modified_base 15: mod_base = cm
  modified_base 16: mod_base = OTHER; note = 2'-fluoroadenosine
  modified_base 17: mod_base = cm
  modified_base 18: mod_base = um
  modified_base 19: mod_base = um
  modified_base 20: mod_base = gm
  modified_base 21: mod_base = OTHER; note = 2'-O-methylguanosine with a 3'-phosphorothioate linkage
  modified_base 22: mod_base = OTHER; note = 2'-O-methyladenosine with a 3'-phosphorothioate linkage
  modified_base 23: mod_base = gm
  source 1..23: mol_type = other RNA
tgcctaatgagccacacttggag

SEQ ID NO: 136 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine with a 3'-phosphorothioate linked to C6-NH2
  source 1..21: mol_type = other RNA
tgcaaatagtctacaaaccaa

SEQ ID NO: 137 - RNA, 23 nt, synthetic construct
  misc_feature 1..23: note = Description of Artificial Sequence: Synthetic oligonucleotide
  modified_base 1: mod_base = OTHER; note = 2'-O-methyluridine with a 3'-phosphorothioate linkage and a 5'-vinylphosphonate substitution
  modified_base 2: mod_base = OTHER; note = 2'-fluorouridine with a 3'-phosphorothioate linkage
  modified_base 3: mod_base = gm
  modified_base 4: mod_base = gm
  modified_base 5: mod_base = OTHER; note = 2'-fluorouridine
  modified_base 6: mod_base = um
  modified_base 7: mod_base = um
  modified_base 8: mod_base = OTHER; note = 2'-fluoroguanosine
  modified_base 9: mod_base = um
  modified_base 10: mod_base = OTHER; note = 2'-O-methyladenosine
  modified_base 11: mod_base = gm
  modified_base 12: mod_base = OTHER; note = 2'-O-methyladenosine
  modified_base 13: mod_base = cm
  modified_base 14: mod_base = OTHER; note = 2'-fluorouridine
  modified_base 15: mod_base = OTHER; note = 2'-O-methyladenosine
  modified_base 16: mod_base = OTHER; note = 2'-fluorouridine
  modified_base 17: mod_base = um
  modified_base 18: mod_base = um
  modified_base 19: mod_base = gm
  modified_base 20: mod_base = cm
  modified_base 21: mod_base = OTHER; note = 2'-O-methyladenosine with a 3'-phosphorothioate linkage
  modified_base 22: mod_base = OTHER; note = 2'-O-methylcytidine with a 3'-phosphorothioate linkage
  modified_base 23: mod_base = cm
  source 1..23: mol_type = other RNA
ttggtttgtagactatttgcacc

SEQ ID NO: 138 - protein, 447 aa, synthetic construct
EVQLVESGGGLVKPGGSLRLSCVASGFTFSSYSMNWVRQAPGKGLEWVSSISSSSSYIYYADSVKGRFTISRDNAKNSLYLQMNSLRAEDTAVYYCARRHGYSNSDAFDNWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVSTLPPSQEEMTKNQVSLMCLVYGFYPSDIAVEWESNGQPENNYKTTPPVLDSDGSFFLYSVLTVDKSRWQEGNVFSCSVMHEALHNHYTQKSLSLSLG

SEQ ID NO: 139 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQGDMTKNQVQLTCLVKGFYPSDIAVEWESNGQPENNYKTTPPVLDSDGSFFLASRLTVDKSRWQEGNVFSCSVMHEALHNHYTQKSLSLSLG

SEQ ID NO: 140 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 141 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 142 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage and a 5'-iAb substitution
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagtgctcagttcca

SEQ ID NO: 143 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = OTHER; note = 2'-fluoroadenosine
  modified_base 8: mod_base = gm
  modified_base 9: mod_base = OTHER; note = 2'-fluorouridine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = gm
  modified_base 16: mod_base = um
  modified_base 17: mod_base = um
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgtacaagtgctcagttccaa

SEQ ID NO: 144 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = OTHER; note = 2'-O-methyladenosine
  modified_base 5: mod_base = cm
  modified_base 6: mod_base = um
  modified_base 7: mod_base = OTHER; note = 2'-fluoroadenosine
  modified_base 8: mod_base = cm
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorocytidine
  modified_base 12: mod_base = um
  modified_base 13: mod_base = um
  modified_base 14: mod_base = OTHER; note = 2'-O-methyladenosine
  modified_base 15: mod_base = um
  modified_base 16: mod_base = um
  modified_base 17: mod_base = um
  modified_base 18: mod_base = cm
  modified_base 19: mod_base = OTHER; note = 2'-O-methyluridine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtgactaccacttatttctaa

SEQ ID NO: 145 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyladenosine with a 3'-phosphorothioate linkage
  modified_base 3: mod_base = gm
  modified_base 4: mod_base = cm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = um
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluorocytidine
  modified_base 12: mod_base = OTHER; note = 2'-O-methyladenosine
  modified_base 13: mod_base = OTHER; note = 2'-O-methyladenosine
  modified_base 14: mod_base = OTHER; note = 2'-O-methyladenosine
  modified_base 15: mod_base = um
  modified_base 16: mod_base = gm
  modified_base 17: mod_base = um
  modified_base 18: mod_base = um
  modified_base 19: mod_base = OTHER; note = 2'-O-methylguanosine with a 3'-phosphorothioate linkage
  modified_base 20: mod_base = OTHER; note = 2'-O-methylguanosine with a 3'-phosphorothioate linkage
  modified_base 21: mod_base = OTHER; note = 2'-O-methyladenosine
  source 1..21: mol_type = other RNA
gagcaagtgacaaatgttgga

SEQ ID NO: 146 - RNA, 21 nt, synthetic construct
  misc_feature 1..21: note = Description of Artificial Sequence: Synthetic oligonucleotide
  modified_base 1: mod_base = OTHER; note = 2'-O-methyladenosine with a 3'-phosphorothioate linkage
  modified_base 2: mod_base = OTHER; note = 2'-O-methylguanosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = cm
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = cm
  modified_base 13: mod_base = um
  modified_base 14: mod_base = um
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
  source 1..21: mol_type = other RNA
agtgactaccacttatttcta

SEQ ID NO: 147 - RNA, 21 nt, synthetic construct
  misc_feature 1..21: note = Description of Artificial Sequence: Synthetic oligonucleotide
  modified_base 1: mod_base = OTHER; note = 2'-O-methyladenosine with a 3'-phosphorothioate linkage and a 5'-iAb substitution
  modified_base 2: mod_base = OTHER; note = 2'-O-methylguanosine with a 3'-phosphorothioate linkage
  modified_base 3: mod_base = um
  modified_base 4: mod_base = gm
  modified_base 5: mod_base = OTHER; note = 2'-O-methyladenosine
  modified_base 6: mod_base = cm
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluorocytidine
  modified_base 11: mod_base = OTHER; note = 2'-fluoroadenosine
  modified_base 12: mod_base = cm
  modified_base 13: mod_base = um
  modified_base 14: mod_base = um
  modified_base 15: mod_base = OTHER; note = 2'-O-methyladenosine
  modified_base 16: mod_base = um
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methyluridine with a 3'-phosphorothioate linkage
  modified_base 21: mod_base = OTHER; note = 2'-O-methyladenosine
  source 1..21: mol_type = other RNA
agtgactaccacttatttcta

SEQ ID NO: 148 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = abasic nucleotide
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagngctcagttcca

SEQ ID NO: 149 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = um
  modified_base 5: mod_base = OTHER; note = 2'-O-methyladenosine
  modified_base 6: mod_base = cm
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = abasic nucleotide
  modified_base 11: mod_base = OTHER; note = 2'-fluoroguanosine
  modified_base 12: mod_base = cm
  modified_base 13: mod_base = um
  modified_base 14: mod_base = cm
  modified_base 15: mod_base = OTHER; note = 2'-O-methyladenosine
  modified_base 16: mod_base = gm
  modified_base 17: mod_base = um
  modified_base 18: mod_base = um
  modified_base 19: mod_base = OTHER; note = 2'-O-methylcytidine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ctgtacaagngctcagttcca

SEQ ID NO: 150 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = um
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtggaagtaaaatctgagaaa

SEQ ID NO: 151 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = OTHER; note = 2'-fluoroguanosine
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ccaagtgtggctcattaggca

SEQ ID NO: 152 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluorouridine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 153 - RNA, 21 nt, synthetic construct
  misc_feature 1..21: note = Description of Artificial Sequence: Synthetic oligonucleotide
  modified_base 1: mod_base = OTHER; note = 2'-O-methylguanosine with a 3'-phosphorothioate linkage
  modified_base 2: mod_base = OTHER; note = 2'-O-methyluridine with a 3'-phosphorothioate linkage
  modified_base 3: mod_base = gm
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = um
  modified_base 9: mod_base = OTHER; note = 2'-fluoroadenosine
  modified_base 10: mod_base = OTHER; note = 2'-fluoroadenosine
  modified_base 11: mod_base = OTHER; note = 2'-fluoroadenosine
  modified_base 12: mod_base = OTHER; note = 2'-O-methyladenosine
  modified_base 13: mod_base = um
  modified_base 14: mod_base = cm
  modified_base 15: mod_base = um
  modified_base 16: mod_base = gm
  modified_base 17: mod_base = OTHER; note = 2'-O-methyladenosine
  modified_base 18: mod_base = gm
  modified_base 19: mod_base = OTHER; note = 2'-O-methyladenosine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
gtggaagtaaaatctgagaaa

SEQ ID NO: 154 - RNA, 21 nt, synthetic construct
  misc_feature 1..21: note = Description of Artificial Sequence: Synthetic oligonucleotide
  modified_base 1: mod_base = OTHER; note = 2'-O-methylcytidine with a 3'-phosphorothioate linkage
  modified_base 2: mod_base = OTHER; note = 2'-O-methylcytidine with a 3'-phosphorothioate linkage
  modified_base 3: mod_base = OTHER; note = 2'-O-methyladenosine
  modified_base 4: mod_base = OTHER; note = 2'-O-methyladenosine
  modified_base 5: mod_base = gm
  modified_base 6: mod_base = um
  modified_base 7: mod_base = gm
  modified_base 8: mod_base = um
  modified_base 9: mod_base = OTHER; note = 2'-fluoroguanosine
  modified_base 10: mod_base = OTHER; note = 2'-fluoroguanosine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = um
  modified_base 16: mod_base = um
  modified_base 17: mod_base = OTHER; note = 2'-O-methyladenosine
  modified_base 18: mod_base = gm
  modified_base 19: mod_base = OTHER; note = 2'-O-methylguanosine with a 3'-phosphorothioate linkage
  modified_base 20: mod_base = OTHER; note = 2'-O-methylcytidine with a 3'-phosphorothioate linkage
  modified_base 21: mod_base = OTHER; note = 2'-O-methyladenosine
  source 1..21: mol_type = other RNA
ccaagtgtggctcattaggca

SEQ ID NO: 155 - RNA, 21 nt, synthetic construct
  misc_feature 1..21: note = Description of Artificial Sequence: Synthetic oligonucleotide
  modified_base 1: mod_base = OTHER; note = 2'-O-methyluridine with a 3'-phosphorothioate linkage
  modified_base 2: mod_base = OTHER; note = 2'-O-methylguanosine with a 3'-phosphorothioate linkage
  modified_base 3: mod_base = cm
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = OTHER; note = 2'-O-methyladenosine
  modified_base 14: mod_base = cm
  modified_base 15: mod_base = OTHER; note = 2'-O-methyladenosine
  modified_base 16: mod_base = OTHER; note = 2'-O-methyladenosine
  modified_base 17: mod_base = OTHER; note = 2'-O-methyladenosine
  modified_base 18: mod_base = cm
  modified_base 19: mod_base = OTHER; note = 2'-O-methylcytidine with a 3'-phosphorothioate linkage
  modified_base 20: mod_base = OTHER; note = 2'-O-methyladenosine with a 3'-phosphorothioate linkage
  modified_base 21: mod_base = OTHER; note = 2'-O-methyladenosine
  source 1..21: mol_type = other RNA
tgcaaatagtctacaaaccaa

SEQ ID NO: 156 - DNA, 6644 bp, Homo sapiens
  source 1..6644: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 157 - protein, 776 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 158 - DNA, 5552 bp, Homo sapiens
  source 1..5552: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 159 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 160 - DNA, 5372 bp, Homo sapiens
  source 1..5372: mol_type = genomic DNA
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 161 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 162 - protein, 5 aa, Homo sapiens
FEDLY

SEQ ID NO: 163 - protein, 21 aa, Homo sapiens
LFGNMEEGDCPSDWKTDSTCR

SEQ ID NO: 164 - protein, 5 aa, Homo sapiens
AGKIT

SEQ ID NO: 165 - protein, 7 aa, Homo sapiens
VEKLTLD

SEQ ID NO: 166 - protein, 447 aa, synthetic construct
EVQLVESGGGLVKPGGSLRLSCVASGFTFSSYSMNWVRQAPGKGLEWVSSISRSSSYIYYADSVKGRFTISRDNAKNSLYLQMNSLRAEDTAVYYCARIHGYSNSDAFDKWGQGTLVTVSSASTKGPCVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 167 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLG

(19) United States
(12) Patent Application Publication
(10) Pub. No.: US 2024/0059784 A1
(43) Pub. Date: Feb. 22, 2024

(54) TRANSFERRIN RECEPTOR BINDING PROTEINS AND CONJUGATES

(71) Applicant: Eli Lilly and Company, Indianaplis, IN (US)

(72) Inventors: Riazul Alam, Fishers, IN (US); Alberto Alvarado, San Diego, CA (US); Forest Hoyt Andrews, Carmel, IN (US); Nicholas Alan Babb, Noblesville, IN (US); Deepa Balasubramaniam, San Diego, CA (US); Guillermo S. Cortez, Indianapolis, IN (US); Johnny Eugene Croy, Fishers, IN (US); David Albert Driver, Solana Beach, CA (US); Ross Edward Fellows, Westfield, IN (US); Daniel Girard, San Diego, CA (US); Lacie Chauvigne-Hines, Plainfield, IN (US); Feng Liu, Foster City, CA (US); Hiroaki Tani, Indianapolis, IN (US); Isabel C. Gonzalez Valcarcel, Indianapolis, IN (US); Scott Alan Lawrence, Carmel, IN (US); Nalini Hosahalli Kulkarni, Westfield, IN (US); Jeremy S York, Noblesville, IN (US)

(21) Appl. No.: 18/366,061

(22) Filed: Aug. 7, 2023

Related U.S. Application Data
(60) Provisional application No. 63/496,465, filed on Apr. 17, 2023.
(60) Provisional application No. 63/396,065, filed on Aug. 8, 2022.

(51) Int. Cl. C07K 16/28; C12N 15/113; A61P 25/28

(52) U.S. Cl. CPC C07K 16/2881; C12N 15/113; A61P 25/28; C07K 2317/565; C07K 2317/94; C07K 2317/526; C07K 2317/569; C12N 2310/11; C12N 2310/32; C12N 2310/315

(57) ABSTRACT

Provided herein are proteins comprising one monovalent human TfR binding domain (“human TfR binding proteins”), proteins comprising one monovalent mouse TfR binding domain (“mouse TfR binding proteins”), conjugates comprising such human or mouse TfR binding proteins, e.g., human TfR binding proteins-dsRNA conjugates, pharmaceutical compositions comprising human TfR binding proteins or conjugates, and methods of treating CNS diseases (e.g., neurodegenerative disease such as neurodegenerative synucleinopathy or tauopathy) using human TfR binding proteins or conjugates.

### SEQUENCE LISTING

The present application is being filed along with a Sequence Listing in ST.26 XML format. The Sequence Listing is provided as a file titled “30369 US” created 21 Jul. 2023 and is 667 kilobytes in size. The Sequence Listing information in the ST.26 XML format is incorporated herein by reference in its entirety.

### BACKGROUND

The blood brain barrier (BBB) is a selective semipermeable border of capillary endothelial cells that prevents solutes, including pathogens, from passing into the central nervous system (CNS). The BBB allows the passage of some small molecules by passive diffusion and the cells of BBB actively transport metabolic products crucial to neural function such as glucose and amino acids across the barrier using specific transport proteins. The BBB has neuroprotective function by tightly controlling access to the brain; but it also impedes access of therapeutic agents to CNS.

BBB shuttles for improving passage of the therapeutic agents across the blood brain barrier and into the CNS have been described. For example, WO2003/009815 describes the use of antibodies directed to transferrin receptor (“TfR”) for modulating blood brain barrier transport. However, attempts at using anti-TfR antibodies to shuttle therapeutic agents across the BBB have proven challenging. To date, there are no approved TfR shuttles or conjugates for the treatment of CNS diseases.

Therefore, there remains a need for TfR binding proteins and conjugates that can deliver therapeutic agents across the BBB into the CNS for the treatment of various CNS diseases.

### SUMMARY OF INVENTION

Provided herein are proteins comprising one monovalent human TfR binding domain (“human TfR binding proteins”), proteins comprising one monovalent mouse TfR binding domain (“mouse TfR binding proteins”), conjugates comprising such human or mouse TfR binding proteins, e.g., human TfR binding proteins-dsRNA conjugates, pharmaceutical compositions comprising human TfR binding proteins or conjugates, and methods of treating CNS diseases (e.g., neurodegenerative disease such as neurodegenerative synucleinopathy or tauopathy) using human TfR binding proteins or conjugates.

In one aspect, provided herein are proteins comprising one and only one monovalent human TfR binding domain (“human TfR binding proteins”). In some embodiments, the monovalent human TfR binding domain comprises a heavy chain variable region (VH) and a light chain variable region (VL), and the VH comprises heavy chain complementarity determining regions HCDR1, HCDR2, and HCDR3, and the VL comprises light chain complementarity determining regions LCDR1, LCDR2, and LCDR3. In some embodiments, the monovalent human TfR binding domain comprises a VH comprising HCDR1, HCDR2, and HCDR3 selected from Table 1, and/or a VL comprising LCDR1, LCDR2, and LCDR3 selected from Table 2. In some embodiments, the monovalent human TfR binding domain comprises a VH and/or a VL selected from Table 3.

In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 21, HCDR3 comprises SEQ ID NO: 22, LCDR1 comprises SEQ ID NO: 23, LCDR2 comprises SEQ ID NO: 5, and LCDR3 comprises SEQ ID NO: 24; or
(b) HCDR1 comprises SEQ ID NO: 13, HCDR2 comprises SEQ ID NO: 25, HCDR3 comprises SEQ ID NO: 26, LCDR1 comprises SEQ ID NO: 16, LCDR2 comprises SEQ ID NO: 17, and LCDR3 comprises SEQ ID NO: 18.

In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6;
(b) HCDR1 comprises SEQ ID NO: 1, HCDR2 comprises SEQ ID NO: 2, HCDR3 comprises SEQ ID NO: 7, LCDR1 comprises SEQ ID NO: 4, LCDR2 comprises SEQ ID NO: 5, and LCDR3 comprises SEQ ID NO: 6;
(c) HCDR1 comprises SEQ ID NO: 1, HCDR2 comprises SEQ ID NO: 2, HCDR3 comprises SEQ ID NO: 8, LCDR1 comprises SEQ ID NO: 9, LCDR2 comprises SEQ ID NO: 5, and LCDR3 comprises SEQ ID NO: 6;
(d) HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12;
(e) HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 17, and LCDR3 comprises SEQ ID NO: 18;
(f) HCDR1 comprises SEQ ID NO: 13, HCDR2 comprises SEQ ID NO: 19, HCDR3 comprises SEQ ID NO: 15, LCDR1 comprises SEQ ID NO: 16, LCDR2 comprises SEQ ID NO: 17, and LCDR3 comprises SEQ ID NO: 18; or
(g) HCDR1 comprises SEQ ID NO: 13, HCDR2 comprises SEQ ID NO: 19, HCDR3 comprises SEQ ID NO: 20, LCDR1 comprises SEQ ID NO: 16, LCDR2 comprises SEQ ID NO: 17, and LCDR3 comprises SEQ ID NO: 18.

In some embodiments, provided herein are proteins comprising one monovalent human TfR binding domain, wherein the human TfR binding domain comprises a VH and a VL, and wherein the VH and VL comprise the following sequences:
(a) VH comprises a sequence having at least 95% sequence identity to SEQ ID NO: 27 and VL comprises a sequence having at least 95% sequence identity to SEQ ID NO: 28;
(b) VH comprises a sequence having at least 95% sequence identity to SEQ ID NO: 29 and VL comprises a sequence having at least 95% sequence identity to SEQ ID NO: 28;
(c) VH comprises a sequence having at least 95% sequence identity to SEQ ID NO: 30 and VL comprises a sequence having at least 95% sequence identity to SEQ ID NO: 31;
(d) VH comprises a sequence having at least 95% sequence identity to SEQ ID NO: 32 and VL comprises a sequence having at least 95% sequence identity to SEQ ID NO: 33;
(e) VH comprises a sequence having at least 95% sequence identity to SEQ ID NO: 34 and VL comprises a sequence having at least 95% sequence identity to SEQ ID NO: 35;
(f) VH comprises a sequence having at least 95% sequence identity to SEQ ID NO: 36 and VL comprises a sequence having at least 95% sequence identity to SEQ ID NO: 37; or
(g) VH comprises a sequence having at least 95% sequence identity to SEQ ID NO: 38 and VL comprises a sequence having at least 95% sequence identity to SEQ ID NO: 37.

In some embodiments, provided herein are proteins comprising one monovalent human TfR binding domain, wherein the human TfR binding domain comprises a VH and a VL, and wherein the VH and VL comprise the following sequences:
(a) VH comprises SEQ ID NO: 27 and VL comprises SEQ ID NO: 28;
(b) VH comprises SEQ ID NO: 29 and VL comprises SEQ ID NO: 28;
(c) VH comprises SEQ ID NO: 30 and VL comprises SEQ ID NO: 31;
(d) VH comprises SEQ ID NO: 32 and VL comprises SEQ ID NO: 33;
(e) VH comprises SEQ ID NO: 34 and VL comprises SEQ ID NO: 35;
(f) VH comprises SEQ ID NO: 36 and VL comprises SEQ ID NO: 37; or
(g) VH comprises SEQ ID NO: 38 and VL comprises SEQ ID NO: 37.

In some embodiments, the monovalent human TfR binding domain is an antibody fragment, e.g., Fab, scFv, Fv, or scFab (single chain Fab). In some embodiments, the monovalent human TfR binding domain is Fab. In some embodiments, the human TfR binding domain further comprises a heavy chain constant region and/or a light chain constant region.

In some embodiments, the human TfR binding proteins describe herein further comprise a half-life extender, e.g., an immunoglobulin Fc region or a VHH that binds human serum albumin (HSA).

In some embodiments, the human TfR binding proteins described herein comprise one or more engineered cysteine residues for conjugation. In some embodiments, the human TfR binding proteins described herein comprise one or more native cysteine residues for conjugation.

In some embodiments, the human TfR binding protein described herein is any one of the human TfR binding proteins in Table 6a and 6b. In some embodiments, the human TfR binding protein described herein has one heavy chain (HC) and one light chain (LC), e.g., TBP1, TBP2, TBP3, TBP4, TBP5, TBP6, TBP7, TBP8, or TBP9. In some embodiments, the human TfR binding protein has two heavy chains (HC1 and HC2) and two light chains (LC1 and LC2). In some embodiments, the human TfR binding protein described herein has a heterodimeric antibody format, e.g., TBP10, TBP11, TBP12, or TBP13.

In some embodiments, provided herein are proteins comprising one monovalent human transferrin receptor (TfR) binding domain, wherein the human TfR binding domain binds an epitope comprising one or more residues in (a) residues 346-364 FGNMEGDCPSDWKTDSTCR (SEQ ID NO: 119), (b) residues 243-247 FEDLY (SEQ ID NO: 162) and residues 345-364 LFGNMEEGDCPSDWKTDSTCR) (SEQ ID NO: 163), or (c) residues 243-247 FEDLY (SEQ ID NO: 162), residues 259-263 AGKIT (SEQ ID NO: 164), and residues 532-538 (VEKLTLD) (SEQ ID NO: 165), of human TfR.

In another aspect, provided herein are proteins comprising one monovalent mouse TfR binding domain (“mouse TfR binding proteins”). These mouse TfR binding proteins can serve as surrogate molecules to the human TfR binding proteins in mouse models. In some embodiments, provided herein are proteins comprising one monovalent mouse TfR binding domain, wherein the mouse TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 71, HCDR2 comprises SEQ ID NO: 72, HCDR3 comprises SEQ ID NO: 73, LCDR1 comprises SEQ ID NO: 74, LCDR2 comprises SEQ ID NO: 75, and LCDR3 comprises SEQ ID NO: 76. In some embodiments, provided herein are proteins comprising one monovalent mouse TfR binding domain, wherein the mouse TfR binding domain comprises a VH comprising SEQ ID NO: 77 and a VL comprising SEQ ID NO: 78.

Also provided herein are antibodies comprising a VH comprising HCDR1, HCDR2, and HCDR3 selected from Table 1, and/or a VL comprising LCDR1, LCDR2, and LCDR3 selected from Table 2. In some embodiments, such antibodies comprise a VH and/or a VL selected from Table 3.

In another aspect, provided herein are conjugates comprising human or mouse TfR binding proteins described herein and a therapeutic agent. In some embodiments, the therapeutic agent is selected from a double stranded RNA (e.g., siRNA, saRNA), oligonucleotide (e.g., antisense oligonucleotide), peptide, small molecule, nanoparticle, lipid nanoparticle, exosome, antibody or antigen binding fragment thereof, or a combination thereof. In some embodiments, the therapeutic agent is a double stranded RNA (dsRNA). In some embodiments, the dsRNA comprises a sense strand and an antisense stand, wherein the antisense strand is complementary to a target mRNA selected from SNCA, MAPT, APP, ATXN2, ATXN3, SARM1, APOE, BACE1, FMR1, LRRK2, HTT, SOD1, SCN10A, SCN9A or CACNA1B mRNA. In some embodiments, the therapeutic agent to protein ratio is about 1:1 to 3:1. In some embodiments, the therapeutic agent to protein ratio is about 1:1. In some embodiments, the therapeutic agent to protein ratio is about 2:1. In some embodiments, the therapeutic agent to protein ratio is about 3:1.

In some embodiments, the therapeutic agent is linked to the human or mouse TfR binding protein through a linker. In some embodiments, the linker is a Mal-Tet-TCO linker, SMCC linker, or GDM linker (structures of these linkers shown in Table 8).

In some embodiments, provided herein are conjugates of Formula (I): R-L-P, wherein R is a double stranded RNA (dsRNA) comprising a sense stand and an antisense strand; wherein P is a protein comprising one monovalent human or mouse TfR binding domain; and wherein L is a linker, or optionally absent. In some embodiments, P is a human or mouse TfR binding protein described herein. In some embodiments, the R to P ratio is about 1:1 to 3:1. In some embodiments, the R to P ratio is about 1:1. In some embodiments, the R to P ratio is about 2:1. In some embodiments, the R to P ratio is about 3:1.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human or mouse TfR binding domain; wherein L is a linker, or optionally absent, and wherein n is 1 to 3. In some embodiments, n is 1. In some embodiments, n is 2. In some embodiments, n is 3.

In some embodiments, provided herein are conjugates of Formula (I): R-L-P, wherein R is a double stranded RNA (dsRNA) comprising a sense stand and an antisense strand; wherein P is a protein comprising one monovalent human TfR binding domain; and wherein L is a linker, or optionally absent, herein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 21, HCDR3 comprises SEQ ID NO: 22, LCDR1 comprises SEQ ID NO: 23, LCDR2 comprises SEQ ID NO: 5, and LCDR3 comprises SEQ ID NO: 24; or
(b) HCDR1 comprises SEQ ID NO: 13, HCDR2 comprises SEQ ID NO: 25, HCDR3 comprises SEQ ID NO: 26, LCDR1 comprises SEQ ID NO: 16, LCDR2 comprises SEQ ID NO: 17, and LCDR3 comprises SEQ ID NO: 18.

In some embodiments, provided herein are conjugates of Formula (I): R-L-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6;
(b) HCDR1 comprises SEQ ID NO: 1, HCDR2 comprises SEQ ID NO: 2, HCDR3 comprises SEQ ID NO: 7, LCDR1 comprises SEQ ID NO: 4, LCDR2 comprises SEQ ID NO: 5, and LCDR3 comprises SEQ ID NO: 6;
(c) HCDR1 comprises SEQ ID NO: 1, HCDR2 comprises SEQ ID NO: 2, HCDR3 comprises SEQ ID NO: 8, LCDR1 comprises SEQ ID NO: 9, LCDR2 comprises SEQ ID NO: 5, and LCDR3 comprises SEQ ID NO: 6;
(d) HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12;
(e) HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 17, and LCDR3 comprises SEQ ID NO: 18;
(f) HCDR1 comprises SEQ ID NO: 13, HCDR2 comprises SEQ ID NO: 19, HCDR3 comprises SEQ ID NO: 15, LCDR1 comprises SEQ ID NO: 16, LCDR2 comprises SEQ ID NO: 17, and LCDR3 comprises SEQ ID NO: 18; or
(g) HCDR1 comprises SEQ ID NO: 13, HCDR2 comprises SEQ ID NO: 19, HCDR3 comprises SEQ ID NO: 20, LCDR1 comprises SEQ ID NO: 16, LCDR2 comprises SEQ ID NO: 17, and LCDR3 comprises SEQ ID NO: 18.

In some embodiments, provided herein are conjugates of Formula (I): R-L-P, wherein R is a double stranded RNA (dsRNA) comprising a sense stand and an antisense strand; wherein P is a protein comprising one monovalent human TfR binding domain; and wherein L is a linker, or optionally absent, herein the human TfR binding domain comprises a heavy chain variable region (VH) and a light chain variable region (VL), wherein the VH and VL comprise the following sequences:
(a) VH comprises a sequence having at least 95% sequence identity to SEQ ID NO: 27 and VL comprises a sequence having at least 95% sequence identity to SEQ ID NO: 28;
(b) VH comprises a sequence having at least 95% sequence identity to SEQ ID NO: 29 and VL comprises a sequence having at least 95% sequence identity to SEQ ID NO: 28;
(c) VH comprises a sequence having at least 95% sequence identity to SEQ ID NO: 30 and VL comprises a sequence having at least 95% sequence identity to SEQ ID NO: 31;
(d) VH comprises a sequence having at least 95% sequence identity to SEQ ID NO: 32 and VL comprises a sequence having at least 95% sequence identity to SEQ ID NO: 33;
(e) VH comprises a sequence having at least 95% sequence identity to SEQ ID NO: 34 and VL comprises a sequence having at least 95% sequence identity to SEQ ID NO: 35;
(f) VH comprises a sequence having at least 95% sequence identity to SEQ ID NO: 36 and VL comprises a sequence having at least 95% sequence identity to SEQ ID NO: 37; or
(g) VH comprises a sequence having at least 95% sequence identity to SEQ ID NO: 38 and VL comprises a sequence having at least 95% sequence identity to SEQ ID NO: 37.

In some embodiments, provided herein are conjugates of Formula (I): R-L-P, wherein R is a double stranded RNA (dsRNA) comprising a sense stand and an antisense strand; wherein P is a protein comprising one monovalent human TfR binding domain; and wherein L is a linker, or optionally absent, herein the human TfR binding domain comprises a heavy chain variable region (VH) and a light chain variable region (VL), wherein the VH and VL comprise the following sequences:
(a) VH comprises SEQ ID NO: 27 and VL comprises SEQ ID NO: 28;
(b) VH comprises SEQ ID NO: 29 and VL comprises SEQ ID NO: 28;
(c) VH comprises SEQ ID NO: 30 and VL comprises SEQ ID NO: 31;
(d) VH comprises SEQ ID NO: 32 and VL comprises SEQ ID NO: 33;
(e) VH comprises SEQ ID NO: 34 and VL comprises SEQ ID NO: 35;
(f) VH comprises SEQ ID NO: 36 and VL comprises SEQ ID NO: 37; or
(g) VH comprises SEQ ID NO: 38 and VL comprises SEQ ID NO: 37.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, herein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 21, HCDR3 comprises SEQ ID NO: 22, LCDR1 comprises SEQ ID NO: 23, LCDR2 comprises SEQ ID NO: 5, and LCDR3 comprises SEQ ID NO: 24; or
(b) HCDR1 comprises SEQ ID NO: 13, HCDR2 comprises SEQ ID NO: 25, HCDR3 comprises SEQ ID NO: 26, LCDR1 comprises SEQ ID NO: 16, LCDR2 comprises SEQ ID NO: 17, and LCDR3 comprises SEQ ID NO: 18, and wherein n is 1 to 3.

In some embodiments, n is 1. In some embodiments, n is 2. In some embodiments, n is 3.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6;
(b) HCDR1 comprises SEQ ID NO: 1, HCDR2 comprises SEQ ID NO: 2, HCDR3 comprises SEQ ID NO: 7, LCDR1 comprises SEQ ID NO: 4, LCDR2 comprises SEQ ID NO: 5, and LCDR3 comprises SEQ ID NO: 6;
(c) HCDR1 comprises SEQ ID NO: 1, HCDR2 comprises SEQ ID NO: 2, HCDR3 comprises SEQ ID NO: 8, LCDR1 comprises SEQ ID NO: 9, LCDR2 comprises SEQ ID NO: 5, and LCDR3 comprises SEQ ID NO: 6;
(d) HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12;
(e) HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 17, and LCDR3 comprises SEQ ID NO: 18;
(f) HCDR1 comprises SEQ ID NO: 13, HCDR2 comprises SEQ ID NO: 19, HCDR3 comprises SEQ ID NO: 15, LCDR1 comprises SEQ ID NO: 16, LCDR2 comprises SEQ ID NO: 17, and LCDR3 comprises SEQ ID NO: 18; or
(g) HCDR1 comprises SEQ ID NO: 13, HCDR2 comprises SEQ ID NO: 19, HCDR3 comprises SEQ ID NO: 20, LCDR1 comprises SEQ ID NO: 16, LCDR2 comprises SEQ ID NO: 17, and LCDR3 comprises SEQ ID NO: 18, and wherein n is 1 to 3.

In some embodiments, n is 1. In some embodiments, n is 2. In some embodiments, n is 3.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH and VL comprise the following sequences:
(a) VH comprises a sequence having at least 95% sequence identity to SEQ ID NO: 27 and VL comprises a sequence having at least 95% sequence identity to SEQ ID NO: 28;
(b) VH comprises a sequence having at least 95% sequence identity to SEQ ID NO: 29 and VL comprises a sequence having at least 95% sequence identity to SEQ ID NO: 28;
(c) VH comprises a sequence having at least 95% sequence identity to SEQ ID NO: 30 and VL comprises a sequence having at least 95% sequence identity to SEQ ID NO: 31;
(d) VH comprises a sequence having at least 95% sequence identity to SEQ ID NO: 32 and VL comprises a sequence having at least 95% sequence identity to SEQ ID NO: 33;
(e) VH comprises a sequence having at least 95% sequence identity to SEQ ID NO: 34 and VL comprises a sequence having at least 95% sequence identity to SEQ ID NO: 35;
(f) VH comprises a sequence having at least 95% sequence identity to SEQ ID NO: 36 and VL comprises a sequence having at least 95% sequence identity to SEQ ID NO: 37; or
(g) VH comprises a sequence having at least 95% sequence identity to SEQ ID NO: 38 and VL comprises a sequence having at least 95% sequence identity to SEQ ID NO: 37, and wherein n is 1 to 3.

In some embodiments, n is 1. In some embodiments, n is 2. In some embodiments, n is 3.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH and VL comprise the following sequences:
(a) VH comprises SEQ ID NO: 27 and VL comprises SEQ ID NO: 28;
(b) VH comprises SEQ ID NO: 29 and VL comprises SEQ ID NO: 28;
(c) VH comprises SEQ ID NO: 30 and VL comprises SEQ ID NO: 31;
(d) VH comprises SEQ ID NO: 32 and VL comprises SEQ ID NO: 33;
(e) VH comprises SEQ ID NO: 34 and VL comprises SEQ ID NO: 35;
(f) VH comprises SEQ ID NO: 36 and VL comprises SEQ ID NO: 37; or
(g) VH comprises SEQ ID NO: 38 and VL comprises SEQ ID NO: 37, and wherein n is 1 to 3.

In some embodiments, n is 1. In some embodiments, n is 2. In some embodiments, n is 3.

In some embodiments, the linker (L) is a Mal-Tet-TCO linker, SMCC linker, or GDM linker (see Table 8).

In some embodiments, the dsRNA comprises an antisense strand complementary to a target mRNA selected from SNCA, MAPT, APP, ATXN2, ATXN3, SARM1, APOE, BACE1, FMR1, LRRK2, HTT, SOD1, SCN10A, SCN9A or CACNA1B mRNA. In some embodiments, the dsRNA comprises an antisense strand complementary to SNCA mRNA. In some embodiments, the dsRNA comprises an antisense strand complementary to MAPT mRNA.

Exemplary unmodified sense strand and antisense strand sequences of dsRNA targeting human SNCA mRNA are provided in Table 9a. In some embodiments, the sense strand and the antisense strand of the dsRNA comprise a pair of nucleic acid sequences selected from the group consisting of:
(a) the sense strand comprises SEQ ID NO: 81, and the antisense strand comprises SEQ ID NO: 82;
(b) the sense strand comprises SEQ ID NO: 83, and the antisense strand comprises SEQ ID NO: 84;
(c) the sense strand comprises SEQ ID NO: 85, and the antisense strand comprises SEQ ID NO: 86;
(d) the sense strand comprises SEQ ID NO: 87, and the antisense strand comprises SEQ ID NO: 88;
(e) the sense strand comprises SEQ ID NO: 89, and the antisense strand comprises SEQ ID NO: 90; and
(f) the sense strand comprises SEQ ID NO: 91, and the antisense strand comprises SEQ ID NO: 92;
(g) the sense strand comprises SEQ ID NO: 116, and the antisense strand comprises SEQ ID NO: 82,
wherein optionally one or more nucleotides of the sense strand and the antisense strand are independently modified nucleotides, and wherein optionally one or more internucleotide linkages of the sense strand and the antisense strand are modified internucleotide linkages. In some embodiments, the sense strand comprises SEQ ID NO: 81, and the antisense strand comprises SEQ ID NO: 82.

Exemplary unmodified sense strand and antisense strand sequences of dsRNA targeting human MAPT mRNA are provided in Table 9b. In some embodiments, the sense strand and the antisense strand of the dsRNA comprise a pair of nucleic acid sequences selected from the group consisting of:
(a) the sense strand comprises SEQ ID NO: 120, and the antisense strand comprises SEQ ID NO: 121;
(b) the sense strand comprises SEQ ID NO: 122, and the antisense strand comprises SEQ ID NO: 123; and
(c) the sense strand comprises SEQ ID NO: 124, and the antisense strand comprises SEQ ID NO: 125,
wherein optionally one or more nucleotides of the sense strand and the antisense strand are independently modified nucleotides, and wherein optionally one or more internucleotide linkages of the sense strand and the antisense strand are modified internucleotide linkages.

The dsRNA can include modifications. The modifications can be made to one or more nucleotides of the sense and/or antisense strand or to the internucleotide linkages. In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl modified nucleotide. In some embodiments, each nucleotide of the sense strand and the antisense strand is independently a modified nucleotide, e.g., a 2′-fluoro modified nucleotide, 2′-O-methyl modified nucleotide, or 2′-O-alkyl modified nucleotide.

In some embodiments, the sense strand has four 2′-fluoro modified nucleotides, e.g., at positions 7, 9, 10, 11 from the 5′ end of the sense strand. In some embodiments, the other nucleotides of the sense strand are 2′-O-methyl modified nucleotides. In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, the other nucleotides of the sense strand are 2′-O-methyl modified nucleotides. In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human SNCA mRNA are provided in Table 11a. Exemplary modified sense strand and antisense strand sequences of dsRNA targeting human MAPT mRNA are provided in Table 11b.

In another aspect, provided herein are methods of treating a CNS disease, e.g., a neurodegenerative disease, in a patient in need thereof, and such the method comprises administering to the patient an effective amount of the human TfR binding protein or conjugate or a pharmaceutical composition described herein.

In a further aspect, provided herein are methods of treating a neurodegenerative synucleinopathy in a patient in need thereof, and such the method comprises administering to the patient an effective amount of the human TfR binding proteins or conjugate or a pharmaceutical composition described herein (e.g., a TBP-SNCA siRNA conjugate described herein or a pharmaceutical composition comprising such a TBP-SNCA siRNA conjugate). In some embodiments, the neurodegenerative synucleinopathy is selected from Parkinson's disease, Alzheimer's disease, multiple system atrophy, or Lewy body dementia. The human TfR binding protein or conjugate or a pharmaceutical composition can be administered to the patient intravenously or subcutaneously.

In a further aspect, provided herein are methods of treating a tauopathy in a patient in need thereof, and such the method comprises administering to the patient an effective amount of the human TfR binding proteins or conjugate or a pharmaceutical composition described herein (e.g., a TBP-MAPT siRNA conjugate described herein or a pharmaceutical composition comprising such a TBP-MAPT siRNA conjugate). In some embodiments, the tauopathy is selected from Alzheimer's disease, frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's discase, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT). The human TfR binding protein or conjugate or a pharmaceutical composition can be administered to the patient intravenously or subcutaneously.

In another aspect, provided herein are human TfR binding proteins or conjugates described herein or pharmaceutical compositions comprising such human TfR binding proteins or conjugates for use in a therapy. Also provided herein are human TfR binding proteins or conjugates described herein or pharmaceutical compositions comprising such human TfR binding proteins or conjugates (e.g., a TBP-SNCA siRNA conjugate described herein or a pharmaceutical composition comprising such a TBP-SNCA siRNA conjugate) for use in the treatment of a neurodegenerative synucleinopathy, e.g., Parkinson's disease, Alzheimer's disease, multiple system atrophy, or Lewy body dementia. Also provided herein are human TfR binding proteins or conjugates described herein or pharmaceutical compositions comprising such human TfR binding proteins or conjugates (e.g., a TBP-MAPT siRNA conjugate described herein or a pharmaceutical composition comprising such a TBP-MAPT siRNA conjugate) for use in the treatment of a tauopathy, e.g., Alzheimer's disease, frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's discase, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT).

In another aspect, provided herein are uses of human TfR binding proteins or conjugates described herein in the manufacture of a medicament for treating a CNS disease, e.g., a neurodegenerative disease. In some embodiments, the neurodegenerative disease is a neurodegenerative synucleinopathy, e.g., Parkinson's disease, Alzheimer's disease, multiple system atrophy, or Lewy body dementia. In some embodiments, the neurodegenerative disease is a tauopathy, e.g., Alzheimer's disease, frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's discase, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT).

### BRIEF DESCRIPTION OF THE DRAWINGS

FIG. 1A shows an exemplary analytical anion exchange (aAEX) chromatogram of DAR profile for TBP11-dsRNA conjugate before purification. FIG. 1B shows an exemplary aAEX chromatogram of DAR profile for TBP14-dsRNA conjugate after purification.

FIG. 1C shows an exemplary aAEX chromatogram of DAR profile for TBP15-dsRNA conjugate before purification. FIG. 1D shows an exemplary aAEX chromatogram of DAR profile for TBP15-dsRNA conjugate after purification. FIG. 1E shows exemplary diagrams of TBP-dsRNA conjugates of DAR2 (top) or DAR1 (bottom).

FIG. 2 shows in vitro binding, internalization and degradation of the indicated molecules in mouse cortical neurons.

FIG. 3 shows in vitro potency of the indicated molecules for knocking down mouse SNCA in primary mouse cortical neurons.

FIG. 4 shows in vitro binding, internalization and degradation assessment of the indicated molecules in SHSY5Y cells.

FIG. 5 shows in vitro potency of the indicated molecules for knocking down human SNCA in SH-SY5Y cells.

FIGS. 6A, 6B and 6C show mouse proof of concept data demonstrating pharmacodynamic efficacy of mTBP2-SNCA siRNA conjugate with multiple intravenous (IV) dosing at a single time point (28 days), showing SNCA mRNA and protein reduction in mouse brain (FIG. 6A) and SNCA mRNA reduction in spinal cord (FIG. 6B) and lumbar dorsal root ganglia (FIG. 6C).

FIGS. 7A and 7B show mouse Proof of Concept pharmacodynamic efficacy time course data of mTBP2-SNCA siRNA conjugate following a single IV dosing with mice sacrificed at multiple time points following dose (7 days, 28 days, 70 days and 120 days), showing Pharmacodynamic time course of SNCA mRNA and protein reduction in mouse brain (FIG. 7A) and SNCA mRNA and protein reduction in spinal cord (FIG. 7B). The error bars in FIGS. 7A and 7B are Standard Deviations and statistical analysis was performed with a one-way Anova with Dunnett's multiple comparison test against PBS control group. Annotations indicate P values >0.0001=****; >0.001=***; >0.01=**; >0.05=*.

FIG. 8A shows SNCA mRNA reduction in Cynomolgus monkey tissues 29 days after a two successive single IV peripheral doses (given two hours apart) of TBP10-SNCA siRNA (dsRNA No. 8 in Table 11a) conjugate at 4.4 mg/kg siRNA. FIG. 8B shows SNCA mRNA reduction in Cynomolgus monkey tissues 29 days after a two successive single IV peripheral doses (given two hours apart) of TBP11-SNCA siRNA (dsRNA No. 8 in Table 11a) conjugate at 1.3 mg/kg siRNA. The error bars in FIGS. 8A and 8B are Standard Error of the Mean and statistical analysis was performed with a one-way Anova with Dunnett's multiple comparison test against PBS control group. Annotations indicate P values >0.0001 to 0.05=*.

FIG. 8C shows the mouse brain efficacy comparison of mouse TfR binding protein conjugates at NUP equivalent siRNA doses adjusted to body weight. The error bars in FIG. 8C are Standard Deviations and statistical analysis was performed with a one-way Anova with Dunnett's multiple comparison test against PBS control group. Annotations indicate P values >0.0001=****; >0.001=***; >0.01=**; >0.05=*.

FIG. 9A shows SNCA mRNA reduction in Cynomolgus monkey tissues after three monthly peripheral intravenous (IV) administration of TBP14-SNCA siRNA (dsRNA No. 10 in Table 11a) conjugate at 10 mg/kg siRNA. FIG. 9B shows reduction of α-synuclein protein in Cynomolgus monkey tissues after three monthly peripheral IV administration of TBP14-SNCA siRNA (dsRNA No. 10 in Table 11a) conjugate at 10 mg/kg siRNA. FIG. 9C shows SNCA mRNA reduction in Cynomolgus monkey tissues 85 days after a single peripheral IV administration of TBP14-SNCA siRNA (dsRNA No. 10 in Table 11a) conjugate at 10 mg/kg siRNA. FIG. 9D shows reduction of α-synuclein protein in Cynomolgus monkey tissues 85 days after a single peripheral IV administration of TBP14-SNCA siRNA (dsRNA No. 10 in Table 11a) conjugate at 10 mg/kg siRNA. FIG. 9E shows SNCA mRNA reduction in the gastrocnemius muscle after a single or three successive monthly peripheral IV administrations of TBP14-SNCA siRNA (dsRNA No. 10 in Table 11a) conjugate at 10 mg/kg siRNA.

FIG. 10A shows MAPT mRNA reduction in Cynomolgus monkey tissues after three monthly peripheral IV administrations of TBP14-MAPT siRNA (dsRNA No. 38 in Table 11 b) conjugate at 10 mg/kg siRNA. FIG. 10B shows reduction of Tau protein in Cynomolgus monkey tissues after three monthly peripheral IV administrations of TBP14-MAPT siRNA (dsRNA No. 38 in Table 11b) conjugate at 10 mg/kg siRNA.

FIG. 11A shows MAPT mRNA reduction in Cynomolgus monkey tissues after three monthly peripheral IV administrations of TBP14-MAPT siRNA (dsRNA No. 39 in Table 11b) conjugate at 10 mg/kg. FIG. 11B shows reduction of Tau protein in Cynomolgus monkey tissues after three monthly peripheral IV administrations of TBP14-MAPT siRNA (dsRNA No. 39 in Table 11b) conjugate at 10 mg/kg siRNA.

FIG. 12A shows MAPT mRNA reduction in Cynomolgus monkey tissues after three monthly peripheral IV administrations of TBP14-MAPT siRNA (dsRNA No. 40 in Table 11b) conjugate at 10 mg/kg siRNA. FIG. 12B shows reduction of Tau protein in Cynomolgus monkey tissues after three monthly peripheral IV administrations of TBP14-MAPT siRNA (dsRNA No. 40 in Table 11b) conjugate at 10 mg/kg siRNA.

FIG. 13A shows SNCA mRNA reductions in Cynomolgus monkey tissues one month after a single peripheral IV administration of TBP16-SNCA siRNA (dsRNA No. 10 in Table 11a) conjugate (DAR1) at 1 mg/kg siRNA. FIGS. 13B and 13C show SNCA mRNA reductions in selected Cynomolgus monkey brain tissues one month after a single peripheral IV administration of TBP15-SNCA siRNA (dsRNA No. 10 in Table 11a) conjugate (DAR1) at 1 mg/kg (13B) and 10 mg/kg (13C) siRNA. FIG. 13D shows plasma PK of conjugate associated siRNA following a single peripheral IV administration of either TBP14-SNCA siRNA (dsRNA No. 10 in Table 11a) conjugate (DAR2) at 10 mg/kg siRNA or TBP15-SNCA siRNA (dsRNA No. 10 in Table 11a) conjugate (DAR1) at either 10 mg/kg or 1 mg/kg siRNA. FIG. 13E shows total siRNA concentrations in selected Cynomolgus monkey brain tissues at day 29 following a single peripheral IV administration of TBP15-SNCA siRNA (dsRNA No. 10 in Table 11a) conjugate (DAR1) at either 1 or 10 mg/kg siRNA.

FIG. 14A shows plasma PK of conjugate associated siRNA in human TfR transgenic mice following a single peripheral IV administration of either TBP14-SNCA siRNA (dsRNA No. 10 in Table 11a) conjugate (DAR2) or TBP14-SNCA siRNA (dsRNA No. 10 in Table 11a) conjugate (DAR1) at 10 mg/kg siRNA. FIG. 14B shows brain tissue concentrations of total antisense siRNA at 24 hours in human TfR transgenic mice following a single peripheral IV administration of either TBP14-SNCA siRNA (dsRNA No. 10 in Table 11a) conjugate (DAR2) or TBP14-SNCA siRNA (dsRNA No. 10 in Table 11a) conjugate (DAR1) across varying doses. FIG. 14C shows brain tissue concentrations of total siRNA in human TfR transgenic mice at 24 hours following a single peripheral IV administration of either TBP14-SNCA siRNA (dsRNA No. 10 in Table 11a) conjugate (DAR2) or TBP15-SNCA siRNA (dsRNA No. 10 in Table 11a) conjugate (DAR1) across varying siRNA doses. FIG. 14D shows the reduction in SNCA mRNA levels in total brain homogenates at day 28 in human TfR transgenic mice following a single peripheral IV administration of either TBP14-SNCA siRNA (dsRNA No. 10 in Table 11a) conjugate (DAR2) or TBP15-SNCA siRNA (dsRNA No. 10 in Table 11a) conjugate (DAR1) across varying siRNA doses. The error bars are Standard Deviations and statistical analysis was performed with a one-way Anova with Dunnett's multiple comparison test against PBS control group. Annotations indicate P values >0.0001=****; >0.001=***; >0.01=**; >0.05=*. FIG. 14E shows reductions in SNCA mRNA levels in total brain homogenates at day 28 in human TfR transgenic mice following single subcutaneous administration of TBP15-SNCA siRNA (dsRNA No. 10 in Table 11a) conjugate (DAR1) across varying siRNA doses. The error bars are Standard Deviations and statistical analysis was performed with a one-way Anova with Dunnett's multiple comparison test against PBS control group. Annotations indicate P values >0.0001=****; >0.001=***; >0.01=**; >0.05=*.

### DETAILED DESCRIPTION

Provided herein are proteins comprising one monovalent human TfR binding domain (“human TfR binding proteins”), proteins comprising one monovalent mouse TfR binding domain (“mouse TfR binding proteins”), conjugates comprising such human or mouse TfR binding proteins, e.g., human TfR binding proteins-dsRNA conjugates, pharmaceutical compositions comprising human TfR binding proteins or conjugates, and methods of treating CNS diseases (e.g., neurodegenerative disease such as neurodegenerative synucleinopathy or tauopathy) using human TfR binding proteins or conjugates.

### Human TfR Binding Proteins

In one aspect, provided herein are proteins comprising one monovalent human TfR binding domain (“human TfR binding proteins”). In some embodiments, the monovalent human TfR binding domain comprises a heavy chain variable region (VH) and a light chain variable region (VL), and the VH comprises heavy chain complementarity determining regions HCDR1, HCDR2, and HCDR3, and the VL comprises light chain complementarity determining regions LCDR1, LCDR2, and LCDR3. In some embodiments, the monovalent human TfR binding domain comprises a VH comprising HCDR1, HCDR2, and HCDR3 selected from Table 1. In some embodiments, the monovalent human TfR binding domain comprises a VL comprising LCDR1, LCDR2, and LCDR3 selected from Table 2. In some embodiments, the monovalent human TRR binding domain comprises a VH comprising HCDR1, HCDR2, and HCDR3 selected from Table 1, and/or a VL comprising LCDR1, LCDR2, and LCDR3 selected from Table 2. In some embodiments, the monovalent human TfR binding domain comprises a VH and/or a VL selected from Table 3. In some embodiments, the monovalent human TfR binding domain (“TBD”) is TBD1, TBD2, TBD3, TBD4, TBD5, TBD6, TBD6, TBD7, TBD8, or TBD9. In some embodiments, the monovalent human TfR binding domain is TBD1, TBD2, TBD3, TBD4, TBD5, TBD6, TBD6, or TBD7. In some embodiments, the human TfR binding proteins described herein also bind cynomolgus monkey TfR.

TABLE 1
Exemplary sequences of human TfR binding domain heavy chain CDRs

| Human TfR | | | |
|---|---|---|---|
| binding | | | |
| domain | | | |
| (TBD) | HCDR1 (KABAT) | HCDR2 (KABAT) | HCDR3 (KABAT) |
| TBD1 | SYSMN | SISRSSSYIYYADSVKG | EHGYSNSDAFDI |
| | (SEQ ID NO: 1) | (SEQ ID NO: 2) | (SEQ ID NO: 3) |
| TBD2 | SYSMN | SISRSSSYIYYADSVKG | IHGYSNSDAFDK |
| | (SEQ ID NO: 1) | (SEQ ID NO: 2) | (SEQ ID NO: 7) |
| TBD3 | SYSMN | SISRSSSYIYYADSVKG | IHGYSNSDAFDI |
| | (SEQ ID NO: 1) | (SEQ ID NO: 2) | (SEQ ID NO: 8) |
| TBD4 | SYSMN | SISSSSSYIYYADSVKG | RHGYSNSDAFDN |
| | (SEQ ID NO: 1) | (SEQ ID NO: 10) | (SEQ ID NO: 11) |
| TBD5 | TYWMH | RINGDGSRTNYADSVKG | SSYAFDV |
| | (SEQ ID NO: 13) | (SEQ ID NO: 14) | (SEQ ID NO: 15) |
| TBD6 | TYWMH | RINSDGSRTNYADSVKG | SSYAFDV |
| | (SEQ ID NO: 13) | (SEQ ID NO: 19) | (SEQ ID NO: 15) |
| TBD7 | TYWMH | RINSDGSRTNYADSVKG | SSYAFHV |
| | (SEQ ID NO: 13) | (SEQ ID NO: 19) | (SEQ ID NO: 20) |
| TBD8 | SYSMN | SISXₐₐ₁SSSYIYYADSVKG, | Xₐₐ₁HGYSNSDAFD Xₐₐ₂, |
| (consensus of | (SEQ ID NO: 1) | wherein Xₐₐ₁ = R or S | wherein Xₐₐ₁ = E, I or R; |
| TBD1-4) | | (SEQ ID NO: 21) | Xₐₐ₂ = I, K, or N |
| | | | (SEQ ID NO: 22) |
| TBD9 | TYWMH | RINXₐₐ₁DGSRTNYADSVK | SSYAF Xₐₐ₁V, wherein |
| (consensus of | (SEQ ID NO: 13) | G, wherein Xₐₐ₁ = G or S | Xₐₐ₁ = D or H |
| TBD5-7) | | (SEQ ID NO: 25) | (SEQ ID NO: 26) |

TABLE 2
Exemplary sequences of human TfR binding domain light chain CDRs

| Human TfR | | | |
|---|---|---|---|
| binding | | | |
| domain (TBD) | LCDR1 (KABAT) | LCDR2 (KABAT) | LCDR3 (KABAT) |
| TBD1 | RASQGISNYLA | AASSLQS | LQHNSYPRT |
| | (SEQ ID NO: 4) | (SEQ ID NO: 5) | (SEQ ID NO: 6) |
| TBD2 | RASQGISNYLA | AASSLQS | LQHNSYPRT |
| | (SEQ ID NO: 4) | (SEQ ID NO: 5) | (SEQ ID NO: 6) |
| TBD3 | RASQGISHYLV | AASSLQS | LQHNSYPRT |
| | (SEQ ID NO: 9) | (SEQ ID NO: 5) | (SEQ ID NO: 6) |
| TBD4 | RASQGISHYLV | AASSLQS | LQHNSYPWT |
| | (SEQ ID NO: 9) | (SEQ ID NO: 5) | (SEQ ID NO: 12) |
| TBD5 | RSSQSLLDSDDGSTYLD | LLSNRAS | MQRIEFPLT |
| | (SEQ ID NO: 16) | (SEQ ID NO: 17) | (SEQ ID NO: 18) |
| TBD6 | RSSQSLLDSDDGSTYLD | LLSNRAS | MQRIEFPLT |
| | (SEQ ID NO: 16) | (SEQ ID NO: 17) | (SEQ ID NO: 18) |
| TBD7 | RSSQSLLDSDDGSTYLD | LLSNRAS | MQRIEFPLT |
| | (SEQ ID NO: 16) | (SEQ ID NO: 17) | (SEQ ID NO: 18) |
| TBD8 | RASQGIS Xₐₐ₁YL Xₐₐ₂, | AASSLQS | LQHNSYP Xₐₐ₁T, |
| (consensus of | wherein | (SEQ ID NO: 5) | wherein |
| TBD1-4) | Xₐₐ₁ = N or H; | | Xₐₐ₁ = R or W |
| | Xₐₐ₂ = A or V | | (SEQ ID NO: 24) |
| | (SEQ ID NO: 23) | | |
| TBD9 | RSSQSLLDSDDGSTYLD | LLSNRAS | MQRIEFPLT |
| (consensus of | (SEQ ID NO: 16) | (SEQ ID NO: 17) | (SEQ ID NO: 18) |
| TBD5-7) | | | |

TABLE 3
Exemplary sequences of human
TfR binding domain VH and VL

| Human | | |
|---|---|---|
| TfR | | |
| binding | | |
| domain | | |
| (TBD) | VH | VL |
| TBD1 | EVQLVESGGGLVKPG | DIQMTQSPSAMSASV |
| | GSLRLSCVASGFTFS | GDRVTITCRASQGIS |
| | SYSMNWVRQAPGKGL | NYLAWFQQKPGKVPK |
| | EWVSSISRSSSYIYY | RLIYAASSLQSGVPS |
| | ADSVKGRFTISRDNA | RFSGSGSGTEFTLTI |
| | KNSLYLQMNSLRAED | SSLQPEDFATYYCLQ |
| | TAVYYCAREHGYSNS | HNSYPRTFGQGTKVE |
| | DAFDIWGQGTLVTVS | IK |
| | S | (SEQ ID NO: 28) |
| | (SEQ ID NO: 27) | |
| TBD2 | EVQLVESGGGLVKPG | DIQMTQSPSAMSASV |
| | GSLRLSCVASGFTFS | GDRVTITCRASQGIS |
| | SYSMNWVRQAPGKGL | NYLAWFQQKPGKVPK |
| | EWVSSISRSSSYIYY | RLIYAASSLQSGVPS |
| | ADSVKGRFTISRDNA | RFSGSGSGTEFTLTI |
| | KNSLYLQMNSLRAED | SSLQPEDFATYYCLQ |
| | TAVYYCARIHGYSNS | HNSYPRTFGQGTKVE |
| | DAFDKWGQGTLVTVS | IK |
| | S | (SEQ ID NO: 28) |
| | (SEQ ID NO: 29) | |
| TBD3 | EVQLVESGGGLVKPG | DIQMTQSPSAMSASV |
| | GSLRLSCVASGFTFS | GDRVTITCRASQGIS |
| | SYSMNWVRQAPGKGL | HYLVWFQQKPGKVPK |
| | EWVSSISRSSSYIYY | RLIYAASSLQSGVPS |
| | ADSVKGRFTISRDNA | RFSGSGSGTEFTLTI |
| | KNSLYLQMNSLRAED | SSLQPEDFATYYCLQ |
| | TAVYYCARIHGYSNS | HNSYPRTFGQGTKVE |
| | DAFDIWGQGTLVTVS | IK |
| | S | (SEQ ID NO: 31) |
| | (SEQ ID NO: 30) | |
| TBD4 | EVQLVESGGGLVKPG | DIQMTQSPSAMSASV |
| | GSLRLSCVASGFTFS | GDRVTITCRASQGIS |
| | SYSMNWVRQAPGKGL | HYLVWFQQKPGKVPK |
| | EWVSSISSSSSYIYY | RLIYAASSLQSGVPS |
| | ADSVKGRFTISRDNA | RFSGSGSGTEFTLTI |
| | KNSLYLQMNSLRAED | SSLQPEDFATYYCLQ |
| | TAVYYCARRHGYSNS | HNSYPWTFGQGTKV |
| | DAFDNWGQGTLVTVS | EIK |
| | S | (SEQ ID NO: 33) |
| | (SEQ ID NO: 32) | |
| TBD5 | EVQLVESGGGLVQPG | DVVMTQTPLSLPVTP |
| | GSLRLSCAASGFTFR | GEPASISCRSSQSLL |
| | TYWMHWVRQAPGKGL | DSDDGSTYLDWYLQK |
| | LWVSRINGDGSRTNY | PGQSPQLLIYLLSNR |
| | ADSVKGRFTISRDNA | ASGVPDRFSGSGSGT |
| | KKTLYLQMNSLRAED | VFTLKISSVEAADVG |
| | TAVYFCARSSYAFDV | VYYCMQRIEFPLTFG |
| | WGQGTMVTVSS | GGTKVEIK |
| | (SEQ ID NO: 34) | (SEQ ID NO: 35) |
| TBD6 | EVQLVESGGGLVQPG | DIVMTQTPLSLPVTP |
| | GSLRLSCAASGFTFR | GEPASISCRSSQSLL |
| | TYWMHWVRQAPGKGL | DSDDGSTYLDWYLQK |
| | VWVSRINSDGSRTNY | PGQSPQLLIYLLSNR |
| | ADSVKGRFTISRDNA | ASGVPDRFSGSGSGT |
| | KNTLYLQMNSLRAED | DFTLKISRVEAEDVG |
| | TAVYYCARSSYAFDV | VYYCMQRIEFPLTFG |
| | WGQGTLVTVSS | GGTKVEIK |
| | (SEQ ID NO: 36) | (SEQ ID NO: 37) |
| TBD7 | EVQLVESGGGLVQPG | DIVMTQTPLSLPVTP |
| | GSLRLSCAASGFTFR | GEPASISCRSSQSLL |
| | TYWMHWVRQAPGKGL | DSDDGSTYLDWYLQK |
| | VWVSRINSDGSRTNY | PGQSPQLLIYLLSNR |
| | ADSVKGRFTISRDNA | ASGVPDRFSGSGSGT |
| | KNTLYLQMNSLRAED | DFTLKISRVEAEDVG |
| | TAVYYCARSSYAFHV | VYYCMQRIEFPLTFG |
| | WGQGTLVTVSS | GGTKVEIK |
| | (SEQ ID NO: 38) | (SEQ ID NO: 37) |

In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein the HCDR1, HCDR2, HCDR3, LCDR1, LCDR2, and LCDR3 comprise the following sequences:
(a) HCDR1 comprises SEQ TD NO: 1, HCDR2 comprises SEQ ID NO: 21, HCDR3 comprises SEQ ID NO: 22, LCDR1 comprises SEQ ID NO: 23, LCDR2 comprises SEQ TD NO: 5, and LCDR3 comprises SEQ ID NO: 24; or
(b) HCDR1 comprises SEQ TD NO: 13, HCDR2 comprises SEQ ID NO: 25, HCDR3 comprises SEQ ID NO: 26, LCDR1 comprises SEQ TD NO: 16, LCDR2 comprises SEQ TD NO: 17, and LCDR3 comprises SEQ TD NO: 18.

In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 1, HCDR2 comprises SEQ ID NO: 21, HCDR3 comprises SEQ ID NO: 22, LCDR1 comprises SEQ ID NO: 23, LCDR2 comprises SEQ ID NO: 5, and LCDR3 comprises SEQ ID NO: 24. In some embodiments, provided herein are proteins comprising one monovalent human transferrin receptor (TfR) binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 13, HCDR2 comprises SEQ ID NO: 25, HCDR3 comprises SEQ ID NO: 26, LCDR1 comprises SEQ ID NO: 16, LCDR2 comprises SEQ ID NO: 17, and LCDR3 comprises SEQ ID NO: 18.

In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6;
(b) HCDR1 comprises SEQ ID NO: 1, HCDR2 comprises SEQ ID NO: 2, HCDR3 comprises SEQ ID NO: 7, LCDR1 comprises SEQ ID NO: 4, LCDR2 comprises SEQ ID NO: 5, and LCDR3 comprises SEQ ID NO: 6;
(c) HCDR1 comprises SEQ ID NO: 1, HCDR2 comprises SEQ ID NO: 2, HCDR3 comprises SEQ ID NO: 8, LCDR1 comprises SEQ ID NO: 9, LCDR2 comprises SEQ ID NO: 5, and LCDR3 comprises SEQ ID NO: 6;
(d) HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12;
(e) HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 17, and LCDR3 comprises SEQ ID NO: 18;
(f) HCDR1 comprises SEQ ID NO: 13, HCDR2 comprises SEQ ID NO: 19, HCDR3 comprises SEQ ID NO: 15, LCDR1 comprises SEQ ID NO: 16, LCDR2 comprises SEQ ID NO: 17, and LCDR3 comprises SEQ ID NO: 18; or
(g) HCDR1 comprises SEQ ID NO: 13, HCDR2 comprises SEQ ID NO: 19, HCDR3 comprises SEQ ID NO: 20, LCDR1 comprises SEQ ID NO: 16, LCDR2 comprises SEQ ID NO: 17, and LCDR3 comprises SEQ ID NO: 18.

In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6. In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 1, HCDR2 comprises SEQ ID NO: 2, HCDR3 comprises SEQ ID NO: 7, LCDR1 comprises SEQ ID NO: 4, LCDR2 comprises SEQ ID NO: 5, and LCDR3 comprises SEQ ID NO: 6. In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 1, HCDR2 comprises SEQ ID NO: 2, HCDR3 comprises SEQ ID NO: 8, LCDR1 comprises SEQ ID NO: 9, LCDR2 comprises SEQ ID NO: 5, and LCDR3 comprises SEQ ID NO: 6. In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12. In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 17, and LCDR3 comprises SEQ ID NO: 18. In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 13, HCDR2 comprises SEQ ID NO: 19, HCDR3 comprises SEQ ID NO: 15, LCDR1 comprises SEQ ID NO: 16, LCDR2 comprises SEQ ID NO: 17, and LCDR3 comprises SEQ ID NO: 18. In some embodiments, provided herein are proteins comprising one monovalent human TfR binding domain, wherein the human TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 13, HCDR2 comprises SEQ ID NO: 19, HCDR3 comprises SEQ ID NO: 20, LCDR1 comprises SEQ ID NO: 16, LCDR2 comprises SEQ ID NO: 17, and LCDR3 comprises SEQ ID NO: 18.

In some embodiments, provided herein are proteins comprising one monovalent human TfR binding domain, wherein the human TfR binding domain comprises a VH and a VL, and wherein the VH and VL comprise the following sequences:
(a) VH comprises a sequence having at least 95% sequence identity to SEQ ID NO: 27 and VL comprises a sequence having at least 95% sequence identity to SEQ ID NO: 28;
(b) VH comprises a sequence having at least 95% sequence identity to SEQ ID NO: 29 and VL comprises a sequence having at least 95% sequence identity to SEQ ID NO: 28;
(c) VH comprises a sequence having at least 95% sequence identity to SEQ ID NO: 30 and VL comprises a sequence having at least 95% sequence identity to SEQ ID NO: 31;
(d) VH comprises a sequence having at least 95% sequence identity to SEQ ID NO: 32 and VL comprises a sequence having at least 95% sequence identity to SEQ ID NO: 33;
(e) VH comprises a sequence having at least 95% sequence identity to SEQ ID NO: 34 and VL comprises a sequence having at least 95% sequence identity to SEQ ID NO: 35;
(f) VH comprises a sequence having at least 95% sequence identity to SEQ ID NO: 36 and VL comprises a sequence having at least 95% sequence identity to SEQ ID NO: 37; or
(g) VH comprises a sequence having at least 95% sequence identity to SEQ ID NO: 38 and VL comprises a sequence having at least 95% sequence identity to SEQ ID NO: 37.

In some embodiments, provided herein are proteins comprising one monovalent human TfR binding domain, wherein the human TfR binding domain comprises a VH and a VL, and wherein the VH and VL comprise the following sequences:
(a) VH comprises SEQ ID NO: 27 and VL comprises SEQ ID NO: 28;
(b) VH comprises SEQ ID NO: 29 and VL comprises SEQ ID NO: 28;
(c) VH comprises SEQ ID NO: 30 and VL comprises SEQ ID NO: 31;
(d) VH comprises SEQ ID NO: 32 and VL comprises SEQ ID NO: 33;
(e) VH comprises SEQ ID NO: 34 and VL comprises SEQ ID NO: 35;
(f) VH comprises SEQ ID NO: 36 and VL comprises SEQ ID NO: 37; or
(g) VH comprises SEQ ID NO: 38 and VL comprises SEQ ID NO: 37.

In some embodiments, provided herein are proteins comprising one monovalent human TfR binding domain, wherein the human TfR binding domain comprises a VH and a VL, and wherein VH comprises SEQ ID NO: 27 and VL comprises SEQ ID NO: 28. In some embodiments, provided herein are proteins comprising one monovalent human TfR binding domain, wherein the human TfR binding domain comprises a VH and a VL, and wherein VH comprises SEQ ID NO: 29 and VL comprises SEQ ID NO: 28. In some embodiments, provided herein are proteins comprising one monovalent human TfR binding domain, wherein the human TfR binding domain comprises a VH and a VL, and wherein VH comprises SEQ ID NO: 30 and VL comprises SEQ ID NO: 31. In some embodiments, provided herein are proteins comprising one monovalent human TfR binding domain, wherein the human TfR binding domain comprises a VH and a VL, and wherein VH comprises SEQ ID NO: 32 and VL comprises SEQ ID NO: 33. In some embodiments, provided herein are proteins comprising one monovalent human TfR binding domain, wherein the human TfR binding domain comprises a VH and a VL, and wherein VH comprises SEQ ID NO: 34 and VL comprises SEQ ID NO: 35. In some embodiments, provided herein are proteins comprising one monovalent human TfR binding domain, wherein the human TfR binding domain comprises a VH and a VL, and wherein VH comprises SEQ ID NO: 36 and VL comprises SEQ ID NO: 37. In some embodiments, provided herein are proteins comprising one monovalent human TfR binding domain, wherein the human TfR binding domain comprises a VH and a VL, and wherein VH comprises SEQ ID NO: 38 and VL comprises SEQ ID NO: 37.

In some embodiments, the monovalent human TfR binding domain is an antibody fragment, e.g., Fab, scFv, Fv, or scFab (single chain Fab). In some embodiments, the monovalent human TfR binding domain is Fab. In some embodiments, the human TfR binding domain further comprises a heavy chain constant region and/or a light chain constant region.

In some embodiments, the human TfR binding proteins describe herein further comprise a half-life extender, e.g., an immunoglobulin Fc region or a VHH that binds human serum albumin (HSA).

In some embodiments, the human TfR binding proteins describe herein further comprise an immunoglobulin Fc region, e.g., a modified human IgG4 Fc region, or a modified human IgG1 Fc region. In some embodiments, the human TfR binding proteins describe herein further comprise a modified human IgG4 Fc region comprising proline at residue 228, and alanine at residues 234 and 235 (all residues are numbered according to the EU Index numbering, also called hIgG4PAA Fc region). In some embodiments, the human TfR binding proteins describe herein further comprise a modified human IgG1 Fc region comprising alanine at residues 234, 235, and 329, serine at position 265, aspartic acid at position 436 (all residues are numbered according to the EU Index numbering, also called hIgG1 effector null or hIgG1EN Fc region). In some embodiments, the human TfR binding proteins describe herein comprise a modified human IgG1 or IgG4 Fc region, wherein the Fc region comprises a first Fc CH3 domain comprising a serine at position 349, a methionine at position 366, a tyrosine at position 370, and a valine at position 409; and a second Fc CH3 domain comprising a glycine at position 356, an aspartic acid at position 357, a glutamine at position 364, and an alanine at position 407 (all residues are numbered according to the EU Index numbering). In some embodiments, the human TfR binding proteins described herein comprise a modified human IgG1 or IgG4 Fc region comprising a first Fc CH3 domain comprising leucine at residue 405, and a second Fc CH3 domain comprising arginine at residue 409 (all residues are numbered according to the EU Index numbering).

In some embodiments, the human TfR binding proteins describe herein further comprise a VHH that binds human HSA. In some embodiments, the VHH also binds mouse, rat, and/or cynomolgus monkey albumin. An exemplary VHH that binds human HSA is shown in Table 4. In some embodiments, such a VHH comprises CDR1 comprising SEQ ID NO: 39, CDR2 comprising SEQ ID NO: 40, and CDR3 comprising SEQ ID NO: 41. In some embodiments, such a VHH comprises SEQ ID NO: 42. In some embodiments, the VHH is linked to the TfR binding domain through a peptide linker, e.g., (GGGGQ)₄ (SEQ ID NO: 70).

TABLE 4
Exemplary sequences of VHH that binds
human serum albumin (HSA)

| | | SEQ |
|---|---|---|
| | | ID |
| Region | Sequence | NO |
| CDR1 | ETAVA | 39 |
| (KABAT) | | |
| CDR2 | GIGGGVDITYYADSVKG | 40 |
| (KABAT) | | |
| CDR3 | RPGRPLITSKVADLYPY | 41 |
| (KABAT) | | |
| VHH full | EVQLLESGGGLVQPGGS | 42 |
| length | LRLSCAASGRYIDETAV | |
| | AWFRQAPGKGREFVAGI | |
| | GGGVDITYYADSVKGRF | |
| | TISRDNSKNTLYLQMNS | |
| | LRPEDTAVYYCGARPGR | |
| | PLITSKVADLYPYWGQG | |
| | TLVTVSSPP | |
| Optional | GGGGQGGGGQGGGGQGG | 70 |
| linker | GGQ | |

In some embodiments, the human TfR binding proteins described herein are heterodimeric antibodies that comprise a first arm comprising one monovalent human TfR binding domain and a second arm that is a null arm, e.g., an arm that does not bind any known human target (e.g., an isotype arm). Heterodimeric antibodies such as heteromab, orthomab or duobody have been described in WO2014150973, WO2016118742, WO2018118616, WO2011131746. In some embodiments, the first arm comprises any one of the monovalent human TfR binding domains described herein. In some embodiments, the second arm is a null arm that does not bind any known human target (e.g., an isotype arm) comprises the sequences in Table 5. In some embodiments, the second arm comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 43, HCDR2 comprises SEQ ID NO: 44, HCDR3 comprises SEQ ID NO: 45, LCDR1 comprises SEQ ID NO: 46, LCDR2 comprises SEQ ID NO: 47, and LCDR3 comprises SEQ ID NO: 48. In some embodiments, the second arm comprises a VH and a VL, wherein the VH comprises SEQ ID NO: 49, and the VL comprises SEQ ID NO: 50. In some embodiments, the second arm comprises a heavy chain (HC) and a light chain (LC), wherein the HC comprises SEQ ID NO: 51, and the LC comprises SEQ ID NO: 52.

In some embodiments, the human TfR binding proteins described herein comprise heterodimeric mutations. In some embodiments, the human TfR binding proteins described herein comprise a modified Fc region comprising a first Fc CH3 domain comprising serine at residue 349, methionine at residue 366, tyrosine at residue 370, and valine at residue 409, and a second Fc CH3 domain comprising glycine at residue 356, aspartic acid at residue 357, glutamine at residue 364 and alanine at residue 407 (all residues are numbered according to the EU Index numbering). In some embodiments, the human TfR binding proteins described herein comprise a modified Fc region comprising a first Fc CH3 domain comprising leucine at residue 405, and a second Fc CH3 domain comprising arginine at residue 409 (all residues are numbered according to the EU Index numbering).

TABLE 5
Exemplary sequences of an isotype arm or
null arm that does not bind any known
target (Isotype Ab)

| | | SEQ ID |
|---|---|---|
| Region | Sequence | NO |
| HCDR1 | SYAIE | 43 |
| (KABAT) | | |
| HCDR2 | GILPGSGTINYNEKFKG | 44 |
| (KABAT) | | |
| HCDR3 | MSSNSDQGFDL | 45 |
| (KABAT) | | |
| LCDR1 | KASQGISRFLS | 46 |
| (KABAT) | | |
| LCDR2 | AVSSLVD | 47 |
| (KABAT) | | |
| LCDR3 | VQYNSYPYG | 48 |
| (KABAT) | | |
| VH | QVQLVQSGAEVKKPGSSVKV | 49 |
| | SCKASGYTFSSYAIEWVRQA | |
| | PGQGLEWMGGILPGSGTINY | |
| | NEKFKGRVTITADKSTSTAY | |
| | MELSSLRSEDTAVYYCARMS | |
| | SNSDQGFDLWGQGTLVTVSS | |
| VL | DIQMTQSPSSLSASVGDRVT | 50 |
| | ITCKASQGISRFLSWFQQKP | |
| | GKAPKSLIYAVSSLVDGVPS | |
| | RFSGSGSGTDFTLTISSLOP | |
| | EDFATYYCVQYNSYPYGFGG | |
| | GTKVEIK | |
| HC | QVQLVQSGAEVKKPGSSVKV | 51 |
| (hIgG4PAA) | SCKASGYTFSSYAIEWVRQA | |
| | PGQGLEWMGGILPGSGTINY | |
| | NEKFKGRVTITADKSTSTAY | |
| | MELSSLRSEDTAVYYCARMS | |
| | SNSDQGFDLWGQGTLVTVSS | |
| | ASTKGPXVFPLAPCSRSTSE | |
| | STAALGCLVKDYFPEPVTVS | |
| | WNSGALTSGVHTFPAVLQSS | |
| | GLYSLSSVVTVPSSSLGTKT | |
| | YTCNVDHKPSNTKVDKRVES | |
| | KYGPPCPPCPAPEAAGGPSV | |
| | FLFPPKPKDTLMISRTPEVT | |
| | CVVVDVSQEDPEVQFNWYVD | |
| | GVEVHNAKTKPREEQFNSTY | |
| | RVVSVLTVLHQDWLNGKEYK | |
| | CKVSNKGLPSSIEKTISKAK | |
| | GQPREPQVYTLPPSQEEMTK | |
| | NQVSLTCLVKGFYPSDIAVE | |
| | WESNGQPENNYKTTPPVLDS | |
| | DGSFLLYSKLTVDKSRWQEG | |
| | NVFSCSVMHEALHNHYTQKS | |
| | LSLSLG, | |
| | wherein X is S or C. | |
| LC | DIQMTQSPSSLSASVGDRVT | 52 |
| (human kappa) | ITCKASQGISRFLSWFQQKP | |
| | GKAPKSLIYAVSSLVDGVPS | |
| | RFSGSGSGTDFTLTISSLQP | |
| | EDFATYYCVQYNSYPYGFGG | |
| | GTKVEIKRTVAAPSVFIFPP | |
| | SDEQLKSGTASVVCLLNNFY | |
| | PREAKVQWKVDNALQSGNSQ | |
| | ESVTEQDSKDSTYSLSSTLT | |
| | LSKADYEKHKVYACEVTHQG | |
| | LSSPVTKSFNRGEC | |

In some embodiments, the human TfR binding proteins described herein comprise one or more native cysteine residues, which can be used for conjugation. For example, in some embodiments, the human TfR binding protein described herein comprises a native cysteine at position 220 of the light chain and/or a native cysteine at position 226 of the heavy chain, which can be used for conjugation (all residues according to the EU Index numbering).

In some embodiments, the human TfR binding proteins described herein comprise engineered cysteine residues for conjugation. The approach of including engineered cysteines as a means for conjugation has been described in WO 2018/232088. In some embodiments, the human TfR binding proteins described herein comprise a heavy chain comprising one or more cysteines at the following residues: 124, 157, 162, 262, 373, 375, 378, 397, 415 (all residues according to the EU Index numbering). In some embodiments, the human TfR binding proteins described herein comprise a light chain (e.g., a kappa light chain) comprising one or more cysteines at the following residues: 156, 171, 191, 193, 202, 208 (all residues according to the EU Index numbering). In some embodiments, the human TfR binding proteins described herein comprise a heavy chain constant region comprising cysteine at residue 124 (according to the EU Index numbering). In some embodiments, the human TfR binding proteins described herein comprise a light chain constant region comprising cysteine at residue 156 (according to the EU Index numbering). In some embodiments, the human TfR binding proteins described herein comprise an immunoglobulin Fc region comprising cysteine at residue 378 (according to the EU Index numbering).

In some embodiments, the human TfR binding protein described herein is any one of the human TfR binding proteins in Table 6a and 6b. In some embodiments, the human TfR binding protein described herein has one heavy chain (HC) and one light chain (LC), e.g., TBP1, TBP2, TBP3, TBP4, TBP5, TBP6, TBP7, TBP8, or TBP9 (see Table 6a).

In some embodiments, the human TfR binding protein described herein has a Fab-Fc format, e.g., TBP1, TBP2, TBP3, TBP4, TBP5, TBP6, or TBP7. In some embodiments, provided herein are human TfR binding proteins comprise one HC and one LC, wherein the HC comprises SEQ ID NO: 53 and the LC comprises SEQ ID NO: 54. In some embodiments, provided herein are human TfR binding proteins comprise one HC and one LC, wherein the HC comprises SEQ ID NO: 55 and the LC comprises SEQ ID NO: 54. In some embodiments, provided herein are human TfR binding proteins comprise one HC and one LC, wherein the HC comprises SEQ ID NO: 56 and the LC comprises SEQ ID NO: 57. In some embodiments, provided herein are human TfR binding proteins comprise one HC and one LC, wherein the HC comprises SEQ ID NO: 58 and the LC comprises SEQ ID NO: 59. In some embodiments, provided herein are human TfR binding proteins comprise one HC and one LC, wherein the HC comprises SEQ ID NO: 60 and the LC comprises SEQ ID NO: 61. In some embodiments, provided herein are human TfR binding proteins comprise one HC and one LC, wherein the HC comprises SEQ ID NO: 62 and the LC comprises SEQ ID NO: 63. In some embodiments, provided herein are human TfR binding proteins comprise one HC and one LC, wherein the HC comprises SEQ ID NO: 64 and the LC comprises SEQ TD NO: 63.

In some embodiments, the human TfR binding protein described herein has a Fab format, e.g., TBP8. In some embodiments, provided herein are human TfR binding proteins comprise one HC and one LC, and wherein the HC comprises SEQ ID NO: 65 and the LC comprises SEQ ID NO: 59.

In some embodiments, the human TfR binding protein described herein has a Fab-VHH format, e.g., TBP9. In some embodiments, provided herein are human TfR binding proteins comprise one HC and one LC, wherein the HC comprises SEQ ID NO: 66 and the LC comprises SEQ ID NO: 67.

TABLE 6a
Exemplary sequences of human TfR
binding proteins (one HC and one LC)

| Human | | |
|---|---|---|
| TfR | | |
| binding | | |
| protein | | |
| (TBP) | HC | LC |
| TBP1 | EVQLVESGGGLVKPG | DIQMTQSPSAMSASV |
| (TBD1 | GSLRLSCVASGFTFS | GDRVTITCRASQGIS |
| Fab- | SYSMNWVRQAPGKGL | NYLAWFQQKPGKVPK |
| hIgG4PAA | EWVSSISRSSSYIYY | RLIYAASSLQSGVPS |
| Fc) | ADSVKGRFTISRDNA | RFSGSGSGTEFTLTI |
| | KNSLYLQMNSLRAED | SSLQPEDFATYYCLQ |
| | TAVYYCAREHGYSNS | HNSYPRTFGQGTKVE |
| | DAFDIWGQGTLVTVS | IKRTVAAPSVFIFPP |
| | SASTKGPSVFPLAPC | SDEQLKSGTASVVCL |
| | SRSTSESTAALGCLV | LNNFYPREAKVQWKV |
| | KDYFPEPVTVSWNSG | DNALQSGNSQESVTE |
| | ALTSGVHTFPAVLQS | QDSKDSTYSLSSTLT |
| | SGLYSLSSVVTVPSS | LSKADYEKHKVYACE |
| | SLGTKTYTCNVDHKP | VTHQGLSSPVTKSFN |
| | SNTKVDKRVESKYGP | RGEC |
| | PCPPCPAPEAAGGPS | (SEQ ID NO: 54) |
| | VFLFPPKPKDTLMIS | |
| | RTPEVTCVVVDVSQE | |
| | DPEVQFNWYVDGVEV | |
| | HNAKTKPREEQFNST | |
| | YRVVSVLTVLHQDWL | |
| | NGKEYKCKVSNKGLP | |
| | SSIEKTISKAKGQPR | |
| | EPQVYTLPPSQEEMT | |
| | KNQVSLTCLVKGFYP | |
| | SDIAVEWESNGQPEN | |
| | NYKTTPPVLDSDGSF | |
| | FLYSRLTVDKSRWQE | |
| | GNVFSCSVMHEALHN | |
| | HYTQKSLSLSLG | |
| | (SEQ ID NO: 53) | |
| TBP2 | EVQLVESGGGLVKPG | DIQMTQSPSAMSASV |
| (TBD2 | GSLRLSCVASGFTFS | GDRVTITCRASQGIS |
| Fab- | SYSMNWVRQAPGKGL | NYLAWFQQKPGKVPK |
| hIgG4PAA | EWVSSISRSSSYIYY | RLIYAASSLQSGVPS |
| Fc) | ADSVKGRFTISRDNA | RFSGSGSGTEFTLTI |
| | KNSLYLQMNSLRAED | SSLQPEDFATYYCLQ |
| | TAVYYCARIHGYSNS | HNSYPRTFGQGTKVE |
| | DAFDKWGQGTLVTVS | IKRTVAAPSVFIFPP |
| | SASTKGPXVFPLAPC | SDEQLKSGTASVVCL |
| | SRSTSESTAALGCLV | LNNFYPREAKVQWKV |
| | KDYFPEPVTVSWNSG | DNALQSGNSQESVTE |
| | ALTSGVHTFPAVLQS | QDSKDSTYSLSSTLT |
| | SGLYSLSSVVTVPSS | LSKADYEKHKVYACE |
| | SLGTKTYTCNVDHKP | VTHQGLSSPVTKSFN |
| | SNTKVDKRVESKYGP | RGEC |
| | PCPPCPAPEAAGGPS | (SEQ ID NO: 54) |
| | VFLFPPKPKDTLMIS | |
| | RTPEVTCVVVDVSQE | |
| | DPEVQFNWYVDGVEV | |
| | HNAKTKPREEQFNST | |
| | YRVVSVLTVLHQDWL | |
| | NGKEYKCKVSNKGLP | |
| | SSIEKTISKAKGQPR | |
| | EPQVYTLPPSQEEMT | |
| | KNQVSLTCLVKGFYP | |
| | SDIAVEWESNGQPEN | |
| | NYKTTPPVLDSDGSF | |
| | FLYSRLTVDKSRWQE | |
| | GNVFSCSVMHEALHN | |
| | HYTQKSLSLSLG, | |
| | wherein X is S | |
| | or C. | |
| | (SEQ ID NO: 55) | |
| TBP3 | EVQLVESGGGLVKPG | DIQMTQSPSAMSASV |
| (TBD3 | GSLRLSCVASGFTFS | GDRVTITCRASQGIS |
| Fab- | SYSMNWVRQAPGKGL | HYLVWFQQKPGKVPK |
| hIgG4PAA | EWVSSISRSSSYIYY | RLIYAASSLQSGVPS |
| Fc) | ADSVKGRFTISRDNA | RFSGSGSGTEFTLTI |
| | KNSLYLQMNSLRAED | SSLQPEDFATYYCLQ |
| | TAVYYCARIHGYSNS | HNSYPRTFGQGTKVE |
| | DAFDIWGQGTLVTVS | IKRTVAAPSVFIFPP |
| | SASTKGPSVFPLAPC | SDEQLKSGTASVVCL |
| | SRSTSESTAALGCLV | LNNFYPREAKVQWKV |
| | KDYFPEPVTVSWNSG | DNALQSGNSQESVTE |
| | ALTSGVHTFPAVLQS | QDSKDSTYSLSSTLT |
| | SGLYSLSSVVTVPSS | LSKADYEKHKVYACE |
| | SLGTKTYTCNVDHKP | VTHQGLSSPVTKSFN |
| | SNTKVDKRVESKYGP | RGEC |
| | PCPPCPAPEAAGGPS | (SEQ ID NO: 57) |
| | VFLFPPKPKDTLMIS | |
| | RTPEVTCVVVDVSQE | |
| | DPEVQFNWYVDGVEV | |
| | HNAKTKPREEQFNST | |
| | YRVVSVLTVLHQDWL | |
| | NGKEYKCKVSNKGLP | |
| | SSIEKTISKAKGQPR | |
| | EPQVYTLPPSQEEMT | |
| | KNQVSLTCLVKGFYP | |
| | SDIAVEWESNGQPEN | |
| | NYKTTPPVLDSDGSF | |
| | FLYSRLTVDKSRWQE | |
| | GNVFSCSVMHEALHN | |
| | HYTQKSLSLSLG | |
| | (SEQ ID NO: 56) | |
| TBP4 | EVQLVESGGGLVKPG | DIQMTQSPSAMSASV |
| (TBD4 | GSLRLSCVASGFTFS | GDRVTITCRASQGIS |
| Fab- | SYSMNWVRQAPGKGL | HYLVWFQQKPGKVPK |
| hIgG4PAA | EWVSSISSSSSYIYY | RLIYAASSLQSGVPS |
| Fc) | ADSVKGRFTISRDNA | RFSGSGSGTEFTLTI |
| | KNSLYLQMNSLRAED | SSLQPEDFATYYCLQ |
| | TAVYYCARRHGYSNS | HNSYPWTFGQGTKVE |
| | DAFDNWGQGTLVTVS | IKRTVAAPSVFIFPP |
| | SASTKGPXVFPLAPC | SDEQLKSGTASVVCL |
| | SRSTSESTAALGCLV | LNNFYPREAKVQWKV |
| | KDYFPEPVTVSWNSG | DNALQSGNSQESVTE |
| | ALTSGVHTFPAVLQS | QDSKDSTYSLSSTLT |
| | SGLYSLSSVVTVPSS | LSKADYEKHKVYACE |
| | SLGTKTYTCNVDHKP | VTHQGLSSPVTKSFN |
| | SNTKVDKRVESKYGP | RGEC |
| | PCPPCPAPEAAGGPS | (SEQ ID NO: 59) |
| | VFLFPPKPKDTLMIS | |
| | RTPEVTCVVVDVSQE | |
| | DPEVQFNWYVDGVEV | |
| | HNAKTKPREEQFNST | |
| | YRVVSVLTVLHQDWL | |
| | NGKEYKCKVSNKGLP | |
| | SSIEKTISKAKGQPR | |
| | EPQVYTLPPSQEEMT | |
| | KNQVSLTCLVKGFYP | |
| | SDIAVEWESNGQPEN | |
| | NYKTTPPVLDSDGSF | |
| | FLYSRLTVDKSRWQE | |
| | GNVFSCSVMHEALHN | |
| | HYTQKSLSLSLG, | |
| | wherein X is | |
| | S or C. | |
| | (SEQ ID NO: 58) | |
| TBP5 | EVQLVESGGGLVQPG | DVVMTQTPLSLPVTP |
| (TBD5 | GSLRLSCAASGFTFR | GEPASISCRSSQSLL |
| Fab- | TYWMHWVRQAPGKGL | DSDDGSTYLDWYLQK |
| hIgG4PAA | LWVSRINGDGSRTNY | PGQSPQLLIYLLSNR |
| Fc) | ADSVKGRFTISRDNA | ASGVPDRFSGSGSGT |
| | KKTLYLQMNSLRAED | VFTLKISSVEAADVG |
| | TAVYFCARSSYAFDV | VYYCMQRIEFPLTFG |
| | WGQGTMVTVSSASTK | GGTKVEIKRTVAAPS |
| | GPSVFPLAPCSRSTS | VFIFPPSDEQLKSGT |
| | ESTAALGCLVKDYFP | ASVVCLLNNFYPREA |
| | EPVTVSWNSGALTSG | KVQWKVDNALQSGNS |
| | VHTFPAVLQSSGLYS | QESVTEQDSKDSTYS |
| | LSSVVTVPSSSLGTK | LSSTLTLSKADYEKH |
| | TYTCNVDHKPSNTKV | KVYACEVTHQGLSSP |
| | DKRVESKYGPPCPPC | VTKSFNRGEC |
| | PAPEAAGGPSVFLFP | (SEQ |
| | PKPKDTLMISRTPEV | ID NO: 61) |
| | TCVVVDVSQEDPEVQ | |
| | FNWYVDGVEVHNAKT | |
| | KPREEQFNSTYRVVS | |
| | VLTVLHQDWLNGKEY | |
| | KCKVSNKGLPSSIEK | |
| | TISKAKGQPREPQVY | |
| | TLPPSQEEMTKNQVS | |
| | LTCLVKGFYPSDIAV | |
| | EWESNGQPENNYKTT | |
| | PPVLDSDGSFFLYSR | |
| | LTVDKSRWQEGNVFS | |
| | CSVMHEALHNHYTQK | |
| | SLSLSLG | |
| | (SEQ ID NO: 60) | |
| TBP6 | EVQLVESGGGLVQPG | DIVMTQTPLSLPVTP |
| (TBD6 | GSLRLSCAASGFTFR | GEPASISCRSSQSLL |
| Fab- | TYWMHWVRQAPGKGL | DSDDGSTYLDWYLQK |
| hIgG4PAA | VWVSRINSDGSRTNY | PGQSPQLLIYLLSNR |
| Fc) | ADSVKGRFTISRDNA | ASGVPDRFSGSGSGT |
| | KNTLYLQMNSLRAED | DFTLKISRVEAEDVG |
| | TAVYYCARSSYAFDV | VYYCMQRIEFPLTFG |
| | WGQGTLVTVSSASTK | GGTKVEIKRTVAAPS |
| | GPSVFPLAPCSRSTS | VFIFPPSDEQLKSGT |
| | ESTAALGCLVKDYFP | ASVVCLLNNFYPREA |
| | EPVTVSWNSGALTSG | KVQWKVDNALQSGNS |
| | VHTFPAVLQSSGLYS | QESVTEQDSKDSTYS |
| | LSSVVTVPSSSLGTK | LSSTLTLSKADYEKH |
| | TYTCNVDHKPSNTKV | KVYACEVTHQGLSSP |
| | DKRVESKYGPPCPPC | VTKSFNRGEC |
| | PAPEAAGGPSVFLFP | (SEQ ID NO: 63) |
| | PKPKDTLMISRTPEV | |
| | TCVVVDVSQEDPEVQ | |
| | FNWYVDGVEVHNAKT | |
| | KPREEQFNSTYRVVS | |
| | VLTVLHQDWLNGKEY | |
| | KCKVSNKGLPSSIEK | |
| | TISKAKGQPREPQVY | |
| | TLPPSQEEMTKNQVS | |
| | LTCLVKGFYPSDIAV | |
| | EWESNGQPENNYKTT | |
| | PPVLDSDGSFFLYSR | |
| | LTVDKSRWQEGNVFS | |
| | CSVMHEALHNHYTQK | |
| | SLSLSLG | |
| | (SEQ ID NO: 62) | |
| TBP7 | EVQLVESGGGLVQPG | DIVMTQTPLSLPVTP |
| (TBD7 | GSLRLSCAASGFTFR | GEPASISCRSSQSLL |
| Fab- | TYWMHWVRQAPGKGL | DSDDGSTYLDWYLQK |
| hIgG4PAA | VWVSRINSDGSRTNY | PGQSPQLLIYLLSNR |
| Fc) | ADSVKGRFTISRDNA | ASGVPDRFSGSGSGT |
| | KNTLYLQMNSLRAED | DFTLKISRVEAEDVG |
| | TAVYYCARSSYAFHV | VYYCMQRIEFPLTFG |
| | WGQGTLVTVSSASTK | GGTKVEIKRTVAAPS |
| | GPXVFPLAPCSRSTS | VFIFPPSDEQLKSGT |
| | ESTAALGCLVKDYFP | ASVVCLLNNFYPREA |
| | EPVTVSWNSGALTSG | KVQWKVDNALQSGNS |
| | VHTFPAVLQSSGLYS | QESVTEQDSKDSTYS |
| | LSSVVTVPSSSLGTK | LSSTLTLSKADYEKH |
| | TYTCNVDHKPSNTKV | KVYACEVTHQGLSSP |
| | DKRVESKYGPPCPPC | VTKSFNRGEC |
| | PAPEAAGGPSVFLFP | (SEQ ID NO: 63) |
| | PKPKDTLMISRTPEV | |
| | TCVVVDVSQEDPEVQ | |
| | FNWYVDGVEVHNAKT | |
| | KPREEQFNSTYRVVS | |
| | VLTVLHQDWLNGKEY | |
| | KCKVSNKGLPSSIEK | |
| | TISKAKGQPREPQVY | |
| | TLPPSQEEMTKNQVS | |
| | LTCLVKGFYPSDIAV | |
| | EWESNGQPENNYKTT | |
| | PPVLDSDGSFFLYSR | |
| | LTVDKSRWQEGNVFS | |
| | CSVMHEALHNHYTQK | |
| | SLSLSLG,wherein | |
| | X is S or C. | |
| | (SEQ ID NO: 64) | |
| TBP8 | EVQLVESGGGLVKPG | DIQMTQSPSAMSASV |
| (TBD4 | GSLRLSCVASGFTFS | GDRVTITCRASQGIS |
| Fab) | SYSMNWVRQAPGKGL | HYLVWFQQKPGKVPK |
| | EWVSSISSSSSYIYY | RLIYAASSLQSGVPS |
| | ADSVKGRFTISRDNA | RFSGSGSGTEFTLTI |
| | KNSLYLQMNSLRAED | SSLQPEDFATYYCLQ |
| | TAVYYCARRHGYSNS | HNSYPWTFGQGTKVE |
| | DAFDNWGQGTLVTVS | IKRTVAAPSVFIFPP |
| | SASTKGPSVFPLAPS | SDEQLKSGTASVVCL |
| | SKSTSGGTAALGCLV | LNNFYPREAKVQWKV |
| | KDYFPEPVTVSWNSG | DNALQSGNSQESVTE |
| | ALTSGVHTFPAVLQS | QDSKDSTYSLSSTLT |
| | SGLYSLSSVVTVPSS | LSKADYEKHKVYACE |
| | SLGTQTYICNVNHKP | VTHQGLSSPVTKSFN |
| | SNTKVDKRVEPKC | RGEC |
| | (S | (SEQ ID NO: 59) |
| | EQ ID NO: 65) | |
| TBP9 | EVQLVESGGGLVKPG | DIQMTQSPSAMSASV |
| (TBD4 | GSLRLSCVASGFTFS | GDRVTITCRASQGIS |
| Fab- | SYSMNWVRQAPGKGL | HYLVWFQQKPGKVPK |
| VHH) | EWVSSISSSSSYIYY | RLIYAASSLQSGVPS |
| | ADSVKGRFTISRDNA | RFSGSGSGTEFTLTI |
| | KNSLYLQMNSLRAED | SSLQPEDFATYYCLQ |
| | TAVYYCARRHGYSNS | HNSYPWTFGQGTKVE |
| | DAFDNWGQGTLVTVS | IKRTVAAPSVFIFPP |
| | SASTKGPCVFPLAPS | SDEQLKSGTASVVCL |
| | SKSTSGGTAALGCLV | LNNFYPREAKVQWKV |
| | KDYFPEPVTVSWNSG | DNALQCGNSQESVTE |
| | ALTSGVHTFPAVLQS | QDSKDSTYSLSSTLT |
| | SGLYSLSSVVTVPSS | LSKADYEKHKVYACE |
| | SLGTQTYICNVNHKP | VTHQGLSSPVTKSFN |
| | SNTKVDKRVEPKCDK | RGEC |
| | THTGGGGQGGGGQGG | (SEQ ID NO: 67) |
| | GGQGGGGQGGGGQEV | |
| | QLLESGGGLVQPGGS | |
| | LRLSCAASGRYIDET | |
| | AVAWFRQAPGKGREF | |
| | VAGIGGGVDITYYAD | |
| | SVKGRFTISRDNSKN | |
| | TLYLQMNSLRPEDTA | |
| | VYYCGARPGRPLITS | |
| | KVADLYPYWGQGTLV | |
| | TVSSPP | |
| | (SEQ ID NO: 66) | |

In some embodiments, the human TfR binding protein described herein has more than one heavy chain (HC) and/or more than one light chain (see Table 6b). In some embodiments, the human TfR binding protein has two heavy chains (HC1 and HC2) and two light chains (LC1 and LC2). In some embodiments, the human TfR binding protein described herein has a heterodimeric antibody format, e.g., TBP10, TBP11, TBP12, or TBP13.

In some embodiments, provided herein are human TfR binding proteins comprise two heavy chains HC1 and HC2 and two light chains LC1 and LC2, wherein HC1 comprises SEQ ID NO: 64, LC1 comprises SEQ ID NO: 63, HC2 comprises SEQ ID NO: 51, and LC2 comprises SEQ ID NO: 52. In some embodiments, provided herein are human TfR binding proteins comprise two heavy chains HC1 and HC2 and two light chains LC1 and LC2, wherein HC1 comprises SEQ ID NO: 55, LC1 comprises SEQ ID NO: 54, HC2 comprises SEQ ID NO: 51, and LC2 comprises SEQ ID NO: 52. In some embodiments, provided herein are human TfR binding proteins comprise two heavy chains HC1 and HC2 and two light chains LC1 and LC2, wherein HC1 comprises SEQ ID NO: 56, LC1 comprises SEQ ID NO: 57, HC2 comprises SEQ ID NO: 51, and LC2 comprises SEQ ID NO: 52. In some embodiments, provided herein are human TfR binding proteins comprise two heavy chains HC1 and HC2 and two light chains LC1 and LC2, wherein HC1 comprises SEQ ID NO: 58, LC1 comprises SEQ ID NO: 59, HC2 comprises SEQ ID NO: 51, and LC2 comprises SEQ ID NO: 52.

In some embodiments, the human TfR binding protein has two heavy chains (HC1 and HC2) and one light chain (LC1), e.g., TBP14, TBP15, TBP16. In some embodiments, provided herein are human TfR binding proteins comprise two heavy chains HC1 and HC2 and one light chain LC1, wherein HC1 comprises SEQ ID NO: 68, LC1 comprises SEQ ID NO: 59, HC2 comprises SEQ ID NO: 69. In some embodiments, provided herein are human TfR binding proteins comprise two heavy chains HC1 and HC2 and one light chain LC1, wherein HC1 comprises SEQ ID NO: 138, LC1 comprises SEQ ID NO: 59, HC2 comprises SEQ ID NO: 139. In some embodiments, provided herein are human TfR binding proteins comprise two heavy chains HC1 and HC2 and one light chain LC1, wherein HC1 comprises SEQ ID NO: 166, LC1 comprises SEQ ID NO: 54, HC2 comprises SEQ ID NO: 167.

TABLE 6b
Exemplary sequences of human TfR binding
proteins (multiple HC and/or LC)

| Human TfR | | | | |
|---|---|---|---|---|
| binding | | | | |
| protein | | | | |
| (TBP) | HC1 | LC1 | HC2 | LC2 |
| TBP10 | SEQ | SEQ | SEQ | SEQ |
| (TBD7- | ID | ID | ID | ID |
| isotype | NO: | NO: | NO: | NO: |
| heterodimeric | 64 | 63 | 51 | 52 |
| Ab) | | | | |
| TBP11 | SEQ | SEQ | SEQ | SEQ |
| (TBD2- | ID | ID | ID | ID |
| isotype | NO: | NO: | NO: | NO: |
| heterodimeric | SEQ | SEQ | SEQ | SEQ |
| Ab) | 55 | 54 | 51 | 52 |
| TBP12 | SEQ | SEQ | SEQ | SEQ |
| (TBD3- | ID | ID | ID | ID |
| isotype | NO: | NO: | NO: | NO: |
| heterodimeric | 56 | 57 | 51 | 52 |
| Ab) | | | | |
| TBP13 | SEQ | SEQ | SEQ | SEQ |
| (TBD4- | ID | ID | ID | ID |
| heterodimeric | NO: | NO: | NO: | NO: |
| Ab) | 58 | 59 | 51 | 52 |
| TBP14 | SEQ | SEQ | SEQ | N/A* |
| (TBD4-one | ID | ID | ID | |
| arm | NO: | NO: | NO: | |
| heteromab, | 68 | 59 | 69 | |
| A378C) | | | | |
| TBP15 | SEQ | SEQ | SEQ | N/A* |
| (TBD4-one | ID | ID | ID | |
| arm | NO: | NO: | NO: | |
| heteromab2, | 138 | 59 | 139 | |
| S124C) | | | | |
| TBP16 | SEQ | SEQ | SEQ | N/A* |
| (TBD2-one | ID | ID | ID | |
| arm | NO: | NO: | NO: | |
| heteromab, | 166 | 54 | 167 | |
| S124C) | | | | |

| | |
|---|---|
| SEQ ID NO: 68 | EVQLVESGGGLVKPGGSLRL |
| | SCVASGFTFSSYSMNWVRQA |
| | PGKGLEWVSSISSSSSYIYY |
| | ADSVKGRFTISRDNAKNSLY |
| | LQMNSLRAEDTAVYYCARRH |
| | GYSNSDAFDNWGQGTLVTVS |
| | SASTKGPSVFPLAPCSRSTS |
| | ESTAALGCLVKDYFPEPVTV |
| | SWNSGALTSGVHTFPAVLQS |
| | SGLYSLSSVVTVPSSSLGTK |
| | TYTCNVDHKPSNTKVDKRVE |
| | SKYGPPCPPCPAPEAAGGPS |
| | VFLFPPKPKDTLMISRTPEV |
| | TCVVVDVSQEDPEVQFNWYV |
| | DGVEVHNAKTKPREEQFNST |
| | YRVVSVLTVLHQDWLNGKEY |
| | KCKVSNKGLPSSIEKTISKA |
| | KGQPREPQVSTLPPSQEEMT |
| | KNQVSLMCLVYGFYPSDICV |
| | EWESNGQPENNYKTTPPVLD |
| | SDGSFFLYSVLTVDKSRWQE |
| | GNVFSCSVMHEALHNHYTQK |
| | SLSLSLG |
| SEQ ID NO: 69 | ESKYGPPCPPCPAPEAAGGP |
| | SVFLFPPKPKDTLMISRTPE |
| | VTCVVVDVSQEDPEVQFNWY |
| | VDGVEVHNAKTKPREEQFNS |
| | TYRVVSVLTVLHQDWLNGKE |
| | YKCKVSNKGLPSSIEKTISK |
| | AKGQPREPQVYTLPPSQGDM |
| | TKNQVQLTCLVKGFYPSDIC |
| | VEWESNGQPENNYKTTPPVL |
| | DSDGSFFLASRLTVDKSRWQ |
| | EGNVFSCSVMHEALHNHYTQ |
| | KSLSLSLG |
| SEQ ID NO: 138 | EVQLVESGGGLVKPGGSLRL |
| | SCVASGFTFSSYSMNWVRQA |
| | PGKGLEWVSSISSSSSYIYY |
| | ADSVKGRFTISRDNAKNSLY |
| | LQMNSLRAEDTAVYYCARRH |
| | GYSNSDAFDNWGQGTLVTVS |
| | SASTKGPCVFPLAPCSRSTS |
| | ESTAALGCLVKDYFPEPVTV |
| | SWNSGALTSGVHTFPAVLQS |
| | SGLYSLSSVVTVPSSSLGTK |
| | TYTCNVDHKPSNTKVDKRVE |
| | SKYGPPCPPCPAPEAAGGPS |
| | VFLFPPKPKDTLMISRTPEV |
| | TCVVVDVSQEDPEVQFNWYV |
| | DGVEVHNAKTKPREEQFNST |
| | YRVVSVLTVLHQDWLNGKEY |
| | KCKVSNKGLPSSIEKTISKA |
| | KGQPREPQVSTLPPSQEEMT |
| | KNQVSLMCLVYGFYPSDIAV |
| | EWESNGQPENNYKTTPPVLD |
| | SDGSFFLYSVLTVDKSRWQE |
| | GNVFSCSVMHEALHNHYTQK |
| | SLSLSLG |
| SEQ ID NO: 139 | ESKYGPPCPPCPAPEAAGGP |
| | SVFLFPPKPKDTLMISRTPE |
| | VTCVVVDVSQEDPEVQFNWY |
| | VDGVEVHNAKTKPREEQFNS |
| | TYRVVSVLTVLHQDWLNGKE |
| | YKCKVSNKGLPSSIEKTISK |
| | AKGQPREPQVYTLPPSQGDM |
| | TKNQVQLTCLVKGFYPSDIA |
| | VEWESNGQPENNYKTTPPVL |
| | DSDGSFFLASRLTVDKSRWQ |
| | EGNVFSCSVMHEALHNHYTQ |
| | KSLSLSLG |
| SEQ ID NO: 166 | EVQLVESGGGLVKPGGSLRL |
| | SCVASGFTFSSYSMNWVRQA |
| | PGKGLEWVSSISRSSSYIYY |
| | ADSVKGRFTISRDNAKNSLY |
| | LQMNSLRAEDTAVYYCARIH |
| | GYSNSDAFDKWGQGTLVTVS |
| | SASTKGPCVFPLAPCSRSTS |
| | ESTAALGCLVKDYFPEPVTV |
| | SWNSGALTSGVHTFPAVLQS |
| | SGLYSLSSVVTVPSSSLGTK |
| | TYTCNVDHKPSNTKVDKRVE |
| | SKYGPPCPPCPAPEAAGGPS |
| | VFLFPPKPKDTLMISRTPEV |
| | TCVVVDVSQEDPEVQFNWYV |
| | DGVEVHNAKTKPREEQFNST |
| | YRVVSVLTVLHQDWLNGKEY |
| | KCKVSNKGLPSSIEKTISKA |
| | KGQPREPQVYTLPPSQEEMT |
| | KNQVSLTCLVKGFYPSDIAV |
| | EWESNGQPENNYKTTPPVLD |
| | SDGSFFLYSRLTVDKSRWQE |
| | GNVFSCSVMHEALHNHYTQK |
| | SLSLSLG |
| SEQ ID NO: 167 | ESKYGPPCPPCPAPEAAGGP |
| | SVFLFPPKPKDTLMISRTPE |
| | VTCVVVDVSQEDPEVQFNWY |
| | VDGVEVHNAKTKPREEQFNS |
| | TYRVVSVLTVLHQDWLNGKE |
| | YKCKVSNKGLPSSIEKTISK |
| | AKGQPREPQVYTLPPSQEEM |
| | TKNQVSLTCLVKGFYPSDIA |
| | VEWESNGQPENNYKTTPPVL |
| | DSDGSFLLYSKLTVDKSRWQ |
| | EGNVFSCSVMHEALHNHYTQ |
| | KSLSLSLG |

*N/A = not applicable, which means the TBP does not have that heavy or light chain.

In some embodiments, provided herein are proteins comprising one monovalent human transferrin receptor (TfR) binding domain, wherein the human TfR binding domain binds an epitope comprising one or more residues in (a) residues 346-364 FGNMEGDCPSDWKTDSTCR (SEQ TD NO: 119), (b) residues 243-247 FEDLY (SEQ TD NO: 162) and residues 345-364 LFGNMEEGDCPSDWKTDSTCR) (SEQ ID NO: 163), or (c) residues 243-247 FEDLY (SEQ TD NO: 162), residues 259-263 AGKIT (SEQ ID NO: 164), and residues 532-538 (VEKLTLD) (SEQ ID NO: 165), of human TfR.

Also provided herein are antibodies comprising a VH comprising HCDR1, HCDR2, and HCDR3 selected from Table 1, and/or a VL comprising LCDR1, LCDR2, and LCDR3 selected from Table 2. In some embodiments, such antibodies comprise a VH and/or a VL selected from Table 3.

The TfR binding proteins or antibodies described herein can be recombinantly produced in a host cell, for example, using an expression vector. For example, an expression vector may include a sequence that encodes one or more signal peptides that facilitate secretion of the polypeptide(s) from a host cell. Expression vectors containing a polynucleotide of interest (e.g., a polynucleotide encoding a heavy chain or light chain of the TfR binding proteins or antibodies) may be transferred into a host cell by well-known methods. Additionally, expression vectors may contain one or more selection markers, e.g., tetracycline, neomycin, and dihydrofolate reductase, to aide in detection of host cells transformed with the desired polynucleotide sequences.

A host cell includes cells stably or transiently transfected, transformed, transduced or infected with one or more expression vectors expressing all or a portion of the TfR binding proteins or antibodies described herein. According to some embodiments, a host cell may be stably or transiently transfected, transformed, transduced or infected with an expression vector expressing HC polypeptides and an expression vector expressing LC polypeptides of the TfR binding proteins or antibodies described herein. In some embodiments, a host cell may be stably or transiently transfected, transformed, transduced or infected with an expression vector expressing HC and LC polypeptides of the TfR binding proteins or antibodies described herein. The TfR binding proteins or antibodies may be produced in mammalian cells such as CHO, NSO, HEK293 or COS cells according to techniques well known in the art.

Medium, into which the TfR binding proteins or antibodies has been secreted, may be purified by conventional techniques, such as mixed-mode methods of ion-exchange and hydrophobic interaction chromatography. For example, the medium may be applied to and eluted from a Protein A or G column using conventional methods; mixed-mode methods of ion-exchange and hydrophobic interaction chromatography may also be used. Soluble aggregate and multimers may be effectively removed by common techniques, including size exclusion, hydrophobic interaction, ion exchange, or hydroxyapatite chromatography. Various methods of protein purification may be employed, and such methods are known in the art and described, for example, in Deutscher, Methods in Enzymology 182: 83-89 (1990) and Scopes, Protein Purification: Principles and Practice, 3rd Edition, Springer, NY (1994).

### Mouse TfR Binding Proteins

In another aspect, provided herein are proteins comprising one monovalent mouse TfR binding domain (“mouse TfR binding proteins” or mTBP). These mouse TfR binding proteins can serve as surrogate molecules as the human TfR binding proteins described above in mouse models. In some embodiments, the monovalent mouse TfR binding domain comprises a heavy chain variable region (VH) and a light chain variable region (VL), and the VH comprises heavy chain complementarity determining regions HCDR1, HCDR2, and HCDR3, and the VL comprises light chain complementarity determining regions LCDR1, LCDR2, and LCDR3. In some embodiments, the monovalent mouse TfR binding domain comprises a VH comprising HCDR1, HCDR2, and HCDR3 selected from Table 7a, and/or a VL comprising LCDR1, LCDR2, and LCDR3 selected from Table 7a. In some embodiments, the monovalent human TfR binding domain comprises a VH and/or a VL selected from Table 7a.

In some embodiments, provided herein are proteins comprising one monovalent mouse TfR binding domain, wherein the mouse TfR binding domain comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 71, HCDR2 comprises SEQ ID NO: 72, HCDR3 comprises SEQ ID NO: 73, LCDR1 comprises SEQ ID NO: 74, LCDR2 comprises SEQ ID NO: 75, and LCDR3 comprises SEQ ID NO: 76. In some embodiments, provided herein are proteins comprising one monovalent mouse TfR binding domain, wherein the mouse TfR binding domain comprises a VH comprising SEQ ID NO: 77 and a VL comprising SEQ ID NO: 78.

In some embodiments, the mouse TfR binding protein described herein has one heavy chain (HC) and one light chain, e.g., mTBP1 in Table 7b. In some embodiments, the mouse TfR binding protein has two heavy chains (HC1 and HC2) and two light chains (LC1 and LC2), e.g., mTBP2 in Table 7b.

In some embodiments, provided herein are proteins comprising one monovalent mouse TfR binding domain, wherein the mouse TfR binding domain comprises a heavy chain (HC) comprising SEQ ID NO: 79 and a light chain (LC) comprising SEQ ID NO: 80.

In some embodiments, the mouse TfR binding proteins described herein are heterodimeric antibodies that comprise a first arm comprising one monovalent mouse TfR binding domain and a second arm that is a null arm that does not bind any known human target (e.g., an isotype arm). In some embodiments, provided herein are mouse TfR binding proteins comprise two heavy chains HC1 and HC2 and two light chains LC1 and LC2, wherein HC1 comprises SEQ ID NO: 79, LC1 comprises SEQ TD NO: 80, HC2 comprises SEQ TD NO: 51, and LC2 comprises SEQ ID NO: 52.

Also provided herein are antibodies comprising a VH comprising HCDR1, HCDR2, and HCDR3 selected from Table 7a, and/or a VL comprising LCDR1, LCDR2, and LCDR3 selected from Table 7a. In some embodiments, such antibodies comprise a VH and/or a VL selected from Table 7a.

TABLE 7a
Exemplary sequences of mouse
TfR binding domain

| | | SEQ |
|---|---|---|
| | | ID |
| Region | Sequence | NO |
| HCDR1 | GSYWIC | 71 |
| (KABAT) | | |
| HCDR2 | CIYSTSGGRTYYASWVKG | 72 |
| (KABAT) | | |
| HCDR3 | GDDSISDAYFDL | 73 |
| (KABAT) | | |
| LCDR1 | QSSQSVYNNNRLA | 74 |
| (KABAT) | | |
| LCDR2 | DASTLAS | 75 |
| (KABAT) | | |
| LCDR3 | QGTYFSSGWSWA | 76 |
| (KABAT) | | |
| VH | QSLEESGGDLVKPEGSLTLTCTASGFSFSGSYWICW | 77 |
| | VRQAPGKGLEWIGCIYSTSGGRTYYASWVKGRFTIS | |
| | KTSSTTVTLQMTSLTAADTATYFCARGDDSISDAYF | |
| | DLWGPGTLVTVSS | |
| VL | ALDMTQTASPVSAAVGGTVTINCQSSQSVYNNNRL | 78 |
| | AWYQQKPGQPPKLLIYDASTLASGVPSRFKGSGSG | |
| | TQFTLTISGVQSDDSATYYCQGTYFSSGWSWAFGG | |
| | GTEVVVK | |

TABLE 7b
Exemplary sequences of mouse
TfR binding proteins

| Mouse TfR | | | | |
|---|---|---|---|---|
| binding | | | | |
| protein | | | | |
| (mTBP) | HC1 | LC1 | HC2 | LC2 |
| mTBP1 | SEQ ID | SEQ ID | N/A | N/A |
| | NO: 79 | NO: 80 | | |
| mTBP2 | SEQ ID | SEQ ID | SEQ ID | SEQ ID |
| | NO: 79 | NO: 80 | NO: 51 | NO: 52 |

| | |
|---|---|
| SEQ ID | QSLEESGGDLVKPEGSLTLTCTASGFSFSG |
| NO: 79 | SYWICWVRQAPGKGLEWIGCIYSTSGGRTY |
| | YASWVKGRFTISKTSSTTVTLQMTSLTAAD |
| | TATYFCARGDDSISDAYFDLWGPGTLVTVS |
| | SASTKGPCVFPLAPCSRSTSESTAALGCLV |
| | KDYFPEPVTVSWNSGALTSGVHTFPAVLQS |
| | SGLYSLSSVVTVPSSSLGTKTYTCNVDHKP |
| | SNTKVDKRVESKYGPPCPPCPAPEAAGGPS |
| | VFLFPPKPKDTLMISRTPEVTCVVVDVSQE |
| | DPEVQFNWYVDGVEVHNAKTKPREEQFNST |
| | YRVVSVLTVLHQDWLNGKEYKCKVSNKGLP |
| | SSIEKTISKAKGQPREPQVYTLPPSQEEMT |
| | KNQVSLTCLVKGFYPSDIAVEWESNGQPEN |
| | NYKTTPPVLDSDGSFFLYSRLTVDKSRWQE |
| | GNVFSCSVMHEALHNHYTQKSLSLSLG |
| SEQ ID | ALDMTQTASPVSAAVGGTVTINCQSSQSVY |
| NO: 80 | NNNRLAWYQQKPGQPPKLLIYDASTLASGV |
| | PSRFKGSGSGTQFTLTISGVQSDDSATYYC |
| | QGTYFSSGWSWAFGGGTEVVVKRTVAAPSV |
| | FIFPPSDEQLKSGTASVVCLLNNFYPREAK |
| | VQWKVDNALQSGNSQESVTEQDSKDSTYSL |
| | SSTLTLSKADYEKHKVYACEVTHQGLSSPV |
| | TKSFNRGEC |

*N/A = not applicable, which means the TBP does not have that heavy or light chain.

### Conjugates Comprising Human or Mouse TfR Binding Protein

In another aspect, provided herein are conjugates comprising human or mouse TfR binding proteins or antibodies described herein and a therapeutic agent. In some embodiments, the therapeutic agent is selected from a double stranded RNA (e.g., siRNA, saRNA), oligonucleotide (e.g., antisense oligonucleotide), peptide, small molecule, nanoparticle, lipid nanoparticle, exosome, antibody or antigen binding fragment thereof, or a combination thereof. In some embodiments, the therapeutic agent is a double stranded RNA (dsRNA). In some embodiments, the dsRNA comprises a sense strand and an antisense stand, wherein the antisense strand is complementary to a target mRNA selected from SNCA, MAPT, APP, ATXN2, ATXN3, SARM1, APOE, BACE1, FMR1, LRRK2, HTT, SOD1, SCN10A, SCN9A or CACNA1B mRNA. In some embodiments, the dsRNA comprises a sense strand and an antisense stand, wherein the antisense strand is complementary to SNCA mRNA. In some embodiments, the dsRNA comprises a sense strand and an antisense stand, wherein the antisense strand is complementary to MAPT mRNA.

In some embodiments, the therapeutic agent to protein ratio is about 1 to 3. In some embodiments, the therapeutic agent to protein ratio is about 1. In some embodiments, the therapeutic agent to protein ratio is about 2. In some embodiments, the therapeutic agent to protein ratio is about 3.

In some embodiments, the human TfR binding proteins described herein comprise one or more native cysteine residues, which can be used for conjugation. For example, in some embodiments, the human TfR binding protein described herein comprises a native cysteine at position 220 of the light chain and/or a native cysteine at position 226 of the heavy chain, which can be used for conjugation (all residues according to the EU Index numbering).

In some embodiments, the human TfR binding proteins described herein comprise one or more engineered cysteine residues for conjugation. The approach of including engineered cysteines as a means for conjugation has been described in WO 2018/232088. In some embodiments, the human TfR binding proteins described herein comprise a heavy chain comprising one or more cysteines at the following residues: 124, 157, 162, 262, 373, 375, 378, 397, 415 (all residues according to the EU Index numbering). In some embodiments, the human TfR binding proteins described herein comprise a light chain (e.g., a kappa light chain) comprising one or more cysteines at the following residues: 156, 171, 191, 193, 202, 208 (all residues according to the EU Index numbering). In some embodiments, the human TfR binding proteins described herein comprise a heavy chain constant region comprising cysteine at residue 124 (according to the EU Index numbering). In some embodiments, the human TfR binding proteins described herein comprise a light chain constant region comprising cysteine at residue 156 (according to the EU Index numbering). In some embodiments, the human TfR binding proteins described herein comprise an immunoglobulin Fc region comprising cysteine at residue 378 (according to the EU Index numbering).

In some embodiments, the therapeutic agent is linked to the human or mouse TfR binding protein through a linker. In some embodiments, the linker is a Mal-Tet-TCO linker, SMCC linker, or GDM linker (structures of these linkers shown in Table 8).

TABLE 8
Exemplary linker structures

| Linker | Structure |
|---|---|
| 1 | Mal-Tet-TCO linker 1 |
| | 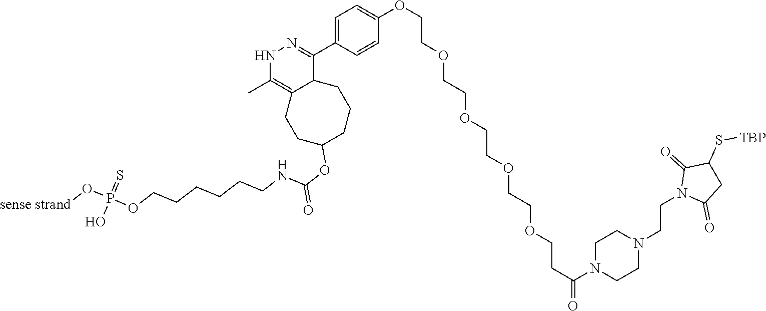 |
| 2 | SMCC linker 1 |
| | 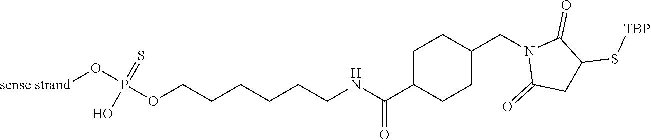 |
| 3 | GDM linker 1 |
| | 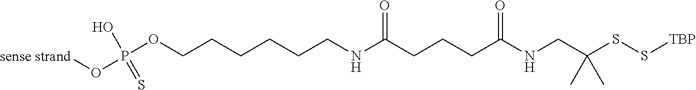 |
| 4 | Mal-Tet-TCO linker 2 |
| | 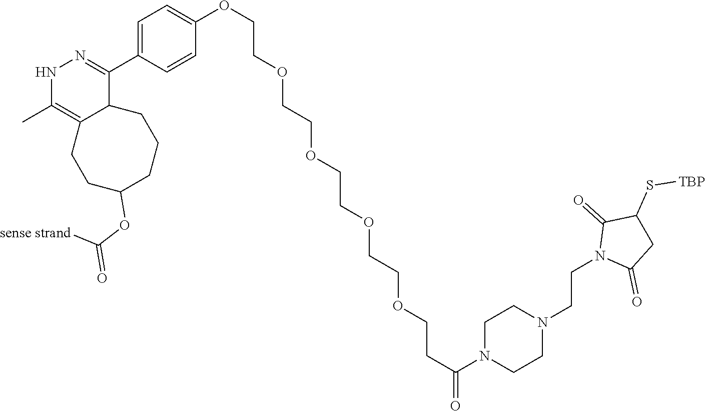 |
| 5 | SMCC linker 2 |
| | 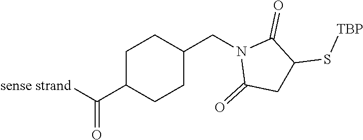 |
| 6 | GDM linker 2 |
| | 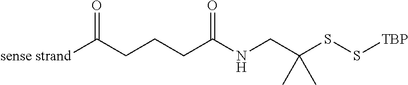 |
| 7 | Hydrolyzed ring open form of Mal-Tet-TCO linker 2 |
| | 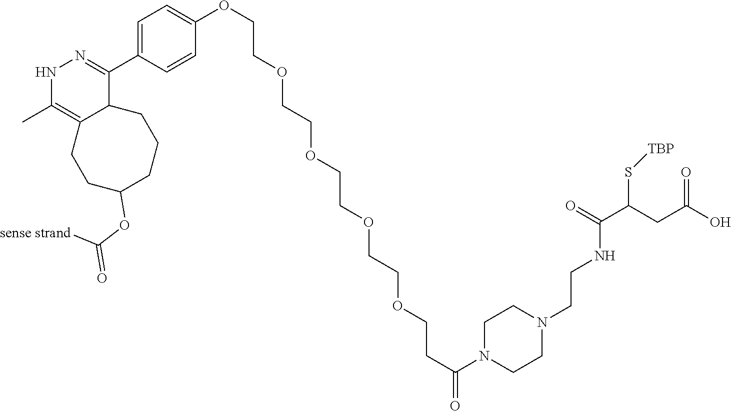 |
| 8 | Hydrolyzed ring open form of Mal-Tet-TCO linker 3 |
| | 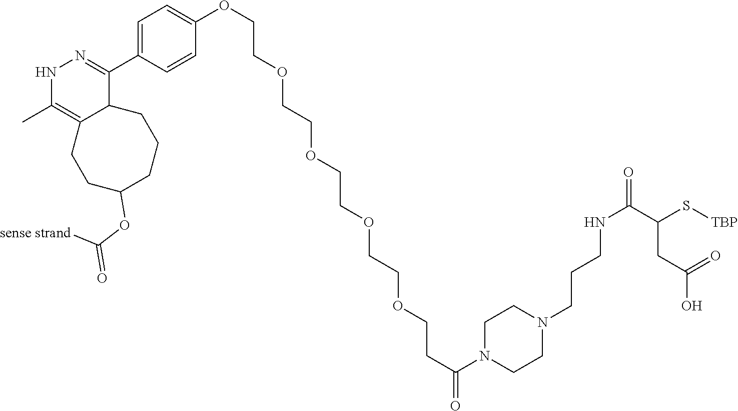 |
| 9 | Hydrolyzed ring open form of SMCC linker 1 |
| | 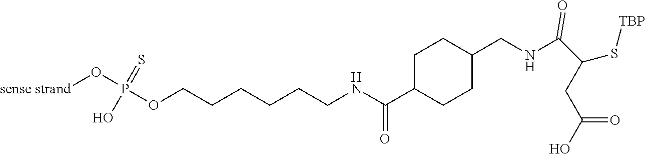 |
| 10 | Hydrolyzed ring open form of SMCC linker 2 |
| | 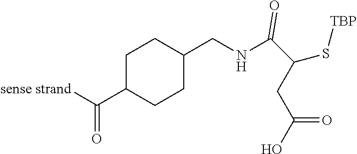 |

The conjugates described herein can be made by a variety of procedures known to one of ordinary skill in the art, some of which are illustrated in the preparations and examples below, e.g., in Example 3. One of ordinary skill in the art recognizes that the specific synthetic steps for each of the routes described may be combined in different ways, or in conjunction with steps from different schemes, to prepare conjugates. The product of each step can be recovered by conventional methods well known in the art, including extraction, evaporation, precipitation, chromatography, filtration, trituration, and crystallization. The reagents and starting materials are readily available to one of ordinary skill in the art.

In some embodiments, the TfR binding proteins with native or engineered cysteines described herein can be first treated with a reducing agent, e.g., DTT, and then re-oxidized with an oxidizing agent, e.g., DHAA. The resulting oxidized TfR binding proteins are then incubated with a linker functionalized therapeutic agent, e.g., linker-dsRNA, to produce the conjugates.

### Human TfR Binding Proteins-dsRNA Conjugates

In some embodiments, provided herein are conjugates of Formula (I): R-L-P, wherein R is a double stranded RNA (dsRNA) comprising a sense stand and an antisense strand; wherein P is a protein comprising one monovalent human or mouse TfR binding domain; and wherein L is a linker, or optionally absent. In some embodiments, P is a human or mouse TfR binding protein described herein. In some embodiments, the R to P ratio is about 1 to 3. In some embodiments, the R to P ratio is about 1. In some embodiments, the R to P ratio is about 2. In some embodiments, the R to P ratio is about 3.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human or mouse TfR binding domain; wherein L is a linker, or optionally absent, and wherein n is 1 to 3. In some embodiments, n is 1. In some embodiments, n is 2. In some embodiments, n is 3.

In some embodiments, provided herein are conjugates of Formula (I): R-L-P, wherein R is a double stranded RNA (dsRNA) comprising a sense stand and an antisense strand; wherein P is a protein comprising one monovalent human TfR binding domain; and wherein L is a linker, or optionally absent, herein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 21, HCDR3 comprises SEQ ID NO: 22, LCDR1 comprises SEQ ID NO: 23, LCDR2 comprises SEQ ID NO: 5, and LCDR3 comprises SEQ ID NO: 24; or
(b) HCDR1 comprises SEQ ID NO: 13, HCDR2 comprises SEQ ID NO: 25, HCDR3 comprises SEQ ID NO: 26, LCDR1 comprises SEQ ID NO: 16, LCDR2 comprises SEQ ID NO: 17, and LCDR3 comprises SEQ ID NO: 18.

In some embodiments, provided herein are conjugates of Formula (I): R-L-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6;
(b) HCDR1 comprises SEQ ID NO: 1, HCDR2 comprises SEQ ID NO: 2, HCDR3 comprises SEQ ID NO: 7, LCDR1 comprises SEQ ID NO: 4, LCDR2 comprises SEQ ID NO: 5, and LCDR3 comprises SEQ ID NO: 6;
(c) HCDR1 comprises SEQ ID NO: 1, HCDR2 comprises SEQ ID NO: 2, HCDR3 comprises SEQ ID NO: 8, LCDR1 comprises SEQ ID NO: 9, LCDR2 comprises SEQ ID NO: 5, and LCDR3 comprises SEQ ID NO: 6;
(d) HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12;
(e) HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 17, and LCDR3 comprises SEQ ID NO: 18;
(f) HCDR1 comprises SEQ ID NO: 13, HCDR2 comprises SEQ ID NO: 19, HCDR3 comprises SEQ ID NO: 15, LCDR1 comprises SEQ ID NO: 16, LCDR2 comprises SEQ ID NO: 17, and LCDR3 comprises SEQ ID NO: 18; or
(g) HCDR1 comprises SEQ ID NO: 13, HCDR2 comprises SEQ ID NO: 19, HCDR3 comprises SEQ ID NO: 20, LCDR1 comprises SEQ ID NO: 16, LCDR2 comprises SEQ ID NO: 17, and LCDR3 comprises SEQ ID NO: 18.

In some embodiments, provided herein are conjugates of Formula (I): R-L-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH and VL comprise the following sequences:
(a) VH comprises a sequence having at least 95% sequence identity to SEQ ID NO: 27 and VL comprises a sequence having at least 95% sequence identity to SEQ ID NO: 28;
(b) VH comprises a sequence having at least 95% sequence identity to SEQ ID NO: 29 and VL comprises a sequence having at least 95% sequence identity to SEQ ID NO: 28;
(c) VH comprises a sequence having at least 95% sequence identity to SEQ ID NO: 30 and VL comprises a sequence having at least 95% sequence identity to SEQ ID NO: 31;
(d) VH comprises a sequence having at least 95% sequence identity to SEQ ID NO: 32 and VL comprises a sequence having at least 95% sequence identity to SEQ ID NO: 33;
(e) VH comprises a sequence having at least 95% sequence identity to SEQ ID NO: 34 and VL comprises a sequence having at least 95% sequence identity to SEQ ID NO: 35;
(f) VH comprises a sequence having at least 95% sequence identity to SEQ ID NO: 36 and VL comprises a sequence having at least 95% sequence identity to SEQ ID NO: 37; or
(g) VH comprises a sequence having at least 95% sequence identity to SEQ ID NO: 38 and VL comprises a sequence having at least 95% sequence identity to SEQ ID NO: 37.

In some embodiments, provided herein are conjugates of Formula (I): R-L-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH and VL comprise the following sequences:
(a) VH comprises SEQ ID NO: 27 and VL comprises SEQ ID NO: 28;
(b) VH comprises SEQ ID NO: 29 and VL comprises SEQ ID NO: 28;
(c) VH comprises SEQ ID NO: 30 and VL comprises SEQ ID NO: 31;
(d) VH comprises SEQ ID NO: 32 and VL comprises SEQ ID NO: 33;
(e) VH comprises SEQ ID NO: 34 and VL comprises SEQ ID NO: 35;
(f) VH comprises SEQ ID NO: 36 and VL comprises SEQ ID NO: 37; or
(g) VH comprises SEQ ID NO: 38 and VL comprises SEQ ID NO: 37.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, herein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 21, HCDR3 comprises SEQ ID NO: 22, LCDR1 comprises SEQ ID NO: 23, LCDR2 comprises SEQ ID NO: 5, and LCDR3 comprises SEQ ID NO: 24; or
(b) HCDR1 comprises SEQ ID NO: 13, HCDR2 comprises SEQ ID NO: 25, HCDR3 comprises SEQ ID NO: 26, LCDR1 comprises SEQ ID NO: 16, LCDR2 comprises SEQ ID NO: 17, and LCDR3 comprises SEQ ID NO: 18, and wherein n is 1 to 3.

In some embodiments, n is 1. In some embodiments, n is 2. In some embodiments, n is 3.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6;
(b) HCDR1 comprises SEQ ID NO: 1, HCDR2 comprises SEQ ID NO: 2, HCDR3 comprises SEQ ID NO: 7, LCDR1 comprises SEQ ID NO: 4, LCDR2 comprises SEQ ID NO: 5, and LCDR3 comprises SEQ ID NO: 6;
(c) HCDR1 comprises SEQ ID NO: 1, HCDR2 comprises SEQ ID NO: 2, HCDR3 comprises SEQ ID NO: 8, LCDR1 comprises SEQ ID NO: 9, LCDR2 comprises SEQ ID NO: 5, and LCDR3 comprises SEQ ID NO: 6;
(d) HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12;
(e) HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 17, and LCDR3 comprises SEQ ID NO: 18;
(f) HCDR1 comprises SEQ ID NO: 13, HCDR2 comprises SEQ ID NO: 19, HCDR3 comprises SEQ ID NO: 15, LCDR1 comprises SEQ ID NO: 16, LCDR2 comprises SEQ ID NO: 17, and LCDR3 comprises SEQ ID NO: 18; or
(g) HCDR1 comprises SEQ ID NO: 13, HCDR2 comprises SEQ ID NO: 19, HCDR3 comprises SEQ ID NO: 20, LCDR1 comprises SEQ ID NO: 16, LCDR2 comprises SEQ ID NO: 17, and LCDR3 comprises SEQ ID NO: 18, and wherein n is 1 to 3.

In some embodiments, n is 1. In some embodiments, n is 2. In some embodiments, n is 3.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH and VL comprise the following sequences:
(a) VH comprises a sequence having at least 95% sequence identity to SEQ ID NO: 27 and VL comprises a sequence having at least 95% sequence identity to SEQ ID NO: 28;
(b) VH comprises a sequence having at least 95% sequence identity to SEQ ID NO: 29 and VL comprises a sequence having at least 95% sequence identity to SEQ ID NO: 28;
(c) VH comprises a sequence having at least 95% sequence identity to SEQ ID NO: 30 and VL comprises a sequence having at least 95% sequence identity to SEQ ID NO: 31;
(d) VH comprises a sequence having at least 95% sequence identity to SEQ ID NO: 32 and VL comprises a sequence having at least 95% sequence identity to SEQ ID NO: 33;
(e) VH comprises a sequence having at least 95% sequence identity to SEQ ID NO: 34 and VL comprises a sequence having at least 95% sequence identity to SEQ ID NO: 35;
(f) VH comprises a sequence having at least 95% sequence identity to SEQ ID NO: 36 and VL comprises a sequence having at least 95% sequence identity to SEQ ID NO: 37; or
(g) VH comprises a sequence having at least 95% sequence identity to SEQ ID NO: 38 and VL comprises a sequence having at least 95% sequence identity to SEQ ID NO: 37, and wherein n is 1 to 3.

In some embodiments, n is 1. In some embodiments, n is 2. In some embodiments, n is 3.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH and VL comprise the following sequences:
(a) VH comprises SEQ ID NO: 27 and VL comprises SEQ ID NO: 28;
(b) VH comprises SEQ ID NO: 29 and VL comprises SEQ ID NO: 28;
(c) VH comprises SEQ ID NO: 30 and VL comprises SEQ ID NO: 31;
(d) VH comprises SEQ ID NO: 32 and VL comprises SEQ ID NO: 33;
(e) VH comprises SEQ ID NO: 34 and VL comprises SEQ ID NO: 35;
(f) VH comprises SEQ ID NO: 36 and VL comprises SEQ ID NO: 37; or
(g) VH comprises SEQ ID NO: 38 and VL comprises SEQ ID NO: 37, and wherein n is 1 to 3.

In some embodiments, n is 1. In some embodiments, n is 2. In some embodiments, n is 3.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12, and wherein n is 1 to 3. In some embodiments, n is 1. In some embodiments, n is 2. In some embodiments, n is 3.

In some embodiments, provided herein are conjugates of Formula (II): (R-L)ₙ-P, wherein R is a double stranded RNA (dsRNA) comprising a sense stand and an antisense strand; wherein P is a protein comprising one monovalent human TfR binding domain; and wherein L is a linker, or optionally absent, wherein the human TfR binding domain comprises a heavy chain variable region (VH) and a light chain variable region (VL), wherein the VH comprises SEQ ID NO: 32 and VL comprises SEQ ID NO: 33, and wherein n is 1 to 3. In some embodiments, n is 1. In some embodiments, n is 2. In some embodiments, n is 3.

In some embodiments, the protein (P) also binds cynomolgus monkey TfR. In some embodiments, the human TfR binding domain of the protein (P) is a Fab, scFv, Fv, or scFab. In some embodiments, the human TfR binding domain of the protein (P) is a Fab. In some embodiments, the human TfR binding domain the protein (P) further comprises a heavy chain constant region comprising cysteine at residue 124 (according to the EU Index numbering). In some embodiments, the human TfR binding domain the protein (P) further comprises a light chain constant region comprising cysteine at residue 156 (according to the EU Index numbering).

In some embodiments, the protein (P) further comprises a half-life extender, e.g., an immunoglobulin Fc region or a VHH that binds human serum albumin (HSA). In some embodiments, the protein (P) comprises an immunoglobulin Fc region, e.g., a modified human IgG4 Fc region or a modified human IgG1 Fc region. In some embodiments, the protein (P) comprises a modified human IgG4 Fc region comprising proline at residue 228, and alanine at residues 234 and 235 (all residues are numbered according to the EU Index numbering, also called hIgG4PAA Fc region). In some embodiments, the protein (P) comprises a modified human IgG1 Fc region comprising alanine at residues 234, 235, and 329, serine at position 265, aspartic acid at position 436 (all residues are numbered according to the EU Index numbering, also called hIgG1 effector null or hIgG1EN Fc region). In some embodiments, the protein (P) comprise a modified human IgG1 or IgG4 Fc region, wherein the Fc region comprises a first Fc CH3 domain comprising a serine at position 349, a methionine at position 366, a tyrosine at position 370, and a valine at position 409; and a second Fc CH3 domain comprising a glycine at position 356, an aspartic acid at position 357, a glutamine at position 364, and an alanine at position 407 (all residues are numbered according to the EU Index numbering). In some embodiments, the protein (P) comprises a modified human IgG1 or IgG4 Fc region comprising a first Fc CH3 domain comprising leucine at residue 405, and a second Fc CH3 domain comprising arginine at residue 409 (all residues are numbered according to the EU Index numbering).

In some embodiments, the protein (P) comprises a VHH that binds human HSA. In some embodiments, the VHH also binds mouse, rat, and/or cynomolgus monkey albumin. In some embodiments, such a VHH comprises CDR1 comprising SEQ ID NO: 39, CDR2 comprising SEQ ID NO: 40, and CDR3 comprising SEQ ID NO: 41. In some embodiments, such a VHH comprises SEQ ID NO: 42. In some embodiments, the VHH is linked to the TfR binding domain through a peptide linker, e.g., (GGGGQ)₄ (SEQ ID NO: 70).

In some embodiments, the protein (P) comprises one heavy chain (HC) and one light chain (LC), wherein the HC and LC comprise the following sequences:
(a) HC comprises SEQ ID NO: 53 and LC comprises SEQ ID NO: 54;
(b) HC comprises SEQ ID NO: 55 and LC comprises SEQ ID NO: 54;
(c) HC comprises SEQ ID NO: 56 and LC comprises SEQ ID NO: 57;
(d) HC comprises SEQ ID NO: 58 and LC comprises SEQ ID NO: 59;
(e) HC comprises SEQ ID NO: 60 and LC comprises SEQ ID NO: 61;
(f) HC comprises SEQ ID NO: 62 and LC comprises SEQ ID NO: 63; or
(g) HC comprises SEQ ID NO: 64 and LC comprises SEQ ID NO: 63.

In some embodiments, the protein (P) comprises one HC and one LC, and wherein the HC comprises SEQ ID NO: 65 and the LC comprises SEQ ID NO: 59.

In some embodiments, the protein (P) comprises one HC and one LC, and wherein the HC comprises SEQ ID NO: 66 and the LC comprises SEQ ID NO: 67.

In some embodiments, the protein (P) comprises two heavy chains HC1 and HC2 and one light chain LC1, wherein HC1 comprises SEQ ID NO: 68, LC1 comprises SEQ ID NO: 59, HC2 comprises SEQ ID NO: 69.

In some embodiments, the protein (P) comprises two heavy chains HC1 and HC2 and one light chain LC1, wherein HC1 comprises SEQ ID NO: 138, LC1 comprises SEQ ID NO: 59, HC2 comprises SEQ ID NO: 139.

In some embodiments, the protein (P) comprises two heavy chains HC1 and HC2 and one light chain LC1, wherein HC1 comprises SEQ ID NO: 166, LC1 comprises SEQ ID NO: 54, HC2 comprises SEQ ID NO: 167.

In some embodiments, the protein (P) is a heterodimeric antibody that comprises a first arm comprising one monovalent human TfR binding domain and a second arm that is a null arm, e.g., an arm that does not bind any known human target, e.g., the isotype arm in Table 5.

In some embodiments, the protein (P) comprises two heavy chains HC1 and HC2 and two light chains LC1 and LC2, wherein HC1, LC1, HC2, and LC2 comprise the following sequences:
(a) HC1 comprises SEQ ID NO: 64, LC1 comprises SEQ ID NO: 63, HC2 comprises SEQ ID NO: 51, and LC2 comprises SEQ ID NO: 52;
(b) HC1 comprises SEQ ID NO: 55, LC1 comprises SEQ ID NO: 54, HC2 comprises SEQ ID NO: 51, and LC2 comprises SEQ ID NO: 52;
(c) HC1 comprises SEQ ID NO: 56, LC1 comprises SEQ ID NO: 57, HC2 comprises SEQ ID NO: 51, and LC2 comprises SEQ ID NO: 52; or
(d) HC1 comprises SEQ ID NO: 58, LC1 comprises SEQ ID NO: 59, HC2 comprises SEQ ID NO: 51, and LC2 comprises SEQ ID NO: 52.

In some embodiments, the linker (L) is present and selected from: a Mal-Tet-TCO linker, SMCC linker, or GDM linker (see Table 8). In some embodiments, the linker (L) is absent.

In some embodiments, the protein (P) is linked to the 3′ end of the sense strand of the dsRNA. In some embodiments, the protein (P) is linked to the 5′ end of the sense strand of the dsRNA. In some embodiments, the protein (P) is linked to an internal position of the sense strand of the dsRNA. In some embodiments, the protein (P) is linked to the 3′ end of the antisense strand of the dsRNA. In some embodiments, the protein (P) is linked to an internal position of the antisense strand of the dsRNA.

In some embodiments, the dsRNA comprises an antisense strand complementary to a target mRNA selected from SNCA, MAPT, APP, ATXN2, ATXN3, SARM1, APOE, BACE1, FMR1, LRRK2, HTT, SOD1, SCN10A, SCN9A or CACNA1B mRNA. In some embodiments, the dsRNA comprises an antisense strand complementary to SNCA mRNA. In some embodiments, the dsRNA comprises an antisense strand complementary to MAPT mRNA.

In some embodiments, the sense strand and the antisense strand of the dsRNA are each 15-30 nucleotides in length, e.g., 20-25 nucleotides in length. In some embodiments, the dsRNA has a sense strand of 21 nucleotides and an antisense strand of 23 nucleotides. In some embodiments, the sense strand and antisense strand of the dsRNA may have overhangs at either the 5′ end or the 3′ end (i.e., 5′ overhang or 3′ overhang). For example, the sense strand and the antisense strand may have 5′ or 3′ overhangs of 1 to 5 nucleotides or 1 to 3 nucleotides. In some embodiments, the antisense strand comprises a 3′ overhang of two nucleotides.

Exemplary unmodified sense strand and antisense strand sequences of dsRNA targeting human SNCA mRNA are provided in Table 9a. Exemplary unmodified sense strand and antisense strand sequences of dsRNA targeting human MAPT mRNA are provided in Table 9b.

TABLE 9a
Unmodified Nucleic Acid Sequences of
dsRNA targeting human SNCA mRNA
(SNCA siRNA)

| | | | | | Start |
|---|---|---|---|---|---|
| | | | | | posi- |
| | | | | | tion |
| | | | | | of |
| | | | | | target |
| | | | | | region |
| | | | | | on |
| | | | | | human |
| | | | | | SNCA |
| | | | | | tran- |
| | Sense | SEQ | Antisense | SEQ | script |
| dsRNA | Strand | ID | Strand | ID | NM_000 |
| No. | (5′ to 3′) | NO | (5′ to 3′) | NO | 345.4 |
| 1 | CUGUAC | 81 | UGGAAC | 82 | 701 |
| | AAGUG | | UGAGCA | | |
| | CUCAGU | | CUUGUA | | |
| | UCCA | | CAGGA | | |
| 2 | UGUACA | 83 | UUGGAA | 84 | 702 |
| | AGUGC | | CUGAGC | | |
| | UCAGUU | | ACUUGU | | |
| | CCAA | | ACAGG | | |
| 3 | GAGCAA | 85 | UCCAAC | 86 | 408 |
| | GUGAC | | AUUUGU | | |
| | AAAUGU | | CACUUG | | |
| | UGGA | | CUCUU | | |
| 4 | UUCCAA | 87 | UCAUGA | 88 | 717 |
| | UGUGC | | CUGGGC | | |
| | CCAGUC | | ACAUUG | | |
| | AUGA | | GAACU | | |
| 5 | AGUGAC | 89 | UAGAAA | 90 | 926 |
| | UACCA | | UAAGUG | | |
| | CUUAUU | | GUAGUC | | |
| | UCUA | | ACUUA | | |
| 6 | GUGACU | 91 | UUAGAA | 92 | 927 |
| | ACCAC | | AUAAGU | | |
| | UUAUUU | | GGUAGU | | |
| | CUAA | | CACUU | | |
| 7 | CUGUAC | 116 | UGGAAC | 82 | 701 |
| | AAGnG | | UGAGCA | | |
| | CUCAGU | | CUUGUA | | |
| | UCCA, | | CAGGA | | |
| | wherein | | | | |
| | n is | | | | |
| | an abasic | | | | |
| | moiety. | | | | |

TABLE 9b
Unmodified Nucleic Acid Sequences
of dsRNA targeting human MAPT mRNA
(MAPT siRNA)

| | | | | | Start |
|---|---|---|---|---|---|
| | | | | | position |
| | | | | | of target |
| | | | | | region on |
| | | | | | human |
| | | | | | MAPT |
| | Sense | SEQ | Antisense | SEQ | transcript |
| dsRNA | Strand | ID | Strand | ID | NM_0011 |
| No. | (5′ to 3′) | NO | (5′ to 3′) | NO | 23067.4 |
| 20 | GUGGAAGU | 120 | UUUCUCAGA | 121 | 1070 |
| | AAAAUCUG | | UUUUACUUC | | |
| | AGAAA | | CACCU | | |
| 21 | CCAAGUGU | 122 | UGCCUAAUG | 123 | 1020 |
| | GGCUCAUU | | AGCCACACU | | |
| | AGGCA | | UGGAG | | |
| 22 | UGCAAAUA | 124 | UUGGUUUGU | 125 | 978* |
| | GUCUACAA | | AGACUAUUU | | |
| | ACCAA | | GCACC | | |

*The last nucleotide does not match the transcript.

In some embodiments, the dsRNA targets SNCA mRNA. In some embodiments, the sense strand and the antisense strand of the dsRNA comprise a pair of nucleic acid sequences selected from the group consisting of:
(a) the sense strand comprises a first nucleic acid sequence having at least 90% sequence identity to SEQ ID NO: 81, and the antisense strand comprises a second nucleic acid sequence having at least 90% sequence identity to SEQ ID NO: 82;
(b) the sense strand comprises a first nucleic acid sequence having at least 90% sequence identity to SEQ ID NO: 83, and the antisense strand comprises a second nucleic acid sequence having at least 90% sequence identity to SEQ ID NO: 84;
(c) the sense strand comprises a first nucleic acid sequence having at least 90% sequence identity to SEQ ID NO: 85, and the antisense strand comprises a second nucleic acid sequence having at least 90% sequence identity to SEQ ID NO: 86;
(d) the sense strand comprises a first nucleic acid sequence having at least 90% sequence identity to SEQ ID NO: 87, and the antisense strand comprises a second nucleic acid sequence having at least 90% sequence identity to SEQ ID NO: 88;
(e) the sense strand comprises a first nucleic acid sequence having at least 90% sequence identity to SEQ ID NO: 89, and the antisense strand comprises a second nucleic acid sequence having at least 90% sequence identity to SEQ ID NO: 90;
(f) the sense strand comprises a first nucleic acid sequence having at least 90% sequence identity to SEQ ID NO: 91, and the antisense strand comprises a second nucleic acid sequence having at least 90% sequence identity to SEQ ID NO: 92; and
(g) the sense strand comprises a first nucleic acid sequence having at least 90% sequence identity to SEQ ID NO: 116, and the antisense strand comprises a second nucleic acid sequence having at least 90% sequence identity to SEQ ID NO: 82, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand of the dsRNA comprise a pair of nucleic acid sequences selected from the group consisting of:
(a) the sense strand comprises a first nucleic acid sequence having at least 95% sequence identity to SEQ ID NO: 81, and the antisense strand comprises a second nucleic acid sequence having at least 95% sequence identity to SEQ ID NO: 82;
(b) the sense strand comprises a first nucleic acid sequence having at least 95% sequence identity to SEQ ID NO: 83, and the antisense strand comprises a second nucleic acid sequence having at least 95% sequence identity to SEQ ID NO: 84;
(c) the sense strand comprises a first nucleic acid sequence having at least 95% sequence identity to SEQ ID NO: 85, and the antisense strand comprises a second nucleic acid sequence having at least 95% sequence identity to SEQ ID NO: 86;
(d) the sense strand comprises a first nucleic acid sequence having at least 95% sequence identity to SEQ ID NO: 87, and the antisense strand comprises a second nucleic acid sequence having at least 95% sequence identity to SEQ ID NO: 88;
(e) the sense strand comprises a first nucleic acid sequence having at least 95% sequence identity to SEQ ID NO: 89, and the antisense strand comprises a second nucleic acid sequence having at least 95% sequence identity to SEQ ID NO: 90;
(f) the sense strand comprises a first nucleic acid sequence having at least 95% sequence identity to SEQ ID NO: 91, and the antisense strand comprises a second nucleic acid sequence having at least 95% sequence identity to SEQ ID NO: 92; and
(g) the sense strand comprises a first nucleic acid sequence having at least 95% sequence identity to SEQ ID NO: 116, and the antisense strand comprises a second nucleic acid sequence having at least 95% sequence identity to SEQ ID NO: 82, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand of the dsRNA comprise a pair of nucleic acid sequences selected from the group consisting of:
(a) the sense strand comprises SEQ ID NO: 81, and the antisense strand comprises SEQ ID NO: 82;
(b) the sense strand comprises SEQ ID NO: 83, and the antisense strand comprises SEQ ID NO: 84;
(c) the sense strand comprises SEQ ID NO: 85, and the antisense strand comprises SEQ ID NO: 86;
(d) the sense strand comprises SEQ ID NO: 87, and the antisense strand comprises SEQ ID NO: 88;
(e) the sense strand comprises SEQ ID NO: 89, and the antisense strand comprises SEQ ID NO: 90;
(f) the sense strand comprises SEQ ID NO: 91, and the antisense strand comprises SEQ ID NO: 92; and
(g) the sense strand comprises SEQ ID NO: 116, and the antisense strand comprises SEQ ID NO: 82, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the dsRNA targets MAPT mRNA. In some embodiments, the sense strand and the antisense strand of the dsRNA comprise a pair of nucleic acid sequences selected from the group consisting of:
(a) the sense strand comprises a first nucleic acid sequence having at least 90% sequence identity to SEQ ID NO: 120, and the antisense strand comprises a second nucleic acid sequence having at least 90% sequence identity to SEQ ID NO: 121;
(b) the sense strand comprises a first nucleic acid sequence having at least 90% sequence identity to SEQ ID NO: 122, and the antisense strand comprises a second nucleic acid sequence having at least 90% sequence identity to SEQ ID NO: 123; and
(c) the sense strand comprises a first nucleic acid sequence having at least 90% sequence identity to SEQ ID NO: 124, and the antisense strand comprises a second nucleic acid sequence having at least 90% sequence identity to SEQ ID NO: 125, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand of the dsRNA comprise a pair of nucleic acid sequences selected from the group consisting of:
(a) the sense strand comprises a first nucleic acid sequence having at least 95% sequence identity to SEQ ID NO: 120, and the antisense strand comprises a second nucleic acid sequence having at least 95% sequence identity to SEQ ID NO: 121;
(b) the sense strand comprises a first nucleic acid sequence having at least 95% sequence identity to SEQ ID NO: 122, and the antisense strand comprises a second nucleic acid sequence having at least 95% sequence identity to SEQ ID NO: 123; and
(c) the sense strand comprises a first nucleic acid sequence having at least 95% sequence identity to SEQ ID NO: 124, and the antisense strand comprises a second nucleic acid sequence having at least 95% sequence identity to SEQ ID NO: 125, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, the sense strand and the antisense strand of the dsRNA comprise a pair of nucleic acid sequences selected from the group consisting of:
(a) the sense strand comprises SEQ ID NO: 120, and the antisense strand comprises SEQ ID NO: 121;
(b) the sense strand comprises SEQ ID NO: 122, and the antisense strand comprises SEQ ID NO: 123; and
(c) the sense strand comprises SEQ ID NO: 124, and the antisense strand comprises SEQ ID NO: 125, wherein optionally one or more nucleotides of the sense strand and the antisense strand are independently modified nucleotides, and wherein optionally one or more internucleotide linkages of the sense strand and the antisense strand are modified internucleotide linkages.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 81, and the antisense strand comprises SEQ ID NO: 82; wherein P is a protein comprising one monovalent human TfR binding domain; and wherein L is a linker or absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 81, and the antisense strand comprises SEQ ID NO: 82; wherein P is a protein comprising one monovalent human TfR binding domain; and wherein L is a linker or absent, wherein the human TfR binding domain comprises a heavy chain variable region (VH) and a light chain variable region (VL), wherein the VH comprises SEQ ID NO: 32 and VL comprises SEQ ID NO: 33, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 81, and the antisense strand comprises SEQ ID NO: 82; wherein P is a protein comprising one monovalent human TfR binding domain, wherein P comprises two heavy chains HC1 and HC2 and one light chain LC1, wherein HC1 comprises SEQ ID NO: 68, LC1 comprises SEQ ID NO: 59, HC2 comprises SEQ ID NO: 69; and wherein L is a linker or absent, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 81, and the antisense strand comprises SEQ ID NO: 82; wherein P is a protein comprising one monovalent human TfR binding domain, wherein P comprises two heavy chains HC1 and HC2 and one light chain LC1, wherein HC1 comprises SEQ ID NO: 138, LC1 comprises SEQ ID NO: 59, HC2 comprises SEQ ID NO: 139; and wherein L is a linker or absent, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 120, and the antisense strand comprises SEQ ID NO: 121; wherein P is a protein comprising one monovalent human TfR binding domain; and wherein L is a linker or absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 120, and the antisense strand comprises SEQ ID NO: 121; wherein P is a protein comprising one monovalent human TfR binding domain; and wherein L is a linker or absent, wherein the human TfR binding domain comprises a heavy chain variable region (VH) and a light chain variable region (VL), wherein the VH comprises SEQ ID NO: 32 and VL comprises SEQ ID NO: 33, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 120, and the antisense strand comprises SEQ ID NO: 121; wherein P is a protein comprising one monovalent human TfR binding domain, wherein P comprises two heavy chains HC1 and HC2 and one light chain LC1, wherein HC1 comprises SEQ ID NO: 68, LC1 comprises SEQ ID NO: 59, HC2 comprises SEQ ID NO: 69; and wherein L is a linker or absent, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 120, and the antisense strand comprises SEQ ID NO: 121; wherein P is a protein comprising one monovalent human TfR binding domain, wherein P comprises two heavy chains HC1 and HC2 and one light chain LC1, wherein HC1 comprises SEQ ID NO: 138, LC1 comprises SEQ ID NO: 59, HC2 comprises SEQ ID NO: 139; and wherein L is a linker or absent, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 122, and the antisense strand comprises SEQ ID NO: 123; wherein P is a protein comprising one monovalent human TfR binding domain; and wherein L is a linker or absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 122, and the antisense strand comprises SEQ ID NO: 123; wherein P is a protein comprising one monovalent human TfR binding domain; and wherein L is a linker or absent, wherein the human TfR binding domain comprises a heavy chain variable region (VH) and a light chain variable region (VL), wherein the VH comprises SEQ ID NO: 32 and VL comprises SEQ ID NO: 33, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 122, and the antisense strand comprises SEQ ID NO: 123; wherein P is a protein comprising one monovalent human TfR binding domain, wherein P comprises two heavy chains HC1 and HC2 and one light chain LC1, wherein HC1 comprises SEQ ID NO: 68, LC1 comprises SEQ ID NO: 59, HC2 comprises SEQ ID NO: 69; and wherein L is a linker or absent, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 122, and the antisense strand comprises SEQ ID NO: 123; wherein P is a protein comprising one monovalent human TfR binding domain, wherein P comprises two heavy chains HC1 and HC2 and one light chain LC1, wherein HC1 comprises SEQ ID NO: 138, LC1 comprises SEQ ID NO: 59, HC2 comprises SEQ ID NO: 139; and wherein L is a linker or absent, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 124, and the antisense strand comprises SEQ ID NO: 125; wherein P is a protein comprising one monovalent human TfR binding domain; and wherein L is a linker or absent, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 124, and the antisense strand comprises SEQ ID NO: 125; wherein P is a protein comprising one monovalent human TfR binding domain; and wherein L is a linker or absent, wherein the human TfR binding domain comprises a heavy chain variable region (VH) and a light chain variable region (VL), wherein the VH comprises SEQ ID NO: 32 and VL comprises SEQ ID NO: 33, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 124, and the antisense strand comprises SEQ ID NO: 125; wherein P is a protein comprising one monovalent human TfR binding domain, wherein P comprises two heavy chains HC1 and HC2 and one light chain LC1, wherein HC1 comprises SEQ ID NO: 68, LC1 comprises SEQ ID NO: 59, HC2 comprises SEQ ID NO: 69; and wherein L is a linker or absent, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

In some embodiments, provided herein are conjugates of Formula (II): (R-L)ₙ-P, wherein R is a double stranded RNA (dsRNA) comprising a sense stand and an antisense strand, wherein the sense strand comprises SEQ ID NO: 124, and the antisense strand comprises SEQ ID NO: 125; wherein P is a protein comprising one monovalent human TfR binding domain, wherein P comprises two heavy chains HC1 and HC2 and one light chain LC1, wherein HC1 comprises SEQ ID NO: 138, LC1 comprises SEQ ID NO: 59, HC2 comprises SEQ ID NO: 139; and wherein L is a linker or absent, and wherein n is 1 to 3. In some embodiments, n is 1. In some embodiments, n is 2. In some embodiments, n is 3. In some embodiments, L is a linker in Table 8. In some embodiments, L is a SMCC linker in Table 8.

The dsRNA can include modifications. The modifications can be made to one or more nucleotides of the sense and/or antisense strand or to the internucleotide linkages, which are the bonds between two nucleotides in the sense or antisense strand. For example, some 2′-modifications of ribose or deoxyribose can increase RNA or DNA stability and half-life. Such 2′-modifications can be 2′-fluoro, 2′-O-methyl (i.e., 2′-methoxy), or 2′-O-alkyl.

In some embodiments, one or more nucleotides of the sense strand and/or the antisense strand are independently modified nucleotides, which means the sense strand and the antisense strand can have different modified nucleotides. In some embodiments, each nucleotide of the sense strand is a modified nucleotide. In some embodiments, each nucleotide of the antisense strand is a modified nucleotide. In some embodiments, the modified nucleotide is a 2′-fluoro modified nucleotide, 2′-O-methyl modified nucleotide, or 2′-O-alkyl modified nucleotide. In some embodiments, each nucleotide of the sense strand and the antisense strand is independently a modified nucleotide, e.g., a 2′-fluoro modified nucleotide, 2′-O-methyl modified nucleotide, or 2′-O-alkyl modified nucleotide.

In some embodiments, the sense strand has four 2′-fluoro modified nucleotides, e.g., at positions 7, 9, 10, 11 from the 5′ end of the sense strand. In some embodiments, the other nucleotides of the sense strand are 2′-O-methyl modified nucleotides. In some embodiments, the antisense strand has four 2′-fluoro modified nucleotides, e.g., at positions 2, 6, 14, 16 from the 5′ end of the antisense strand. In some embodiments, the other nucleotides of the antisense strand are 2′-O-methyl modified nucleotides.

In some embodiments, the sense strand has three 2′-fluoro modified nucleotides, e.g., at positions 9, 10, 11 from the 5′ end of the sense strand. In some embodiments, the other nucleotides of the sense strand are 2′-O-methyl modified nucleotides. In some embodiments, the antisense strand has five 2′-fluoro modified nucleotides, e.g., at positions 2, 5, 7, 14, 16 from the 5′ end of the antisense strand. In some embodiments, the antisense strand has five 2′-fluoro modified nucleotides, e.g., at positions 2, 5, 8, 14, 16 from the 5′ end of the antisense strand. In some embodiments, the antisense strand has five 2′-fluoro modified nucleotides, e.g., at positions 2, 3, 7, 14, 16 from the 5′ end of the antisense strand. In some embodiments, the other nucleotides of the antisense strand are 2′-O-methyl modified nucleotides.

In some embodiments, the 5′ end of the antisense strand has a phosphate analog, e.g., 5′-vinylphosphonate (5′-VP).

In some embodiments, the sense strand or the antisense strand comprises an abasic moiety or inverted abasic moiety, e.g., a moiety shown in Table 10. In some embodiments, the sense strand comprises an abasic moiety at position 10.

TABLE 10
Abasic or inverted abasic (iAb) moieties

| | Structure |
|---|---|
| 1 (abasic) | 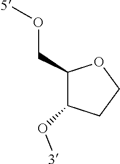 |
| 2 (iAb) | 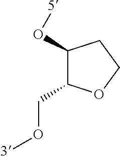 |

“5′” and “3′” indicate the 5′ to 3′ direction of the sequences.

In some embodiments, the sense strand and the antisense strand have one or more modified internucleotide linkages. In some embodiments, the modified internucleotide linkage is phosphorothioate linkage. In some embodiments, the sense strand has four or five phosphorothioate linkages. In some embodiments, the antisense strand has four or five phosphorothioate linkages. In some embodiments, the sense strand and the antisense strand each has four or five phosphorothioate linkages. In some embodiments, the sense strand has four phosphorothioate linkages and the antisense strand has five phosphorothioate linkages.

Exemplary modified sense strand and antisense strand sequences of dsRNA targeting human SNCA mRNA are provided in Table 11a. Exemplary modified sense strand and antisense strand sequences of dsRNA targeting human MAPT mRNA are provided in Table 11b.

In some embodiments, the dsRNA comprises a sense strand that comprises a sequence that has 1, 2, or 3 differences from a sense stand sequence in Table 9a or 11a. In some embodiments, the dsRNA comprises an antisense strand that comprises a sequence that has 1, 2, or 3 differences from an antisense stand sequence in Table 9a or 11a.

In some embodiments, the dsRNA comprises a sense strand that comprises a sequence that has 1, 2, or 3 differences from a sense stand sequence in Table 9b or 11b. In some embodiments, the dsRNA comprises an antisense strand that comprises a sequence that has 1, 2, or 3 differences from an antisense stand sequence in Table 9b or 11b.

TABLE 11a
Modified Nucleic Acid Sequences of dsRNA targeting human SNCA mRNA
(SNCA siRNA)

| dsRNA | | | SEQ ID |
|---|---|---|---|
| No. | Strand | Oligo Sequence 5′ to 3′ | NO |
| 8 | S | mC*mU*mGmUmAmCfAmAfGfUfGmCmUmCmAmGmUmUmC*mC*mA*(C6 amino) | 93 |
| | AS | [VPmU]*fG*mGmAmAfCmUmGmAmGmCmAmCfUmUfGmUmAmCmAmG*mG*mA | 94 |
| 9 | S | mC*mU*mGmUmAmCmAmAfGfUfGmCmUmCmAmGmUmUmC*mC*mA*(C6 amino) | 95 |
| | AS | [VPmU]*fG*mGmAfAmCfUmGmAmGmCmAmCfUmUfGmUmAmCmAmG*mG*mA | 96 |
| 10 | S | mC*mU*mGmUmAmCmAmAfGfUfGmCmUmCmAmGmUmUmC*mC*mA*(C6 amino) | 95 |
| | AS | [VPmU]*fG*mGmAfAmCmUfGmAmGmCmAmCfUmUfGmUmAmCmAmG*mG*mA | 97 |
| 11 | S | mC*mU*mGmUmAmCmAmAfGfUfGmCmUmCmAmGmUmUmC*mC*mA*(C6 amino) | 95 |
| | AS | [VPmU]*fG*fGmAmAmCfUmGmAmGmCmAmCfUmUfGmUmAmCmAmG*mG*mA | 98 |
| 12 | S | iAbmC*mU*mGmUmAmCfAmAfGfUfGmCmUmCmAmGmUmUmC*mC*mA*(C6 amino) | 99 |
| | AS | [VPmU]*fG*mGmAmAfCmUmGmAmGmCmAmCfUmUfGmUmAmCmAmG*mG*mA | 94 |
| 13 | S | mU*mG*mUmAmCmAfAmGfUfGfCmUmCmAmGmUmUmCmC*mA*mA*(C6 amino) | 100 |
| | AS | [VPmU]*fU*mGmGmAfAmCmUmGmAmGmCmAfCmUfUmGmUmAmCmA*mG*mG | 101 |
| 14 | S | mG*mU*mGmAmCmUfAmCfCfAfCmUmUmAmUmUmUmCmU*mA*mA*(C6 amino) | 102 |
| | AS | [VPmU]*fU*mAmGmAfAmAmUmAmAmGmUmGfGmUfAmGmUmCmAmC*mU*mU | 103 |
| 15 | S | mG*mA*mGmCmAmAfGmUfGfAfCmAmAmAmUmGmUmUmG*mG*mA*(C6 amino) | 104 |
| | AS | [VPmU]*fC*mCmAmAfCmAmUmUmUmGmUmCfAmCfUmUmGmCmUmC*mU*mU | 105 |
| 16 | S | mA*mG*mUmGmAmCfUmAfCfCfAmCmUmUmAmUmUmUmC*mU*mA*(C6 amino) | 106 |
| | AS | [VPmU]*fA*mGmAmAfAmUmAmAmGmUmGmGfUmAfGmUmCmAmCmU*mU*mA | 107 |
| 17 | S | iAbmA*mG*mUmGmAmCfUmAfCfCfAmCmUmUmAmUmUmUmC*mU*mA*(C6 amino) | 108 |
| | AS | [VPmU]*fA*mGmAmAfAmUmAmAmGmUmGmGfUmAfGmUmCmAmCmU*mU*mA | 107 |
| 18 | S | mC*mU*mGmUmAmCmAmAfGnfGmCmUmCmAmGmUmUmC*mC*mA*(C6 amino), | 117 |
| | | wherein n is the abasic moiety in Table 10. | |
| | AS | [VPmU]*fG*mGmAfAmCmUfGmAmGmCmAmCfUmUfGmUmAmCmAmG*mG*mA | 97 |
| 19 | S | mC*mU*mGmUmAmCfAmAfGnfGmCmUmCmAmGmUmUmC*mC*mA*(C6 amino), | 118 |
| | | wherein n is the abasic moiety in Table 10. | |
| | AS | [VPmU]*fG*mGmAfAmCmUfGmAmGmCmAmCfUmUfGmUmAmCmAmG*mG*mA | 97 |
| 23 | S | mC*mU*mGmUmAmCfAmAfGfUfGmCmUmCmAmGmUmUmC*mC*mA | 140 |
| | AS | [VPmU]*fG*mGmAmAfCmUmGmAmGmCmAmCfUmUfGmUmAmCmAmG*mG*mA | 94 |
| 24 | S | mC*mU*mGmUmAmCmAmAfGfUfGmCmUmCmAmGmUmUmC*mC*mA | 141 |
| | AS | [VPmU]*fG*mGmAfAmCfUmGmAmGmCmAmCfUmUfGmUmAmCmAmG*mG*mA | 96 |
| 25 | S | mC*mU*mGmUmAmCmAmAfGfUfGmCmUmCmAmGmUmUmC*mC*mA | 141 |
| | AS | [VPmU]*fG*mGmAfAmCmUfGmAmGmCmAmCfUmUfGmUmAmCmAmG*mG*mA | 97 |
| 26 | S | mC*mU*mGmUmAmCmAmAfGfUfGmCmUmCmAmGmUmUmC*mC*mA | 141 |
| | AS | [VPmU]*fG*fGmAmAmCfUmGmAmGmCmAmCfUmUfGmUmAmCmAmG*mG*mA | 98 |
| 27 | S | iAbmC*mU*mGmUmAmCfAmAfGfUfGmCmUmCmAmGmUmUmC*mC*mA | 142 |
| | AS | [VPmU]*fG*mGmAmAfCmUmGmAmGmCmAmCfUmUfGmUmAmCmAmG*mG*mA | 94 |
| 28 | S | mU*mG*mUmAmCmAfAmGfUfGfCmUmCmAmGmUmUmCmC*mA*mA | 143 |
| | AS | [VPmU]*fU*mGmGmAfAmCmUmGmAmGmCmAfCmUfUmGmUmAmCmA*mG*mG | 101 |
| 29 | S | mG*mU*mGmAmCmUfAmCfCfAfCmUmUmAmUmUmUmCmU*mA*mA | 144 |
| | AS | [VPmU]*fU*mAmGmAfAmAmUmAmAmGmUmGfGmUfAmGmUmCmAmC*mU*mU | 103 |
| 30 | S | mG*mA*mGmCmAmAfGmUfGfAfCmAmAmAmUmGmUmUmG*mG*mA | 145 |
| | AS | [VPmU]*fC*mCmAmAfCmAmUmUmUmGmUmCfAmCfUmUmGmCmUmC*mU*mU | 105 |
| 31 | S | mA*mG*mUmGmAmCfUmAfCfCfAmCmUmUmAmUmUmUmC*mU*mA | 146 |
| | AS | [VPmU]*fA*mGmAmAfAmUmAmAmGmUmGmGfUmAfGmUmCmAmCmU*mU*mA | 107 |
| 32 | S | iAbmA*mG*mUmGmAmCfUmAfCfCfAmCmUmUmAmUmUmUmC*mU*mA | 147 |
| | AS | [VPmU]*fA*mGmAmAfAmUmAmAmGmUmGmGfUmAfGmUmCmAmCmU*mU*mA | 107 |
| 33 | S | mC*mU*mGmUmAmCmAmAfGnfGmCmUmCmAmGmUmUmC*mC*mA, wherein n is the | 148 |
| | | abasic moiety in Table 10. | |
| | AS | [VPmU]*fG*mGmAfAmCmUfGmAmGmCmAmCfUmUfGmUmAmCmAmG*mG*mA | 97 |
| 34 | S | mC*mU*mGmUmAmCfAmAfGnfGmCmUmCmAmGmUmUmC*mC*mA, wherein n is the | 149 |
| | | abasic moiety in Table 10. | |
| | AS | [VPmU]*fG*mGmAfAmCmUfGmAmGmCmAmCfUmUfGmUmAmCmAmG*mG*mA | 97 |

Abbreviations-“m” indicates 2′-OMe; “f” indicated 2′-fluoro; “*” indicates phosphorothioate linkage; “VP” indicates 5′-vinylphosphonate; “iAb” indicates inverted abasic moiety in Table 10; “S” means the sense strand; “AS” means the antisense strand.

TABLE 11b
Modified Nucleic Acid Sequences of dsRNA targeting human MAPT mRNA
(MAPT siRNA)

| dsRNA | | | SEQ |
|---|---|---|---|
| No. | Strand | Oligo Sequence 5′ to 3′ | ID NO |
| 35 | S | mG*mU*mGmGmAmAfGmUfAfAfAmAmUmCmUmGmAmGmA*mA*mA* (C6 amino) | 126 |
| | AS | [VPmU]*fU*mUmCmUfCmAmGmAmUmUmUmUfAmCfUmUmCmCmAmC*mC*mU | 127 |
| 36 | S | mC*mC*mAmAmGmUfGmUfGfGfCmUmCmAmUmUmAmGmG*mC*mA* (C6 amino) | 128 |
| | AS | [VPmU]*fG*mCmCmUfAmAmUmGmAmGmCmCfAmCfAmCmUmUmGmG*mA*mG | 129 |
| 37 | S | mU*mG*mCmAmAmAfUmAfGfUfCmUmAmCmAmAmAmCmC*mA*mA* (C6 amino) | 130 |
| | AS | [VPmU]*fU*mGmGmUfUmUmGmUmAmGmAmCfUmAfUmUmUmGmCmA*mC*mC | 131 |
| 38 | S | mG*mU*mGmGmAmAmGmUfAfAfAmAmUmCmUmGmAmGmA*mA*mA*(C6 amino) | 132 |
| | AS | [VPmU]*fU*mUmCfUmCmAfGmAmUmUmUmUfAmCfUmUmCmCmAmC*mC*mU | 133 |
| 39 | S | mC*mC*mAmAmGmUmGmUfGfGfCmUmCmAmUmUmAmGmG*mC*mA*(C6 amino) | 134 |
| | AS | [VPmU]*fG*mCmCfUmAmAfUmGmAmGmCmCfAmCfAmCmUmUmGmG*mA*mG | 135 |
| 40 | S | mU*mG*mCmAmAmAmUmAfGfUfCmUmAmCmAmAmAmCmC*mA*mA*(C6 amino) | 136 |
| | AS | [VPmU]*fU*mGmGfUmUmUfGmUmAmGmAmCfUmAfUmUmUmGmCmA*mC*mC | 137 |
| 41 | S | mG*mU*mGmGmAmAfGmUfAfAfAmAmUmCmUmGmAmGmA*mA*mA | 150 |
| | AS | [VPmU]*fU*mUmCmUfCmAmGmAmUmUmUmUfAmCfUmUmCmCmAmC*mC*mU | 127 |
| 42 | S | mC*mC*mAmAmGmUfGmUfGfGfCmUmCmAmUmUmAmGmG*mC*mA | 151 |
| | AS | [VPmU]*fG*mCmCmUfAmAmUmGmAmGmCmCfAmCfAmCmUmUmGmG*mA*mG | 129 |
| 43 | S | mU*mG*mCmAmAmAfUmAfGfUfCmUmAmCmAmAmAmCmC*mA*mA | 152 |
| | AS | [VPmU]*fU*mGmGmUfUmUmGmUmAmGmAmCfUmAfUmUmUmGmCmA*mC*mC | 131 |
| 44 | S | mG*mU*mGmGmAmAmGmUfAfAfAmAmUmCmUmGmAmGmA*mA*mA | 153 |
| | AS | [VPmU]*fU*mUmCfUmCmAfGmAmUmUmUmUfAmCfUmUmCmCmAmC*mC*mU | 133 |
| 45 | S | mC*mC*mAmAmGmUmGmUfGfGfCmUmCmAmUmUmAmGmG*mC*mA | 154 |
| | AS | [VPmU]*fG*mCmCfUmAmAfUmGmAmGmCmCfAmCfAmCmUmUmGmG*mA*mG | 135 |
| 46 | S | mU*mG*mCmAmAmAmUmAfGfUfCmUmAmCmAmAmAmCmC*mA*mA | 155 |
| | AS | [VPmU]*fU*mGmGfUmUmUfGmUmAmGmAmCfUmAfUmUmUmGmCmA*mC*mC | 137 |

Abbreviations-“m” indicates 2′-OMe; “f” indicated 2′-fluoro; “*” indicates phosphorothioate linkage; “VP” indicates 5′-vinylphosphonate; “S” means the sense strand; “AS” means the antisense strand.

In some embodiments, the dsRNA targets SNCA mRNA. In some embodiments, the sense strand and the antisense strand of the dsRNA comprise a pair of nucleic acid sequences selected from the group consisting of:
(a) the sense strand comprises SEQ ID NO: 93 or 140, and the antisense strand comprises SEQ ID NO: 94;
(b) the sense strand comprises SEQ ID NO: 95 or 141, and the antisense strand comprises SEQ ID NO: 96;
(c) the sense strand comprises SEQ ID NO: 95 or 141, and the antisense strand comprises SEQ ID NO: 97;
(d) the sense strand comprises SEQ ID NO: 95 or 141, and the antisense strand comprises SEQ ID NO: 98;
(e) the sense strand comprises SEQ ID NO: 99 or 142, and the antisense strand comprises SEQ ID NO: 94;
(f) the sense strand comprises SEQ ID NO: 100 or 143, and the antisense strand comprises SEQ ID NO: 101;
(g) the sense strand comprises SEQ ID NO: 102 or 144, and the antisense strand comprises SEQ ID NO: 103;
(h) the sense strand comprises SEQ ID NO: 104 or 145, and the antisense strand comprises SEQ ID NO: 105;
(i) the sense strand comprises SEQ ID NO: 106 or 146, and the antisense strand comprises SEQ ID NO: 107;
(j) the sense strand comprises SEQ ID NO: 108 or 147, and the antisense strand comprises SEQ ID NO: 107;
(k) the sense strand comprises SEQ ID NO: 117 or 148, and the antisense strand comprises SEQ ID NO: 97; and
(l) the sense strand comprises SEQ ID NO: 118 or 149, and the antisense strand comprises SEQ ID NO: 97.

In some embodiments, the sense strand and the antisense strand of the dsRNA have a pair of nucleic acid sequences selected from the group consisting of:
(a) the sense strand consists of SEQ ID NO: 93 or 140, and the antisense strand consists of SEQ ID NO: 94;
(b) the sense strand consists of SEQ ID NO: 95 or 141, and the antisense strand consists of SEQ ID NO: 96;
(c) the sense strand consists of SEQ ID NO: 95 or 141, and the antisense strand consists of SEQ ID NO: 97;
(d) the sense strand consists of SEQ ID NO: 95 or 141, and the antisense strand consists of SEQ ID NO: 98;
(e) the sense strand consists of SEQ ID NO: 99 or 142, and the antisense strand consists of SEQ ID NO: 94;
(f) the sense strand consists of SEQ ID NO: 100 or 143, and the antisense strand consists of SEQ ID NO: 101;
(g) the sense strand consists of SEQ ID NO: 102 or 144, and the antisense strand consists of SEQ ID NO: 103;
(h) the sense strand consists of SEQ ID NO: 104 or 145, and the antisense strand consists of SEQ ID NO: 105;
(i) the sense strand consists of SEQ ID NO: 106 or 146, and the antisense strand consists of SEQ ID NO: 107;
(j) the sense strand consists of SEQ ID NO: 108 or 147, and the antisense strand consists of SEQ ID NO: 107;
(k) the sense strand consists of SEQ ID NO: 117 or 148, and the antisense strand consists of SEQ ID NO: 97; and
(l) the sense strand consists of SEQ ID NO: 118 or 149, and the antisense strand consists of SEQ ID NO: 97.

In some embodiments, the dsRNA targets MAPT mRNA. In some embodiments, the sense strand and the antisense strand of the dsRNA comprise a pair of nucleic acid sequences selected from the group consisting of:
(a) the sense strand comprises SEQ ID NO: 126 or 150, and the antisense strand comprises SEQ ID NO: 127;
(b) the sense strand comprises SEQ ID NO: 128 or 151, and the antisense strand comprises SEQ ID NO: 129;
(c) the sense strand comprises SEQ ID NO: 130 or 152, and the antisense strand comprises SEQ ID NO: 131;
(d) the sense strand comprises SEQ ID NO: 132 or 153, and the antisense strand comprises SEQ ID NO: 133;
(e) the sense strand comprises SEQ ID NO: 134 or 154, and the antisense strand comprises SEQ ID NO: 135; and
(f) the sense strand comprises SEQ ID NO: 136 or 155, and the antisense strand comprises SEQ ID NO: 137.

In some embodiments, the sense strand and the antisense strand of the dsRNA have a pair of nucleic acid sequences selected from the group consisting of:
(a) the sense strand consists of SEQ ID NO: 126 or 150, and the antisense strand consists of SEQ ID NO: 127;
(b) the sense strand consists of SEQ ID NO: 128 or 151, and the antisense strand consists of SEQ ID NO: 129;
(c) the sense strand consists of SEQ ID NO: 130 or 152, and the antisense strand consists of SEQ ID NO: 131;
(d) the sense strand consists of SEQ ID NO: 132 or 153, and the antisense strand consists of SEQ ID NO: 133;
(e) the sense strand consists of SEQ ID NO: 134 or 154, and the antisense strand consists of SEQ ID NO: 135; and
(f) the sense strand consists of SEQ ID NO: 136 or 155, and the antisense strand consists of SEQ ID NO: 137.

The sense strand and antisense strand of dsRNA can be synthesized using any nucleic acid polymerization methods known in the art, for example, solid-phase synthesis by employing phosphoramidite chemistry methodology (e.g., Current Protocols in Nucleic Acid Chemistry, Beaucage, S. L. et al. (Edrs.), John Wiley & Sons, Inc., New York, NY, USA), H-phosphonate, phosphortriester chemistry, or enzymatic synthesis. Automated commercial synthesizers can be used, for example, MerMade™ 12 from LGC Biosearch Technologies, or other synthesizers from BioAutomation or Applied Biosystems. Phosphorothioate linkages can be introduced using a sulfurizing reagent such as phenylacetyl disulfide or DDTT (((dimethylaminomethylidene) amino)-3H-1,2,4-dithiazaoline-3-thione). It is well known to use similar techniques and commercially available modified amidites and controlled-pore glass (CPG) products to synthesize modified oligonucleotides or conjugated oligonucleotides.

Purification methods can be used to exclude the unwanted impurities from the final oligonucleotide product. Commonly used purification techniques for single stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC). After purification, oligonucleotides can be analyzed by mass spectrometry and quantified by spectrophotometry at a wavelength of 260 nm. The sense strand and antisense strand can then be annealed to form a dsRNA.

### Pharmaceutical Composition

In another aspect, provided herein are pharmaceutical compositions comprising any of the human TfR binding proteins or conjugates described herein and a pharmaceutically acceptable carrier. Such pharmaceutical compositions can also comprise one or more pharmaceutically acceptable excipient, diluent, or carrier. Pharmaceutical compositions can be prepared by methods well known in the art (e.g., Remington: The Science and Practice of Pharmacy, 23rd edition (2020), A. Loyd et al., Academic Press).

### Method of Treatment and Therapeutic Use

In another aspect, provided herein are methods of treating a CNS disease, e.g., a neurodegenerative disease, in a patient in need thereof, and such the method comprises administering to the patient an effective amount of the human TfR binding protein or conjugate or a pharmaceutical composition described herein.

In a further aspect, provided herein are methods of treating a neurodegenerative synucleinopathy in a patient in need thereof, and such the method comprises administering to the patient an effective amount of the human TfR binding proteins or conjugate or a pharmaceutical composition described herein, e.g., a TBP-SNCA siRNA conjugate described herein or a pharmaceutical composition comprising such a TBP-SNCA siRNA conjugate. Exemplary neurodegenerative synucleinopathy includes, but are not limited to, Parkinson's disease; multiple system atrophy; Lewy body dementia or dementia with Lewy bodies; pure autonomic failure; Alzheimer's disease; Lewy body dysphagia; and incidental Lewy body disease. In some embodiments, the neurodegenerative synucleinopathy is selected from Parkinson's disease, Alzheimer's disease, multiple system atrophy, or Lewy body dementia. The human TfR binding protein or conjugate or a pharmaceutical composition can be administered to the patient intravenously or subcutaneously.

In a further aspect, provided herein are methods of treating a tauopathy in a patient in need thereof, and such the method comprises administering to the patient an effective amount of the human TfR binding proteins or conjugate or a pharmaceutical composition described herein, e.g., a TBP-MAPT siRNA conjugate described herein or a pharmaceutical composition comprising such a TBP-MAPT siRNA conjugate. Exemplary tauopathy includes, but are not limited to, Alzheimer's disease, frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's discase, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT). The human TfR binding protein or conjugate or a pharmaceutical composition can be administered to the patient intravenously or subcutaneously.

Human TfR binding protein or conjugate dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Dosage values may vary with the type and severity of the condition to be alleviated. It is further understood that for any particular subject, specific dosage regimens should be adjusted over time according to the individual need and the professional judgment of the person administering or supervising the administration of the compositions.

In another aspect, provided herein are human TfR binding proteins or conjugates described herein or pharmaceutical compositions comprising such human TfR binding proteins or conjugates for use in a therapy. Also provided herein are human TfR binding proteins or conjugates described herein or pharmaceutical compositions comprising such human TfR binding proteins or conjugates (e.g., a TBP-SNCA siRNA conjugate described herein or a pharmaceutical composition comprising such a TBP-SNCA siRNA conjugate) for use in the treatment of a neurodegenerative synucleinopathy, e.g., Parkinson's disease, Alzheimer's disease, multiple system atrophy, or Lewy body dementia.

Also provided herein are human TfR binding proteins or conjugates described herein or pharmaceutical compositions comprising such human TfR binding proteins or conjugates (e.g., a TBP-MAPT siRNA conjugate described herein or a pharmaceutical composition comprising such a TBP-MAPT siRNA conjugate) for use in the treatment of a tauopathy, e.g., Alzheimer's disease, frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's discase, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT).

In another aspect, provided herein are uses of human TfR binding proteins or conjugates described herein in the manufacture of a medicament for treating a CNS disease, e.g., a neurodegenerative disease. In some embodiments, the neurodegenerative disease is a neurodegenerative synucleinopathy, e.g., Parkinson's disease, Alzheimer's disease, multiple system atrophy, or Lewy body dementia. In some embodiments, the neurodegenerative disease is a tauopathy, e.g., Alzheimer's disease, frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's discase, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT).

### Definitions

As used herein, the terms “a,” “an,” “the,” and similar terms used in the context of the present disclosure (especially in the context of the claims) are to be construed to cover both the singular and plural unless otherwise indicated herein or clearly contradicted by the context.

As used herein, the term “alkyl” means saturated linear or branched-chain monovalent hydrocarbon radical, containing the indicated number of carbon atoms. For example, “C₁-C₂₀ alkyl” means a radical having 1-20 carbon atoms in a linear or branched arrangement.

The term “antibody,” as used herein, refers to a molecule that binds an antigen. Embodiments of an antibody include a monoclonal antibody, polyclonal antibody, human antibody, humanized antibody, chimeric antibody, heterodimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)2, Fv fragments, scFv antibody fragments, scFab, disulfide-linked Fvs (sdFv), a Fd fragment.

The term “antigen binding domain”, as used herein, refers to a portion of an antibody or antibody fragment that binds an antigen or an epitope of the antigen. For example. “TfR binding domain” refers to a portion of an antibody or antibody fragment that binds TfR or an epitope of TfR.

The term “heterodimeric antibody”, as used herein, refers to an antibody that comprises two distinct antigen-binding domains.

As used herein, “antisense strand” means a single-stranded oligonucleotide that is complementary to a region of a target sequence. Likewise, and as used herein, “sense strand” means a single-stranded oligonucleotide that is complementary to a region of an antisense strand.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

As used herein, “complementary” means a structural relationship between two nucleotides (e.g., on two opposing nucleic acids or on opposing regions of a single nucleic acid strand, e.g., a hairpin) that permits the two nucleotides to form base pairs with one another. For example, a purine nucleotide of one nucleic acid that is complementary to a pyrimidine nucleotide of an opposing nucleic acid may base pair together by forming hydrogen bonds with one another. Complementary nucleotides can base pair in the Watson-Crick manner or in any other manner that allows for the formation of stable duplexes. Likewise, two nucleic acids may have regions of multiple nucleotides that are complementary with each other to form regions of complementarity, as described herein.

As used herein, “duplex,” in reference to nucleic acids or oligonucleotides, means a structure formed through complementary base pairing of two antiparallel sequences of nucleotides (i.e., in opposite directions), whether formed by two separate nucleic acid strands or by a single, folded strand (e.g., via a hairpin).

An “effective amount” refers to an amount necessary (for periods of time and for the means of administration) to achieve the desired therapeutic result. An effective amount of a protein or conjugate may vary according to factors such as the disease state, age, sex, and weight of the individual, and the ability of the protein or conjugate to elicit a desired response in the individual. An effective amount is also one in which any toxic or detrimental effects of the protein or conjugate are outweighed by the therapeutically beneficial effects.

As referred to herein, the term “epitope” refers to the amino acid residues, of an antigen, that are bound by an antibody. An epitope can be a linear epitope, a conformational epitope, or a hybrid epitope. The term “epitope” may be used in reference to a structural epitope. A structural epitope, according to some embodiments, may be used to describe the region of an antigen which is covered by an antibody or antigen binding protein. In some embodiments, a structural epitope may describe the amino acid residues of the antigen that are within a specified proximity (e.g., within a specified number of Angstroms) of an amino acid residue of the antibody or antigen binding protein. The term “epitope” may also be used in reference to a functional epitope. A functional epitope, according to some embodiments, may be used to describe amino acid residues of the antigen that interact with amino acid residues of the antibody or antigen binding protein in a manner contributing to the binding energy between the antigen and the antibody or antigen binding protein.

An epitope can be determined according to different experimental techniques, also called “epitope mapping techniques.” It is understood that the determination of an epitope may vary based on the different epitope mapping techniques used and may also vary with the different experimental conditions used, e.g., due to the conformational changes or cleavages of the antigen induced by specific experimental conditions. Epitope mapping techniques are known in the art (e.g., Rockberg and Nilvebrant, Epitope Mapping Protocols: Methods in Molecular Biology, Humana Press, 3^{rd} ed. 2018), including but not limited to, X-ray crystallography, nuclear magnetic resonance (NMR) spectroscopy, site-directed mutagenesis, species swap mutagenesis, alanine-scanning mutagenesis, hydrogen-deuterium exchange (HDX) and cross-blocking assays.

The term “Fc region” as used herein refers to a polypeptide comprising the CH2 and CH3 domains of a constant region of an immunoglobulin, e.g., IgG1, IgG2, IgG3, or IgG4. Optionally, the Fc region may include a portion of the hinge region or the entire hinge region of an immunoglobulin, e.g., IgG1, IgG2, IgG3, or IgG4. In some embodiments, the Fc region is a human IgG Fc region, e.g., a human IgG1 Fc region, human IgG2 Fc region, human IgG3 Fc region or human IgG4 Fc region. In some embodiments, the Fc region is a modified IgG Fc region with reduced or eliminated effector functions compared to the corresponding wild type IgG Fc region. The numbering of the residues in the Fc region is based on the EU index as described in Kabat (Kabat et al, Sequences of Proteins of Immunological Interest, 5th edition, Bethesda, MD: U.S. Dept. of Health and Human Services, Public Health Service, National Institutes of Health, 1991). The boundaries of the Fc region of an immunoglobulin heavy chain might vary, and the human IgG heavy chain Fc region is usually defined as the stretch from the N-terminus of the CH2 domain (e.g., the amino acid residue at position 231 according to the EU index numbering) to the C-terminus of the CH3 domain (or the C-terminus of the immunoglobulin).

The term “knockdown” or “expression knockdown” refers to reduced mRNA or protein expression of a gene after treatment of a reagent.

As used herein, “modified internucleotide linkage” means an internucleotide linkage having one or more chemical modifications when compared with a reference internucleotide linkage having a phosphodiester bond. A modified internucleotide linkage can be a non-naturally occurring linkage. In some embodiments, the modified internucleotide linkage is phosphorothioate linkage.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide. In some embodiments, the modified nucleotide is a 2′-fluoro modified nucleotide, 2′-O-methyl modified nucleotide, or 2′-O-alkyl modified nucleotide. In some embodiments, the modified nucleotide has a phosphate analog, e.g., 5′-vinylphosphonate. In some embodiments, the modified nucleotide has an abasic moiety or inverted abasic moiety, e.g., a moiety shown in Table 10.

As used herein, the term “neurodegenerative synucleinopathy” refers to a neurodegenerative disorder characterized by fibrillary aggregates of alpha-synuclein protein in the cytoplasm of selective populations of neurons and glia in the central and/or peripheral nervous systems.

As used herein, “nucleotide” means an organic compound having a nucleoside (a nucleobase, e.g., adenine, cytosine, guanine, thymine, or uracil, and a pentose sugar, e.g., ribose or 2′-deoxyribose) linked to a phosphate group. A “nucleotide” can serve as a monomeric unit of nucleic acid polymers such as deoxyribonucleic acid (DNA) and ribonucleic acid (RNA).

As used herein, a “null arm” means an antibody arm that does not bind any known human target.

As used herein, “oligonucleotide” means a polymer of linked nucleotides, each of which can be modified or unmodified. An oligonucleotide is typically less than about 100 nucleotides in length.

As used herein, “overhang” means the unpaired nucleotide or nucleotides that protrude from the duplex structure of a double stranded oligonucleotide. An overhang may include one or more unpaired nucleotides extending from a duplex region at the 5′ terminus or 3′ terminus of a double stranded oligonucleotide. The overhang can be a 3′ or 5′ overhang on the antisense strand or sense strand of a double stranded oligonucleotide.

The term “patient”, as used herein, refers to a human patient.

As used herein, “phosphate analog” means a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. A 5′ phosphate analog can include a phosphatase-resistant linkage. Examples of phosphate analogs include 5′ methylene phosphonate (5′-MP) and 5′-(E)-vinylphosphonate (5′-VP). In some embodiments, the phosphate analog is 5′-VP.

The term “% sequence identity” or “percentage sequence identity” with respect to a reference nucleic acid sequence is defined as the percentage of nucleotides, nucleosides, or nucleobases in a candidate sequence that are identical with the nucleotides, nucleosides, or nucleobases in the reference nucleic acid sequence, after optimally aligning the sequences and introducing gaps or overhangs, if necessary, to achieve the maximum percent sequence identity. Alignment for purposes of determining percent nucleic acid sequence identity can be achieved in various ways that are within the skill in the art, for instance, using publicly available computer software programs, for example, those described in Current Protocols in Molecular Biology (Ausubel et al., eds., 1987, Supp. 30, section 7.7.18, Table 7.7.1), and including BLAST, BLAST-2, ALIGN, Megalign (DNASTAR), Clustal W2.0 or Clustal X2.0 software. Those skilled in the art can determine appropriate parameters for measuring alignment, including any algorithms needed to achieve maximal alignment over the full length of the sequences being compared. Percentage of “sequence identity” can be determined by comparing two optimally aligned sequences over a comparison window, where the fragment of the nucleic acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical nucleotide, nucleoside, or nucleobase occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

The term “polypeptide” or “protein”, as used herein, refers to a polymer of amino acid residues. The term applies to polymers comprising naturally occurring amino acids and polymers comprising one or more non-naturally occurring amino acids.

As used herein, “strand” refers to a single, contiguous sequence of nucleotides linked together through internucleotide linkages (e.g., phosphodiester linkages or phosphorothioate linkages). A strand can have two free ends (e.g., a 5′ end and a 3′ end).

As used herein, “SNCA” refers to an alpha-synuclein (SNCA) mRNA, protein, or polypeptide. The nucleic acid sequence of a human SNCA mRNA transcript can be found at NM_000345.4:

```
(SEQ ID NO: 109)
      1 GGCGACGACC AGAAGGGGCC CAAGAGAGGG GGCGAGCGAC CGAGCGCCGC GACGCGGAAG
    61 TGAGGTGCGT GCGGGCTGCA GCGCAGACCC CGGCCCGGCC CCTCCGAGAG CGTCCTGGGC
  121 GCTCCCTCAC GCCTTGCCTT CAAGCCTTCT GCCTTTCCAC CCTCGTGAGC GGAGAACTGG
  181 GAGTGGCCAT TCGACGACAG TGTGGTGTAA AGGAATTCAT TAGCCATGGA TGTATTCATG
  241 AAAGGACTTT CAAAGGCCAA GGAGGGAGTT GTGGCTGCTG CTGAGAAAAC CAAACAGGGT
  301 GTGGCAGAAG CAGCAGGAAA GACAAAAGAG GGTGTTCTCT ATGTAGGCTC CAAAACCAAG
  361 GAGGGAGTGG TGCATGGTGT GGCAACAGTG GCTGAGAAGA CCAAAGAGCA AGTGACAAAT
  421 GTTGGAGGAG CAGTGGTGAC GGGTGTGACA GCAGTAGCCC AGAAGACAGT GGAGGGAGCA
  481 GGGAGCATTG CAGCAGCCAC TGGCTTTGTC AAAAAGGACC AGTTGGGCAA GAATGAAGAA
  541 GGAGCCCCAC AGGAAGGAAT TCTGGAAGAT ATGCCTGTGG ATCCTGACAA TGAGGCTTAT
  601 GAAATGCCTT CTGAGGAAGG GTATCAAGAC TACGAACCTG AAGCCTAAGA AATATCTTTG
  661 CTCCCAGTTT CTTGAGATCT GCTGACAGAT GTTCCATCCT GTACAAGTGC TCAGTTCCAA
  721 TGTGCCCAGT CATGACATTT CTCAAAGTTT TTACAGTGTA TCTCGAAGTC TTCCATCAGC
  781 AGTGATTGAA GTATCTGTAC CTGCCCCCAC TCAGCATTTC GGTGCTTCCC TTTCACTGAA
  841 GTGAATACAT GGTAGCAGGG TCTTTGTGTG CTGTGGATTT TGTGGCTTCA ATCTACGATG
  901 TTAAAACAAA TTAAAAACAC CTAAGTGACT ACCACTTATT TCTAAATCCT CACTATTTTT
  961 TTGTTGCTGT TGTTCAGAAG TTGTTAGTGA TTTGCTATCA TATATTATAA GATTTTTAGG
1021 TGTCTTTTAA TGATACTGTC TAAGAATAAT GACGTATTGT GAAATTTGTT AATATATATA
1081 ATACTTAAAA ATATGTGAGC ATGAAACTAT GCACCTATAA ATACTAAATA TGAAATTTTA
1141 CCATTTTGCG ATGTGTTTTA TTCACTTGTG TTTGTATATA AATGGTGAGA ATTAAAATAA
1201 AACGTTATCT CATTGCAAAA ATATTTTATT TTTATCCCAT CTCACTTTAA TAATAAAAAT
1261 CATGCTTATA AGCAACATGA ATTAAGAACT GACACAAAGG ACAAAAATAT AAAGTTATTA
1321 ATAGCCATTT GAAGAAGGAG GAATTTTAGA AGAGGTAGAG AAAATGGAAC ATTAACCCTA
1381 CACTCGGAAT TCCCTGAAGC AACACTGCCA GAAGTGTGTT TTGGTATGCA CTGGTTCCTT
1441 AAGTGGCTGT GATTAATTAT TGAAAGTGGG GTGTTGAAGA CCCCAACTAC TATTGTAGAG
1501 TGGTCTATTT CTCCCTTCAA TCCTGTCAAT GTTTGCTTTA CGTATTTTGG GGAACTGTTG
1561 TTTGATGTGT ATGTGTTTAT AATTGTTATA CATTTTTAAT TGAGCCTTTT ATTAACATAT
1621 ATTGTTATTT TTGTCTCGAA ATAATTTTTT AGTTAAAATC TATTTTGTCT GATATTGGTG
1681 TGAATGCTGT ACCTTTCTGA CAATAAATAA TATTCGACCA TGAATAAAAA AAAAAAAAAA
1741 GTGGGTTCCC GGGAACTAAG CAGTGTAGAA GATGATTTTG ACTACACCCT CCTTAGAGAG
1801 CCATAAGACA CATTAGCACA TATTAGCACA TTCAAGGCTC TGAGAGAATG TGGTTAACTT
1861 TGTTTAACTC AGCATTCCTC ACTTTTTTTT TTTAATCATC AGAAATTCTC TCTCTCTCTC
1921 TCTCTTTTTC TCTCGCTCTC TTTTTTTTTT TTTTTTTACA GGAAATGCCT TTAAACATCG
1981 TTGGAACTAC CAGAGTCACC TTAAAGGAGA TCAATTCTCT AGACTGATAA AAATTTCATG
2041 GCCTCCTTTA AATGTTGCCA AATATATGAA TTCTAGGATT TTTCCTTAGG AAAGGTTTTT
2101 CTCTTTCAGG GAAGATCTAT TAACTCCCCA TGGGTGCTGA AAATAAACTT GATGGTGAAA
2161 AACTCTGTAT AAATTAATTT AAAAATTATT TGGTTTCTCT TTTTAATTAT TCTGGGGCAT
2221 AGTCATTTCT AAAAGTCACT AGTAGAAAGT ATAATTTCAA GACAGAATAT TCTAGACATG
2281 CTAGCAGTTT ATATGTATTC ATGAGTAATG TGATATATAT TGGGCGCTGG TGAGGAAGGA
2341 AGGAGGAATG AGTGACTATA AGGATGGTTA CCATAGAAAC TTCCTTTTTT ACCTAATTGA
2401 AGAGAGACTA CTACAGAGTG CTAAGCTGCA TGTGTCATCT TACACTAGAG AGAAATGGTA
2461 AGTTTCTTGT TTTATTTAAG TTATGTTTAA GCAAGGAAAG GATTTGTTAT TGAACAGTAT
2521 ATTTCAGGAA GGTTAGAAAG TGGCGGTTAG GATATATTTT AAATCTACCT AAAGCAGCAT
2581 ATTTTAAAAA TTTAAAAGTA TTGGTATTAA ATTAAGAAAT AGAGGACAGA ACTAGACTGA
2641 TAGCAGTGAC CTAGAACAAT TTGAGATTAG GAAAGTTGTG ACCATGAATT TAAGGATTTA
2701 TGTGGATACA AATTCTCCTT TAAAGTGTTT CTTCCCTTAA TATTTATCTG ACGGTAATTT
2761 TTGAGCAGTG AATTACTTTA TATATCTTAA TAGTTTATTT GGGACCAAAC ACTTAAACAA
2821 AAAGTTCTTT AAGTCATATA AGCCTTTTCA GGAAGCTTGT CTCATATTCA CTCCCGAGAC
2881 ATTCACCTGC CAAGTGGCCT GAGGATCAAT CCAGTCCTAG GTTTATTTTG CAGACTTACA
2941 TTCTCCCAAG TTATTCAGCC TCATATGACT CCACGGTCGG CTTTACCAAA ACAGTTCAGA
3001 GTGCACTTTG GCACACAATT GGGAACAGAA CAATCTAATG TGTGGTTTGG TATTCCAAGT
3061 GGGGTCTTTT TCAGAATCTC TGCACTAGTG TGAGATGCAA ACATGTTTCC TCATCTTTCT
3121 GGCTTATCCA GTATGTAGCT ATTTGTGACA TAATAAATAT ATACATATAT GAAAATA.
```

The amino acid sequence of a human SNCA protein can be found at NP_000336.1:

```
(SEQ ID NO: 110)
    1 MDVFMKGLSK AKEGVVAAAE KTKQGVAEAA
        GKTKEGVLYV GSKTKEGVVH GVATVAEKTK
  61 EQVTNVGGAV VTGVTAVAQK TVEGAGSIAA
        ATGFVKKDQL GKNEEGAPQE GILEDMPVDP
121 DNEAYEMPSE EGYQDYEPEA.
```

The nucleic acid sequence of a mouse SNCA mRNA transcript can be found at NM_001042451.2; and the amino acid sequence of a mouse SNCA protein can be found at NP_001035916.1. The nucleic acid sequence of a rat SNCA mRNA transcript can be found at NM_019169.3; and the amino acid sequence of a rat SNCA protein can be found at NP_062042.1. The nucleic acid sequence of a monkey SNCA mRNA transcript can be found at XM_005555422.2; and the amino acid sequence of a monkey SNCA protein can be found at XP_005555479.1.

As used herein, “MAPT” refers to a human MAPT mRNA transcript, encoding a microtubule associated protein Tau. The nucleotide sequences of human MAPT transcript variants and amino acid sequences of human Tau protein isoforms can be found at:
i. MAPT transcript variant 1→Tau protein isoform 1: NM_016835.5 (nucleotide sequence)→NP_058519.3 (amino acid sequence);
ii. MAPT transcript variant 2→Tau protein isoform 2: NM_005910.6 (nucleotide sequence)→NP_005901.2 (amino acid sequence);
iii. MAPT transcript variant 3→Tau protein isoform 3: NM_016834.5 (nucleotide sequence)→NP_058518.1 (amino acid sequence);
iv. MAPT transcript variant 4→Tau protein isoform 4: NM_016841.5 (nucleotide sequence)→NP_058525.1 (amino acid sequence);
v. MAPT transcript variant 5→Tau protein isoform 5: NM_001123067.4 (nucleotide sequence)→NP_001116539.1 (amino acid sequence);
vi. MAPT transcript variant 6→Tau protein isoform 6: NM_001123066.4 (nucleotide sequence)→NP_001116538.2 (amino acid sequence);
vii. MAPT transcript variant 7→Tau protein isoform 7: NM_001203251.2 (nucleotide sequence)→NP_001190180.1 (amino acid sequence);
viii. MAPT transcript variant 8→Tau protein isoform 8: NM_001203252.2 (nucleotide sequence)→NP_001190181.1 (amino acid sequence);
ix. MAPT transcript variant 9→Tau protein isoform 9: NM_001377265.1 (nucleotide sequence)→NP_001364194.1 (amino acid sequence);
x. MAPT transcript variant 10→Tau protein isoform 10: NM_001377266.1 (nucleotide sequence)→NP_001364195.1 (amino acid sequence);
xi. MAPT transcript variant 11→Tau protein isoform 11: NM_001377267.1 (nucleotide sequence)→NP_001364196.1 (amino acid sequence);
xii. MAPT transcript variant 12→Tau protein isoform 4: NM_001377268.1 (nucleotide sequence)→NP_001364197.1 (amino acid sequence).

The nucleotide sequence of the human MAPT transcript variant 6 (encoding 2N4R Tau) can be found at NM_001123066.4:

```
(SEQ ID NO: 156)
      1 GCAGTCACCG CCACCCACCA GCTCCGGCAC CAACAGCAGC GCCGCTGCCA CCGCCCACCT
    61 TCTGCCGCCG CCACCACAGC CACCTTCTCC TCCTCCGCTG TCCTCTCCCG TCCTCGCCTC
  121 TGTCGACTAT CAGGTGAACT TTGAACCAGG ATGGCTGAGC CCCGCCAGGA GTTCGAAGTG
  181 ATGGAAGATC ACGCTGGGAC GTACGGGTTG GGGGACAGGA AAGATCAGGG GGGCTACACC
  241 ATGCACCAAG ACCAAGAGGG TGACACGGAC GCTGGCCTGA AAGAATCTCC CCTGCAGACC
  301 CCCACTGAGG ACGGATCTGA GGAACCGGGC TCTGAAACCT CTGATGCTAA GAGCACTCCA
  361 ACAGCGGAAG ATGTGACAGC ACCCTTAGTG GATGAGGGAG CTCCCGGCAA GCAGGCTGCC
  421 GCGCAGCCCC ACACGGAGAT CCCAGAAGGA ACCACAGCTG AAGAAGCAGG CATTGGAGAC
  481 ACCCCCAGCC TGGAAGACGA AGCTGCTGGT CACGTGACCC AAGAGCCTGA AAGTGGTAAG
  541 GTGGTCCAGG AAGGCTTCCT CCGAGAGCCA GGCCCCCCAG GTCTGAGCCA CCAGCTCATG
  601 TCCGGCATGC CTGGGGCTCC CCTCCTGCCT GAGGGCCCCA GAGAGGCCAC ACGCCAACCT
  661 TCGGGGACAG GACCTGAGGA CACAGAGGGC GGCCGCCACG CCCCTGAGCT GCTCAAGCAC
  721 CAGCTTCTAG GAGACCTGCA CCAGGAGGGG CCGCCGCTGA AGGGGGCAGG GGGCAAAGAG
  781 AGGCCGGGGA GCAAGGAGGA GGTGGATGAA GACCGCGACG TCGATGAGTC CTCCCCCCAA
  841 GACTCCCCTC CCTCCAAGGC CTCCCCAGCC CAAGATGGGC GGCCTCCCCA GACAGCCGCC
  901 AGAGAAGCCA CCAGCATCCC AGGCTTCCCA GCGGAGGGTG CCATCCCCCT CCCTGTGGAT
  961 TTCCTCTCCA AAGTTTCCAC AGAGATCCCA GCCTCAGAGC CCGACGGGCC CAGTGTAGGG
1021 CGGGCCAAAG GGCAGGATGC CCCCCTGGAG TTCACGTTTC ACGTGGAAAT CACACCCAAC
1081 GTGCAGAAGG AGCAGGCGCA CTCGGAGGAG CATTTGGGAA GGGCTGCATT TCCAGGGGCC
1141 CCTGGAGAGG GGCCAGAGGC CCGGGGCCCC TCTTTGGGAG AGGACACAAA AGAGGCTGAC
1201 CTTCCAGAGC CCTCTGAAAA GCAGCCTGCT GCTGCTCCGC GGGGGAAGCC CGTCAGCCGG
1261 GTCCCTCAAC TCAAAGCTCG CATGGTCAGT AAAAGCAAAG ACGGGACTGG AAGCGATGAC
1321 AAAAAAGCCA AGACATCCAC ACGTTCCTCT GCTAAAACCT TGAAAAATAG GCCTTGCCTT
1381 AGCCCCAAAC ACCCCACTCC TGGTAGCTCA GACCCTCTGA TCCAACCCTC CAGCCCTGCT
1441 GTGTGCCCAG AGCCACCTTC CTCTCCTAAA TACGTCTCTT CTGTCACTTC CCGAACTGGC
1501 AGTTCTGGAG CAAAGGAGAT GAAACTCAAG GGGGCTGATG GTAAAACGAA GATCGCCACA
1561 CCGCGGGGAG CAGCCCCTCC AGGCCAGAAG GGCCAGGCCA ACGCCACCAG GATTCCAGCA
1621 AAAACCCCGC CCGCTCCAAA GACACCACCC AGCTCTGCGA CTAAGCAAGT CCAGAGAAGA
1681 CCACCCCCTG CAGGGCCCAG ATCTGAGAGA GGTGAACCTC CAAAATCAGG GGATCGCAGC
1741 GGCTACAGCA GCCCCGGCTC CCCAGGCACT CCCGGCAGCC GCTCCCGCAC CCCGTCCCTT
1801 CCAACCCCAC CCACCCGGGA GCCCAAGAAG GTGGCAGTGG TCCGTACTCC ACCCAAGTCG
1861 CCGTCTTCCG CCAAGAGCCG CCTGCAGACA GCCCCCGTGC CCATGCCAGA CCTGAAGAAT
1921 GTCAAGTCCA AGATCGGCTC CACTGAGAAC CTGAAGCACC AGCCGGGAGG CGGGAAGGTG
1981 CAGATAATTA ATAAGAAGCT GGATCTTAGC AACGTCCAGT CCAAGTGTGG CTCAAAGGAT
2041 AATATCAAAC ACGTCCCGGG AGGCGGCAGT GTGCAAATAG TCTACAAACC AGTTGACCTG
2101 AGCAAGGTGA CCTCCAAGTG TGGCTCATTA GGCAACATCC ATCATAAACC AGGAGGTGGC
2161 CAGGTGGAAG TAAAATCTGA GAAGCTTGAC TTCAAGGACA GAGTCCAGTC GAAGATTGGG
2221 TCCCTGGACA ATATCACCCA CGTCCCTGGC GGAGGAAATA AAAAGATTGA AACCCACAAG
2281 CTGACCTTCC GCGAGAACGC CAAAGCCAAG ACAGACCACG GGGCGGAGAT CGTGTACAAG
2341 TCGCCAGTGG TGTCTGGGGA CACGTCTCCA CGGCATCTCA GCAATGTCTC CTCCACCGGC
2401 AGCATCGACA TGGTAGACTC GCCCCAGCTC GCCACGCTAG CTGACGAGGT GTCTGCCTCC
2461 CTGGCCAAGC AGGGTTTGTG ATCAGGCCCC TGGGGCGGTC AATAATTGTG GAGAGGAGAG
2521 AATGAGAGAG TGTGGAAAAA AAAAGAATAA TGACCCGGCC CCCGCCCTCT GCCCCCAGCT
2581 GCTCCTCGCA GTTCGGTTAA TTGGTTAATC ACTTAACCTG CTTTTGTCAC TCGGCTTTGG
2641 CTCGGGACTT CAAAATCAGT GATGGGAGTA AGAGCAAATT TCATCTTTCC AAATTGATGG
2701 GTGGGCTAGT AATAAAATAT TTAAAAAAAA ACATTCAAAA ACATGGCCAC ATCCAACATT
2761 TCCTCAGGCA ATTCCTTTTG ATTCTTTTTT CTTCCCCCTC CATGTAGAAG AGGGAGAAGG
2821 AGAGGCTCTG AAAGCTGCTT CTGGGGGATT TCAAGGGACT GGGGGTGCCA ACCACCTCTG
2881 GCCCTGTTGT GGGGGTGTCA CAGAGGCAGT GGCAGCAACA AAGGATTTGA AACTTGGTGT
2941 GTTCGTGGAG CCACAGGCAG ACGATGTCAA CCTTGTGTGA GTGTGACGGG GGTTGGGGTG
3001 GGGCGGGAGG CCACGGGGGA GGCCGAGGCA GGGGCTGGGC AGAGGGGAGA GGAAGCACAA
3061 GAAGTGGGAG TGGGAGAGGA AGCCACGTGC TGGAGAGTAG ACATCCCCCT CCTTGCCGCT
3121 GGGAGAGCCA AGGCCTATGC CACCTGCAGC GTCTGAGCGG CCGCCTGTCC TTGGTGGCCG
3181 GGGGTGGGGG CCTGCTGTGG GTCAGTGTGC CACCCTCTGC AGGGCAGCCT GTGGGAGAAG
3241 GGACAGCGGG TAAAAAGAGA AGGCAAGCTG GCAGGAGGGT GGCACTTCGT GGATGACCTC
3301 CTTAGAAAAG ACTGACCTTG ATGTCTTGAG AGCGCTGGCC TCTTCCTCCC TCCCTGCAGG
3361 GTAGGGGGCC TGAGTTGAGG GGCTTCCCTC TGCTCCACAG AAACCCTGTT TTATTGAGTT
3421 CTGAAGGTTG GAACTGCTGC CATGATTTTG GCCACTTTGC AGACCTGGGA CTTTAGGGCT
3481 AACCAGTTCT CTTTGTAAGG ACTTGTGCCT CTTGGGAGAC GTCCACCCGT TTCCAAGCCT
3541 GGGCCACTGG CATCTCTGGA GTGTGTGGGG GTCTGGGAGG CAGGTCCCGA GCCCCCTGTC
3601 CTTCCCACGG CCACTGCAGT CACCCCGTCT GCGCCGCTGT GCTGTTGTCT GCCGTGAGAG
3661 CCCAATCACT GCCTATACCC CTCATCACAC GTCACAATGT CCCGAATTCC CAGCCTCACC
3721 ACCCCTTCTC AGTAATGACC CTGGTTGGTT GCAGGAGGTA CCTACTCCAT ACTGAGGGTG
3781 AAATTAAGGG AAGGCAAAGT CCAGGCACAA GAGTGGGACC CCAGCCTCTC ACTCTCAGTT
3841 CCACTCATCC AACTGGGACC CTCACCACGA ATCTCATGAT CTGATTCGGT TCCCTGTCTC
3901 CTCCTCCCGT CACAGATGTG AGCCAGGGCA CTGCTCAGCT GTGACCCTAG GTGTTTCTGC
3961 CTTGTTGACA TGGAGAGAGC CCTTTCCCCT GAGAAGGCCT GGCCCCTTCC TGTGCTGAGC
4021 CCACAGCAGC AGGCTGGGTG TCTTGGTTGT CAGTGGTGGC ACCAGGATGG AAGGGCAAGG
4081 CACCCAGGGC AGGCCCACAG TCCCGCTGTC CCCCACTTGC ACCCTAGCTT GTAGCTGCCA
4141 ACCTCCCAGA CAGCCCAGCC CGCTGCTCAG CTCCACATGC ATAGTATCAG CCCTCCACAC
4201 CCGACAAAGG GGAACACACC CCCTTGGAAA TGGTTCTTTT CCCCCAGTCC CAGCTGGAAG
4261 CCATGCTGTC TGTTCTGCTG GAGCAGCTGA ACATATACAT AGATGTTGCC CTGCCCTCCC
4321 CATCTGCACC CTGTTGAGTT GTAGTTGGAT TTGTCTGTTT ATGCTTGGAT TCACCAGAGT
4381 GACTATGATA GTGAAAAGAA AAAAAAAAAA AAAAAAGGAC GCATGTATCT TGAAATGCTT
4441 GTAAAGAGGT TTCTAACCCA CCCTCACGAG GTGTCTCTCA CCCCCACACT GGGACTCGTG
4501 TGGCCTGTGT GGTGCCACCC TGCTGGGGCC TCCCAAGTTT TGAAAGGCTT TCCTCAGCAC
4561 CTGGGACCCA ACAGAGACCA GCTTCTAGCA GCTAAGGAGG CCGTTCAGCT GTGACGAAGG
4621 CCTGAAGCAC AGGATTAGGA CTGAAGCGAT GATGTCCCCT TCCCTACTTC CCCTTGGGGC
4681 TCCCTGTGTC AGGGCACAGA CTAGGTCTTG TGGCTGGTCT GGCTTGCGGC GCGAGGATGG
4741 TTCTCTCTGG TCATAGCCCG AAGTCTCATG GCAGTCCCAA AGGAGGCTTA CAACTCCTGC
4801 ATCACAAGAA AAAGGAAGCC ACTGCCAGCT GGGGGGATCT GCAGCTCCCA GAAGCTCCGT
4861 GAGCCTCAGC CACCCCTCAG ACTGGGTTCC TCTCCAAGCT CGCCCTCTGG AGGGGCAGCG
4921 CAGCCTCCCA CCAAGGGCCC TGCGACCACA GCAGGGATTG GGATGAATTG CCTGTCCTGG
4981 ATCTGCTCTA GAGGCCCAAG CTGCCTGCCT GAGGAAGGAT GACTTGACAA GTCAGGAGAC
5041 ACTGTTCCCA AAGCCTTGAC CAGAGCACCT CAGCCCGCTG ACCTTGCACA AACTCCATCT
5101 GCTGCCATGA GAAAAGGGAA GCCGCCTTTG CAAAACATTG CTGCCTAAAG AAACTCAGCA
5161 GCCTCAGGCC CAATTCTGCC ACTTCTGGTT TGGGTACAGT TAAAGGCAAC CCTGAGGGAC
5221 TTGGCAGTAG AAATCCAGGG CCTCCCCTGG GGCTGGCAGC TTCGTGTGCA GCTAGAGCTT
5281 TACCTGAAAG GAAGTCTCTG GGCCCAGAAC TCTCCACCAA GAGCCTCCCT GCCGTTCGCT
5341 GAGTCCCAGC AATTCTCCTA AGTTGAAGGG ATCTGAGAAG GAGAAGGAAA TGTGGGGTAG
5401 ATTTGGTGGT GGTTAGAGAT ATGCCCCCCT CATTACTGCC AACAGTTTCG GCTGCATTTC
5461 TTCACGCACC TCGGTTCCTC TTCCTGAAGT TCTTGTGCCC TGCTCTTCAG CACCATGGGC
5521 CTTCTTATAC GGAAGGCTCT GGGATCTCCC CCTTGTGGGG CAGGCTCTTG GGGCCAGCCT
5581 AAGATCATGG TTTAGGGTGA TCAGTGCTGG CAGATAAATT GAAAAGGCAC GCTGGCTTGT
5641 GATCTTAAAT GAGGACAATC CCCCCAGGGC TGGGCACTCC TCCCCTCCCC TCACTTCTCC
5701 CACCTGCAGA GCCAGTGTCC TTGGGTGGGC TAGATAGGAT ATACTGTATG CCGGCTCCTT
5761 CAAGCTGCTG ACTCACTTTA TCAATAGTTC CATTTAAATT GACTTCAGTG GTGAGACTGT
5821 ATCCTGTTTG CTATTGCTTG TTGTGCTATG GGGGGAGGGG GGAGGAATGT GTAAGATAGT
5881 TAACATGGGC AAAGGGAGAT CTTGGGGTGC AGCACTTAAA CTGCCTCGTA ACCCTTTTCA
5941 TGATTTCAAC CACATTTGCT AGAGGGAGGG AGCAGCCACG GAGTTAGAGG CCCTTGGGGT
6001 TTCTCTTTTC CACTGACAGG CTTTCCCAGG CAGCTGGCTA GTTCATTCCC TCCCCAGCCA
6061 GGTGCAGGCG TAGGAATATG GACATCTGGT TGCTTTGGCC TGCTGCCCTC TTTCAGGGGT
6121 CCTAAGCCCA CAATCATGCC TCCCTAAGAC CTTGGCATCC TTCCCTCTAA GCCGTTGGCA
6181 CCTCTGTGCC ACCTCTCACA CTGGCTCCAG ACACACAGCC TGTGCTTTTG GAGCTGAGAT
6241 CACTCGCTTC ACCCTCCTCA TCTTTGTTCT CCAAGTAAAG CCACGAGGTC GGGGCGAGGG
6301 CAGAGGTGAT CACCTGCGTG TCCCATCTAC AGACCTGCAG CTTCATAAAA CTTCTGATTT
6361 CTCTTCAGCT TTGAAAAGGG TTACCCTGGG CACTGGCCTA GAGCCTCACC TCCTAATAGA
6421 CTTAGCCCCA TGAGTTTGCC ATGTTGAGCA GGACTATTTC TGGCACTTGC AAGTCCCATG
6481 ATTTCTTCGG TAATTCTGAG GGTGGGGGGA GGGACATGAA ATCATCTTAG CTTAGCTTTC
6541 TGTCTGTGAA TGTCTATATA GTGTATTGTG TGTTTTAACA AATGATTTAC ACTGACTGTT
6601 GCTGTAAAAG TGAATTTGGA AATAAAGTTA TTACTCTGAT TAAA.
```

The corresponding amino acid sequence of human Tau protein isoform 6 can be found at NP_001116538.2:

```
(SEQ ID NO: 157)
    1 MAEPRQEFEV MEDHAGTYGL GDRKDQGGYT
        MHQDQEGDTD AGLKESPLQT PTEDGSEEPG
  61 SETSDAKSTP TAEDVTAPLV DEGAPGKQAA
        AQPHTEIPEG TTAEEAGIGD TPSLEDEAAG
121 HVTQEPESGK VVQEGFLREP GPPGLSHQLM
        SGMPGAPLLP EGPREATRQP SGTGPEDTEG
181 GRHAPELLKH QLLGDLHQEG PPLKGAGGKE
        RPGSKEEVDE DRDVDESSPQ DSPPSKASPA
241 QDGRPPQTAA REATSIPGFP AEGAIPLPVD
        FLSKVSTEIP ASEPDGPSVG RAKGQDAPLE
301 FTFHVEITPN VQKEQAHSEE HLGRAAFPGA
        PGEGPEARGP SLGEDTKEAD LPEPSEKQPA
361 AAPRGKPVSR VPQLKARMVS KSKDGTGSDD
        KKAKTSTRSS AKTLKNRPCL SPKHPTPGSS
421 DPLIQPSSPA VCPEPPSSPK YVSSVTSRTG
        SSGAKEMKLK GADGKTKIAT PRGAAPPGQK
481 GQANATRIPA KTPPAPKTPP SSATKQVQRR
        PPPAGPRSER GEPPKSGDRS GYSSPGSPGT
541 PGSRSRTPSL PTPPTREPKK VAVVRTPPKS
        PSSAKSRLQT APVPMPDLKN VKSKIGSTEN
601 LKHQPGGGKV QIINKKLDLS NVQSKCGSKD
        NIKHVPGGGS VQIVYKPVDL SKVTSKCGSL
661 GNIHHKPGGG QVEVKSEKLD FKDRVQSKIG
        SLDNITHVPG GGNKKIETHK LTFRENAKAK
721 TDHGAEIVYK SPVVSGDTSP RHLSNVSSTG
        SIDMVDSPQL ATLADEVSAS LAKQGL
```

The nucleotide sequence of a human MAPT transcript variant 5 (encoding 1N4R Tau) can be found at NM_001123067.4:

```
(SEQ ID NO: 158)
      1 GCAGTCACCG CCACCCACCA GCTCCGGCAC CAACAGCAGC GCCGCTGCCA CCGCCCACCT
    61 TCTGCCGCCG CCACCACAGC CACCTTCTCC TCCTCCGCTG TCCTCTCCCG TCCTCGCCTC
  121 TGTCGACTAT CAGGTGAACT TTGAACCAGG ATGGCTGAGC CCCGCCAGGA GTTCGAAGTG
  181 ATGGAAGATC ACGCTGGGAC GTACGGGTTG GGGGACAGGA AAGATCAGGG GGGCTACACC
  241 ATGCACCAAG ACCAAGAGGG TGACACGGAC GCTGGCCTGA AAGAATCTCC CCTGCAGACC
  301 CCCACTGAGG ACGGATCTGA GGAACCGGGC TCTGAAACCT CTGATGCTAA GAGCACTCCA
  361 ACAGCGGAAG CTGAAGAAGC AGGCATTGGA GACACCCCCA GCCTGGAAGA CGAAGCTGCT
  421 GGTCACGTGA CCCAAGCTCG CATGGTCAGT AAAAGCAAAG ACGGGACTGG AAGCGATGAC
  481 AAAAAAGCCA AGGGGGCTGA TGGTAAAACG AAGATCGCCA CACCGCGGGG AGCAGCCCCT
  541 CCAGGCCAGA AGGGCCAGGC CAACGCCACC AGGATTCCAG CAAAAACCCC GCCCGCTCCA
  601 AAGACACCAC CCAGCTCTGG TGAACCTCCA AAATCAGGGG ATCGCAGCGG CTACAGCAGC
  661 CCCGGCTCCC CAGGCACTCC CGGCAGCCGC TCCCGCACCC CGTCCCTTCC AACCCCACCC
  721 ACCCGGGAGC CCAAGAAGGT GGCAGTGGTC CGTACTCCAC CCAAGTCGCC GTCTTCCGCC
  781 AAGAGCCGCC TGCAGACAGC CCCCGTGCCC ATGCCAGACC TGAAGAATGT CAAGTCCAAG
  841 ATCGGCTCCA CTGAGAACCT GAAGCACCAG CCGGGAGGCG GGAAGGTGCA GATAATTAAT
  901 AAGAAGCTGG ATCTTAGCAA CGTCCAGTCC AAGTGTGGCT CAAAGGATAA TATCAAACAC
  961 GTCCCGGGAG GCGGCAGTGT GCAAATAGTC TACAAACCAG TTGACCTGAG CAAGGTGACC
1021 TCCAAGTGTG GCTCATTAGG CAACATCCAT CATAAACCAG GAGGTGGCCA GGTGGAAGTA
1081 AAATCTGAGA AGCTTGACTT CAAGGACAGA GTCCAGTCGA AGATTGGGTC CCTGGACAAT
1141 ATCACCCACG TCCCTGGCGG AGGAAATAAA AAGATTGAAA CCCACAAGCT GACCTTCCGC
1201 GAGAACGCCA AAGCCAAGAC AGACCACGGG GCGGAGATCG TGTACAAGTC GCCAGTGGTG
1261 TCTGGGGACA CGTCTCCACG GCATCTCAGC AATGTCTCCT CCACCGGCAG CATCGACATG
1321 GTAGACTCGC CCCAGCTCGC CACGCTAGCT GACGAGGTGT CTGCCTCCCT GGCCAAGCAG
1381 GGTTTGTGAT CAGGCCCCTG GGGCGGTCAA TAATTGTGGA GAGGAGAGAA TGAGAGAGTG
1441 TGGAAAAAAA AAGAATAATG ACCCGGCCCC CGCCCTCTGC CCCCAGCTGC TCCTCGCAGT
1501 TCGGTTAATT GGTTAATCAC TTAACCTGCT TTTGTCACTC GGCTTTGGCT CGGGACTTCA
1561 AAATCAGTGA TGGGAGTAAG AGCAAATTTC ATCTTTCCAA ATTGATGGGT GGGCTAGTAA
1621 TAAAATATTT AAAAAAAAAC ATTCAAAAAC ATGGCCACAT CCAACATTTC CTCAGGCAAT
1681 TCCTTTTGAT TCTTTTTTCT TCCCCCTCCA TGTAGAAGAG GGAGAAGGAG AGGCTCTGAA
1741 AGCTGCTTCT GGGGGATTTC AAGGGACTGG GGGTGCCAAC CACCTCTGGC CCTGTTGTGG
1801 GGGTGTCACA GAGGCAGTGG CAGCAACAAA GGATTTGAAA CTTGGTGTGT TCGTGGAGCC
1861 ACAGGCAGAC GATGTCAACC TTGTGTGAGT GTGACGGGGG TTGGGGTGGG GCGGGAGGCC
1921 ACGGGGGAGG CCGAGGCAGG GGCTGGGCAG AGGGGAGAGG AAGCACAAGA AGTGGGAGTG
1981 GGAGAGGAAG CCACGTGCTG GAGAGTAGAC ATCCCCCTCC TTGCCGCTGG GAGAGCCAAG
2041 GCCTATGCCA CCTGCAGCGT CTGAGCGGCC GCCTGTCCTT GGTGGCCGGG GGTGGGGGCC
2101 TGCTGTGGGT CAGTGTGCCA CCCTCTGCAG GGCAGCCTGT GGGAGAAGGG ACAGCGGGTA
2161 AAAAGAGAAG GCAAGCTGGC AGGAGGGTGG CACTTCGTGG ATGACCTCCT TAGAAAAGAC
2221 TGACCTTGAT GTCTTGAGAG CGCTGGCCTC TTCCTCCCTC CCTGCAGGGT AGGGGGCCTG
2281 AGTTGAGGGG CTTCCCTCTG CTCCACAGAA ACCCTGTTTT ATTGAGTTCT GAAGGTTGGA
2341 ACTGCTGCCA TGATTTTGGC CACTTTGCAG ACCTGGGACT TTAGGGCTAA CCAGTTCTCT
2401 TTGTAAGGAC TTGTGCCTCT TGGGAGACGT CCACCCGTTT CCAAGCCTGG GCCACTGGCA
2461 TCTCTGGAGT GTGTGGGGGT CTGGGAGGCA GGTCCCGAGC CCCCTGTCCT TCCCACGGCC
2521 ACTGCAGTCA CCCCGTCTGC GCCGCTGTGC TGTTGTCTGC CGTGAGAGCC CAATCACTGC
2581 CTATACCCCT CATCACACGT CACAATGTCC CGAATTCCCA GCCTCACCAC CCCTTCTCAG
2641 TAATGACCCT GGTTGGTTGC AGGAGGTACC TACTCCATAC TGAGGGTGAA ATTAAGGGAA
2701 GGCAAAGTCC AGGCACAAGA GTGGGACCCC AGCCTCTCAC TCTCAGTTCC ACTCATCCAA
2761 CTGGGACCCT CACCACGAAT CTCATGATCT GATTCGGTTC CCTGTCTCCT CCTCCCGTCA
2821 CAGATGTGAG CCAGGGCACT GCTCAGCTGT GACCCTAGGT GTTTCTGCCT TGTTGACATG
2881 GAGAGAGCCC TTTCCCCTGA GAAGGCCTGG CCCCTTCCTG TGCTGAGCCC ACAGCAGCAG
2941 GCTGGGTGTC TTGGTTGTCA GTGGTGGCAC CAGGATGGAA GGGCAAGGCA CCCAGGGCAG
3001 GCCCACAGTC CCGCTGTCCC CCACTTGCAC CCTAGCTTGT AGCTGCCAAC CTCCCAGACA
3061 GCCCAGCCCG CTGCTCAGCT CCACATGCAT AGTATCAGCC CTCCACACCC GACAAAGGGG
3121 AACACACCCC CTTGGAAATG GTTCTTTTCC CCCAGTCCCA GCTGGAAGCC ATGCTGTCTG
3181 TTCTGCTGGA GCAGCTGAAC ATATACATAG ATGTTGCCCT GCCCTCCCCA TCTGCACCCT
3241 GTTGAGTTGT AGTTGGATTT GTCTGTTTAT GCTTGGATTC ACCAGAGTGA CTATGATAGT
3301 GAAAAGAAAA AAAAAAAAAA AAAAGGACGC ATGTATCTTG AAATGCTTGT AAAGAGGTTT
3361 CTAACCCACC CTCACGAGGT GTCTCTCACC CCCACACTGG GACTCGTGTG GCCTGTGTGG
3421 TGCCACCCTG CTGGGGCCTC CCAAGTTTTG AAAGGCTTTC CTCAGCACCT GGGACCCAAC
3481 AGAGACCAGC TTCTAGCAGC TAAGGAGGCC GTTCAGCTGT GACGAAGGCC TGAAGCACAG
3541 GATTAGGACT GAAGCGATGA TGTCCCCTTC CCTACTTCCC CTTGGGGCTC CCTGTGTCAG
3601 GGCACAGACT AGGTCTTGTG GCTGGTCTGG CTTGCGGCGC GAGGATGGTT CTCTCTGGTC
3661 ATAGCCCGAA GTCTCATGGC AGTCCCAAAG GAGGCTTACA ACTCCTGCAT CACAAGAAAA
3721 AGGAAGCCAC TGCCAGCTGG GGGGATCTGC AGCTCCCAGA AGCTCCGTGA GCCTCAGCCA
3781 CCCCTCAGAC TGGGTTCCTC TCCAAGCTCG CCCTCTGGAG GGGCAGCGCA GCCTCCCACC
3841 AAGGGCCCTG CGACCACAGC AGGGATTGGG ATGAATTGCC TGTCCTGGAT CTGCTCTAGA
3901 GGCCCAAGCT GCCTGCCTGA GGAAGGATGA CTTGACAAGT CAGGAGACAC TGTTCCCAAA
3961 GCCTTGACCA GAGCACCTCA GCCCGCTGAC CTTGCACAAA CTCCATCTGC TGCCATGAGA
4021 AAAGGGAAGC CGCCTTTGCA AAACATTGCT GCCTAAAGAA ACTCAGCAGC CTCAGGCCCA
4081 ATTCTGCCAC TTCTGGTTTG GGTACAGTTA AAGGCAACCC TGAGGGACTT GGCAGTAGAA
4141 ATCCAGGGCC TCCCCTGGGG CTGGCAGCTT CGTGTGCAGC TAGAGCTTTA CCTGAAAGGA
4201 AGTCTCTGGG CCCAGAACTC TCCACCAAGA GCCTCCCTGC CGTTCGCTGA GTCCCAGCAA
4261 TTCTCCTAAG TTGAAGGGAT CTGAGAAGGA GAAGGAAATG TGGGGTAGAT TTGGTGGTGG
4321 TTAGAGATAT GCCCCCCTCA TTACTGCCAA CAGTTTCGGC TGCATTTCTT CACGCACCTC
4381 GGTTCCTCTT CCTGAAGTTC TTGTGCCCTG CTCTTCAGCA CCATGGGCCT TCTTATACGG
4441 AAGGCTCTGG GATCTCCCCC TTGTGGGGCA GGCTCTTGGG GCCAGCCTAA GATCATGGTT
4501 TAGGGTGATC AGTGCTGGCA GATAAATTGA AAAGGCACGC TGGCTTGTGA TCTTAAATGA
4561 GGACAATCCC CCCAGGGCTG GGCACTCCTC CCCTCCCCTC ACTTCTCCCA CCTGCAGAGC
4621 CAGTGTCCTT GGGTGGGCTA GATAGGATAT ACTGTATGCC GGCTCCTTCA AGCTGCTGAC
4681 TCACTTTATC AATAGTTCCA TTTAAATTGA CTTCAGTGGT GAGACTGTAT CCTGTTTGCT
4741 ATTGCTTGTT GTGCTATGGG GGGAGGGGGG AGGAATGTGT AAGATAGTTA ACATGGGCAA
4801 AGGGAGATCT TGGGGTGCAG CACTTAAACT GCCTCGTAAC CCTTTTCATG ATTTCAACCA
4861 CATTTGCTAG AGGGAGGGAG CAGCCACGGA GTTAGAGGCC CTTGGGGTTT CTCTTTTCCA
4921 CTGACAGGCT TTCCCAGGCA GCTGGCTAGT TCATTCCCTC CCCAGCCAGG TGCAGGCGTA
4981 GGAATATGGA CATCTGGTTG CTTTGGCCTG CTGCCCTCTT TCAGGGGTCC TAAGCCCACA
5041 ATCATGCCTC CCTAAGACCT TGGCATCCTT CCCTCTAAGC CGTTGGCACC TCTGTGCCAC
5101 CTCTCACACT GGCTCCAGAC ACACAGCCTG TGCTTTTGGA GCTGAGATCA CTCGCTTCAC
5161 CCTCCTCATC TTTGTTCTCC AAGTAAAGCC ACGAGGTCGG GGCGAGGGCA GAGGTGATCA
5221 CCTGCGTGTC CCATCTACAG ACCTGCAGCT TCATAAAACT TCTGATTTCT CTTCAGCTTT
5281 GAAAAGGGTT ACCCTGGGCA CTGGCCTAGA GCCTCACCTC CTAATAGACT TAGCCCCATG
5341 AGTTTGCCAT GTTGAGCAGG ACTATTTCTG GCACTTGCAA GTCCCATGAT TTCTTCGGTA
5401 ATTCTGAGGG TGGGGGGAGG GACATGAAAT CATCTTAGCT TAGCTTTCTG TCTGTGAATG
5461 TCTATATAGT GTATTGTGTG TTTTAACAAA TGATTTACAC TGACTGTTGC TGTAAAAGTG
5521 AATTTGGAAA TAAAGTTATT ACTCTGATTA AA.
```

The corresponding amino acid sequence of human Tau protein isoform 5 can be found at NP_001116539.1:

```
(SEQ ID NO: 159)
    1 MAEPRQEFEV MEDHAGTYGL GDRKDQGGYT
        MHQDQEGDTD AGLKESPLQT PTEDGSEEPG
  61 SETSDAKSTP TAEAEEAGIG DTPSLEDEAA
        GHVTQARMVS KSKDGTGSDD KKAKGADGKT
121 KIATPRGAAP PGQKGQANAT RIPAKTPPAP
        KTPPSSGEPP KSGDRSGYSS PGSPGTPGSR
181 SRTPSLPTPP TREPKKVAVV RTPPKSPSSA
        KSRLQTAPVP MPDLKNVKSK IGSTENLKHQ
241 PGGGKVQIIN KKLDLSNVQS KCGSKDNIKH
        VPGGGSVQIV YKPVDLSKVT SKCGSLGNIH
301 HKPGGGQVEV KSEKLDFKDR VQSKIGSLDN
        ITHVPGGGNK KIETHKLTFR ENAKAKTDHG
361 AEIVYKSPVV SGDTSPRHLS NVSSTGSIDM
        VDSPQLATLA DEVSASLAKQ GL
```

The nucleotide sequence of the human MAPT transcript variant 4 (encoding 0N3R Tau) can be found at NM 016841.5:

```
(SEQ ID NO: 160)
      1 GCAGTCACCG CCACCCACCA GCTCCGGCAC CAACAGCAGC GCCGCTGCCA CCGCCCACCT
    61 TCTGCCGCCG CCACCACAGC CACCTTCTCC TCCTCCGCTG TCCTCTCCCG TCCTCGCCTC
  121 TGTCGACTAT CAGGTGAACT TTGAACCAGG ATGGCTGAGC CCCGCCAGGA GTTCGAAGTG
  181 ATGGAAGATC ACGCTGGGAC GTACGGGTTG GGGGACAGGA AAGATCAGGG GGGCTACACC
  241 ATGCACCAAG ACCAAGAGGG TGACACGGAC GCTGGCCTGA AAGCTGAAGA AGCAGGCATT
  301 GGAGACACCC CCAGCCTGGA AGACGAAGCT GCTGGTCACG TGACCCAAGC TCGCATGGTC
  361 AGTAAAAGCA AAGACGGGAC TGGAAGCGAT GACAAAAAAG CCAAGGGGGC TGATGGTAAA
  421 ACGAAGATCG CCACACCGCG GGGAGCAGCC CCTCCAGGCC AGAAGGGCCA GGCCAACGCC
  481 ACCAGGATTC CAGCAAAAAC CCCGCCCGCT CCAAAGACAC CACCCAGCTC TGGTGAACCT
  541 CCAAAATCAG GGGATCGCAG CGGCTACAGC AGCCCCGGCT CCCCAGGCAC TCCCGGCAGC
  601 CGCTCCCGCA CCCCGTCCCT TCCAACCCCA CCCACCCGGG AGCCCAAGAA GGTGGCAGTG
  661 GTCCGTACTC CACCCAAGTC GCCGTCTTCC GCCAAGAGCC GCCTGCAGAC AGCCCCCGTG
  721 CCCATGCCAG ACCTGAAGAA TGTCAAGTCC AAGATCGGCT CCACTGAGAA CCTGAAGCAC
  781 CAGCCGGGAG GCGGGAAGGT GCAAATAGTC TACAAACCAG TTGACCTGAG CAAGGTGACC
  841 TCCAAGTGTG GCTCATTAGG CAACATCCAT CATAAACCAG GAGGTGGCCA GGTGGAAGTA
  901 AAATCTGAGA AGCTTGACTT CAAGGACAGA GTCCAGTCGA AGATTGGGTC CCTGGACAAT
  961 ATCACCCACG TCCCTGGCGG AGGAAATAAA AAGATTGAAA CCCACAAGCT GACCTTCCGC
1021 GAGAACGCCA AAGCCAAGAC AGACCACGGG GCGGAGATCG TGTACAAGTC GCCAGTGGTG
1081 TCTGGGGACA CGTCTCCACG GCATCTCAGC AATGTCTCCT CCACCGGCAG CATCGACATG
1141 GTAGACTCGC CCCAGCTCGC CACGCTAGCT GACGAGGTGT CTGCCTCCCT GGCCAAGCAG
1201 GGTTTGTGAT CAGGCCCCTG GGGCGGTCAA TAATTGTGGA GAGGAGAGAA TGAGAGAGTG
1261 TGGAAAAAAA AAGAATAATG ACCCGGCCCC CGCCCTCTGC CCCCAGCTGC TCCTCGCAGT
1321 TCGGTTAATT GGTTAATCAC TTAACCTGCT TTTGTCACTC GGCTTTGGCT CGGGACTTCA
1381 AAATCAGTGA TGGGAGTAAG AGCAAATTTC ATCTTTCCAA ATTGATGGGT GGGCTAGTAA
1441 TAAAATATTT AAAAAAAAAC ATTCAAAAAC ATGGCCACAT CCAACATTTC CTCAGGCAAT
1501 TCCTTTTGAT TCTTTTTTCT TCCCCCTCCA TGTAGAAGAG GGAGAAGGAG AGGCTCTGAA
1561 AGCTGCTTCT GGGGGATTTC AAGGGACTGG GGGTGCCAAC CACCTCTGGC CCTGTTGTGG
1621 GGGTGTCACA GAGGCAGTGG CAGCAACAAA GGATTTGAAA CTTGGTGTGT TCGTGGAGCC
1681 ACAGGCAGAC GATGTCAACC TTGTGTGAGT GTGACGGGGG TTGGGGTGGG GCGGGAGGCC
1741 ACGGGGGAGG CCGAGGCAGG GGCTGGGCAG AGGGGAGAGG AAGCACAAGA AGTGGGAGTG
1801 GGAGAGGAAG CCACGTGCTG GAGAGTAGAC ATCCCCCTCC TTGCCGCTGG GAGAGCCAAG
1861 GCCTATGCCA CCTGCAGCGT CTGAGCGGCC GCCTGTCCTT GGTGGCCGGG GGTGGGGGCC
1921 TGCTGTGGGT CAGTGTGCCA CCCTCTGCAG GGCAGCCTGT GGGAGAAGGG ACAGCGGGTA
1981 AAAAGAGAAG GCAAGCTGGC AGGAGGGTGG CACTTCGTGG ATGACCTCCT TAGAAAAGAC
2041 TGACCTTGAT GTCTTGAGAG CGCTGGCCTC TTCCTCCCTC CCTGCAGGGT AGGGGGCCTG
2101 AGTTGAGGGG CTTCCCTCTG CTCCACAGAA ACCCTGTTTT ATTGAGTTCT GAAGGTTGGA
2161 ACTGCTGCCA TGATTTTGGC CACTTTGCAG ACCTGGGACT TTAGGGCTAA CCAGTTCTCT
2221 TTGTAAGGAC TTGTGCCTCT TGGGAGACGT CCACCCGTTT CCAAGCCTGG GCCACTGGCA
2281 TCTCTGGAGT GTGTGGGGGT CTGGGAGGCA GGTCCCGAGC CCCCTGTCCT TCCCACGGCC
2341 ACTGCAGTCA CCCCGTCTGC GCCGCTGTGC TGTTGTCTGC CGTGAGAGCC CAATCACTGC
2401 CTATACCCCT CATCACACGT CACAATGTCC CGAATTCCCA GCCTCACCAC CCCTTCTCAG
2461 TAATGACCCT GGTTGGTTGC AGGAGGTACC TACTCCATAC TGAGGGTGAA ATTAAGGGAA
2521 GGCAAAGTCC AGGCACAAGA GTGGGACCCC AGCCTCTCAC TCTCAGTTCC ACTCATCCAA
2581 CTGGGACCCT CACCACGAAT CTCATGATCT GATTCGGTTC CCTGTCTCCT CCTCCCGTCA
2641 CAGATGTGAG CCAGGGCACT GCTCAGCTGT GACCCTAGGT GTTTCTGCCT TGTTGACATG
2701 GAGAGAGCCC TTTCCCCTGA GAAGGCCTGG CCCCTTCCTG TGCTGAGCCC ACAGCAGCAG
2761 GCTGGGTGTC TTGGTTGTCA GTGGTGGCAC CAGGATGGAA GGGCAAGGCA CCCAGGGCAG
2821 GCCCACAGTC CCGCTGTCCC CCACTTGCAC CCTAGCTTGT AGCTGCCAAC CTCCCAGACA
2881 GCCCAGCCCG CTGCTCAGCT CCACATGCAT AGTATCAGCC CTCCACACCC GACAAAGGGG
2941 AACACACCCC CTTGGAAATG GTTCTTTTCC CCCAGTCCCA GCTGGAAGCC ATGCTGTCTG
3001 TTCTGCTGGA GCAGCTGAAC ATATACATAG ATGTTGCCCT GCCCTCCCCA TCTGCACCCT
3061 GTTGAGTTGT AGTTGGATTT GTCTGTTTAT GCTTGGATTC ACCAGAGTGA CTATGATAGT
3121 GAAAAGAAAA AAAAAAAAAA AAAAGGACGC ATGTATCTTG AAATGCTTGT AAAGAGGTTT
3181 CTAACCCACC CTCACGAGGT GTCTCTCACC CCCACACTGG GACTCGTGTG GCCTGTGTGG
3241 TGCCACCCTG CTGGGGCCTC CCAAGTTTTG AAAGGCTTTC CTCAGCACCT GGGACCCAAC
3301 AGAGACCAGC TTCTAGCAGC TAAGGAGGCC GTTCAGCTGT GACGAAGGCC TGAAGCACAG
3361 GATTAGGACT GAAGCGATGA TGTCCCCTTC CCTACTTCCC CTTGGGGCTC CCTGTGTCAG
3421 GGCACAGACT AGGTCTTGTG GCTGGTCTGG CTTGCGGCGC GAGGATGGTT CTCTCTGGTC
3481 ATAGCCCGAA GTCTCATGGC AGTCCCAAAG GAGGCTTACA ACTCCTGCAT CACAAGAAAA
3541 AGGAAGCCAC TGCCAGCTGG GGGGATCTGC AGCTCCCAGA AGCTCCGTGA GCCTCAGCCA
3601 CCCCTCAGAC TGGGTTCCTC TCCAAGCTCG CCCTCTGGAG GGGCAGCGCA GCCTCCCACC
3661 AAGGGCCCTG CGACCACAGC AGGGATTGGG ATGAATTGCC TGTCCTGGAT CTGCTCTAGA
3721 GGCCCAAGCT GCCTGCCTGA GGAAGGATGA CTTGACAAGT CAGGAGACAC TGTTCCCAAA
3781 GCCTTGACCA GAGCACCTCA GCCCGCTGAC CTTGCACAAA CTCCATCTGC TGCCATGAGA
3841 AAAGGGAAGC CGCCTTTGCA AAACATTGCT GCCTAAAGAA ACTCAGCAGC CTCAGGCCCA
3901 ATTCTGCCAC TTCTGGTTTG GGTACAGTTA AAGGCAACCC TGAGGGACTT GGCAGTAGAA
3961 ATCCAGGGCC TCCCCTGGGG CTGGCAGCTT CGTGTGCAGC TAGAGCTTTA CCTGAAAGGA
4021 AGTCTCTGGG CCCAGAACTC TCCACCAAGA GCCTCCCTGC CGTTCGCTGA GTCCCAGCAA
4081 TTCTCCTAAG TTGAAGGGAT CTGAGAAGGA GAAGGAAATG TGGGGTAGAT TTGGTGGTGG
4141 TTAGAGATAT GCCCCCCTCA TTACTGCCAA CAGTTTCGGC TGCATTTCTT CACGCACCTC
4201 GGTTCCTCTT CCTGAAGTTC TTGTGCCCTG CTCTTCAGCA CCATGGGCCT TCTTATACGG
4261 AAGGCTCTGG GATCTCCCCC TTGTGGGGCA GGCTCTTGGG GCCAGCCTAA GATCATGGTT
4321 TAGGGTGATC AGTGCTGGCA GATAAATTGA AAAGGCACGC TGGCTTGTGA TCTTAAATGA
4381 GGACAATCCC CCCAGGGCTG GGCACTCCTC CCCTCCCCTC ACTTCTCCCA CCTGCAGAGC
4441 CAGTGTCCTT GGGTGGGCTA GATAGGATAT ACTGTATGCC GGCTCCTTCA AGCTGCTGAC
4501 TCACTTTATC AATAGTTCCA TTTAAATTGA CTTCAGTGGT GAGACTGTAT CCTGTTTGCT
4561 ATTGCTTGTT GTGCTATGGG GGGAGGGGGG AGGAATGTGT AAGATAGTTA ACATGGGCAA
4621 AGGGAGATCT TGGGGTGCAG CACTTAAACT GCCTCGTAAC CCTTTTCATG ATTTCAACCA
4681 CATTTGCTAG AGGGAGGGAG CAGCCACGGA GTTAGAGGCC CTTGGGGTTT CTCTTTTCCA
4741 CTGACAGGCT TTCCCAGGCA GCTGGCTAGT TCATTCCCTC CCCAGCCAGG TGCAGGCGTA
4801 GGAATATGGA CATCTGGTTG CTTTGGCCTG CTGCCCTCTT TCAGGGGTCC TAAGCCCACA
4861 ATCATGCCTC CCTAAGACCT TGGCATCCTT CCCTCTAAGC CGTTGGCACC TCTGTGCCAC
4921 CTCTCACACT GGCTCCAGAC ACACAGCCTG TGCTTTTGGA GCTGAGATCA CTCGCTTCAC
4981 CCTCCTCATC TTTGTTCTCC AAGTAAAGCC ACGAGGTCGG GGCGAGGGCA GAGGTGATCA
5041 CCTGCGTGTC CCATCTACAG ACCTGCAGCT TCATAAAACT TCTGATTTCT CTTCAGCTTT
5101 GAAAAGGGTT ACCCTGGGCA CTGGCCTAGA GCCTCACCTC CTAATAGACT TAGCCCCATG
5161 AGTTTGCCAT GTTGAGCAGG ACTATTTCTG GCACTTGCAA GTCCCATGAT TTCTTCGGTA
5221 ATTCTGAGGG TGGGGGGAGG GACATGAAAT CATCTTAGCT TAGCTTTCTG TCTGTGAATG
5281 TCTATATAGT GTATTGTGTG TTTTAACAAA TGATTTACAC TGACTGTTGC TGTAAAAGTG
5341 AATTTGGAAA TAAAGTTATT ACTCTGATTA AA.
```

The corresponding amino acid sequence of human Tau protein isoform 4 can be found at NP 058525.1:

```
(SEQ ID NO: 161)
    1 MAEPRQEFEV MEDHAGTYGL GDRKDQGGYT
        MHQDQEGDTD AGLKAEEAGI GDTPSLEDEA
  61 AGHVTQARMV SKSKDGTGSD DKKAKGADGK
        TKIATPRGAA PPGQKGQANA TRIPAKTPPA
121 PKTPPSSGEP PKSGDRSGYS SPGSPGTPGS
        RSRTPSLPTP PTREPKKVAV VRTPPKSPSS
181 AKSRLQTAPV PMPDLKNVKS KIGSTENLKH
        QPGGGKVQIV YKPVDLSKVT SKCGSLGNIH
241 HKPGGGQVEV KSEKLDFKDR VQSKIGSLDN
        ITHVPGGGNK KIETHKLTFR ENAKAKTDHG
301 AEIVYKSPVV SGDTSPRHLS NVSSTGSIDM
        VDSPQLATLA DEVSASLAKQ GL
```

As used herein, the term “tauopathy” refers to a disease associated with abnormal tau protein expression, secretion, phosphorylation, cleavage, and/or aggregation.

As used herein, “TfR” refers to a transferrin receptor protein or polypeptide, e.g., a human or mouse transferrin receptor protein or polypeptide. The amino acid sequence of the human transferrin receptor protein (hTFR) can be found at NP_001121620.1:

```
(SEQ ID NO: 111)
    1 MMDQARSAFS NLFGGEPLSY TRESLARQVD
        GDNSHVEMKL AVDEEENADN NTKANVTKPK
  61 RCSGSICYGT IAVIVFFLIG FMIGYLGYCK
        GVEPKTECER LAGTESPVRE EPGEDFPAAR
121 RLYWDDLKRK LSEKLDSTDF TGTIKLLNEN
        SYVPREAGSQ KDENLALYVE NQFREFKLSK
181 VWRDQHFVKI QVKDSAQNSV IIVDKNGRLV
        YLVENPGGYV AYSKAATVTG KLVHANFGTK
241 KDFEDLYTPV NGSIVIVRAG KITFAEKVAN
        AESLNAIGVL IYMDQTKFPI VNAELSFFGH
301 AHLGTGDPYT PGFPSENHTQ FPPSRSSGLP
        NIPVQTISRA AAEKLEGNME GDCPSDWKTD
361 STCRMVTSES KNVKLTVSNV LKEIKILNIF
        GVIKGFVEPD HYVVVGAQRD AWGPGAAKSG
421 VGTALLLKLA QMFSDMVLKD GFQPSRSIIF
        ASWSAGDEGS VGATEWLEGY LSSLHLKAFT
481 YINLDKAVLG TSNFKVSASP LLYTLIEKTM
        QNVKHPVTGQ FLYQDSNWAS KVEKLTLDNA
541 AFPFLAYSGI PAVSFCFCED TDYPYLGTTM
        DTYKELIERI PELNKVARAA AEVAGQFVIK
601 LTHDVELNLD YERYNSQLLS FVRDLNQYRA
        DIKEMGLSLQ WLYSARGDFF RATSRLTTDE
661 GNAEKTDRFV MKKLNDRVMR VEYHFLSPYV
        SPKESPFRHV FWGSGSHTLP ALLENLKLRK
721 QNNGAFNETL FRNQLALATW TIQGAANALS
        GDVWDIDNEF.
```

The amino acid sequence of the mouse transferrin receptor protein (mTFR) can be found at NP_001344227.1:

```
(SEQ ID NO: 112)
    1 MMDQARSAFS NLFGGEPLSY TRESLARQVD
        GDNSHVEMKL AADEEENADN NMKASVRKPK
  61 RFNGRLCFAA IALVIFFLIG FMSGYLGYCK
        RVEQKEECVK LAETEETDKS ETMETEDVPT
121 SSRLYWADLK TLLSEKLNSI EFADTIKQLS
        QNTYTPREAG SQKDESLAYY IENQFHEFKF
181 SKVWRDEHYV KIQVKSSIGQ NMVTIVQSNG
        NLDPVESPEG YVAFSKPTEV SGKLVHANFG
241 TKKDFEELSY SVNGSLVIVR AGEITFAEKV
        ANAQSFNAIG VLIYMDKNKF PVVEADLALF
301 GHAHLGTGDP YTPGFPSENH TQFPPSQSSG
        LPNIPVQTIS RAAAEKLEGK MEGSCPARWN
361 IDSSCKLELS QNQNVKLIVK NVLKERRILN
        IFGVIKGYEE PDRYVVVGAQ RDALGAGVAA
421 KSSVGTGLLL KLAQVESDMI SKDGFRPSRS
        IIFASWTAGD FGAVGATEWL EGYLSSLHLK
481 AFTYINLDKV VLGTSNFKVS ASPLLYTLMG
        KIMQDVKHPV DGKSLYRDSN WISKVEKLSF
541 DNAAYPFLAY SGIPAVSFCF CEDADYPYLG
        TRLDTYEALT QKVPQLNQMV RTAAEVAGQL
601 IIKLTHDVEL NLDYEMYNSK LLSFMKDLNQ
        FKTDIRDMGL SLQWLYSARG DYFRATSRLT
661 TDFHNAEKTN RFVMREINDR IMKVEYHFLS
        PYVSPRESPF RHIFWGSGSH TLSALVENLK
721 LRQKNITAFN ETLERNQLAL ATWTIQGVAN
        ALSGDIWNID NEF.
```

As used herein, “treatment” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

The following examples are offered to illustrate, but not to limit, the claimed inventions.

### EXAMPLES

### Example 1: Generation and Characterization of TfR Binding Proteins

### Generation of Human or Mouse TfR Binding Proteins

Antibody against mouse TfR was generated by immunizing New Zealand White rabbits with the extracellular domain (ECD) of mouse Transferrin Receptor 1 protein with a His tag (mTfR-ECD-6His, SEQ ID NO: 113, see Table 12). mTfR antigen positive B-cells were sorted from peripheral blood and binding of individual antibodies cloned from those B-cells was verified on his-tagged mTfR.

Antibody against human TfR was generated by immunizing AlivaMab® transgenic mice with the extracellular domains of human Transferrin Receptor 1 protein with a His tag (hTfR-ECD-6His, SEQ TD NO: 114, see Table 12) and mouse Transferrin Receptor protein (mTfR, SEQ ID NO: 110). Antigen positive B-cells were sorted from pooled spleens. Binding of individual antibodies cloned from those B-cells to his-tagged hTfR-ECD was verified.

Additional antibody against human TfR was generated by immunizing AlivaMab® transgenic mice with the apical domain of human Transferrin Receptor 1 protein with a His tag (hTfR-ApD-6His, SEQ TD NO: 115, see Table 12). Antigen positive B-cells were sorted from pooled spleens. Binding of individual antibodies cloned from those B-cells to his-tagged hTfR-ECD was verified.

TABLE 12
Sequences of the immunogens used to generate
human or mouse TfR antibodies.

| Immunogen | Sequence | SEQ ID NO |
|---|---|---|
| mTfR-ECD-6His | HHHHHHCKRVEQKEECVKLA | 113 |
| | ETEETDKSETMETEDVPTSS | |
| | RLYWADLKTLLSEKLNSIEF | |
| | ADTIKQLSQNTYTPREAGSQ | |
| | KDESLAYYIENQFHEFKFSK | |
| | VWRDEHYVKIQVKSSIGQNM | |
| | VTIVQSNGNLDPVESPEGYV | |
| | AFSKPTEVSGKLVHANFGTK | |
| | KDFEELSYSVNGSLVIVRAG | |
| | EITFAEKVANAQSFNAIGVL | |
| | IYMDKNKFPVVEADLALFGH | |
| | AHLGTGDPYTPGFPSFNHTQ | |
| | FPPSQSSGLPNIPVQTISRA | |
| | AAEKLFGKMEGSCPARWNID | |
| | SSCKLELSQNQNVKLIVKNV | |
| | LKERRILNIFGVIKGYEEPD | |
| | RYVVVGAQRDALGAGVAAKS | |
| | SVGTGLLLKLAQVFSDMISK | |
| | DGFRPSRSIIFASWTAGDFG | |
| | AVGATEWLEGYLSSLHLKAF | |
| | TYINLDKVVLGTSNFKVSAS | |
| | PLLYTLMGKIMQDVKHPVDG | |
| | KSLYRDSNWISKVEKLSFDN | |
| | AAYPFLAYSGIPAVSFCFCE | |
| | DADYPYLGTRLDTYEALTQK | |
| | VPQLNQMVRTAAEVAGQLII | |
| | KLTHDVELNLDYEMYNSKLL | |
| | SFMKDLNQFKTDIRDMGLSL | |
| | QWLYSARGDYFRATSRLTTD | |
| | FHNAEKTNRFVMREINDRIM | |
| | KVEYHFLSPYVSPRESPFRH | |
| | IFWGSGSHTLSALVENLKLR | |
| | QKNITAFNETLFRNQLALAT | |
| | WTIQGVANALSGDIWNIDNE | |
| | F | |
| hTfR-ECD-6His | HHHHHHCKGVEPKTECERLA | 114 |
| | GTESPVREEPGEDFPAARRL | |
| | YWDDLKRKLSEKLDSTDFTG | |
| | TIKLLNENSYVPREAGSQKD | |
| | ENLALYVENQFREFKLSKVW | |
| | RDQHFVKIQVKDSAQNSVII | |
| | VDKNGRLVYLVENPGGYVAY | |
| | SKAATVTGKLVHANFGTKKD | |
| | FEDLYTPVNGSIVIVRAGKI | |
| | TFAEKVANAESLNAIGVLIY | |
| | MDQTKFPIVNAELSFFGHAH | |
| | LGTGDPYTPGFPSFNHTQFP | |
| | PSRSSGLPNIPVQTISRAAA | |
| | EKLFGNMEGDCPSDWKTDST | |
| | CRMVTSESKNVKLTVSNVLK | |
| | EIKILNIFGVIKGFVEPDHY | |
| | VVVGAQRDAWGPGAAKSGVG | |
| | TALLLKLAQMFSDMVLKDGF | |
| | QPSRSIIFASWSAGDFGSVG | |
| | ATEWLEGYLSSLHLKAFTYI | |
| | NLDKAVLGTSNFKVSASPLL | |
| | YTLIEKTMQNVKHPVTGQFL | |
| | YQDSNWASKVEKLTLDNAAF | |
| | PFLAYSGIPAVSFCFCEDTD | |
| | YPYLGTTMDTYKELIERIPE | |
| | LNKVARAAAEVAGQFVIKLT | |
| | HDVELNLDYERYNSQLLSFV | |
| | RDLNQYRADIKEMGLSLQWL | |
| | YSARGDFFRATSRLTTDFGN | |
| | AEKTDRFVMKKLNDRVMRVE | |
| | YHFLSPYVSPKESPFRHVFW | |
| | GSGSHTLPALLENLKLRKQN | |
| | NGAFNETLFRNQLALATWTI | |
| | QGAANALSGDVWDIDNEF | |
| hTfR-ApD-6His | HHHHHHHHGKPIPNPLLGLD | 115 |
| | STGGGGSDSAQNSVIIVDKN | |
| | GRLVYLVENPGGYVAYSKAA | |
| | TVTGKLVHANFGTKKDFEDL | |
| | YTPVNGSIVIVRAGKITFAE | |
| | KVANAESLNAIGVLIYMDQT | |
| | KFPIVNAELSFFGHAHLGGG | |
| | GGGLPNIPVQTISRAAAEKL | |
| | FGNMEGDCPSDWKTDSTCRM | |
| | VTSESKNVKLTVS | |

Affinity variants of the generated human or mouse TfR antibodies were made by systematically introducing mutations into individual CDR of each antibody and the resulting variants were subjected to multiple rounds of selection with decreasing concentrations of antigen and/or increasing periods of dissociation to isolate clones with improved affinities. The sequences of individual variants were used to construct a combinatorial library which was subjected to an additional round of selection with increased stringency to identify additive or synergistic mutational pairings between the individual CDR regions. Individual combinatorial clones are sequenced. The heavy chain and light chain CDRs and VH/VL sequences of the human TfR binding domains TBD1-7 are provided in Tables 1-3. The heavy chain and light chain CDRs and VH/VL sequences of the mouse TfR binding protein (mTBP1) are provided in Table 7.

Human or mouse TfR binding proteins were generated by recombinant DNA technology. Such TfR binding proteins can be expressed in a mammalian cell line such as HEK293 or CHO, either transiently or stably transfected with an expression system using an optimal predetermined HC:LC vector ratio or a single vector system encoding both HC and LC. Clarified media, into which the protein has been secreted, can be purified using the commonly used techniques.

### Binding Affinities at 25° C.

The binding affinity and binding stoichiometry of the exemplified mouse TfR binding proteins to mouse TFR was determined using a surface plasmon resonance assay on a Biacore T200 instrument primed with HBS-EP+ (10 mM Hepes pH7.4+150 mM NaCl+3 mM EDTA+0.05% (w/v) surfactant P20) running buffer and analysis temperature set at 25° C. A human Fab capture kit (Cytiva P/N 28958325) was immobilized on a CM5 chip (Cytiva P/N 29104988) using standard NHS-EDC amine coupling on all four flow cells (Fc). Mouse TfR binding proteins were prepared at 10 μg/mL by dilution into running buffer. Target (mouse TFR-mIgG1-Fc) was prepared at final concentrations of 100.0, 25.0, 6.25, 1.56, 0.39, 0.097, 0.024 and 0 (blank) nM by dilution into running buffer.

Each analysis cycle consists of (1) capturing antibody samples on separate flow cells (Fc2, Fc3 and Fc4); (2) injection of the respective concentration of TfR over all Fc at 100 μL/min for 60 seconds followed by return to buffer flow for 1800 seconds to monitor dissociation phase; (3) regeneration of chip surfaces with injection of 10 mM glycine, pH 1.5, for 30 seconds at 10 μL/min over all cells; and (4) equilibration of chip surfaces with a 10 μL (60-sec) injection of HBS-EP+. Data were processed using standard double-referencing and fit to a 1:1 binding model using Biacore T200 Evaluation software, version 2.0.3, to determine the association rate (kₒₙ, M⁻¹s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and is in molar units. Results are provided in Table 13.

TABLE 13
Binding Affinity of Exemplified mTfR Binding
Proteins to mouse TFR at 25° C.

| Mouse TfR | | | | |
|---|---|---|---|---|
| binding protein | | kₒₙ | k_{off} | K_{D} |
| or conjugate | Target | M⁻¹s⁻¹ | s⁻¹ (10⁻⁴) | M |
| mTBP2 | mTFR | 2.1E5 | 1.03E−3 | 4.9E−9 |
| mTBP2-dsRNA | mTFR | 9.2E4 | 1.23E−3 | 1.3E−8 |
| conjugate | | | | |

These results demonstrate the exemplified mouse TfR binding protein and conjugate bind mouse TfR with high affinity at 25° C.

The binding affinity and binding stoichiometry of the exemplified human TfR binding proteins to human and cynomolgus TfR was determined using a surface plasmon resonance assay on a Biacore 8K instrument primed with HBS-EP+ (10 mM Hepes pH7.4+150 mM NaCl+3 mM EDTA+0.05% (w/v) surfactant P20) running buffer and analysis temperature set at 25° C. An anti-His antibody was immobilized on a CM5 chip (Cytiva P/N 29104988) using standard NHS-EDC amine coupling on all four flow cells (Fc). Target (human or cynomolgus TfR ECD) were prepared in the running buffer at final concentration of 500 μg/mL. The TfR binding proteins were prepared at a final concentration of 1, 0.2, 0.04, 0.008 and 0.0016 μM respectively by dilution of stock solution into running buffer.

Binding analysis was performed in a single-cycle kinetics manner. Each analysis cycle consists of (1) capturing the target (His-tagged human or cynomolgus TfR ECD) samples on separate flow cells (Fc2, Fc3 and Fc4); (2) injection of the lowest to highest concentration of antibodies or proteins over all Fc at 30 μL/min for 900 seconds followed by return to buffer flow for 1800 seconds to monitor dissociation phase; (3) regeneration of chip surfaces with injection of 10 mM glycine, pH 1.5, for 30 seconds at 10 L/min over all cells; and (4) equilibration of chip surfaces with a 10 μL (60-sec) injection of HBS-EP+. Data were processed using standard double-referencing and fit to a 2-state binding model using Biacore 8K Evaluation software, to determine the association rate (kₒₙ, M⁻¹s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and is in molar units. Results are provided in Table 14A.

Human endothelial line hCMEC-D3 (EMD Millipore SC066), endogenously expressing human TfR and MDCK cell line (ATCC CCL-34), engineered to express cynomolgus TfR were utilized to evaluate antibody/protein binding to cell-bound TfR. Cells were grown and maintained at submaximal confluence and detached from cultureware using Accutase cell detachment solution, washed, and allocated at 50000 cells per well for assessment of binding. Cells were treated with a viability stain then subsequently incubated with titrated concentrations of TfR binding proteins on ice. Cells were washed and binding of test antibodies or proteins was detected using a PE-labeled secondary reagent. Cells were then washed and read on the same day using a BioRad ZE5 cytometer. Analysis was performed post-acquisition in FlowJo, analyzing fluorescence of single, viable, non-debris events. EC50 values were derived by plotting geometric median PE intensity values across a given sample titration and fitting a sigmoidal (4PL) response curve in GraphPad Prism 8.3.0.

TABLE 14A
Binding Affinity of Exemplified human TfR binding proteins to
human or cynomolgus TfR at 25° C. or 0° C.

| | Human | Cyno | | |
|---|---|---|---|---|
| Human TfR | TfR K_{D} | TfR K_{D} | hCMEC-D3 | Cyno MDCK |
| binding | (Biacore, | (Biacore, | cell EC50 | cell EC50 |
| proteins | nM) at | nM) at | (nM) at | (nM) at |
| (TBP) | 25° C. | 25° C. | 0° C. | 0° C. |
| TBP1 | 0.11 | 145 | 0.47 | 510 |
| TBP2 | 0.27 | 1.1 | 0.65 | 1.08 |
| TBP3 | 0.000048 | 0.015 | 0.14 | 0.15 |
| TBP4 | 0.15 | 0.004 | 0.32 | 1.04 |
| TBP5 | 0.59 | 1.47 | 4.83 | 1.63 |
| TBP6 | 0.06 | 7.7 | 1.2 | 0.76 |
| TBP7 | 0.67 | 0.86 | 0.15 | 0.82 |
| TBP10 | 9.46 | 11.7 | 7.09 | 138 |
| TBP11 | 3.28 | 25.5 | 2.19 | 10.3 |
| TBP13 | 12.1 | 27.6 | 10.85 | 30.65 |
| TBP12 | 0.0015 | 2.92 | 1.55 | 3.38 |
| TBP14-MAPT | N/A | N/A | 764.1 | 453.3 |
| siRNA (dsRNA | | | | |
| No. 38 in | | | | |
| Table 11b) | | | | |
| TBP14-SNCA | N/A | N/A | 298.4 | 225.3 |
| siRNA (dsRNA | | | | |
| No. 10 in | | | | |
| Table 11a) | | | | |
| TBP15-MAPT | N/A | N/A | 75.56 | 57.12 |
| siRNA (dsRNA | | | | |
| No. 39 in | | | | |
| Table 11b) | | | | |

*N/A = not available

### Binding Affinity at 37° C.

Binding affinity and binding stoichiometry of the exemplified human TfR binding proteins to human and cynomolgus TfR was further characterized using a surface plasmon resonance assay on a Biacore 8K instrument primed with HIBS-EP+ (10 mM Hepes pH7.4+150 mM NaCl+3 mM EDTA+0.05% (w/v) surfactant P20) running buffer and analysis temperature set at 37° C. Target human and cynomolgous TfR ECD's were immobilized on a CM4 chip (Cytiva P/N 29104989) using standard NHS-EDC amine coupling. The TfR binding proteins were prepared at a final concentration of 0.3, 0.1, 0.033, 0.01, 0.0033, 0.001, 0.00033, 0.0001 μM respectively by dilution of stock solution into running buffer.

Binding analysis was performed in a multi-cycle kinetics manner. Each analysis cycle consists of (1) injection of the lowest to highest concentration proteins over all Fc at 50 μL/min for 140 seconds followed by return to buffer flow for 400 seconds to monitor dissociation phase; (2) regeneration of chip surfaces with injection of 3M magnesium chloride, for 30 seconds at 100 μL/min over all cells; and (3) equilibration of chip surfaces with a 50 μL (30-sec) injection of HBS-EP+. Data were processed using standard double-referencing and fit to a 2-state binding model using Biacore 8K Evaluation software, to determine the association rate (kₒₙ, M⁻¹s⁻¹ units), dissociation rate (k_{off}, s⁻¹ units), and Rₘₐₓ (RU units). The equilibrium dissociation constant (K_{D}) is calculated from the relationship K_{D}=k_{off}/kₒₙ, and is in molar units. Results are provided in Table 14B.

TABLE 14B
Binding Affinity of Exemplified human TfR binding
proteins to human or cynomolgus TfR at 37° C.

| | | Standard | | Standard |
|---|---|---|---|---|
| | Human | error of | Cyno | error of |
| Human TfR | TfR K_{D} | the mean, | TfR K_{D} | the mean, |
| binding | (Biacore, | Human TfR K_{D} | (Biacore, | Cyno TfR K_{D} |
| proteins | nM) at | (Biacore, | nM) at | (Biacore, |
| (TBP) | 37° C. | nM) n = 3 | 37° C. | nM) n = 3 |
| TBP3 | 0.005 | 0.001 | 0.254 | 0.041 |
| TBP12 | 0.426 | 0.007 | 6.786 | 0.083 |
| TBP4 | 2.030 | 0.771 | 6.935 | 1.222 |
| TBP13 | 32.087 | 11.795 | 66.565 | 11.695 |
| TBP2 | 0.169 | 0.037 | 0.162 | 0.075 |
| TBP11 | 5.318 | 0.030 | 22.507 | 3.970 |
| TBP1 | 0.966 | 0.536 | >1000 | 1135.141 |
| TBP6 | 2.246 | 1.191 | 92.541 | 12.818 |
| TBP5 | 4.246 | 1.085 | 156.268 | 25.216 |
| TBP7 | 0.838 | 0.420 | 3.539 | 0.938 |
| TBP10 | 72.262 | 3.927 | 91.395 | 18.061 |
| TBP14 | 153.642 | 7.949 | 300.180 | 2.565 |
| TBP15 | 0.522 | 0.284 | 502.210 | 8.129 |
| TBP14-MAPT | 258.042 | 87.834 | 448.154 | 16.578 |
| siRNA (dsRNA | | | | |
| No. 38 in | | | | |
| Table 11b) | | | | |
| TBP14-SNCA | 541.002 | 22.259 | >1000 | 376.948 |
| siRNA (dsRNA | | | | |
| No. 10 in | | | | |
| Table 11a) | | | | |
| TBP15-MAPT | 212.593 | 19.286 | 199.114 | 99.147 |
| siRNA (dsRNA | | | | |
| No. 39 in | | | | |
| Table 11b) | | | | |

### Epitope Mapping by Hydrogen Deuterium Exchange Mass Spectrometry (HDX-MS)

Hydrogen deuterium exchange coupled with mass spectrometry (HDX-MS) was performed to determine where the exemplified TfR binding proteins bind human TfR extracellular domain (TfR-ECD).

Peptide identification for human TfR-ECD was performed on a Waters Synapt G2Si (Waters Corporation) instrument using 5 μg of human TfR-ECD protein at zero exchange (1:10 dilution in 0.1× phosphate buffered saline in H₂O) using nepenthesin II (Nep II) for digestion, followed by treatment with PNGaseDj in line. The mass spectrometer was set in HDMSe (Mobility ESI+ mode) using a mass acquisition range of m/z 255.00-1950.00 with a scan time of 0.4 s. Data was processed using PLGS 2.3.02 (Waters Corporation). For the exchange experiments, the complex of human TfR-ECD protein with individual TfR binding protein was prepared at the molar ratio of 1:1.2 in 10 mM sodium phosphate buffer, pH 7.4 containing 150 mM NaCl (1×PBS buffer). The experiment was initiated by adding 25 μL of D20 buffer containing 0.1×PBS to 2.5 μl of TfR-ECD (0.9 mg/mL) or TfR-ECD+protein complex at 15° C. for various amounts of time (0 s, 10 s, 2 min, 10 min and 60 min) using a custom TECAN sample preparation system (Espada et al. 2019, J Am Soc Mass Spectrom. 2019 December; 30(12):2580-2583). The reaction was quenched using equal volume of was 0.32M TCEP, 3 M guanidine HC1, 0.1M phosphate pH 2.5 for two minutes at 4° C. and immediately frozen at −70° C. The sample injection system was comprised of a UR3 robot, a LEAP PAL3 HDX autosampler, and a HPLC system interfaced with a Waters Synapt G2Si (Waters Corporation), with modification as described (Espada et al., 2019, J Am Soc Mass Spectrom. 2019 December; 30(12):2580-2583.). The LC mobile phases consisted of water (A) and acetonitrile (B), each containing 0.2% formic acid. Each sample was thawed using 50 μL of 1.5 M guanidine HC1, 0.1M phosphate pH 2.5, for 1 min and injected on to a Nep II column for digestion at 4° C. with mobile phase A at a flow rate of 250 μL/min for 2.5 minutes. The resulting peptides were trapped on a Waters BEH Vanguard Pre-column at 4° C., and chromatographically separated using a Waters Acquity UPLC BEH C18 analytical column at 4° C. with a flow rate of 200 μL/min and a gradient of 3%-85% mobile phase B over 7 minutes and directed into mass spectrometer for mass analysis. The Synapt G2Si was calibrated with Glu-fibrinopeptide (Waters Corporation) prior to use. Mass spectra were acquired over the m/z range of 255 to 1950 in HDMS mode, with the lock mass m/z of 556.2771 (Leucine Enkephalin, Waters Corporation). The relative deuterium incorporation for each peptide was determined by processing the MS data for deuterated samples along with the undeuterated control using the identified peptide list in DynamX 3.0 (Waters Corporation). The free and bound states of human TfR-ECD were compared for deuterium incorporation differences to identify protected regions indicative of the binding epitope. Overall Sequence coverage for human TFR ECD was 90.4%.

For human TfR binding protein 1 (TBP1), decrease in deuterium uptake upon binding to human TfR-ECD was observed in residues 346-364 FGNMEGDCPSDWKTDSTCR (SEQ ID NO: 119), pointing to the probable epitope region. For human TfR binding protein 13 (TBP13), decrease in deuterium uptake upon binding to human TfR-ECD was observed in residues 243-247 (FEDLY) (SEQ ID NO: 162) and 345-364 (LFGNMEEGDCPSDWKTDSTCR) (SEQ ID NO: 163), pointing to the probable epitope regions. For human TfR binding protein 10 (TBP10), decrease in deuterium uptake upon binding to human TfR-ECD was observed in residues 243-247 (FEDLY) (SEQ ID NO: 162), 259-263 (AGKIT) (SEQ ID NO: 164), and 532-538 (VEKLTLD) (SEQ ID NO: 165), pointing to the probable epitope regions.

### Example 2: Synthesis and Characterization of dsRNAs Targeting SNCA (e.g., siRNA)

Single strands (sense and antisense) of the dsRNA duplexes were synthesized on solid support via a MerMade™ 12 (LGC Biosearch Technologies). The sequences of the sense and antisense strands were shown in Table 11. The sense strands were synthesized using phthalamido amino C6 lcaa CPG 500 Å (Chemgenes) whereas the antisense strands used standard support (LGC Biosearch Technologies). The oligonucleotides were synthesized via phosphoramidite chemistry at either 5, 10, or 50 μmol scales.

Standard reagents were used in the oligo synthesis (Table 16), where 0.1M xanthane hydride in pyridine was used as the sulfurization reagent and 20% DEA in ACN was used as an auxiliary wash post synthesis. All monomers (Table 17) were made at 0.1M in ACN and contained a molecular sieves trap bag.

The oligonucleotides were cleaved and deprotected (C/D) at 45° C. for 20 hours. The sense strands were C/D from the CPG using cold 50% (methylamine/ammonia hydroxide 28-30%) at RT for 3 hrs, whereas 3% DEA in ammonia hydroxide (28-30%, cold) was used for the antisense strands. C/D was determined complete by IP-RP LCMS when the resulting mass data confirmed the identity of sequence. Dependent on scale, the CPG was filtered via 0.45 um PVDF syringeless filter, 0.22 um PVDF Steriflip® vacuum filtration or 0.22 um PVDF Stericup® Quick release. The CPG was back washed/rinsed with either 30% EtOH/RNAse free water then filtered through the same filtering device and combined with the first filtrate. This was repeated twice. The material was then divided evenly into 50 mL falcon tubes to remove organics via Genevac™. After concentration, the crude oligonucleotides were diluted back to synthesized scale with RNAse free water and filtered either by 0.45 μm PVDF syringeless filter, 0.22 μm PVDF Steriflip® vacuum filtration or 0.22 μm PVDF Stericup® Quick release.

The crude oligonucleotides were purified via AKTA™ Pure purification system using anion-exchange (AEX). For AEX, an ES Industry Source™ 15Q column maintaining column temperature at 65° C. with MPA: 20 mM NaH₂PO₄, 15% ACN, pH 7.4 and MPB: 20 mM NaH₂PO₄, 1M NaBr, 15% ACN, pH 7.4. Fractions which contained a mass purity greater than 85% without impurities >5% where combined.

The purified oligonucleotides were desalted using 15 mL 3K MWCO centrifugal spin tubes at 3500×g for ˜30 min. The oligonucleotides were rinsed with RNAse free water until the eluent conductivity reached <100 usemi/cm. After desalting was complete, 2-3 mL of RNAse free water was added then aspirated 10×, the retainment was transferred to a 50 mL falcon tube, this was repeated until complete transfer of oligo by measuring concentration of compound on filter via nanodrop. The final oligonucleotide was then nano filtered 2× via 15 mL 100K MWCO centrifugal spin tubes at 3500×g for 2 min. The final desalted oligonucleotides were analyzed for concentration (nano drop at A260), characterized by IP-RP LC/MS for mass purity (Table 15) and UPLC for UV-purity.

TABLE 15
Exemplary LC/MS data

| | | MW Cal. | MW Obs. |
|---|---|---|---|
| dsRNA No. | Stand | (g/mol) | (g/mol) |
| 8 | S: SEQ ID NO 93 | 7138.86 | 7139.0 |
| | AS: SEQ ID NO 94 | 7825.19 | 7826.3 |
| 9 | S: SEQ ID NO 95 | 7150.9 | 7151.5 |
| | AS: SEQ ID NO 96 | 7813.15 | 7813.7 |
| 10 | S: SEQ ID NO 95 | 7150.89 | 7151.5 |
| | AS: SEQ ID NO 97 | 7813.15 | 7813.14 |
| 11 | S: SEQ ID NO 95 | 7150.89 | 7151.5 |
| | AS: SEQ ID NO 98 | 7801.11 | 7813.8 |
| 12 | S: SEQ ID NO 99 | 7318.95 | 7319.2 |
| | AS: SEQ ID NO 94 | 7825.19 | 7826.3 |
| 13 | S: SEQ ID NO 100 | 7162.88 | 7163.3 |
| | AS: SEQ ID NO 101 | 7802.15 | 7802.1 |
| 14 | S: SEQ ID NO 102 | 7084.8 | 7085.4 |
| | AS: SEQ ID NO 103 | 7772.12 | 7772.6 |
| 15 | S: SEQ ID NO 104 | 7329.03 | 7329.3 |
| | AS: SEQ ID NO 105 | 7557.91 | 7557.91 |
| 16 | S: SEQ ID NO 106 | 7329.03 | 7329.3 |
| | AS: SEQ ID NO 107 | 7795.16 | 7795.6 |
| 17 | S: SEQ ID NO 108 | 7264.9 | 7265.3 |
| | AS: SEQ ID NO 107 | 7795.16 | 7795.6 |
| 18 | S: SEQ ID NO 117 | 7022.83 | 7024 |
| | AS: SEQ ID NO 97 | 7813.15 | 7813.14 |
| 19 | S: SEQ ID NO 118 | 7010.8 | 7011.3 |
| | AS: SEQ ID NO 97 | 7813.15 | 7813.14 |
| 24 | S: SEQ ID NO 141 | 6955.67 | 6956.6 |
| | AS: SEQ ID NO 96 | 7813.15 | 7813.7 |
| 25 | S: SEQ ID NO 141 | 6955.67 | 6956.6 |
| | AS: SEQ ID NO 97 | 7813.15 | 7813.14 |
| 35 | S: SEQ ID NO 126 | 7337.06 | 7338.1 |
| | AS: SEQ ID NO 127 | 7518.87 | 7519.7 |
| 37 | S: SEQ ID NO 130 | 7177 | 7178 |
| | AS: SEQ ID NO 131 | 7677.98 | 7678.8 |
| 38 | S: SEQ ID NO 132 | 7349.09 | 7348.5 |
| | AS: SEQ ID NO 133 | 7506.84 | 7507.4 |
| 39 | S: SEQ ID NO 134 | 7229.96 | 7230.5 |
| | AS: SEQ ID NO 135 | 7749.1 | 7748.2 |
| 40 | S: SEQ ID NO 136 | 7189 | 7188.4 |
| | AS: SEQ ID NO 137 | 7665.95 | 7667.2 |

TABLE 16
Oligonucleotide Synthesis Reagents
Reagents

Activator Solution (0.5M ETT in ACN)
Cap A (Acetic Anhydride, Pyridine in THF, 1:1:8)
Cap B (1-Methylimidazole in THF, 16:84)
Oxidation Solution (0.02M Iodine in THF/Pyridine/Water,
70:20:10)
Deblock Solution, 3% TCA in DCM (w/v)
Acetonitrile (Anhydrosolv, Water max. 10 ppm)
Xanthane Hydride (0.1M in Pyridine)
Diethylamine (20% in Acetonitrile)

TABLE 17
Phosphoramidites

| Phosphoramidite | Abbreviation | Supplier | Catalog # | CAS |
|---|---|---|---|---|
| DMT-2′-F-A(Bz)-CE | fA | Hongene | PD1-001 | 136834-22-5 |
| Phosphoamidite | | | | |
| DMT-2′-F—C(Ac)-CE | fC | Hongene | PD3-001 | 159414-99-0 |
| Phosphoamidite | | | | |
| DMT-2′-F-G(iBu)-CE | fG | Hongene | PD2-002 | 144089-97-4 |
| Phosphoamidite | | | | |
| DMT-2′-F—U-CE | fU | Hongene | PD5-001 | 146954-75-8 |
| Phosphoamidite | | | | |
| DMT-2′-O—Me-A(Bz)- | mA | Hongene | PR1-001 | 110782-31-5 |
| CE Phosphoamidite | | | | |
| DMT-2′-O—Me—C(Ac)- | mC | Hongene | PR3-001 | 199593-09-4 |
| CE Phosphoamidite | | | | |
| DMT-2′-O—Me-G(iBu)- | mG | Hongene | PR2-002 | 150780-67-9 |
| CE Phosphoamidite | | | | |
| DMT-2′-O—Me—U-CE | mU | Hongene | PR5-001 | 110764-79-9 |
| Phosphoamidite | | | | |
| 5′bis(POM) vinyl | POM- | Hongene | PR5-032 | BVPMUP23B2A1 |
| phosphate-2′-Ome- | VPmU | | | |
| U3′CE | | | | |
| phosphoroamidite | | | | |
| Reverse Abasic | iAb | Chemgenes | ANP-1422 | 401813-16-9 |
| phosphoroamidite | | | | |
| Abasic | Aba | Chemgenes | ANP-7058 | 129821-76-7 |
| phosphoroamidite | | | | |

### Example 3: Generation of TfR Binding Proteins-dsRNA Conjugates

Certain abbreviations are defined as follows: “ACN” refers to acetonitrile; “aAEX” refers to analytical anion exchange; “AS” refers to antisense strand; “DAR” refers to drug/siRNA to antibody/protein ratio; “DCM” refers to dichloromethane; “DHAA” refers to dehydroascorbic acid; “DIEA” refers to N,N-diisopropylethylamine; “DMF” refers to dimethylformamide; “dsRNA” refers to double stranded ribonucleic acid; “DTT” refers to dithiothreitol; “EtOAc” refers to ethyl acetate; “FEP” refers to fluorinated ethylene propylene; “FMI” refers to Fluid Metering Inc; “h” refers to hours; “HATU” refers to hexafluorophosphate azabenzotriazole tetramethyl uranium; “HPLC” refers to high-performance liquid chromatography; “LC/MS” refers to liquid chromatography mass spectrometry; “LTQ/MS” refers to linear ion trap mass spectrometer; “min” refers to minutes; “MTBE” refers to methyl tert-butyl ether; “MW” refers to molecular weight; “NHS” refers to N-hydroxysuccinimide; “OD” refers to optical density; “PBS” phosphate-buffered saline; “PEG” refers to polyethylene glycol; “rpm” refers to revolutions per minute; “SEC” refers to size exclusion chromatography; “siRNA” refers to small interfering RNA; “SMCC” refers to succinimidyl-4-(N-maleimidomethyl)cyclohexane-1-carboxylate; “SS” refers to sense strand; “TCO” refers to trans-cyclo-octene; “TEA” refers to triethylamine; “TFA” refers to trifluoroacetic acid; “TfR” refers to transferrin receptor; “THF” refers to tetrahydrofuran; “TRIS” refers to tris(hydroxymethyl)aminomethane; and “UV” refers to ultraviolet.

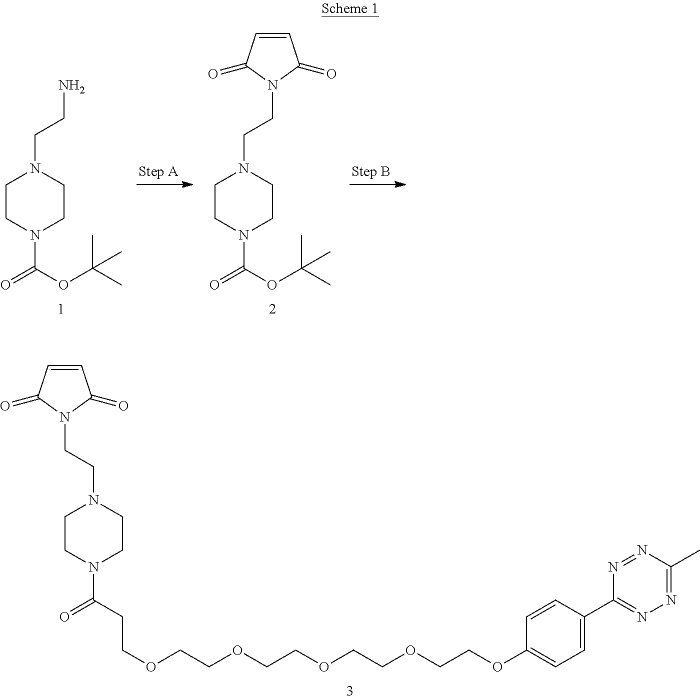 [SMILES: CC(C)(C)OC(=O)N1CCN(CCN)CC1 ; CC(C)(C)OC(=O)N1CCN(CCN2C(=O)C=CC2=O)CC1 ; Cc1nnc(-c2ccc(OCCOCCOCCOCCOCCC(=O)N3CCN(CCN4C(=O)C=CC4=O)CC3)cc2)nn1]

Scheme 1, step A depicts the coupling of compound (1) and furan-2,5-dione in a solvent such as acetic acid followed by treatment with acetic anhydride and sodium acetate in a solvent such as toluene to give compound (2). Step B shows the acidic deprotection of compound (2) with an acid such as TFA in a suitable solvent such as DCM followed by an amide coupling with methyltetrazine-PEG4-acid using an amide coupling reagent such as HATU with an appropriate base such as N,N-diisopropyl amine in a solvent system such as DMF and THE to give compound (3). One skilled in the art will recognize that a variety of coupling reagents, bases, and solvents can be used to perform an amide coupling.

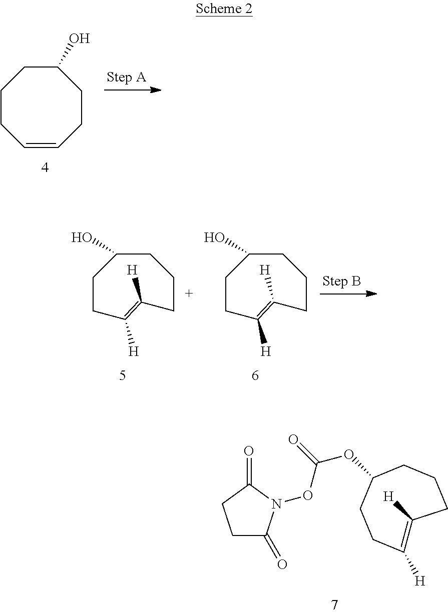

Scheme 2, step A depicts the transformation of a cis-olefin compound (4) to the trans olefin compounds (5) and (6) through using a closed-loop flow apparatus using irradiation and capture on a column of silver nitrate absorbed onto silica gel. Step B shows the reaction of compound (5) with N,N′-disuccinimidyl carbonate using a suitable base such as TEA in a solvent such as ACN to give compound (7).

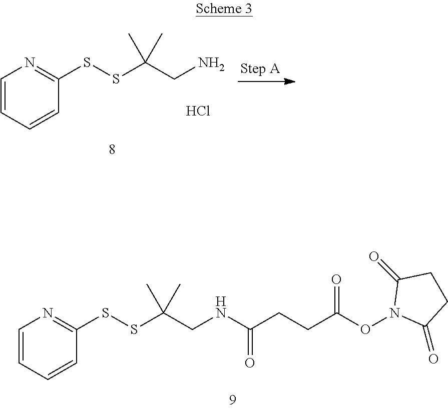 [SMILES: CC(C)(CN)SSc1ccccn1 ; Cl ; CC(C)(CNC(=O)CCC(=O)ON1C(=O)CCC1=O)SSc1ccccn1]

Scheme 3, step A depicts a one pot reaction of compound (8) with glutaric anhydride using an appropriate base such as DIEA in a solvent such as THF followed by an amide coupling with N-hydroxysuccinimide using an appropriate coupling reagent such as 1-(3-dimethylaminopropyl)-3-ethylcarbodiimide hydrochloride with an appropriate base such as 4-dimethylaminopyridine to give compound (9). One skilled in the art will recognize that a variety of coupling reagents, bases, and solvents can be used to perform an amide coupling.

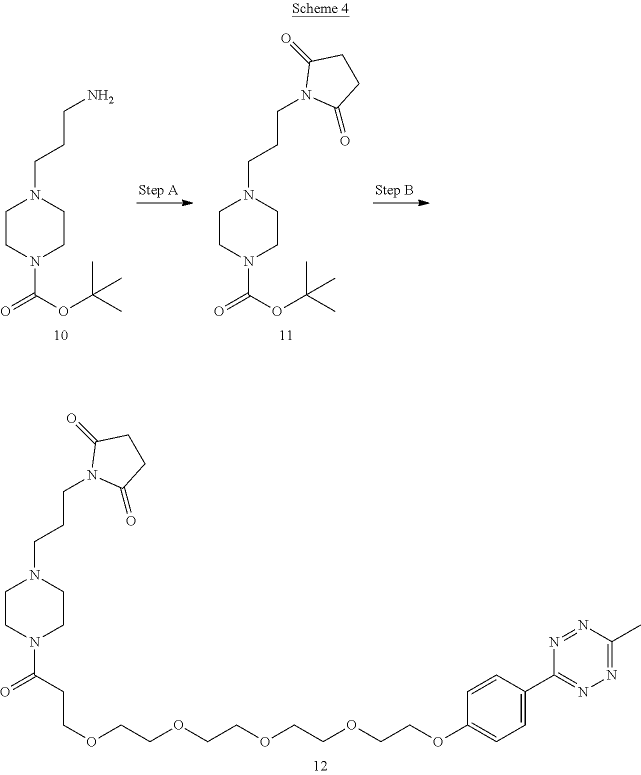 [SMILES: CC(C)(C)OC(=O)N1CCN(CCCN)CC1 ; CC(C)(C)OC(=O)N1CCN(CCCN2C(=O)CCC2=O)CC1 ; Cc1nnc(-c2ccc(OCCOCCOCCOCCOCCC(=O)N3CCN(CCCN4C(=O)CCC4=O)CC3)cc2)nn1]

Scheme 4, step A depicts the coupling of compound (10) and furan-2,5-dione in a solvent such as acetic acid followed by treatment with TEA in a solvent such as toluene to give compound (11). Step B depicts the conversion of compound (11) to compound (12) in a manner essentially analogous to scheme 1, step B.

### Preparation 1

### tert-Butyl 4-[2-(2,5-dioxopyrrol-1-yl)ethyl]piperazine-1-carboxylate

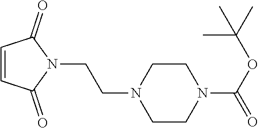 [SMILES: CC(C)(C)OC(=O)N1CCN(CCN2C(=O)C=CC2=O)CC1]

tert-Butyl 4-(2-aminoethyl)piperazine-1-carboxylate (3.00 g, 13.1 mmol) was dissolved in acetic acid (6 mL). Added furan-2,5-dione (1.28 g, 13.1 mmol) and stirred at ambient temperature for 7 h. The mixture was then stored in a refrigerator for 18 h. Removed most of the acetic acid under vacuum at 50° C. Added acetic anhydride (10 mL, 106 mmol) and sodium acetate (1.6 g, 20 mmol) then heated to 80° C. for 2 h. Added toluene and removed most of the acetic anhydride under vacuum. The mixture was taken into saturated aqueous ammonium chloride (60 mL) and extracted with DCM (3×50 mL). The combined organic layers were dried over anhydrous sodium sulfate, filtered, and concentrated under vacuum to give the crude product as a dark oil. Purified via silica gel chromatography eluting with EtOAc/hexane to give the title compound (2.1 g, 52%). LC/MS m z 310.3 (M+H).

### Preparation 2

### tert-Butyl 4-[3-(2,5-dioxopyrrol-1-yl)propyl]piperazine-1-carboxylate

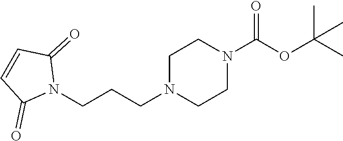 [SMILES: CC(C)(C)OC(=O)N1CCN(CCCN2C(=O)C=CC2=O)CC1]

Furan-2,5-dione (789 mg, 7.97 mmol) was added to a solution of tert-butyl 4-(3-aminopropyl)piperazine-1-carboxylate (2.00 g, 7.97 mmol) in acetic acid (8 mL, 140 mmol). The mixture was stirred at ambient temperature for 12 hours then concentrated under vacuum to give the crude intermediate (Z)-4-[3-(4-tert-butoxycarbonylpiperazin-1-yl)propylamino]-4-oxo-but-2-enoic acid (2.72 g, 7.97 mmol) which was then dissolved in toluene (80 mL). TEA (5.6 mL, 40 mmol) and 4 Å molecular sieves (8.8 g) were added. The flask was equipped with a Dean-Stark trap, and the mixture was heated at 120° C. for 48 hours. After cooling to ambient temperature, the solids were removed by filtration, and washed with DCM (40 mL). The volatiles were removed under reduced pressure to give a residue that was dried under vacuum. The thick residue was purified by normal phase chromatography eluting with (10% MeOH/MTBE)/DCM to give the title compound as a yellow, flaky powder (353 mg, 13.7%). LC/MS m z 324 (M+H).

### Preparation 3

### 1-[2-[4-[3-[2-[2-[2-[2-[4-(6-Methyl-1,2,4,5-tetrazin-3-yl)phenoxy]ethoxy]ethoxy]ethoxy]ethoxy]propanoyl]piperazin-1-yl]ethyl]pyrrole-2,5-dione

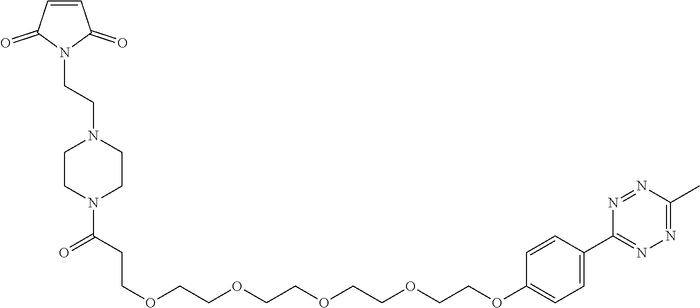 [SMILES: Cc1nnc(-c2ccc(OCCOCCOCCOCCOCCC(=O)N3CCN(CCN4C(=O)C=CC4=O)CC3)cc2)nn1]

tert-Butyl 4-[2-(2,5-dioxopyrrol-1-yl)ethyl]piperazine-1-carboxylate (150 mg, 0.485 mmol) was dissolved in DCM (2 mL). Added TFA (1 mL, 13 mmol) and stirred at ambient temperature for 1 h. Concentrated under vacuum and further dried under high vacuum for 18 h to give the intermediate 1-(2-piperazin-1-ylethyl)pyrrole-2,5-dione trifluoroacetate. This material and methyltetrazine-PEG4-acid (130 mg, 0.283 mmol) were dissolved in DMF (2.0 mL) and THF (2 mL). HATU (380 mg, 0.969 mmol) was then added followed by N,N-diisopropylamine (0.45 mL, 2.6 mmol). Stirred at ambient temperature for 2 h. Diluted with DCM (50 mL) and washed with saturated aqueous ammonium chloride (30 mL). The organic layer was dried over anhydrous sodium sulfate, filtered, and concentrated under vacuum to give crude product as a red solid. Purified via silica gel chromatography eluting with 0-20% MeOH/EtOAc to give the title compound as a red solid (150 mg, 49%). LC/MS m z 628.6 (M+H).

### Preparation 4

### 1-[3-[4-[3-[2-[2-[2-[2-[4-(6-Methyl-1,2,4,5-tetrazin-3-yl)phenoxy]ethoxy]ethoxy]ethoxy]ethoxy]propanoyl]piperazin-1-yl]propyl]pyrrole-2,5-dione

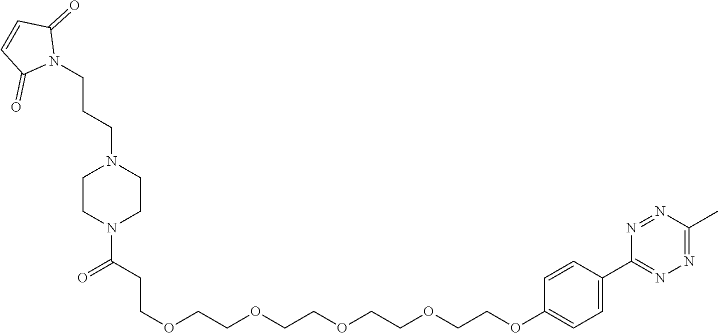 [SMILES: Cc1nnc(-c2ccc(OCCOCCOCCOCCOCCC(=O)N3CCN(CCCN4C(=O)C=CC4=O)CC3)cc2)nn1]

The title compound was prepared using tert-butyl 4-[3-(2,5-dioxopyrrol-1-yl)propyl]piperazine-1-carboxylate in a manner essentially analogous to the methods found in Preparation 3. LC/MS m/z 642 (M+H).

### Preparation 5

### (1R,4E)-Cyclooct-4-en-1-ol (axial) and (1R,4E)-cyclooct-4-en-1-ol (equatorial)

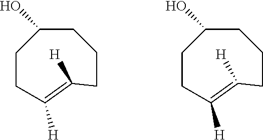

A closed-loop, flow apparatus was assembled that permitted irradiation of a solution of cis-olefin and cycling of said solution through a silver nitrate-absorbed onto silica gel cartridge. Only the trans-olefin is retained in the silica gel, thus the cis olefin is recycled back to irradiation stage.

Equipment: (A) UV Lamp (Pen-Ray 099912-1, 254 nM), power supply 99-0055-01 Lamp Current 18 mA/AC. Per manufacturer's description, this lamp produces between 4400 and 4750 microwatts/cm\hat{ }2 intensity at 0.75″ for 254 nM light. (B) FMI pump set to 10 mL/min that draws the reaction mixture from a Pyrex® round bottom flask (250 mL). This was connected to FEP 1/16″ tubing that was wrapped around a cold finger (total 7 mL loop, air cooling). The UV lamp was placed in the center of the cold finger to irradiate the sample with air cooling. After the irradiation, the sample tubing continued into an ISCO SLM that contained 25 g of silver nitrate impregnated silica gel (See Fox, et. al., Angewandte Chemie, International Edition Engl 2009, 48(38), 7013-7016; Synthesis 2018, 50, 4875).

The following steps were performed. Loaded a 50 g silica gel cartridge with 25 g of silver nitrate absorbed onto silica gel on top, covered in aluminum foil, and conditioned by pumping the 1:1 hexanes/diethyl ether solvent mixture for 1 h. Mixed (4Z)-cyclooct-4-en-1-ol; racemic at hydroxyl position (2.00 g, 15.8 mmol) and methyl benzoate (2.0 mL, 16 mmol) in n-hexane (220 mL) and diethyl ether (220 mL), turned on the UV lamp, and circulated the solution through the coil around the cold finger through the silica gel/silver nitrate cartridge and back through the system at a flow rate of 10 mL/min for 96 h. Flushed the silica cartridge with EtOAc (200 mL) and dried with air. Discarded the filtrate. Rinsed the dried silica cartridge with concentrated NH40H (150 mL) followed by DCM (150 mL). Separated the layers and extracted the aqueous with DCM (2×50 mL). Washed the combined organic layers with saturated aqueous sodium chloride, dried over MgSO₄, filtered, and concentrated under reduced pressure. Purified via silica gel chromatography eluting with 0-45% MTBE/hexane to give the two products as clear liquids. Axial-(1R,4E)-cyclooct-4-en-1-ol (569.8 mg, 28.5%). ¹H NMR (CDCl₃) 5.63-5.55 (m, 1H), 5.44-5.36 (m, 1H), 3.50-3.45 (m, 1H), 2.39-2.32 (m, 3H), 2.00-1.94 (m, 4H), 1.73-1.66 (m, 3H). Equatorial-(1R,4E)-cyclooct-4-en-1-ol (673.6 mg, 33.7%). ¹H NMR (CDCl₃): 5.60-5.57 (m, 2H), 4.05 (dd, J=5.3, 10.2 Hz, 1H), 2.44-2.37 (m, 1H), 2.29-2.22 (m, 2H), 2.18-2.13 (m, 2H), 1.93-1.86 (m, 4H), 1.32-1.25 (m, 1H).

### Preparation 6

### [(1R,4E)-Cyclooct-4-en-1-yl] (2,5-dioxopyrrolidin-1-yl) carbonate

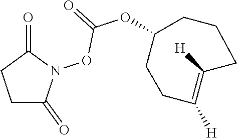

N,N′-disuccinimidyl carbonate (2.79 g, 10.3 mmol) in small portions (˜250-300 mg each addition, five minutes apart) was added to a mixture of 1R,4E)-cyclooct-4-en-1-ol (axial) (569 mg, 4.50 mmol) and TEA (2.5 mL, 18 mmol) in ACN (25 mL). The mixture was covered in aluminum foil and stirred at ambient temperature for 60 h. Solvent was removed under reduced pressure to give an oil that was partitioned between water (20 mL) and diethyl ether (50 mL). The layers were separated and the aqueous was extracted with diethyl ether (2×50 mL). The organic layers were combined and washed with saturated ammonium chloride, then with saturated aqueous sodium chloride, dried over MgSO₄, filtered, and concentrated under reduced pressure. Silica gel chromatography was used to purify and eluted with 0-60% MTBE/hexanes to give the title compound as a colorless residue that formed a white solid (732 mg, 61%). LC/MS m z 324 (M+H).

### Preparation 7

### (2,5-Dioxopyrrolidin-1-yl) 4-[[2-methyl-2-(2-pyridyldisulfanyl)propyl]amino]-4-oxo-butanoate

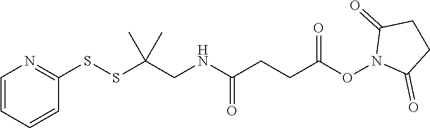 [SMILES: CC(C)(CNC(=O)CCC(=O)ON1C(=O)CCC1=O)SSc1ccccn1]

2-Methyl-2-(2-pyridyldisulfanyl)propan-1-amine hydrochloride (245 mg, 0.976 mmol), glutaric anhydride (112 mg, 0.972 mmol), and DIEA (360 μL, 2.16 mmol) were added together in THE (4 mL) and heated at 45° C. for 12 h with vigorous stirring. After this time, the mixture was cooled to ambient temperature and 1-(3-dimethylaminopropyl)-3-ethylcarbodiimide hydrochloride (224 mg, 1.17 mmol) and 4-dimethylaminopyridine (25 mg, 0.20 mmol) were added. The mixture was stirred at ambient temperature for 5 min before adding add N-hydroxysuccinimide (126 mg, 1.07 mmol) in one portion followed by stirring for 36 h. The mixture was filtered, and the resulting filtrate was loaded directly onto silica gel (2 g). Silica gel chromatography was used to purify and eluted with 75% EtOAc/hexanes to give the title compound as a light, cloudy residue (100.2 mg, 24%). LC/MS m z 426 (M+H) (Hydrolyzed NHS ester).

### TCO-Functionalization SNCA

In a set of 4×50 mL Falcon™ tubes, the sense strand of the SNCA dsRNA with a hexylamine chain attached at the 3′ end (SNCA_SS-3C6A) (measured concentration of SS calculated to be OD/mL of 412.5 or 2 mM, 120 mL, 0.24 mmol) and 20× borate buffer (6 mL) were equally divided (10 mL each) and each were treated with 7.5 mL of a solution of [(1R,4E)-cyclooct-4-en-1-yl] (2,5-dioxopyrrolidin-1-yl) carbonate (1.65 g, 6.17 mmol) dissolved in 1,4-dioxane (100 mL). Mixed at 25° C. at 600 rpm for 30 min. The remainder of the SS sample was divided and reacted in the same way to yield a total of 12 sample vessels, each containing ˜150 mg of crude SS starting material. The dioxane was removed by placing the Falcon™ tubes on a Genevac evaporator. The remaining aqueous solutions were combined and filtered to remove any suspended solids. Purified on an AKTA™ pure chromatography system using 13-45% ACN in 50 mM NaOAc (aq) with a flow rate=40 mL/min. Combined the appropriate fractions, and removed the organics on a SpeedVac™ before desalting and concentrating to yield 214 mL which measured OD/mL of 127.3 equating to 624 μM and a total of 973 mg. LTQ/MS m z 7292; UV purity 99+%.

### TCO-SNCA Duplex

The nanodrop concentrations for the aqueous solutions of each strand (average of 5×) were measured as SS=624 μM, and AS=1094 μM. Mixed 210 mL of SS and 113.7 mL of AS, then shook at ambient temperature for 30 min. The amount of residual SS strand was measured until completion and required adding an additional 21.9 mL of AS. The resulting 345 mL of the solution measured (Nanodrop™ Lite, 6× average, 20× dilution) OD/mL of 159.5 equating to 421 μM and a total of 2.19 g. LTQ/MS m z 7291,7825; UV purity >99%.

SMCC-Functionalization of SNCA dsRNA

A freshly prepared solution of (2,5-dioxopyrrolidin-1-yl) 4-[(2,5-dioxopyrrol-1-yl)methyl]cyclohexanecarboxylate (185 mg, 0.542 mmol) in THE (50 mL) was added to SNCA_SS-3C6A (44 mL, 0.0528 mmol; OD/mL of 250.4, or ˜1200 μM (˜8.8 mg/mL)) in 0.2M phosphate buffer (44 mL). Vortexed vigorously for 2 minutes, and then shook at ambient temperature at 900 rpm for 2 h total. Analysis by LTQ showed about 94-95% conversion. Acidified to pH˜4 with 20-30 drops of 5N HC1, and then removed organics in a Genevac concentrator. Desalted by centrifugal filtration on a 3K spin filter (4×4000 rpm, 30 min), and pooled the retentates. The OD measurement of the solution (average of 3 measurements, 10× dilution) was 266 equating to 1.3 mM and a total of 316 mg. Extinction coefficient was 204.12. LTQ/MS m z 7358.

### SMCC-SNCA Duplex

The nanodrop concentrations of aqueous solutions of each strand (average of 3×) were measure as SS=1322 μM and AS=1108 μM. Mixed 32 mL of SS and 36.2 mL of AS and shook for 30 min at 30° C. The amount of residual SS strand was measured until completion and required adding an additional 360 μL of AS. Removed endotoxins by filtering through a 0.45 μM filter. The resulting 75 mL of solution measured (Nanodrop™ Lite, 5× average, 10× dilution) 217 OD/mL equating to 575 μM and a total of 653 mg. LTQ/MS m z 7358,7825; UV purity 99+%.

### GDM-Functionalization SNCA

In a 15 mL Falcon™ tube, diluted SNCA_SS-3C6A (measured concentration of SS calculated to be OD/mL of 247.6 or 1.21 mM, 3 mL, 0.0036 mmol) with 20× borate buffer (0.3 mL) and water (3 mL, 166.530 mmol) then added (2,5-dioxopyrrolidin-1-yl) 5-[[2-methyl-2-(2-pyridyldisulfanyl)propyl]amino]-5-oxo-pentanoate (3.6 mL, 0.75M in dioxane). Mixed at 200 rpm for 1 h. The organics were removed on a SpeedVac™, desalted, and concentrated three times with water to give SNCA_SS-3C6A-GDM with a total yield of 13.2 mL (OD/mL of 35.88, equating to 175.8 μM and a total of 17.3 mg). Extinction coefficient was 204.12. LTQ1 MS m z 7449 (UV purity 95+%).

Added 2 tris(2-carboxyethyl)phosphine hydrochloride (75 μL of 100 mM solution in water) to SNCA_SS-3C6A-GDM. Shook at 10° C. for 4 h, and then 16 h at ambient temperature. Added additional tris(2-carboxyethyl)phosphine hydrochloride (75 μL of 100 mM solution in water), and shook for an additional 16 hours. Desalted by centrifugal filtration on a 3K spin filter (3×40 min, 4000 rpm), and pooled the retentates to give 10 mL. The OD measurement of the solution (average of 4 measurements, 10× dilution) was 63.6 equating to 311.4 μM and a total of 22.9 mg. Extinction coefficient was 204.12. LTQ/MS m z 7340; UV purity 99+%.

### GDM Annealing Step

The nanodrop concentrations of aqueous solutions of each strand (average of 4×) are SS=311.4 μM and AS=431.3 μM. Mixed 10 mL of SS and 6.7 mL of AS with 5 mL of water and shook for 30 min. The amount of residual SS strand was measured until completion and required adding an additional 560 μL of AS. Concentrated on 3K MW-cut off filter (20 min), then 50 k spin filtration, and further concentrated through a 3K filter. The resulting 6 mL of solution measured (Nanodrop™ Lite, 5× average, 20× dilution) 181.62 OD/mL equating to 486 μM and a total of 44.2 mg. LTQ/MS m z 7340,7825; UV purity 99+%. MAPT dsRNA functionalization and anneal can be performed in the same way as SNCA dsRNA described above.

Conjugation of dsRNA to TfR Binding Proteins

Site-specific native or engineered cysteine amino acid residues in the TfR binding proteins were used to conjugate dsRNA. Cysteines can be engineered into the primary amino acid sequence of the TfR binding proteins. The approach of introducing cysteines as a means for conjugation has been described in WO 2018/232088, which is both incorporated by reference in its entirety and incorporated specifically in relation to conjugation via cysteine residues. For engineered cysteine conjugation, the TfR binding proteins were first reduced with 40 molar equivalents reducing agent dithiothreitol (DTT) at 37° C. for two hours, followed by desalting to remove reducing agent via dialysis or desalting columns. This is followed by re-oxidation of the TfR binding protein to reform the structural disulfides with 10 molar equivalent dehydroascorbic acid (DHAA) incubation at room temperature for two hours. A follow up desalting was performed to remove oxidizing agent.

Conjugation of dsRNA onto TfR binding proteins were done using the following methods.

### Conjugation Scheme 1

In the first method, a bifunctional maleimide-methyl-tetrazine linker was conjugated to the engineered cysteine of the TfR binding proteins at neutral pH by addition of the linker to the TfR binding protein at 20 molar equivalents and incubating at ambient temperature for 1 h. Following which, a desalting step was performed to remove excess linker. Then, trans-cyclo-octene (TCO) functionalized dsRNA was added onto the protein linker at 4 molar equivalents for overnight conjugation at 4° C.

Step 1a: TfR. Binding Protein Conjugation with Maleimide-Methyl-Tetrazine Linker

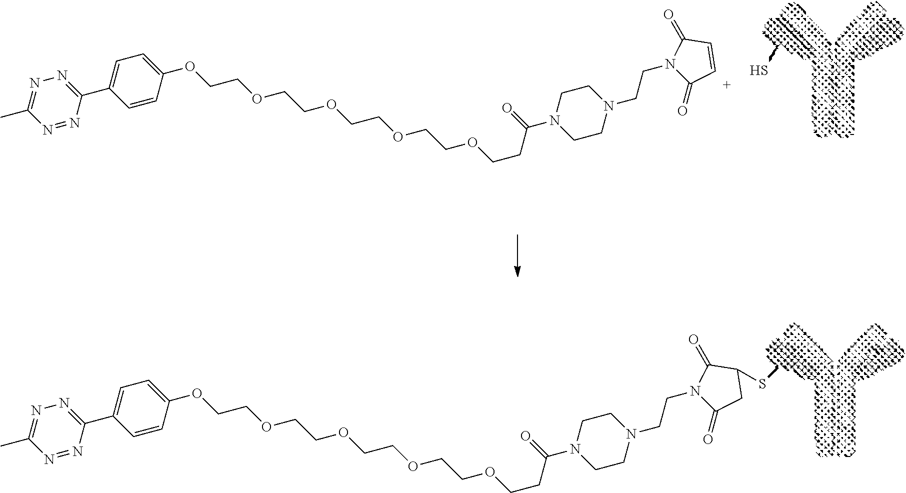

Step 1b: TfR Binding Protein Conjugation with Maleimide-Methyl-Tetrazine Linker Ring Opening

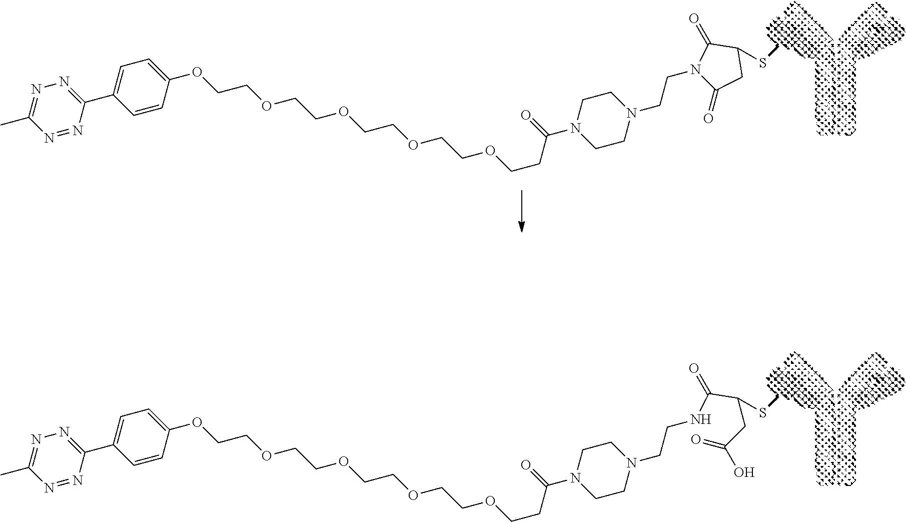 [SMILES: Cc1nnc(-c2ccc(OCCOCCOCCOCCOCCC(=O)N3CCN(CCN4C(=O)CC(S)C4=O)CC3)cc2)nn1 ; Cc1nnc(-c2ccc(OCCOCCOCCOCCOCCC(=O)N3CCN(CCNC(=O)C(S)CC(=O)O)CC3)cc2)nn1]

Step 2a: dsRNA Conjugation with Protein-Linker Intermediate

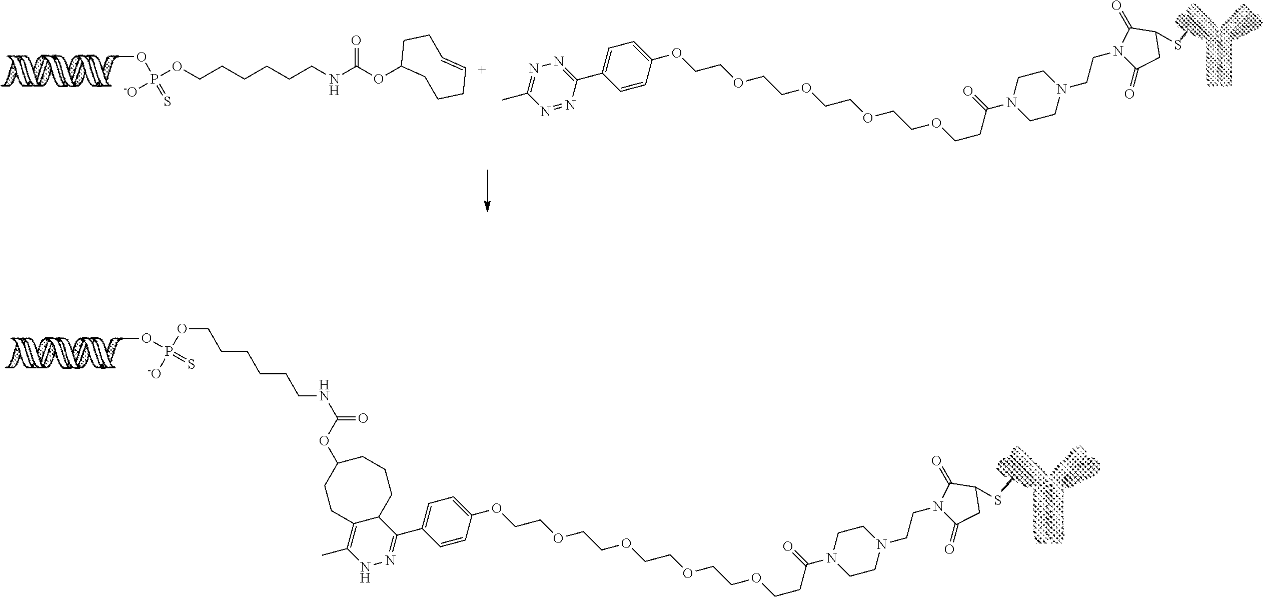

Step 2b: dsRNA Conjugation with Protein-Linker Intermediate (Open Ring)

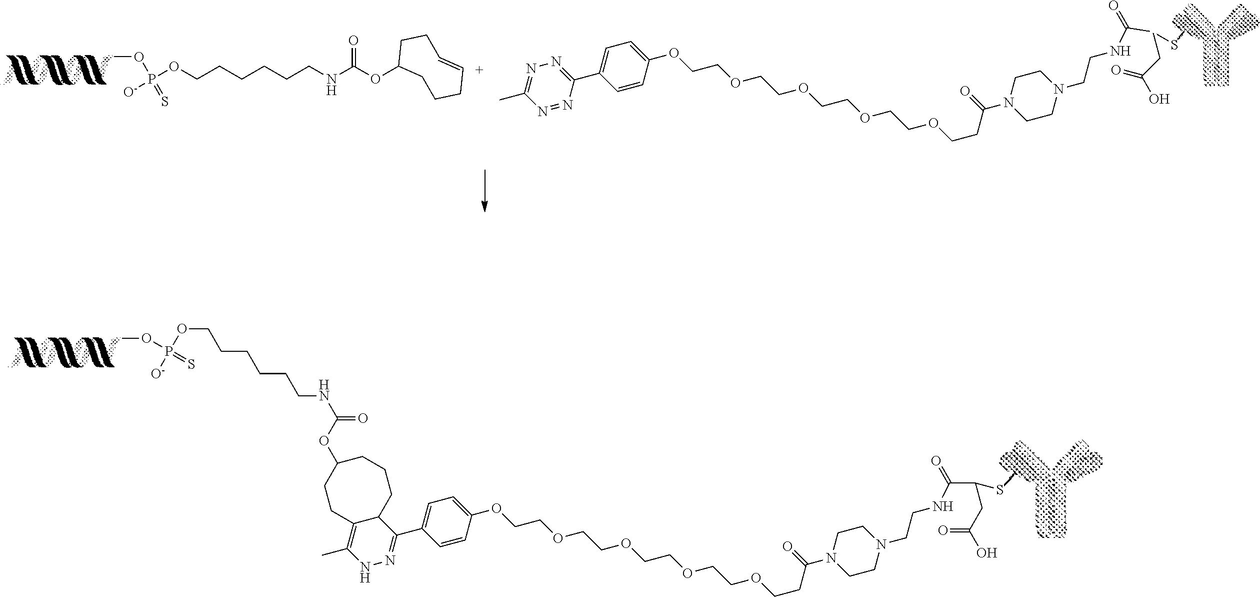

### Conjugation Scheme 2

The second conjugation method utilized the SMCC-functionalized dsRNA for conjugating onto the engineered cysteine of the TfR binding proteins. For this method, TfR binding protein was prepared similarly as above to make the engineered thiol available for conjugation by undergoing a reduction and oxidation process of the TfR binding proteins. This is followed by incubating the SMCC-dsRNA with the TfR binding proteins at 4 molar equivalents for overnight conjugation at 4° C.

Optionally, following conjugation a maleimide hydrolysis step can be done to secure the linker-payload in terminal stage and avoid deconjugation during human body circulation via retro-Michael addition. This succinimide ring hydrolysis process was done by elevating the conjugate pH to 9.0 using 50 mM Arginine (stock solution of 0.7M arginine, pH 9.0 was used) and incubating the solution at 37° C. for 20 hours. The hydrolysis state of the maleimide was confirmed by LCMS characterization of +18 Da that is incurred by the water addition to the succinimide ring.

Step 1a: TfR Binding Protein Conjugation with SMCC Linker

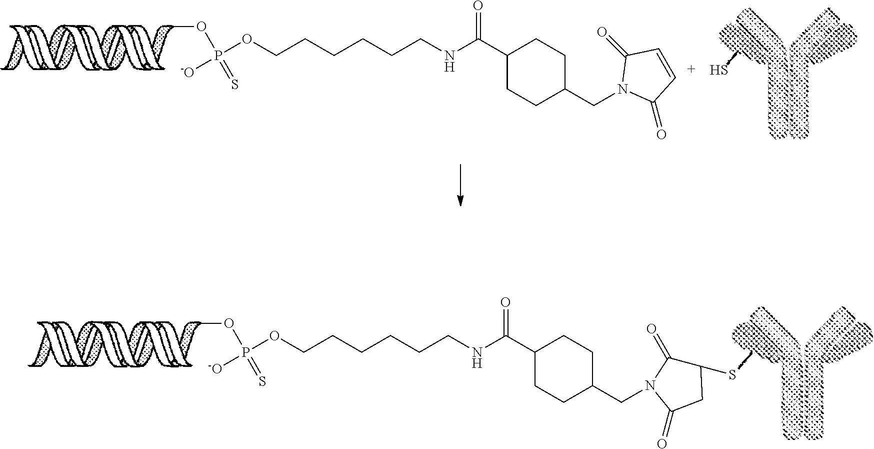

Step 1b: TfR Binding Protein Conjugation with SMCC Linker Ring Opening

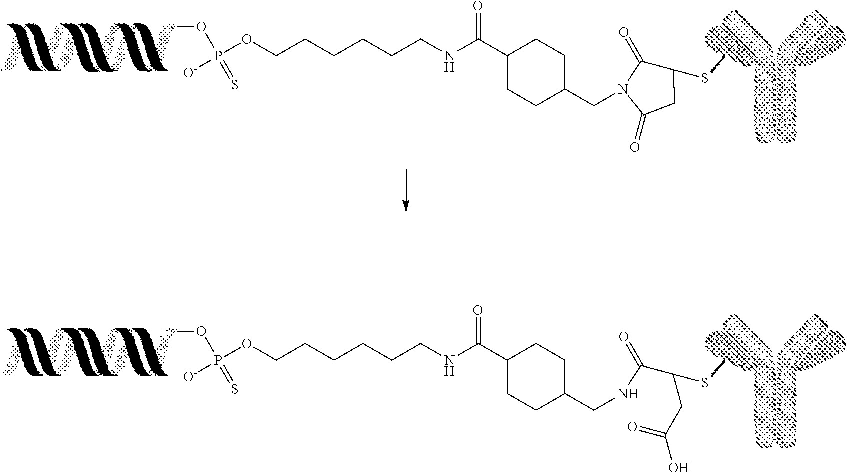 [SMILES: COP([O-])(=S)OCCCCCCNC(=O)C1CCC(CN2C(=O)CC(S)C2=O)CC1 ; COP([O-])(=S)OCCCCCCNC(=O)C1CCC(CNC(=O)C(S)CC(=O)O)CC1]

### Conjugation Scheme 3

The third conjugation method utilized GDM-functionalized dsRNA for conjugating onto the engineered cysteine of the TfR binding protein via disulfide bond. For this method, TfR binding protein was prepared similarly as above to make the engineered thiol available for conjugation by undergoing reduction and oxidation process of the TfR binding protein. Then, dithiobis(5-nitropyridine) was added in as 20 molar equivalents to the protein to generate the intermediate prior to dsRNA conjugation. Excess dithiobis(5-nitropyridine) was removed by desalting. In a second step, GDM-functionalized dsRNA was added to the protein intermediate in a 4 molar equivalents. The dithiobis(5-nitropyridine) acts as a leaving group in this reaction and replaced by the GDM-dsRNA.

Step 1: TfR Binding Protein Conjugation with Dithiobis(5-Nitropyridine) for Intermediate Generation

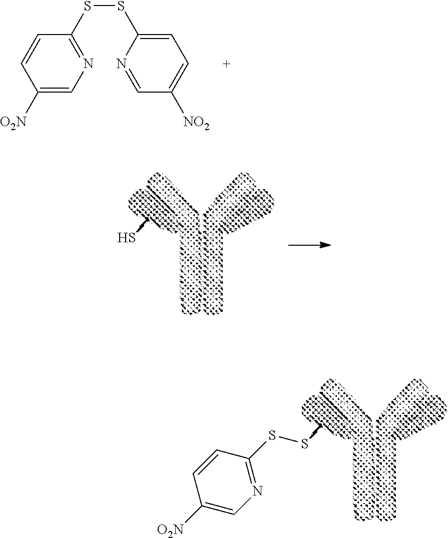

Step 2: dsRNA Conjugation with GDM Functionalized dsRNA

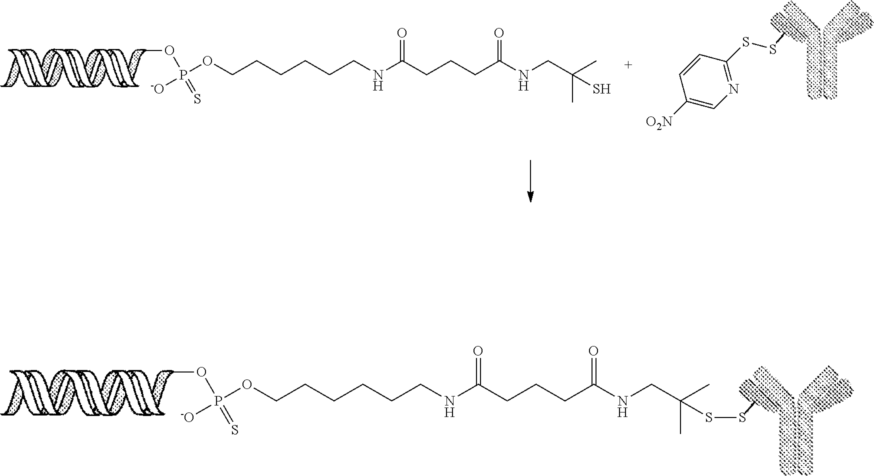

Conjugation was monitored using analytical anion exchange chromatography. A ProPac™ SAX-10 HPLC Column, 10 μm particle, 4 mm diameter, 250 mm length was utilized with the following method. Flow rate of 1 mL/min, Buffer A: 20 mM TRIS pH 7.0, Buffer B: 20 mM TRIS pH 7.0+1.5M NaCl, at 30° C.

TABLE 18A
HPLC gradient used to assess dsRNA conjugation
to TfR binding protein TBP10 and TBP11

| Time [min] | A [%] | B [%] |
|---|---|---|
| 0.00 | 90.0 | 10.0 |
| 16.00 | 20.0 | 80.0 |
| 17.00 | 20.0 | 80.0 |
| 17.20 | 0.0 | 100.0 |
| 18.00 | 0.0 | 100.0 |
| 18.20 | 90.0 | 10.0 |

TABLE 18B
HPLC gradient used to assess dsRNA conjugation
to TfR binding protein TBP14

| Time [min] | A [%] | B [%] |
|---|---|---|
| 0.00 | 85.0 | 15.0 |
| 8.00 | 0.0 | 100.0 |
| 9.00 | 0.0 | 100.0 |
| 9.10 | 85.0 | 15.0 |
| 10.00 | 85.0 | 15.0 |

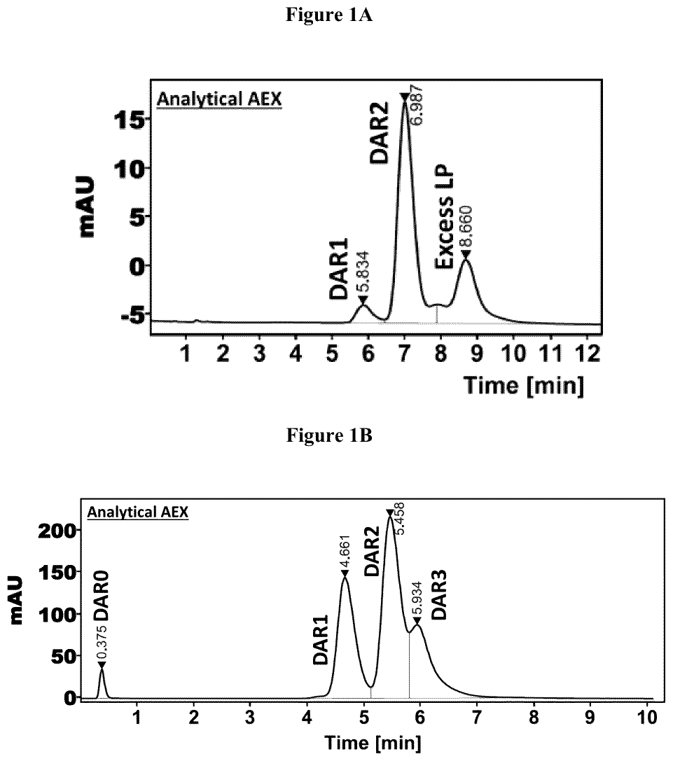

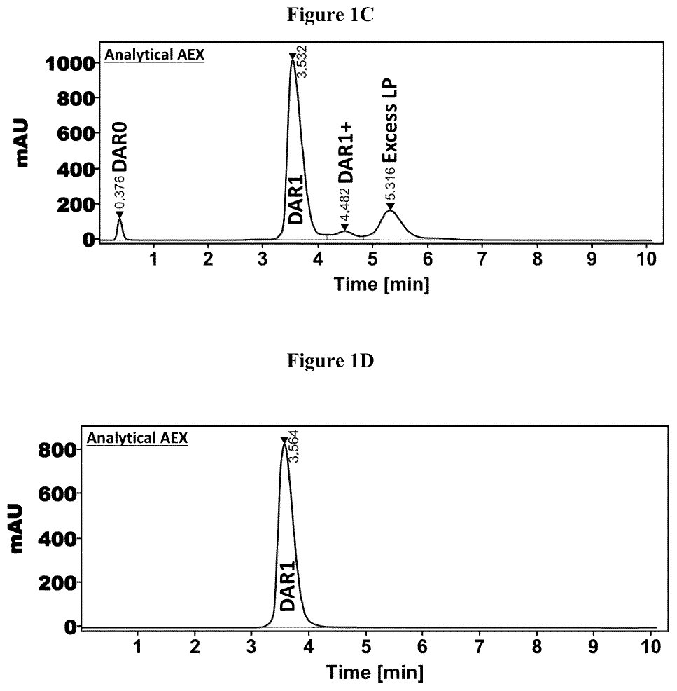

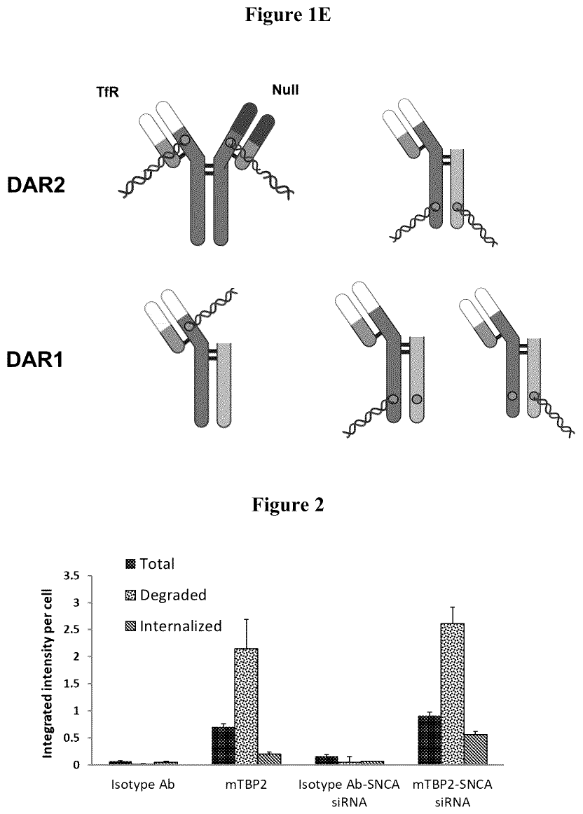

Drug/siRNA to antibody/protein ratio (DAR) was calculated based on peak area % from the analytical anion exchange (aAEX) chromatogram. An illustrative example of a chromatogram of TBP11-dsRNA conjugate before purification is shown in FIG. 1A. FIG. 1C shows an exemplary aAEX chromatogram of DAR profile for TBP15-dsRNA conjugate before purification.

Post conjugation of dsRNA to the TfR binding protein, excess dsRNA and unconjugated protein was removed by further purification. Either preparative size exclusion chromatography (SEC) or preparative anion exchange chromatography was utilized for purification of the final conjugate. Preparative SEC was performed using Cytiva Superdex® 200 in 1×PBS pH 7.2 under an isocratic condition. Alternatively, anion exchange, e.g., ThermoFisher POROS™ XQ, was used with starting buffer of 20 mM TRIS pH 7.0 and eluting with 20 column volume gradient with a buffer containing 20 mM TRIS pH 7.0 and 1M NaCl. These resulted in purified TfR binding protein-dsRNA conjugate devoid of excess dsRNA and minimal unconjugated protein. The resulting conjugate profile was analyzed by analytical anion exchange for final DAR quantitation (see FIGS. 1B and 1D; and Table 19).

An example of a chromatogram of TBP14-dsRNA conjugate after purification is shown in FIG. 1B. FIG. 1D shows an exemplary aAEX chromatogram of DAR profile for TBP15-dsRNA conjugate after purification.

TABLE 19
siRNA/drug to TBP/antibody ratio (DAR)

| | Average | % of | % of | % of | % of |
|---|---|---|---|---|---|
| | DAR | DAR0 | DAR1 | DAR2 | DAR3 |
| TBP14-MAPT | 1.89 | 1.97% | 29.59% | 45.95% | 22.49% |
| siRNA conjugate | | | | | |
| TBP14-SNCA | 1.94 | 2.21% | 27.42% | 45% | 25.37% |
| siRNA conjugate | | | | | |
| TBP15-SNCA | 1.03 | 3.74% | 89.91% | 6.35% | N/A |
| siRNA conjugate | | | | | |
| (before | | | | | |
| purification) | | | | | |
| TBP15-SNCA | 1.0 | N/A | 100% | N/A | N/A |
| siRNA conjugate | | | | | |
| (after | | | | | |
| purification) | | | | | |

### Example 4: In Vitro Characterization of the Mouse TfR Binding Proteins-dsRNA Conjugates

### In Vitro Binding, Internalization and Degradation Assessment in Mouse Cortical Neurons

Fluorescence signal corresponding to total levels, and internalization of TfR binding proteins or TfR binding protein-siRNA conjugates (ARC) was measured by performing a high content live cell imaging assay in primary mouse cortical neurons. Briefly, mouse primary cortical neurons were isolated from wild type C57BL6 mouse embryos at E18. Cells were plated in poly-D-lysine coated 96-well plates at a density of 40,000 cells/well and cultured in NbActiv1 (BrainBits, LLC) containing 1% Antibiotic/Antimycotic (Corning) for 7 days at 37° C. in a tissue culture incubator in a humidified chamber with 5% CO2. On day 7, medium was removed from each well and replaced with culture media with 5 ug/ml (33 nM) of either: (i) Isotype Ab (an isotype control antibody), (ii) mTBP2 (a heterodimeric antibody with a monovalent mouse TfR binding arm and an isotype control arm), (iii) Isotype Ab-SNCA siRNA (Isotype control antibody with dsRNA No. 8 linked to heavy chain constant region 1) or (iv) mTBP2-SNCA siRNA (mTBP2 with dsRNA No. 8 linked to heavy chain constant region 1), together with 10 ug/ml (0.2 uM) of anti-human IgG Fcγ fragment specific Fab fragment (Jackson Immuno #109-007-008) labelled with either DyLight 650 (Thermo Fisher #62266), DL650 together with BHQ3 dye (BioSearch Tech BHQ-3000S-5) or pHAb dye (Promega #G9845) in culture media with 6.7 uM (1 mg/ml) goat gamma globulin (Jackson Immuno #005-000-002) and incubated overnight with live cells grown in a 96 well plate at 37° C.

The following day, cells were washed, incubated for 20 minutes with NucBlue Hoechst dye (Thermo Fisher #R37605), washed again, then imaged with Cytation 5 high content imager (Biotek). DyLight 650 signal measures total TfR binding protein levels, DyLight 650 plus BHQ3 signal measures degradation signal that increases DyLight 650 fluorescence when BHQ3 dye is liberated and FRET quenching is lost, while pHAb pH sensor dye signal measures only internalized fluorescence. Excess goat gamma globulin was added to reduce non-specific binding and uptake of antibodies into the cells. The intensity of the signal in each well was divided by the number of Hoechst-stained nuclei to determine signal intensity per cell. Wells were analyzed in duplicates, and for each well, approximately 20,000 cells were analyzed from images taken with a 4× objective. The background signal was determined from human IgG isotype control and subtracted from the final value.

Results are shown in FIG. 2. High content imaging data demonstrates cellular activity (binding, internalization and degradation properties) of the exemplified mouse TfR binding protein and isotype control antibody. Isotype control antibody and isotype control antibody-dsRNA conjugates lacked activity, while binding, internalization and degradation activity was demonstrated for the exemplified mouse TfR binding protein was demonstrated in mouse primary cortical neurons. In addition, conjugation to dsRNA does not substantially change the activity of the exemplified mouse TfR binding proteins.

### In Vitro Potency Assessment in Mouse Cortical Neurons

Mouse primary cortical neurons were isolated from wild type C57BL6 mouse embryos at E18 and cultured as described above. On day 7, half of the medium was removed from each well and 2× concentration of one of: (i) chol-teg-siSNCA (cholesterol conjugated dsRNA No. 7); (ii) naked SNCA siRNA (unconjugated SNCA siRNA); (iii) Isotype Ab-SNCA siRNA (an isotype control antibody having an dsRNA No. 7 linked at HC Constant region 1) or (iv) mTBP2-SNCA siRNA (mTBP2-dsRNA No. 8 conjugate, dsRNA linked to HC Constant region 1 of mTBP2), in culture media with 2% FBS was added for treatment and incubated with cells for additional 7 days. At the end of treatment, RT-qPCR was performed to quantify targeted mRNA levels using TaqMan Fast Advanced Cell-to-CT kit. Specifically, cells were lysed, cDNA was generated on Mastercycler X50a (Eppendorf), and qPCR was carried out on QuantStudio 7 Flex Real-Time PCR System (Applied Biosystems). Gene expression levels of the SNCA were normalized by β-actin using respective probes (ThermoFisher).

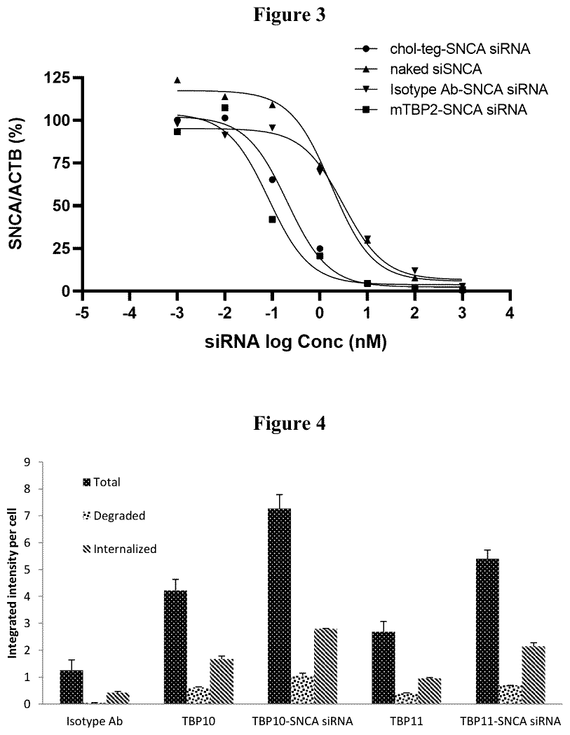

Results are provided FIG. 3 and Table 20. Results provided in Table 20 demonstrate the exemplified mouse TfR binding protein-siRNA conjugates (e.g., mTfR2-dsRNA No. 8 conjugate) successfully targets mouse SNCA and provides potency multiple order of magnitudes greater than the unconjugated siRNA (i.e., naked siRNA) and Isotype Ab-SNCA siRNA, and is equivalent or superior to the potency of cholesterol conjugated siRNA.

TABLE 20
In vitro potency of the indicated molecules for reducing
mouse SNCA mRNA in mouse cortical neurons

| | Naked | Isotype | Cholesterol- | mTBP2-SNCA |
|---|---|---|---|---|
| | SNCA | Ab-SNCA siRNA | SNCA siRNA | siRNA |
| | siRNA | Conjugate | Conjugate | Conjugate |
| IC50 | 1.751 | 3.074 | 0.205 | 0.083 |
| (nM) | | | | |

### Example 5: In Vitro Characterization of the Human TfR Binding Proteins-dsRNA Conjugates In Vitro Binding, Internalization and Degradation Assessment in SHSY5Y Cells

SH-SY5Y cells (ATCC CRL-2266, passage 5-20) were maintained in media that consisted of 225 ml MEM/EBSS (Hyclone:SH30024.02; Gibco 11095-072), 10% heat inactivated fetal bovine serum (Hyclone SH30071.03), 1× Sodium Pyruvate (100×, Hyclone:SH30239.01), 1× Non-Essential Amino Acids (100×, Hyclone SH30238.01) and Na Bicarbonate (7.5%, Hyclone: SH30033.01) and 225 mL HAMs F12 (Corning Cellgro 10-080CV). Cells were plated at 120,000/well and grown for 4 days in a fibronectin coated black 96 well plate (Falcon #353219) at 37° C., 90% humidity in a tissue culture incubator (Thermo Scientific Forma Series 3 Water Jacketed). On day 4, medium was removed from each well and replaced with culture media with 5 ug/ml (33 nM) of either: an isotype control antibody (Isotype Ab), TBP10, TBP11, or the above molecules conjugated to a SNCA siRNA (dsRNA No. 8), together with 10 ug/ml (0.2 uM) of anti-human IgG Fcγ fragment specific Fab fragment (Jackson Immuno #109-007-008) labelled with either DyLight 650 (Thermo Fisher #62266), DL650 together with BHQ3 dye (BioSearch Tech BHQ-30005-5) or pHAb dye (Promega #G9845) in culture media with 6.7 uM (1 mg/ml) goat gamma globulin (Jackson Immuno #005-000-002) and incubated overnight with live cells grown in a 96 well plate at 37 C.

The following day, cells were washed, incubated for 20 min with NucBlue Hoechst dye (Thermo Fisher #R37605), washed again then imaged with Cytation 5 high content imager (Biotek). DyLight 650 signal measures total TfR binding protein levels, DyLight 650 plus BHQ3 signal measures degradation signal that increases DyLight 650 fluorescence when BHQ3 dye is liberated and FRET quenching is lost, while pHAb pH sensor dye signal measures only internalized fluorescence. Excess goat gamma globulin was added to reduce non-specific binding and uptake of antibodies into the cells. The intensity of the signal in each well was divided by the number of Hoechst-stained nuclei to determine signal intensity per cell. Wells were analyzed in duplicates, and for each well, approximately 20,000 cells were analyzed from images taken with a 4× objective. The background signal was determined from human IgG isotype control and subtracted from the final value.

Results are shown in FIG. 4. High content imaging data demonstrates cellular activity (binding, internalization and degradation properties) of the exemplified human TfR binding proteins and Isotype control antibody. Isotype control antibody lacked substantial activity, while binding, internalization and degradation activity was demonstrated for the exemplified human TfR binding proteins on SH-SY5Y cells. In addition, conjugation to dsRNA does not reduce activity of the exemplified human TfR binding proteins.

### In Vitro Potency Assessment in SYSY5Y Cells

SH-SY5Y cells (ATCC CRL-2266, passage 5-20) were maintained as described above. On day 4, medium was removed from each well and replaced with culture media with one of: an isotype control antibody siRNA conjugate (Isotype Ab-SNCA siRNA), TBP10-SNCA siRNA conjugate, or TBP11-SNCA siRNA conjugate in culture media with 2% FBS was added for treatment and incubated with cells for additional 7 days. At the end of treatment, RT-qPCR was performed to quantify target mRNA levels using TaqMan Fast Advanced Cell-to-CT kit. Specifically, cells were lysed, cDNA was generated on Mastercycler X50a (Eppendorf), and qPCR was carried out on QuantStudio 7 Flex Real-Time PCR System (Applied Biosystems). Gene expression levels of the SNCA were normalized by 3-actin using respective probes (ThermoFisher).

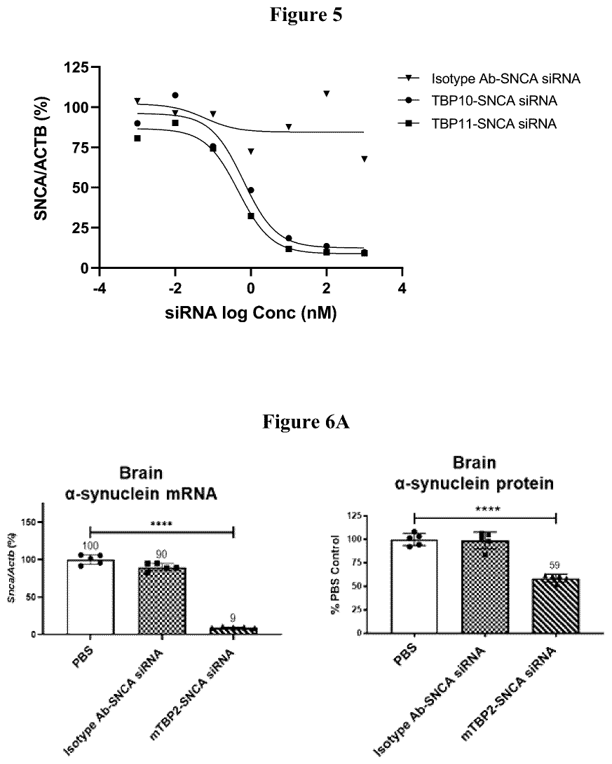

Results are provided in Table 21 and FIG. 5. Results provided in Table 21 demonstrate exemplified human TFR binding protein-siRNA conjugates provide potency for knocking down human SNCA gene while Isotype control antibody conjugate showed low activity.

TABLE 21
In vitro potency for reducing human
SNCA mRNA in SH-SY5Y cells

| | Isotype | TBP10- | TBP11- |
|---|---|---|---|
| | Ab-SNCA siRNA | SNCA siRNA | SNCA siRNA |
| | conjugate | conjugate | conjugate |
| IC50 | N.D.* | 0.64 | 0.47 |
| (nM) | | | |

*N.D. means not determined due to lack of activity precluding accurate assessment.

### Example 6: In Vivo Proof of Concept Demonstration of Pharmacodynamic Efficacy of the Mouse TfR Binding Proteins-dsRNA Conjugates in the CNS with Peripheral Delivery

In Vivo Pharmacodynamic Assessment in Mice with Multiple IV Dosing

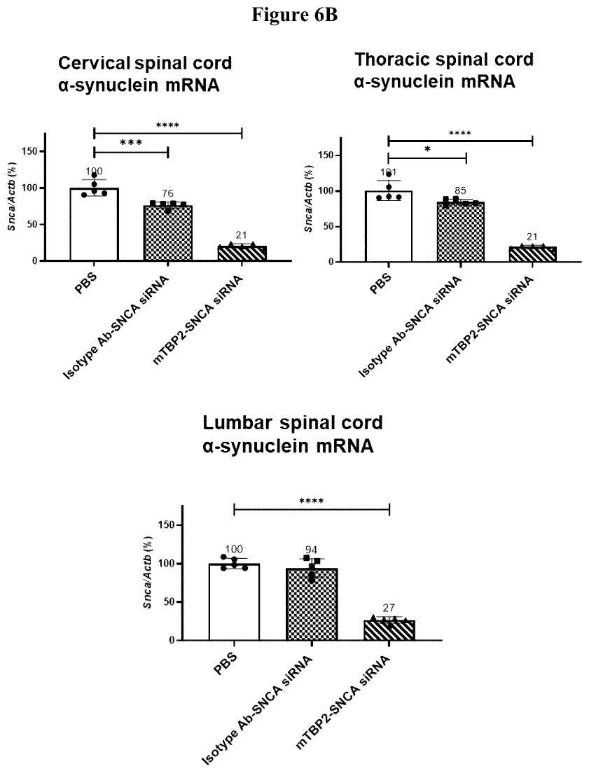

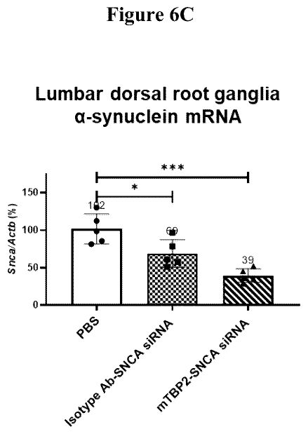

In order to demonstrate that mouse TfR binding protein-siRNA conjugates crosses the BBB and delivers the siRNA cargo to the CNS to reduce SNCA mRNA gene expression, a series of proof of concept studies were conducted to assess Pharmacodynamic efficacy of the constructs with peripheral delivery in mice. PBS control, Isotype Ab-SNCA siRNA, or mTBP2-SNCA siRNA (mTBP2 SNCA-dsRNA No. 8 conjugate) were dosed in 8-week-old FVB mice at 10 mg/kg effective siRNA concentration intravenously either i) weekly dose four times and sacrificed 28 days after the first dose (see FIGS. 6A and 6B), or ii) single dose and sacrificed after 7 days, 28 days, 70 days or 120 days (see FIGS. 6C and 6D). In addition, mouse anti-CD4 antibody (GK1.5) was dosed at 10 mg/kg 2 to 3 days prior to the study to ablate CD4 positive T cells to mitigate undesired pharmacokinetic consequences resulting from spurious anti-drug antibody responses to injected compounds. Mice at designated time points were fully anesthetized then underwent cardiac perfusion with cold PBS (6 ml/min for 5 min) until blood was completely removed to collect brain and spinal cord to assess target mRNA levels by RT-qPCR and target protein levels by ELISA in tissue homogenates. For RT-qPCR, RNA was isolated by using RNeasy Plus Universal Mini Kit (Qiagen 73404). Briefly hemibrain, spinal cord and DRG tissue homogenates were prepared with FastPrep-24 Lysing Matrix D beads to homogenize the tissues with MP Fastprep 24 (MP Biomedical) for at 6 m/s for 40 seconds at 4° C., then centrifuging the vials to collect the supernatant. The RNA was then collected Following determination of RNA quantity with A260/280 ratio with a spectrophotometer, cDNA was generated on Mastercycler X50a (Eppendorf), and qPCR was carried out on QuantStudio 7 Flex Real-Time PCR System (Applied Biosystems). Gene expression levels of the SNCA were normalized by β-actin using respective probes (ThermoFisher).

Results are shown in FIGS. 6A-6C. Multiple doses of IV administration of mTBP2 SNCA-siRNA in mice resulted in robust 91% reduction of SNCA mRNA and 41% reduction of SNCA protein in the brain compared to PBS dosed controls 28 days after the initial dose (FIG. 6A). Importantly, Isotype Ab-SNCA siRNA did not elicit significant reduction in SNCA mRNA demonstrating that active TfR mediated transport was required to deliver siRNA cargo to the CNS, demonstrating BBB crossing and delivery to the brain. Moreover, assessment of the spinal cord 28 days after the initial dose also demonstrated robust reductions in cervical, thoracic, lumber regions with 79%, 79% and 73% SNCA mRNA reductions respectively with mTBP2-SNCA siRNA compared to PBS dosed controls (FIG. 6B). There was also a significant 61% reduction of SNCA mRNA in lumbar dorsal root ganglia (DRG) (FIG. 6C). Interestingly, there was low but significant levels of SNCA mRNA reduction in the cervical and thoracic spinal cord, and in lumbar DRG with Isocontrol Ab-SNCA siRNA, suggesting that there may be limited levels of spinal cord and DRG siRNA delivery without TfR mediated delivery.

### Example 7: In Vivo Proof of Concept Demonstration of Pharmacodynamic Time Course of the Mouse TfR Binding Proteins-dsRNA Conjugates in the CNS with Peripheral Delivery

In Vivo Pharmacodynamic Time Course Assessment in Mice with a Single IV Dosing

High efficacy of mTBP2-SNCA siRNA in the brain and spinal cord with multiple IV dosing suggested that there will likely be significant efficacy with a single dose. Therefore, a follow up Proof of Concept study was performed to determine single IV dose efficacy, and to determine the time course of pharmacodynamic efficacy for SNCA mRNA and protein levels to inform subsequent study design in non-human primates. For each time point 5 mice were sacrificed to collect the tissues for analytics as described above.

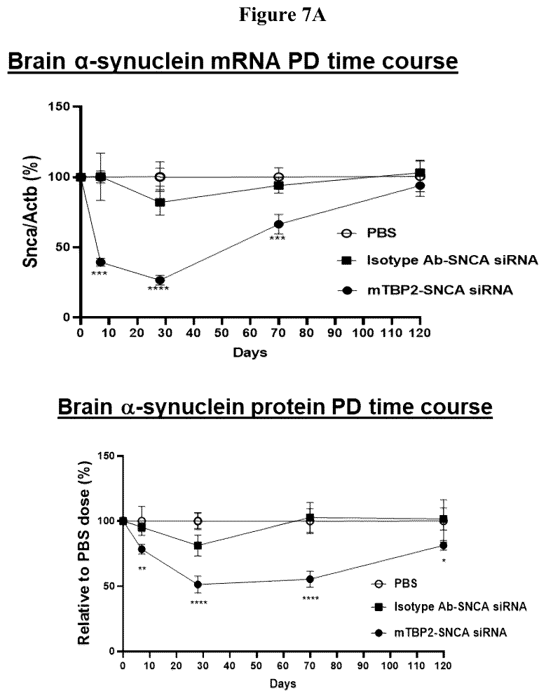

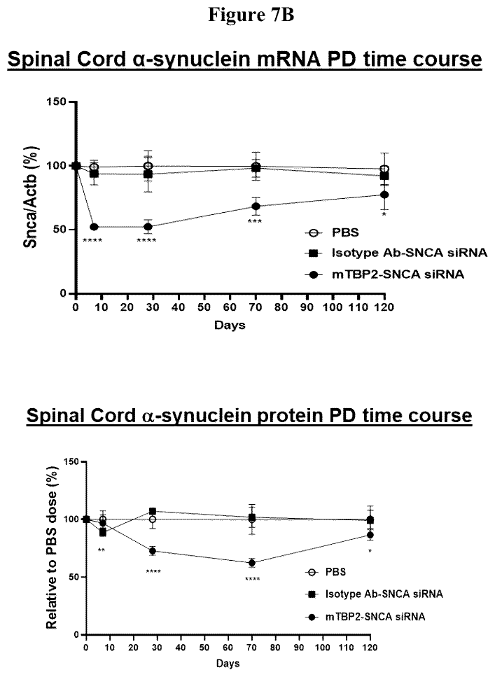

As shown in FIG. 7A, Single IV administration of mTBP2 SNCA-siRNA in mice led to robust reduction of SNCA in the brain compared to PBS dosed control, beginning at 7 days following dosing (60% mRNA reduction, 22% protein reduction) with maximal reduction at 28 days (73% mRNA reduction, 41% protein reduction), followed by persistent reduction at 70 days (34% mRNA reduction, 45% protein reduction) which reverted towards PBS baseline group at 120 days (6% mRNA reduction and 19% protein reduction).

Moreover, as shown in FIG. 7B, single IV administration of mTBP2 SNCA also led to robust SNCA reduction in the spinal cord compared to PBS dosed control group, beginning at 7 days following dosing for mRNA only (48% mRNA reduction, 3% protein reduction) with both mRNA and protein reduction at 28 days (48% mRNA reduction, 27% protein reduction), followed by persistent reduction at 70 days (32% mRNA reduction, 48% protein reduction) which reverted towards PBS baseline group at 120 days (23% mRNA reduction and 14% protein reduction).

### Example 8: In Vivo Characterization of the Human TfR Binding Proteins-dsRNA Conjugates

8A. In Vivo Pharmacodynamic Assessment in Non-Human Primates (NHPs) 29 Days after a Single Dose of Human TfR Binding Proteins-SNCA siRNA Conjugates.

Following robust proof of concept demonstration of peripheral siRNA delivery into the CNS across BBB in mice, Pharmacodynamic properties of human TfR binding protein-SNCA siRNA conjugates were assessed in NHPs according to the following. Cynomolgus monkeys weighing 2-3 kg were dosed intravenously in the Saphenous vein in the thigh with i) PBS (n=8), ii) TBP10-SNCA siRNA (TB10-dsRNA No. 8 conjugate) (n=6) at 8.8 mg/kg effective siRNA concentration, or iii) TBP11-SNCA siRNA (TBP11-dsRNA No. 8 conjugate) (n=6) at 2.6 mg/kg effective siRNA concentration and sacrificed 29 days after the first dose. Deeply anesthetized animals underwent cardiac perfusion, then brain, spinal cord and peripheral tissues were collected. The brain was coronally sectioned, 3 mm punches were collected from indicated subregions and frozen, as well as tissues were collected from spinal cord, liver and muscles to assess target mRNA levels by RT-qPCR in tissue homogenates. The total RNA from NUP tissues were isolated using the RNadvance Tissue kit (Beckman Coulter, Indianapolis, IN) manually or on a Biomek i7 liquid handler (Beckman Coulter), following the manufacturer's procedure with some modifications. In brief, the frozen tissue sections were mixed with one 5 mm stainless steel ball, lysis buffer and proteinase K, homogenized for 5 cycles of 30 s at 1200 rpm, with an interval of 20 s between cycles, on a 2010 GenoGrinder (SPEX SamplePrep, Metuchen, NJ). Tissues from some regions were shaved on dry ice, prior to homogenization. The homogenates were incubated at 37 C for 1 h, then extracted with an equal volume of phenol-chloroform. The RNA in the supernatant were purified with the RNadvance tissue kit, where a 30 min digestion with DNase was included. The concentration and the purity (A260/A280) of the RNA elute were determined by spectrophotometry. RNA was normalized to 15 ng/10 uL PCR, digested again with ezDNase (ds-DNA specific) prior to reverse-transcription using the SSIV VILO kit (Thermo Fisher Scientific, Waltham, MA). The expression of the respective gene targets in the cDNA was determined using TaqMan qPCR assays on the QuantStudio 7 Pro platform (Thermo Fisher Scientific). Gene expression of SNCA was normalized by Gene expression levels of the SNCA were normalized by j-actin using respective probes (ThermoFisher). The tissues analyzed and their acronyms are: Liver; Gastrocnemius Muscle; AN, Arcuate Nucleus; Med Em, Median Eminence; LSC, lumbar spinal cord; Medulla; Pons; CB, Cerebellumn; Midbrain; SN, Substantia Nigra; Caudate; PUT, Putamen; HT, hypothalamus; H, hippocampus, PFC, prefrontal cortex gray matter; PFC, prefrontal cortex white matter.

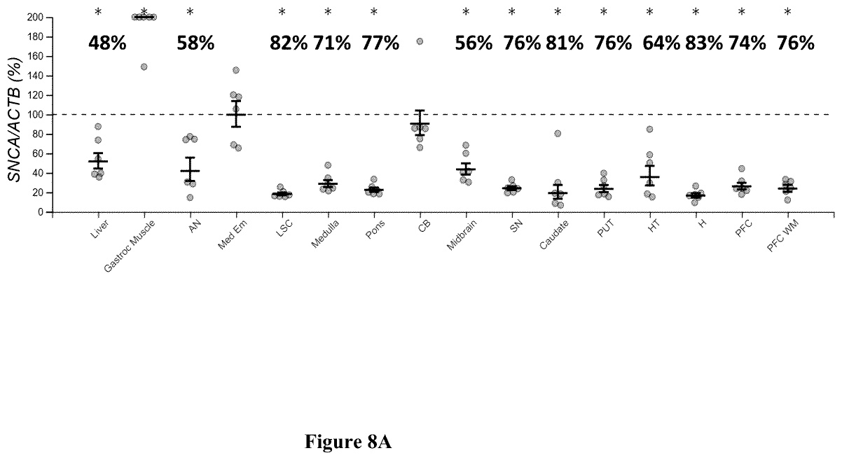

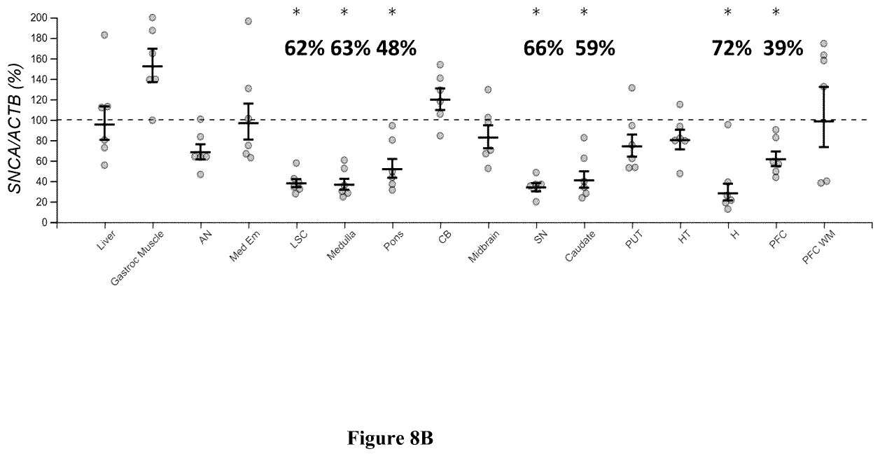

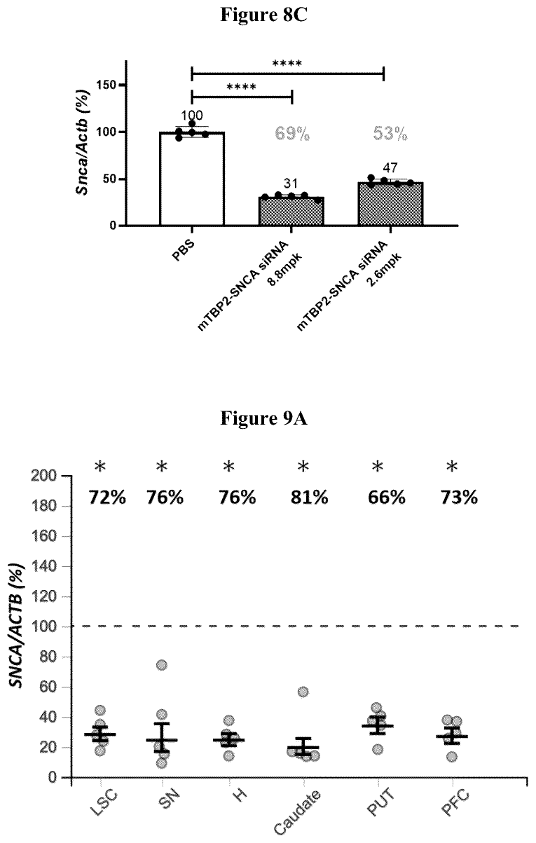

Peripheral IV administration of TBP10-SNCA siRNA at 8.8 mg/kg in NHPs led to significant reduction of SNCA mRNA in key brain regions and lumbar spinal cord compared to PBS treatment group at 29 days following dosing. As shown in FIG. 8A, significant SNCA mRNA reductions were demonstrated in the liver (48%), arcuate nucleus (58%), lumbar spinal cord (82%), medulla (71%), pons (77%), midbrain (56%), substantia nigra (76%), caudate (81%), putamen (76%), hypothalamus (64%), hippocampus (83%), prefrontal cortex gray matter (74%), prefrontal cortex white matter (76%). Other brain regions and tissues assessed did not demonstrate significant reduction in SNCA mRNA as shown (FIG. 8A).

Peripheral IV administration of TBP11-SNCA siRNA at lower 2.6 mg/kg dose in NHPs also led to significant reduction of SNCA mRNA in key brain regions and lumbar spinal cord compared to PBS treatment group at 29 days following dosing. As shown in FIG. 8B, significant SNCA mRNA reductions were demonstrated in the lumbar spinal cord (62%), medulla (63%), pons (48%), substantia nigra (66%), caudate (59%), hippocampus (72%) and prefrontal cortex gray matter (39%). Other brain regions and tissues assessed did not demonstrate significant reduction in SNCA mRNA as shown (FIG. 8B).

In order to determine the expected levels of brain SNCA mRNA reduction at NHP equivalent doses, a cohort of mice were single IV dosed with equivalent 8.8 mg/kg and 2.6 mg/kg concentration of mTBP2-SNCA siRNA and processed as described above to assess translatability in mRNA KD efficacy by RT-qPCR.

Mice dosed intravenously at a dose equivalent to 8.8 mg/kg effective siRNA concentration demonstrated 69% reduction in SNCA mRNA in the brain, whereas dosing at 2.6 mg/kg effective siRNA concentration demonstrated 53% reduction of SNCA mRNA in the brain, demonstrating that similar efficacy is translated from rodents to NHPs (FIG. 8C).

8B. In Vivo Pharmacodynamic Assessment in NHPs 85 Days after a Single Dose or Three Monthly Doses of Human TfR Binding Proteins-SNCA siRNA Conjugates.

A pharmacodynamic study was conducted to determine efficacy of human TfR binding protein-SNCA siRNA conjugates 3 months after a single dose or three monthly doses. Cynomolgus monkeys weighing 2-3 kg were dosed either a single intravenous dose in the Saphenous vein in the thigh with TBP14-SNCA siRNA (dsRNA No. 10 in Table 11a) conjugate (n=5) at 10 mg/kg, or three monthly intravenous doses in the Saphenous vein in the thigh with i) PBS (n=5), or ii) TBP14-SNCA siRNA (dsRNA No. 10 in Table 11a) conjugate (n=5) at 10 mg/kg. All the groups were dosed with anti-CD4 antibody at 30 mg/kg immediately after the dose of the test article for mitigating anti-drug antibody response. 85 days post the single dose or after the first dose in the three monthly dosing regime, deeply anesthetized animals underwent cardiac perfusion, then brain, spinal cord and peripheral tissues were collected.

The brain was coronally sectioned, 4 mm punches were collected from indicated subregions and frozen, as well as tissues were collected from spinal cord, liver, and muscles to assess target mRNA and protein levels by RT-qPCR and ELISA respectively in tissue homogenates. To determine mRNA levels, the total RNA from NHP tissues were isolated using the RNadvance Tissue kit (Beckman Coulter, Indianapolis, IN) manually or on a Biomek i7 liquid handler (Beckman Coulter), following the manufacturer's procedure with some modifications. In brief, the frozen tissue sections were mixed with one 5 mm stainless steel ball, lysis buffer and proteinase K, homogenized for 5 cycles of 30 s at 1200 rpm, with an interval of 20 s between cycles, on a 2010 GenoGrinder (SPEX SamplePrep, Metuchen, NJ). Tissues from some regions were shaved on dry ice, prior to homogenization. The homogenates were incubated at 37° C. for 1 hour, then extracted with an equal volume of phenol-chloroform. The RNA in the supernatant were purified with the Rnadvance tissue kit, where a 30 minute digestion with Dnase was included. The concentration and the purity (A260/A280) of the RNA elute were determined by spectrophotometry. RNA was normalized to 15 ng/10 uL PCR, digested again with ezDNase (ds-DNA specific) prior to reverse-transcription using the SSIV VILO kit (Thermo Fisher Scientific, Waltham, MA). The expression of the respective gene targets in the cDNA was determined using TaqMan qPCR assays on the QuantStudio 7 Pro platform (Thermo Fisher Scientific). Gene expression levels of the SNCA were normalized by j-actin using respective probes (ThermoFisher) for CNS regions and GAPDH for Gastrocnemius Muscles (ThermoFisher).

To determine α-synuclein protein levels, frozen 4 mm-punches of neural tissue biopsies were mixed with cold RIPA buffer (Pierce #89901, Thermo Scientific, Waltham, MA), containing the protease and phosphatase Inhibitors (Halt™ Protease and Phosphatase Inhibitor Cocktail, Thermo Scientific), at a ratio of 20 mL buffer to 1 gram tissue. The tissue-RIPA mixture was homogenized using a 5 mm stainless steel bead on a 2010 GenoGrinder (Spex SamplePrep, Metuchen, NJ). The homogenate was then centrifuged in a refrigerated centrifuge (Eppendorf, Hamburg, Germany), and the supernatant was transferred, made into multiple single-use aliquots, and stored in −80° C. for further analysis.

The protein concentration in the protein lysate was determined using the Pierce™ BCA Protein Assay Kit (Thermo Scientific), following manufacturer's instruction. In particular, the serially diluted bovine serum albumin (BSA) standards were analyzed in duplicate; while each protein lysate sample was diluted by 10 folds, or by 20 folds in water, then analyzed in singlet, respectively. The protein concentration in the undiluted sample, was then obtained by averaging that derived from the 10-fold diluted and that from the 20-fold diluted.

The level of α-synuclein protein in the protein lysate was measured using an in-house developed sandwich ELISA. Briefly, the half-area 96-well flat bottom UV-transparent microplate (Corning, Corning, NY) was coated with the capture antibody (α-synuclein: anti-synuclein antibody, Syn42, Eli Lilly, Indianapolis, IN) at 4° C. overnight with agitation. The wells were blocked with 2% bovine serum albumin (BSA) (Thermo Scientific) in phosphate-buffered saline Tween20™ solution (PBST) (Thermo Scientific) at room temperature (RT) for 60 min. After washing, the wells on each plate for α-Syn ELISA were added protein lysate or the recombinant human alpha-synuclein protein (α-synuclein: rPeptide, Watkinsville, GA) that has been diluted in the PBST containing 2% BSA. The plates were incubated at 4° C. overnight with agitation.

The plates for a-Syn ELISA were washed, then incubated with the detection antibody (Rabbit pAb Anti-α-synuclein. US Biological, Salem, MA) in PBST containing 2% BSA at RT for 3 hours. The plates were washed again, then incubated with the Anti-rabbit HRP-linked Antibody in PBST containing 2% BSA at RT for 1 hour.

To minimize the variation, all the biopsies from the same brain region, as well as a set of serially diluted recombinant human α-synuclein protein standards, were analyzed on the same ELISA plate. All the samples, including the recombinant protein standards, were analyzed in duplicate. The arithmetic mean of the OD450 from the duplicate, after subtraction of the plate blank, was used for further calculation. The standard curve on each ELISA plate was created by fitting the OD450 (Y-axis) and the protein concentration (X-axis) in each of serially diluted protein standards with the logistic 4P nonlinear regression model, using the JMP software (SAS Institute, Cary, NY). The concentration of the respective protein in each diluted sample was then reversely calculated from respective OD450, based on the standard curve. The level of α-synuclein protein in each sample was normalized to the level of total protein, and the remaining α-synuclein protein expression in the treated group was calculated as the percent of remaining α-synuclein protein expression in the treatment group, relative to the average expression of that protein in the aCSF or PBS control group.

The tissues analyzed for mRNA or protein levels and their acronyms are: Gastrocnemius Muscle; LSC, lumbar spinal cord; SN, Substantia Nigra; Caudate; PUT, Putamen; H, hippocampus, PFC, prefrontal cortex gray matter and LDRG, Lumbar DRG.

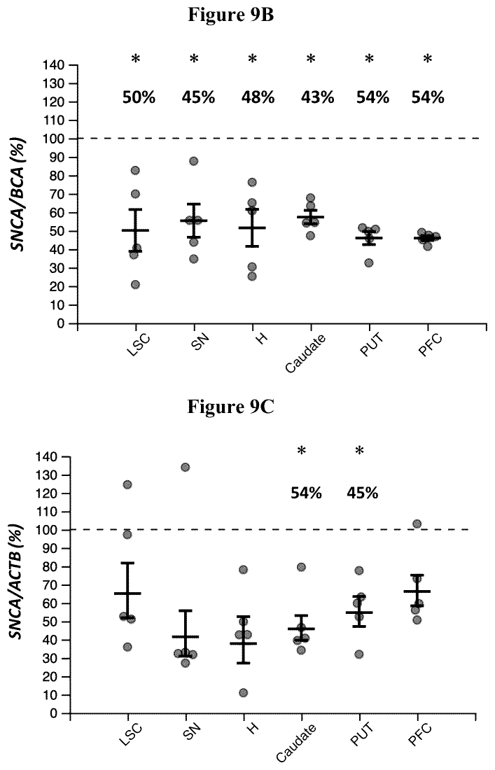

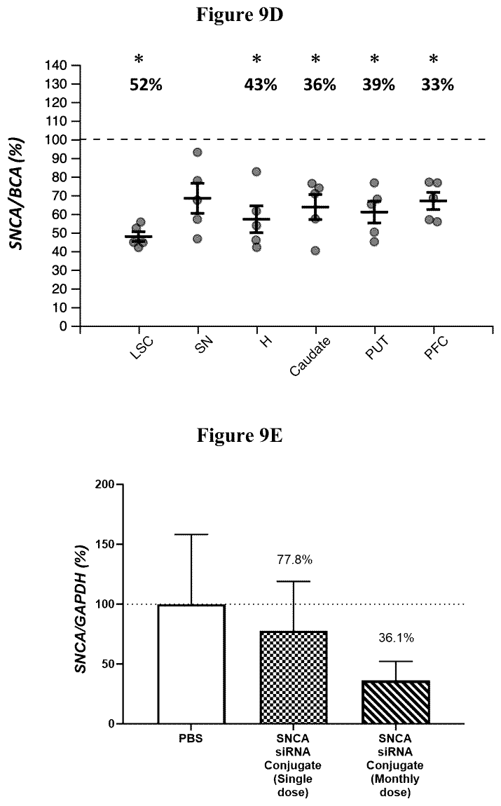

Three monthly peripheral IV administration of TBP14-SNCA siRNA (dsRNA No. 10 in Table 11a) conjugate at 10 mg/kg dose in NHPs led to significant reduction of SNCA mRNA in key brain regions and lumbar spinal cord compared to PBS treatment group at 85 days post first dose. As shown in FIG. 9A, significant SNCA mRNA reductions were demonstrated in the lumbar spinal cord (72%), substantia nigra (76%), caudate (81%), putamen (66%) hippocampus (76%) and prefrontal cortex gray matter (73%). FIG. 9B demonstrates significant reduction of α-synuclein protein in key brain regions and lumbar spinal cord compared to the PBS treated control group 85 days post first dose. As shown in FIG. 9B, significant reduction of α-synuclein protein was observed in Lumbar Spinal Cord (50%), substantia nigra (45%), caudate (43%), putamen (54%), hippocampus (48%) and prefrontal cortex (54%).

Single peripheral IV administration of TBP14-SNCA siRNA (dsRNA No. 10 in Table 11a) conjugate at 10 mg/kg dose in NHPs led to significant reduction of SNCA mRNA in key brain regions compared to PBS treatment group at 85 days following dosing. As shown in FIG. 9C, significant SNCA mRNA reductions were demonstrated in the lumbar caudate (54%) and putamen (45%). Other brain regions and tissues assessed did not demonstrate significant reduction in SNCA mRNA as shown (FIG. 9C). FIG. 9D demonstrates significant reduction of α-synuclein protein in key brain regions and lumbar spinal cord compared to the PBS treated control group 85 days post first dose. As shown in FIG. 9D significant reduction of α-synuclein protein was observed in Lumbar Spinal Cord (52%), caudate (36%), putamen (39%), hippocampus (43%) and prefrontal cortex (33%). Other brain regions and tissues assessed did not demonstrate significant reduction in α-synuclein protein as shown (FIG. 9D).

SNCA mRNA reduction in the Gastrocnemius Muscle after a single or three monthly dosing is shown in FIG. 9E.

8C. In Vivo Pharmacodynamic Assessment in NHPs after Three Monthly Doses of Human TfR Binding Proteins-MAPT siRNA Conjugates.

A pharmacodynamic study was conducted to determine efficacy of human TfR binding protein-MAPT siRNA conjugates after three monthly doses. A group of Cynomolgus monkeys weighing 2-3 kg were dosed monthly intravenously in the Saphenous vein in the thigh with i) PBS (n=5), or ii) TBP14-MAPT siRNA (dsRNA No. 38 in Table 11b) (n=5) at 10 mg/kg effective siRNA concentration. A separate group of Cynomolgus monkeys weighing 2-3 kg were dosed monthly intravenously in the Saphenous vein in the thigh with i) PBS (n=5), ii) TBP14-MAPT siRNA (dsRNA No. 39 in Table 11b) (n=5) at 10 mg/kg effective siRNA concentration, or iii) TBP14-MAPT siRNA (dsRNA No. 40 in Table 11b) (n=5) at 10 mg/kg effective siRNA concentration. All the groups were dosed with anti-CD4 antibody at 30 mg/kg immediately after the dose of the test article for mitigating anti-drug antibody response. About 85 days after the first dose, deeply anesthetized animals underwent cardiac perfusion, then brain, spinal cord and peripheral tissues were collected.

The brain was coronally sectioned, 4 mm punches were collected from indicated subregions and frozen, as well as tissues were collected from spinal cord, liver, and muscles to assess target mRNA and protein levels by RT-qPCR and ELISA respectively in tissue homogenates. To determine mRNA levels the total RNA from NHP tissues were isolated using the RNadvance Tissue kit (Beckman Coulter, Indianapolis, IN) manually or on a Biomek i7 liquid handler (Beckman Coulter), following the manufacturer's procedure with some modifications. In brief, the frozen tissue sections were mixed with one 5 mm stainless steel ball, lysis buffer and proteinase K, homogenized for 5 cycles of 30 s at 1200 rpm, with an interval of 20 s between cycles, on a 2010 GenoGrinder (SPEX SamplePrep, Metuchen, NJ). Tissues from some regions were shaved on dry ice, prior to homogenization. The homogenates were incubated at 37 C for 1 h, then extracted with an equal volume of phenol-chloroform. The RNA in the supernatant were purified with the RNadvance tissue kit, where a 30 min digestion with DNase was included. The concentration and the purity (A260/A280) of the RNA elute were determined by spectrophotometry. RNA was normalized to 15 ng/10 uL PCR, digested again with ezDNase (ds-DNA specific) prior to reverse-transcription using the SSIV VILO kit (Thermo Fisher Scientific, Waltham, MA). The expression of the respective gene targets in the cDNA was determined using TaqMan qPCR assays on the QuantStudio 7 Pro platform (Thermo Fisher Scientific). Gene expression levels of the MAPT were normalized by 3-actin using respective probes (ThermoFisher) for CNS regions and GAPDH for Gastrocnemius Muscles (ThermoFisher).

To determine Tau protein levels, frozen 4 mm-punches of neural tissue biopsies were mixed with cold RIPA buffer (Pierce #89901, Thermo Scientific, Waltham, MA), containing the protease and phosphatase Inhibitors (Halt™ Protease and Phosphatase Inhibitor Cocktail, Thermo Scientific), at a ratio of 20 mL buffer to 1 g tissue. The tissue-RIPA mixture was homogenized using a 5 mm stainless steel bead on a 2010 GenoGrinder (Spex SamplePrep, Metuchen, NJ). The homogenate was then centrifuged in a refrigerated centrifuge (Eppendorf, Hamburg, Germany), and the supernatant was transferred, made into multiple single-use aliquots, and stored in −80° C. for further analysis.

The protein concentration in the protein lysate was determined using the Pierce™ BCA Protein Assay Kit (Thermo Scientific), following manufacturer's instruction. In particular, the serially diluted bovine serum albumin (BSA) standards were analyzed in duplicate; while each protein lysate sample was diluted by 10 folds, or by 20 folds in water, then analyzed in singlet, respectively. The protein concentration in the undiluted sample, was then obtained by averaging that derived from the 10-fold diluted and that from the 20-fold diluted.

The level of Tau protein in the protein lysate was measured using an in-house developed sandwich ELISA. Briefly, the half-area 96-well flat bottom UV-transparent microplate (Corning, Corning, NY) was coated with the capture antibody (Tau: anti-human Tau antibody, Tau5, Eli Lilly, Indianapolis, IN) at 4° C. overnight with agitation. The wells were blocked with 2% bovine serum albumin (BSA) (Thermo Scientific) in phosphate buffered saline Tween20™ solution (PBST) (Thermo Scientific) at room temperature (RT) for 60 min. After washing, the wells on each plate were added with the protein lysate or the recombinant human Tau protein (Tau: Tau441, Eli Lilly) that has been diluted in the PBST containing 2% BSA and the detection antibody (Tau: anti-human Tau antibody, Biotinylated DA9, Eli Lilly). The plates were incubated at 4° C. overnight with agitation.

On the Following day, the plates were washed, then incubated with Pierce™ High Sensitivity Streptavidin-conjugated horseradish peroxidase (HRP) (Thermo Scientific) in PBST containing 2% BSA at RT for 30 min. The HRP enzymatic reaction was visualized with addition of the TMB substrate solution (T0440, Sigma Aldrich, St. Louis, MO), and stopped with addition of sulfuric acid (ELISA Stop solution, Thermo Scientific). Optical density (OD) of the samples were measured at 450 nm (OD450) on an Envision plate reader (PerkinElmer, Waltham, MA).

To minimize the variation, all the biopsies from the same brain region, as well as a set of serially diluted recombinant human Tau protein standards, were analyzed on the same ELISA plate. All the samples, including the recombinant protein standards, were analyzed in duplicate. The arithmetic mean of the OD450 from the duplicate, after subtraction of the plate blank, was used for further calculation. The standard curve on each ELISA plate was created by fitting the OD450 (Y-axis) and the protein concentration (X-axis) in each of serially diluted protein standards with the logistic 4P nonlinear regression model, using the JMP software (SAS Institute, Cary, NY). The concentration of the respective protein in each diluted sample was then reversely calculated from respective OD450, based on the standard curve. The level of Tau protein in each sample was normalized to the level of total protein, and the remaining Tau protein expression in the treated group was calculated as the percent of remaining Tau protein expression in the treatment group, relative to the average expression of that protein in the aCSF or PBS control group.

The tissues analyzed for mRNA or protein levels and their acronyms are: LSC, lumbar spinal cord; SN, Substantia Nigra; Caudate; PUT, Putamen; H, hippocampus, and PFC, prefrontal cortex gray matter.

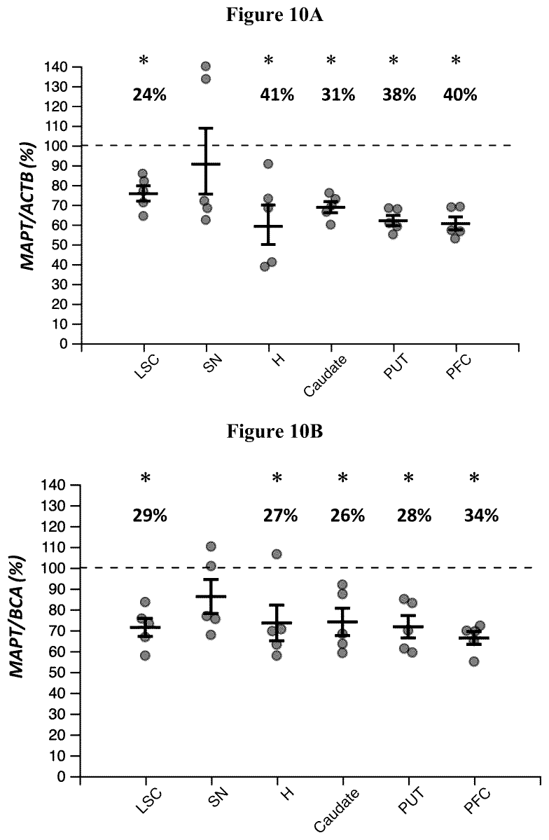

Three monthly peripheral IV administration of TBP14-MAPT siRNA (dsRNA No. 38 in Table 11b) at 10 mg/kg in NHPs led to significant reduction of MAPT mRNA and protein in key brain regions and lumbar spinal cord compared to PBS treatment group at 85 days post first dose. As shown in FIG. 10A, significant MAPT mRNA reductions were demonstrated in the lumbar spinal cord (24%), caudate (31%), putamen (38%), hippocampus (41%), prefrontal cortex gray matter (40%). Other brain regions and tissues assessed did not demonstrate significant reduction in MAPT mRNA as shown (FIG. 10A). FIG. 10B demonstrates significant reduction of Tau protein in key brain regions and lumbar spinal cord compared to the PBS treated control group 85 days post first dose. As shown in FIG. 10B, significant reduction of Tau protein was observed in Lumbar Spinal Cord (29%), caudate (26%), putamen (28%), hippocampus (27%) and prefrontal cortex (34%). Other brain regions and tissues assessed did not demonstrate significant reduction in Tau protein as shown (FIG. 10B).

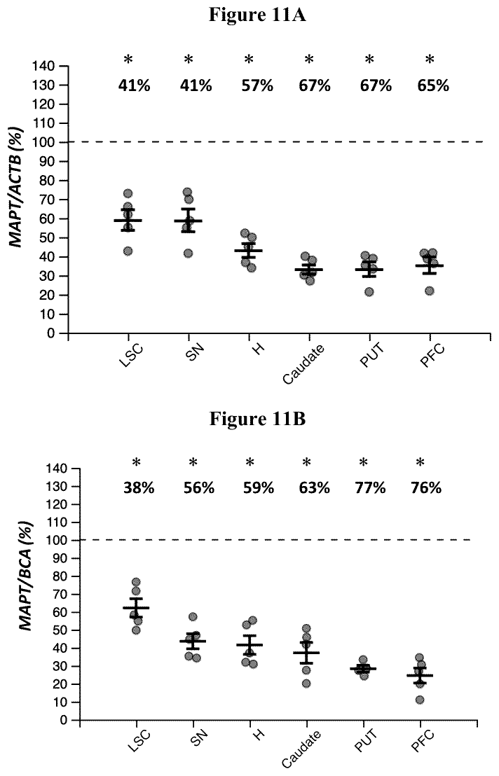

Three monthly peripheral IV administrations of TBP14-MAPT siRNA (dsRNA No. 39 in Table 11b) conjugate at 10 mg/kg in NHPs led to significant reduction of MAPT mRNA in key brain regions and lumbar spinal cord compared to PBS treatment group at 85 days post first dose. As shown in FIG. 11A, significant MAPT mRNA reductions were demonstrated in the lumbar spinal cord (41%), substantia nigra (41%), caudate (67%), putamen (67%), hippocampus (57%), prefrontal cortex gray matter (65%). FIG. 11B demonstrates significant reduction of Tau protein in key brain regions and lumbar spinal cord compared to the PBS treated control group 85 days post first dose. As shown in FIG. 11B, significant reduction of Tau protein was observed in Lumbar Spinal Cord (38%), substantia nigra (56%), caudate (63%), putamen (77%), hippocampus (59%) and prefrontal cortex (76%).

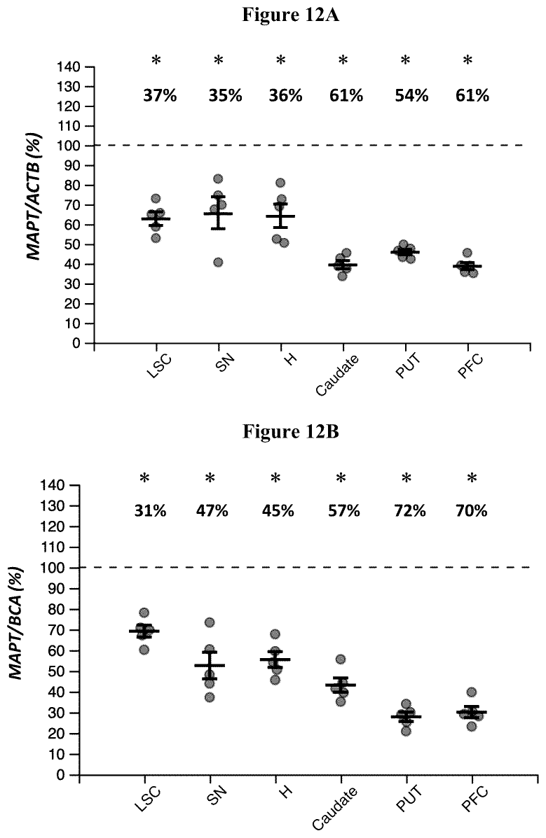

Three monthly peripheral IV administration of TBP14-MAPT siRNA (dsRNA No. 40 in Table 11b) conjugate at 10 mg/kg dose in NHPs led to significant reduction of MAPT mRNA in key brain regions and lumbar spinal cord compared to PBS treatment group at 85 days post first dose. As shown in FIG. 12A, significant MAPT mRNA reductions were demonstrated in the lumbar spinal cord (37%), substantia nigra (35%), caudate (61%), putamen (54%), hippocampus (36%) and prefrontal cortex gray matter (61%). FIG. 12B demonstrates significant reduction of Tau protein in key brain regions and lumbar spinal cord compared to the PBS treated control group 85 days post first dose. As shown in FIG. 12B, significant reduction of Tau protein was observed in Lumbar Spinal Cord (31%), substantia nigra (47%), caudate (57%), putamen (72%), hippocampus (45%) and prefrontal cortex (70%).

8D. In Vivo Pharmacodynamic Assessment in NHPs 1-Month after a Single Dose of BBB Penetrating Antibodies Targeting SNCA Human TfR Binding Proteins-SNCA siRNA Conjugates (DAR1)

Following demonstration of central efficacy with peripheral siRNA delivery in Cynomolgus monkey (Macaca fascicularis) using a DAR2 average human TfR binding proteins-SNCA siRNA conjugates, a 1-month efficacy with DAR1 average human TfR binding proteins-SNCA siRNA conjugates was conducted to determine difference in efficacy. Pharmacodynamic properties of human TfR binding protein-siRNA conjugates were assessed in NHPs according to the following. Cynomolgus monkeys weighing 2-3 kg were dosed one intravenously in the Saphenous vein in the thigh with i) PBS (n=4), ii) TBP16-SNCA siRNA (dsRNA No. 10 in Table 11a) conjugate (DAR1) (N=4) at 1 mg/kg effective siRNA concentration, or iii) TBP15-SNCA siRNA (dsRNA No. 10 in Table 11a) conjugate (DAR1) at 1 mg/kg or 10 mg/kg (N=4 each) effective siRNA and sacrificed 29 days after the first dose. For takedowns, deeply anesthetized animals underwent cardiac perfusion, then brain tissues were collected and processed for RT-qPCR in tissue homogenates.

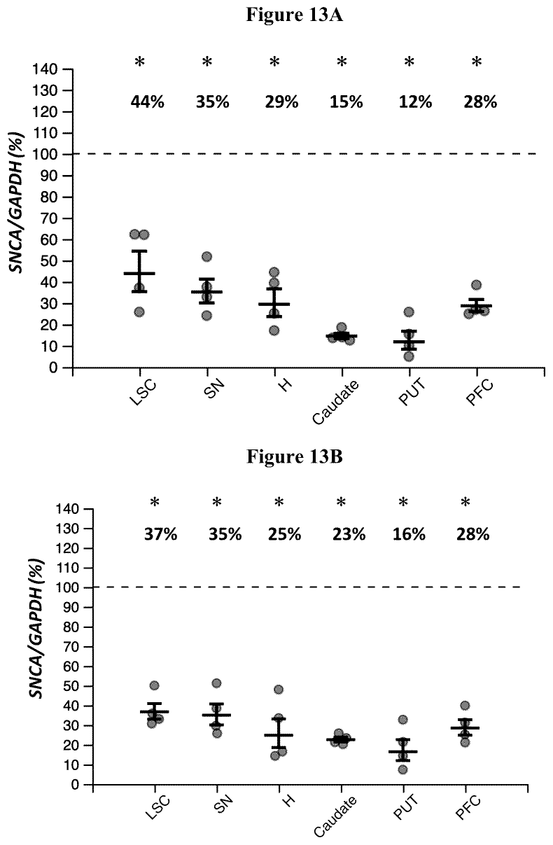

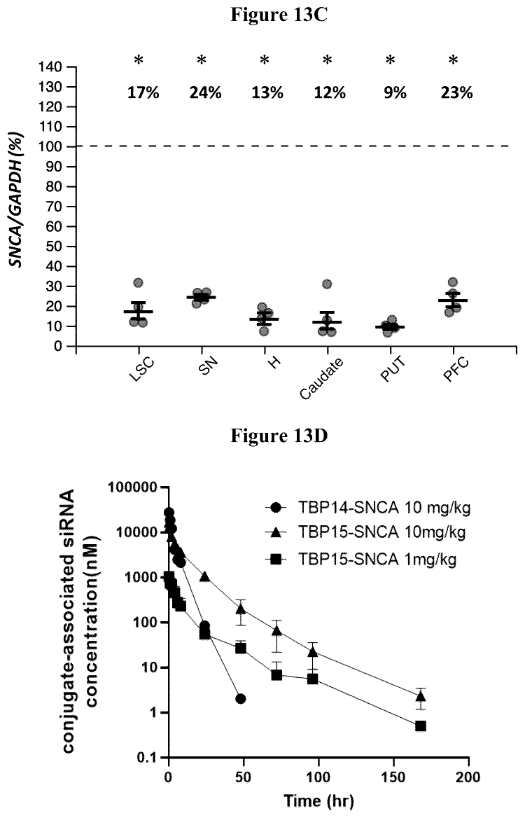

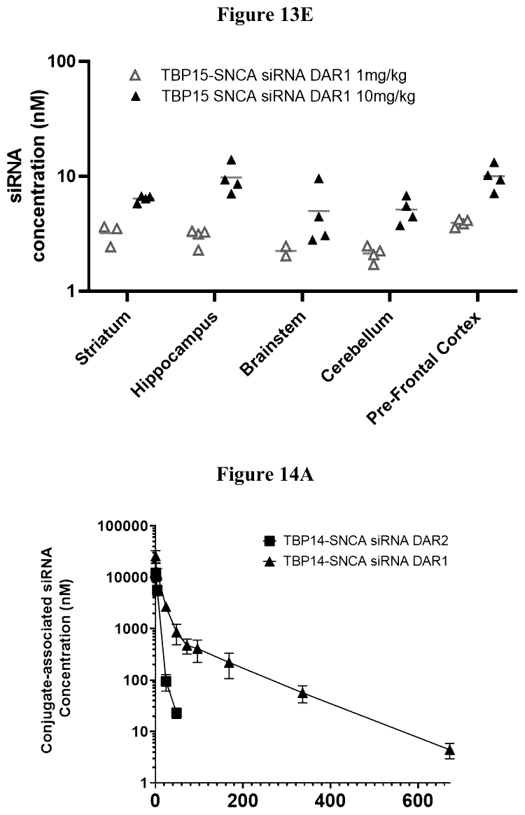

RT-qPCR data showed robust reduction of SNCA mRNA ranging from 60-80% in all key brain regions at 1 mg/kg siRNA dose demonstrating high efficacy of the DAR1 conjugate (FIGS. 13A and 13B). Increasing the dose tenfold to 10 mg/kg only elicited additional 5-10% reduction in mRNA from 1 mg/kg suggesting that TfR-mediated drug delivery is already saturated (FIG. 13C).

### 8E. Exposure Response Relationships

To understand exposure response relationships for the studies described in Examples 8B and 8D above, plasma pharmacokinetics (PK) and biodistribution of siRNA following a single IV dose, plasma samples from the above mentioned study were collected and the exposure of the conjugate associated siRNA in plasma or the total siRNA in tissue was quantified by HR-LC/MS (FIGS. 13D and 13E). Briefly, Liquid chromatography/mass spectrometry (LC/MS) was used to measure conjugate associated or total siRNA levels in Cynomolgus plasma and tissue samples. Plasma standards were prepared by adding in control monkey plasma. Tissue standards were prepared in control tissue homogenate. To control assay variability, an internal standard was added to all standards and samples.

For conjugate associated siRNA, plasma standards and samples were incubated with a biotinylated polyclonal Goat Anti-Human IgG antibody (Southern Biotech, Birmingham, AL) followed by a second incubation with streptavidin beads (Promega, Madison, WI). The IgG-siRNA-streptavidin bead complex was isolated on a magnetic separator and the supernatant was discarded. Samples and standards were washed with phosphate buffered saline solution followed by conjugate-associated siRNA elution from the beads with triethylamine. The standards and samples were injected onto an LC/MS system.

Tissue samples were homogenized in cell lysis buffer. For total siRNA measurements, tissue standards and samples were digested with proteinase K prior to being loaded onto an Oasis Wax micro-elution solid phase extraction (SPE) plate (Waters Inc, Milford, MA) for isolation. The SPE plate was washed with wash buffers and then analytes were eluted with elution buffer. Eluants from the SPE plates were dried, reconstituted, and injected onto an LC/MS system.

The conjugate associated siRNA or total siRNA were measured using a Thermo Orbitrap Exploris 240 (Thermo Scientific, San Jose, CA) mass spectrometer using the antisense strand peak for quantification. The mass spectrometer was operated in negative ion detection mode. All data were processed using Xcalibur version 4.4 (Thermo Scientific, San Jose, CA).

For TBP15-SNCA conjugate (DAR1), based on the AUC(0-168 hr), the Plasma PK appears to be greater than dose proportional (7.8 μM*hr vs 111.6 μM*hr) between the 1 and 10 mg/kg siRNA doses (FIG. 13D). This plasma PK is in agreement with TMDD-mediated clearance. For the DAR2 conjugate at 10 mg/kg siRNA, TBP14-SNCA siRNA (DAR2), the AUC(0-72 hr) was 78 μM*hr, a roughly 1.8 folder lower exposure than observed for TBP15-SNCA siRNA (DAR1) at the same dose.

For TBP15-SNCA siRNA (DAR1), the dose dependent plasma PK translates to brain distribution, albeit with an even less dose proportional profile than the plasma exposure (FIG. 13E). For a given dose, the exposure across different brain regions was similar. Brain exposure for TBP14-SNCA (DAR2) at 3 months was undetectable in agreement with the lower plasma exposure (data not shown).

### Example 9. Further Characterization of the Human TfR Binding Proteins-dsRNA Conjugates in Human TfR (hTfR) Transgenic Mice

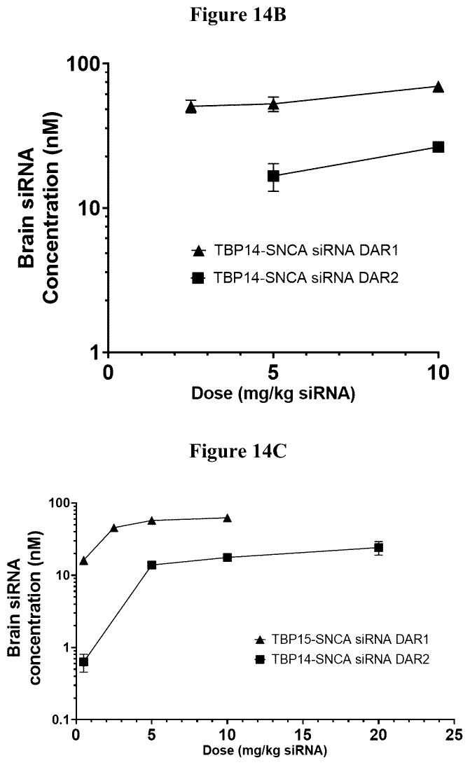

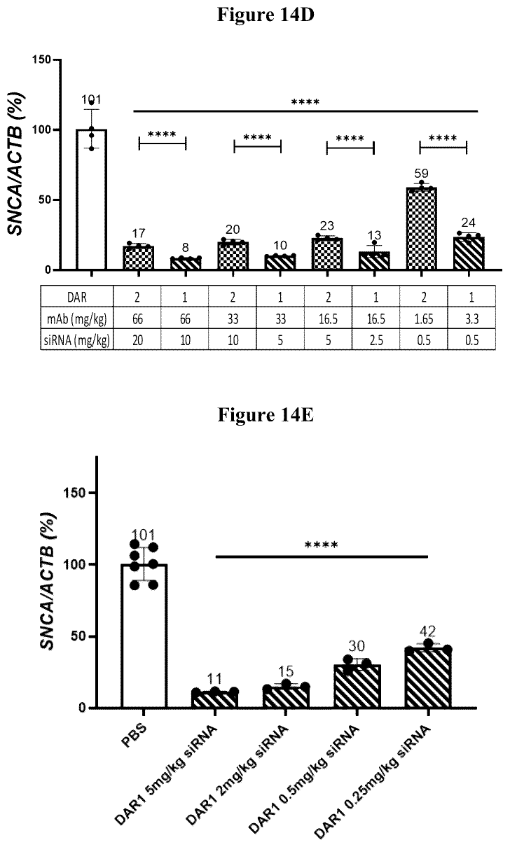

To understand the impact of DAR on the plasma PK and biodistribution of siRNA following a single IV dose of the human TfR binding proteins-dsRNA conjugates, TBP14-SNCA siRNA conjugate (DAR1) and TBP14-SNCA siRNA conjugate (DAR2) were dosed in hTfR transgenic mice at 10 mg/kg and plasma samples were collected and the exposure of the conjugate-associated siRNA was quantified by HR-LC/MS at various times post dose through 1 month (FIG. 14A). Based on the AUC(0-168 hr), the Plasma PK for DAR1 is 3.5 fold greater than DAR2 (323 μM*hr vs 92 μM*hr). This plasma PK agrees with TMDD-mediated clearance. This dose dependent plasma PK translates to brain where exposures up to 3.6 fold higher are observed for DAR1 vs DAR2 (FIG. 14B).

FIG. 14C shows brain tissue concentrations of total siRNA in human TfR transgenic mice at 24 hours following a single peripheral IV administration of either TBP14-SNCA siRNA (dsRNA No. 10 in Table 11a) conjugate (DAR2) or TBP15-SNCA siRNA (dsRNA No. 10 in Table 11a) conjugate (DAR1) across varying siRNA doses.

The pharmacodynamic efficacy relationships of DAR1 and DAR2 of the human TfR binding proteins-dsRNA conjugates were evaluated at various doses at matching antibody and siRNA concentrations to determine the dose lowering impact. TBP15-SNCA siRNA conjugate (DAR1) and TBP14-SNCA siRNA conjugate (DAR2) were dosed in hTfR transgenic mice by a single IV injection at 20, 10, 5, 2.5 and 0.5 mg/kg of siRNA compared to PBS dosed group (n=4 each) as indicated in FIG. 14D. For takedowns, deeply anesthetized animals underwent cardiac perfusion 28 days following IV dosing, then brain tissues were collected and processed for RT-qPCR in tissue homogenates. As shown in FIG. 14D, TBP15-SNCA siRNA conjugate (DAR1) demonstrated higher efficacy of SNCA mRNA KD at all matching dose levels compared to TBP14-SNCA siRNA conjugate (DAR2). Specifically, for TBP15-SNCA siRNA conjugate (DAR1), 10 mg/kg siRNA dose elicited 8% mRNA remaining, 5 mg/kg siRNA dose elicited 10% mRNA remaining, 2.5 mg/kg siRNA dose elicited 13% mRNA remaining and 0.5 mg/kg siRNA dose elicited 24% mRNA remaining. For TBP14-SNCA siRNA conjugate (DAR2), 20 mg/kg siRNA dose elicited 17% mRNA remaining, 10 mg/kg siRNA dose elicited 20% mRNA remaining, 5 mg/kg siRNA dose elicited 23% mRNA remaining and 0.5 mg/kg siRNA dose elicited 59% mRNA remaining. In particular, 10-fold siRNA drug dose lowering efficacy was demonstrated when comparing similar mRNA reductions at 5 mg/kg of TBP14-SNCA siRNA conjugate (DAR2) (23% remaining) compared to TBP15-SNCA siRNA conjugate (DAR1) at 0.5 mg/kg (24% remaining). This trend was observed also at a higher dose when comparing similar mRNA reductions at 20 mg/kg of TBP14-SNCA siRNA conjugate (DAR2) (17% remaining) compared to TBP15-SNCA siRNA conjugate (DAR1) at 2.5 mg/kg (13% remaining).

Having demonstrated high potency of DAR1 of the human TfR binding proteins-dsRNA conjugates by intravenous route of administration, the efficacy of TBP15-SNCA siRNA conjugate (DAR1) delivered by a single subcutaneous (SC) administration at 5, 2, 0.5 and 0.25 mg/kg siRNA doses were evaluated. For takedowns, deeply anesthetized animals underwent cardiac perfusion 28 days following SC dosing, then brain tissues were collected and processed for RT-qPCR in tissue homogenates. Data indicated similarly high efficacy of SC delivery at all doses evaluated, demonstrating 11% mRNA remaining at 5 mg/kg dose, 15% remaining at 2 mg/kg dose, 30% mRNA remaining at 0.5 mg/kg dose and 42% mRNA remaining at 0.25 mg/kg dose (FIG. 14E).

SEQUENCE LISTING

| SEQ | |
|---|---|
| ID | |
| NO | Sequence |
| 1 | SYSMN |
| 2 | SISRSSSYIYYADSVKG |
| 3 | EHGYSNSDAFDI |
| 4 | RASQGISNYLA |
| 5 | AASSLQS |
| 6 | LQHNSYPRT |
| 7 | IHGYSNSDAFDK |
| 8 | IHGYSNSDAFDI |
| 9 | RASQGISHYLV |
| 10 | SISSSSSYIYYADSVKG |
| 11 | RHGYSNSDAFDN |
| 12 | LOHNSYPWT |
| 13 | TYWMH |
| 14 | RINGDGSRTNYADSVKG |
| 15 | SSYAFDV |
| 16 | RSSQSLLDSDDGSTYLD |
| 17 | LLSNRAS |
| 18 | MQRIEFPLT |
| 19 | RINSDGSRTNYADSVKG |
| 20 | SSYAFHV |
| 21 | SISXₐₐ₁SSSYIYYADSVKG, wherein Xₐₐ₁ = R or S |
| 22 | Xₐₐ₁HGYSNSDAFD Xₐₐ₂, |
| | wherein Xₐₐ₁ = E, I or R; Xₐₐ₂ = I, K, or N |
| 23 | RASQGIS Xₐₐ₁ YL Xₐₐ₂, wherein Xₐₐ₁ = N or H; Xₐₐ₂ = A or V |
| 24 | LQHNSYP Xₐₐ₁T, wherein Xₐₐ₁ = R or W |
| 25 | RINXₐₐ₁DGSRTNYADSVKG, wherein Xₐₐ₁ = G or S |
| 26 | SSYAF Xₐₐ₁V, wherein Xₐₐ₁ = D or H |
| 27 | EVQLVESGGGLVKPGGSLRLSCVASGFTFSSYSMNWVRQAPGKGLEWVSSISRSSSYIY |
| | YADSVKGRFTISRDNAKNSLYLQMNSLRAEDTAVYYCAREHGYSNSDAFDIWGQGTLVT |
| | VSS |
| 28 | DIQMTQSPSAMSASVGDRVTITCRASQGISNYLAWFQQKPGKVPKRLIYAASSLQSGVP |
| | SRFSGSGSGTEFTLTISSLQPEDFATYYCLQHNSYPRTFGQGTKVEIK |
| 29 | EVQLVESGGGLVKPGGSLRLSCVASGFTFSSYSMNWVRQAPGKGLEWVSSISRSSSYIY |
| | YADSVKGRFTISRDNAKNSLYLQMNSLRAEDTAVYYCARIHGYSNSDAFDKWGQGTLVT |
| | VSS |
| 30 | EVQLVESGGGLVKPGGSLRLSCVASGFTFSSYSMNWVRQAPGKGLEWVSSISRSSSYIY |
| | YADSVKGRFTISRDNAKNSLYLQMNSLRAEDTAVYYCARIHGYSNSDAFDIWGQGTLVT |
| | VSS |
| 31 | DIQMTQSPSAMSASVGDRVTITCRASQGISHYLVWFQQKPGKVPKRLIYAASSLQSGVP |
| | SRFSGSGSGTEFTLTISSLQPEDFATYYCLQHNSYPRTFGQGTKVEIK |
| 32 | EVQLVESGGGLVKPGGSLRLSCVASGFTFSSYSMNWVRQAPGKGLEWVSSISSSSSYIY |
| | YADSVKGRFTISRDNAKNSLYLQMNSLRAEDTAVYYCARRHGYSNSDAFDNWGQGTLVT |
| | VSS |
| 33 | DIQMTQSPSAMSASVGDRVTITCRASQGISHYLVWFQQKPGKVPKRLIYAASSLQSGVP |
| | SRFSGSGSGTEFTLTISSLQPEDFATYYCLQHNSYPWTFGQGTKVEIK |
| 34 | EVQLVESGGGLVQPGGSLRLSCAASGFTFRTYWMHWVRQAPGKGLLWVSRINGDGSRTN |
| | YADSVKGRFTISRDNAKKTLYLQMNSLRAEDTAVYFCARSSYAFDVWGQGTMVTVSS |
| 35 | DVVMTQTPLSLPVTPGEPASISCRSSQSLLDSDDGSTYLDWYLQKPGQSPQLLIYLLSN |
| | RASGVPDRFSGSGSGTVFTLKISSVEAADVGVYYCMQRIEFPLTFGGGTKVEIK |
| 36 | EVQLVESGGGLVQPGGSLRLSCAASGFTFRTYWMHWVRQAPGKGLVWVSRINSDGSRTN |
| | YADSVKGRFTISRDNAKNTLYLQMNSLRAEDTAVYYCARSSYAFDVWGQGTLVTVSS |
| 37 | DIVMTQTPLSLPVTPGEPASISCRSSQSLLDSDDGSTYLDWYLQKPGQSPQLLIYLLSN |
| | RASGVPDRFSGSGSGTDFTLKISRVEAEDVGVYYCMQRIEFPLTFGGGTKVEIK |
| 38 | EVQLVESGGGLVQPGGSLRLSCAASGFTFRTYWMHWVRQAPGKGLVWVSRINSDGSRTN |
| | YADSVKGRFTISRDNAKNTLYLQMNSLRAEDTAVYYCARSSYAFHVWGQGTLVTVSS |
| 39 | ETAVA |
| 40 | GIGGGVDITYYADSVKG |
| 41 | RPGRPLITSKVADLYPY |
| 42 | EVQLLESGGGLVQPGGSLRLSCAASGRYIDETAVAWFRQAPGKGREFVAGIGGGVDITY |
| | YADSVKGRFTISRDNSKNTLYLQMNSLRPEDTAVYYCGARPGRPLITSKVADLYPYWGQ |
| | GTLVTVSSPP |
| 43 | SYAIE |
| 44 | GILPGSGTINYNEKFKG |
| 45 | MSSNSDQGFDL |
| 46 | KASQGISRFLS |
| 47 | AVSSLVD |
| 48 | VQYNSYPYG |
| 49 | QVQLVQSGAEVKKPGSSVKVSCKASGYTFSSYAIEWVRQAPGQGLEWMGGILPGSGTIN |
| | YNEKFKGRVTITADKSTSTAYMELSSLRSEDTAVYYCARMSSNSDQGFDLWGQGTLVTV |
| | SS |
| 50 | DIQMTQSPSSLSASVGDRVTITCKASQGISRFLSWFQQKPGKAPKSLIYAVSSLVDGVP |
| | SRFSGSGSGTDFTLTISSLQPEDFATYYCVQYNSYPYGFGGGTKVEIK |
| 51 | QVQLVQSGAEVKKPGSSVKVSCKASGYTFSSYAIEWVRQAPGQGLEWMGGILPGSGTIN |
| | YNEKFKGRVTITADKSTSTAYMELSSLRSEDTAVYYCARMSSNSDQGFDLWGQGTLVTV |
| | SSASTKGPXVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEAAG |
| | GPSVFLFPPKPKDTLMISRTPEVTCVVVDVSQEDPEVQFNWYVDGVEVHNAKTKPREEQ |
| | FNSTYRVVSVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFLLYSKLTVD |
| | KSRWQEGNVFSCSVMHEALHNHYTQKSLSLSLG, wherein X is S or C. |
| 52 | DIQMTQSPSSLSASVGDRVTITCKASQGISRFLSWFQQKPGKAPKSLIYAVSSLVDGVP |
| | SRFSGSGSGTDFTLTISSLQPEDFATYYCVQYNSYPYGFGGGTKVEIKRTVAAPSVFIF |
| | PPSDEQLKSGTASVVCLLNNFYPREAKVQWKVDNALQSGNSQESVTEQDSKDSTYSLSS |
| | TLTLSKADYEKHKVYACEVTHQGLSSPVTKSFNRGEC |
| 53 | EVQLVESGGGLVKPGGSLRLSCVASGFTFSSYSMNWVRQAPGKGLEWVSSISRSSSYIY |
| | YADSVKGRFTISRDNAKNSLYLQMNSLRAEDTAVYYCAREHGYSNSDAFDIWGQGTLVT |
| | VSSASTKGPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEAA |
| | GGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVYTLPP |
| | SQEEMTKNQVSLTCLVKGFYPSDIAVEWESNGQPENNYKTTPPVLDSDGSFFLYSRLTV |
| | DKSRWQEGNVFSCSVMHEALHNHYTQKSLSLSLG |
| 54 | DIQMTQSPSAMSASVGDRVTITCRASQGISNYLAWFQQKPGKVPKRLIYAASSLQSGVP |
| | SRFSGSGSGTEFTLTISSLQPEDFATYYCLQHNSYPRTFGQGTKVEIKRTVAAPSVFIF |
| | PPSDEQLKSGTASVVCLLNNFYPREAKVQWKVDNALQSGNSQESVTEQDSKDSTYSLSS |
| | TLTLSKADYEKHKVYACEVTHQGLSSPVTKSFNRGEC |
| 55 | EVQLVESGGGLVKPGGSLRLSCVASGFTFSSYSMNWVRQAPGKGLEWVSSISRSSSYIY |
| | YADSVKGRFTISRDNAKNSLYLQMNSLRAEDTAVYYCARIHGYSNSDAFDKWGQGTLVT |
| | VSSASTKGPXVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEAA |
| | GGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVYTLPP |
| | SQEEMTKNQVSLTCLVKGFYPSDIAVEWESNGQPENNYKTTPPVLDSDGSFFLYSRLTV |
| | DKSRWQEGNVFSCSVMHEALHNHYTQKSLSLSLG, wherein X is S or C. |
| 56 | EVQLVESGGGLVKPGGSLRLSCVASGFTFSSYSMNWVRQAPGKGLEWVSSISRSSSYIY |
| | YADSVKGRFTISRDNAKNSLYLQMNSLRAEDTAVYYCARIHGYSNSDAFDIWGQGTLVT |
| | VSSASTKGPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEAA |
| | GGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVYTLPP |
| | SQEEMTKNQVSLTCLVKGFYPSDIAVEWESNGQPENNYKTTPPVLDSDGSFFLYSRLTV |
| | DKSRWQEGNVFSCSVMHEALHNHYTQKSLSLSLG |
| 57 | DIQMTQSPSAMSASVGDRVTITCRASQGISHYLVWFQQKPGKVPKRLIYAASSLQSGVP |
| | SRFSGSGSGTEFTLTISSLQPEDFATYYCLQHNSYPRTFGQGTKVEIKRTVAAPSVFIF |
| | PPSDEQLKSGTASVVCLLNNFYPREAKVQWKVDNALQSGNSQESVTEQDSKDSTYSLSS |
| | TLTLSKADYEKHKVYACEVTHQGLSSPVTKSFNRGEC |
| 58 | EVQLVESGGGLVKPGGSLRLSCVASGFTFSSYSMNWVRQAPGKGLEWVSSISSSSSYIY |
| | YADSVKGRFTISRDNAKNSLYLQMNSLRAEDTAVYYCARRHGYSNSDAFDNWGQGTLVT |
| | VSSASTKGPXVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEAA |
| | GGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVYTLPP |
| | SQEEMTKNQVSLTCLVKGFYPSDIAVEWESNGQPENNYKTTPPVLDSDGSFFLYSRLTV |
| | DKSRWQEGNVFSCSVMHEALHNHYTQKSLSLSLG, wherein X is S or C. |
| 59 | DIQMTQSPSAMSASVGDRVTITCRASQGISHYLVWFQQKPGKVPKRLIYAASSLQSGVP |
| | SRFSGSGSGTEFTLTISSLQPEDFATYYCLQHNSYPWTFGQGTKVEIKRTVAAPSVFIF |
| | PPSDEQLKSGTASVVCLLNNFYPREAKVQWKVDNALQSGNSQESVTEQDSKDSTYSLSS |
| | TLTLSKADYEKHKVYACEVTHQGLSSPVTKSFNRGEC |
| 60 | EVQLVESGGGLVQPGGSLRLSCAASGFTFRTYWMHWVRQAPGKGLLWVSRINGDGSRTN |
| | YADSVKGRFTISRDNAKKTLYLQMNSLRAEDTAVYFCARSSYAFDVWGQGTMVTVSSAS |
| | TKGPSVFPLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPPCPAPEAAGGPSV |
| | FLFPPKPKDTLMISRTPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRW |
| | QEGNVFSCSVMHEALHNHYTQKSLSLSLG |
| 61 | DVVMTQTPLSLPVTPGEPASISCRSSQSLLDSDDGSTYLDWYLQKPGQSPQLLIYLLSN |
| | RASGVPDRFSGSGSGTVFTLKISSVEAADVGVYYCMQRIEFPLTFGGGTKVEIKRTVAA |
| | PSVFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC |
| 62 | EVQLVESGGGLVQPGGSLRLSCAASGFTFRTYWMHWVRQAPGKGLVWVSRINSDGSRTN |
| | YADSVKGRFTISRDNAKNTLYLQMNSLRAEDTAVYYCARSSYAFDVWGQGTLVTVSSAS |
| | TKGPSVFPLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPPCPAPEAAGGPSV |
| | FLFPPKPKDTLMISRTPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRW |
| | QEGNVFSCSVMHEALHNHYTQKSLSLSLG |
| 63 | DIVMTQTPLSLPVTPGEPASISCRSSQSLLDSDDGSTYLDWYLQKPGQSPQLLIYLLSN |
| | RASGVPDRFSGSGSGTDFTLKISRVEAEDVGVYYCMQRIEFPLTFGGGTKVEIKRTVAA |
| | PSVFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC |
| 64 | EVQLVESGGGLVQPGGSLRLSCAASGFTFRTYWMHWVRQAPGKGLVWVSRINSDGSRTN |
| | YADSVKGRFTISRDNAKNTLYLQMNSLRAEDTAVYYCARSSYAFHVWGQGTLVTVSSAS |
| | TKGPXVFPLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPPCPAPEAAGGPSV |
| | FLFPPKPKDTLMISRTPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRW |
| | QEGNVFSCSVMHEALHNHYTQKSLSLSLG, wherein X is S or C. |
| 65 | EVQLVESGGGLVKPGGSLRLSCVASGFTFSSYSMNWVRQAPGKGLEWVSSISSSSSYIY |
| | YADSVKGRFTISRDNAKNSLYLQMNSLRAEDTAVYYCARRHGYSNSDAFDNWGQGTLVT |
| | VSSASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKRVEPKC |
| 66 | EVQLVESGGGLVKPGGSLRLSCVASGFTFSSYSMNWVRQAPGKGLEWVSSISSSSSYIY |
| | YADSVKGRFTISRDNAKNSLYLQMNSLRAEDTAVYYCARRHGYSNSDAFDNWGQGTLVT |
| | VSSASTKGPCVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKRVEPKCDKTHTGGGGQGGG |
| | GQGGGGQGGGGQGGGGQEVQLLESGGGLVQPGGSLRLSCAASGRYIDETAVAWFRQAPG |
| | KGREFVAGIGGGVDITYYADSVKGRFTISRDNSKNTLYLQMNSLRPEDTAVYYCGARPG |
| | RPLITSKVADLYPYWGQGTLVTVSSPP |
| 67 | DIQMTQSPSAMSASVGDRVTITCRASQGISHYLVWFQQKPGKVPKRLIYAASSLQSGVP |
| | SRFSGSGSGTEFTLTISSLQPEDFATYYCLQHNSYPWTFGQGTKVEIKRTVAAPSVFIF |
| | PPSDEQLKSGTASVVCLLNNFYPREAKVQWKVDNALQCGNSQESVTEQDSKDSTYSLSS |
| | TLTLSKADYEKHKVYACEVTHQGLSSPVTKSFNRGEC |
| 68 | EVQLVESGGGLVKPGGSLRLSCVASGFTFSSYSMNWVRQAPGKGLEWVSSISSSSSYIY |
| | YADSVKGRFTISRDNAKNSLYLQMNSLRAEDTAVYYCARRHGYSNSDAFDNWGQGTLVT |
| | VSSASTKGPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEAA |
| | GGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVSTLPP |
| | SQEEMTKNQVSLMCLVYGFYPSDICVEWESNGQPENNYKTTPPVLDSDGSFFLYSVLTV |
| | DKSRWQEGNVFSCSVMHEALHNHYTQKSLSLSLG |
| 69 | ESKYGPPCPPCPAPEAAGGPSVFLFPPKPKDTLMISRTPEVTCVVVDVSQEDPEVQFNW |
| | YVDGVEVHNAKTKPREEQFNSTYRVVSVLTVLHQDWLNGKEYKCKVSNKGLPSSIEKTI |
| | SKAKGQPREPQVYTLPPSQGDMTKNQVQLTCLVKGFYPSDICVEWESNGQPENNYKTTP |
| | PVLDSDGSFFLASRLTVDKSRWQEGNVFSCSVMHEALHNHYTQKSLSLSLG |
| 70 | GGGGQGGGGQGGGGQGGGGQ |
| 71 | GSYWIC |
| 72 | CIYSTSGGRTYYASWVKG |
| 73 | GDDSISDAYFDL |
| 74 | QSSQSVYNNNRLA |
| 75 | DASTLAS |
| 76 | QGTYFSSGWSWA |
| 77 | QSLEESGGDLVKPEGSLTLTCTASGFSFSGSYWICWVRQAPGKGLEWIGCIYSTSGGRT |
| | YYASWVKGRFTISKTSSTTVTLQMTSLTAADTATYFCARGDDSISDAYFDLWGPGTLVT |
| | VSS |
| 78 | ALDMTQTASPVSAAVGGTVTINCQSSQSVYNNNRLAWYQQKPGQPPKLLIYDASTLASG |
| | VPSRFKGSGSGTQFTLTISGVQSDDSATYYCQGTYFSSGWSWAFGGGTEVVVK |
| 79 | QSLEESGGDLVKPEGSLTLTCTASGFSFSGSYWICWVRQAPGKGLEWIGCIYSTSGGRT |
| | YYASWVKGRFTISKTSSTTVTLQMTSLTAADTATYFCARGDDSISDAYFDLWGPGTLVT |
| | VSSASTKGPCVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEAA |
| | GGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVYTLPP |
| | SQEEMTKNQVSLTCLVKGFYPSDIAVEWESNGQPENNYKTTPPVLDSDGSFFLYSRLTV |
| | DKSRWQEGNVFSCSVMHEALHNHYTQKSLSLSLG |
| 80 | ALDMTQTASPVSAAVGGTVTINCQSSQSVYNNNRLAWYQQKPGQPPKLLIYDASTLASG |
| | VPSRFKGSGSGTQFTLTISGVQSDDSATYYCQGTYFSSGWSWAFGGGTEVVVKRTVAAP |
| | SVFIFPPSDEQLKSGTASVVCLLNNFYPREAKVQWKVDNALQSGNSQESVTEQDSKDST |
| | YSLSSTLTLSKADYEKHKVYACEVTHQGLSSPVTKSFNRGEC |
| 81 | CUGUACAAGUGCUCAGUUCCA |
| 82 | UGGAACUGAGCACUUGUACAGGA |
| 83 | UGUACAAGUGCUCAGUUCCAA |
| 84 | UUGGAACUGAGCACUUGUACAGG |
| 85 | GAGCAAGUGACAAAUGUUGGA |
| 86 | UCCAACAUUUGUCACUUGCUCUU |
| 87 | UUCCAAUGUGCCCAGUCAUGA |
| 88 | UCAUGACUGGGCACAUUGGAACU |
| 89 | AGUGACUACCACUUAUUUCUA |
| 90 | UAGAAAUAAGUGGUAGUCACUUA |
| 91 | GUGACUACCACUUAUUUCUAA |
| 92 | UUAGAAAUAAGUGGUAGUCACUU |
| 93 | mC*mU*mGmUmAmCfAmAfGfUfGmCmUmCmAmGmUmUmC*mC*mA*(C6 amino) |
| 94 | [VPmU]*fG*mGmAmAfCmUmGmAmGmCmAmCfUmUfGmUmAmCmAmG*mG*mA |
| 95 | mC*mU*mGmUmAmCmAmAfGfUfGmCmUmCmAmGmUmUmC*mC*mA*(C6 amino) |
| 96 | [VPmU]*fG*mGmAfAmCfUmGmAmGmCmAmCfUmUfGmUmAmCmAmG*mG*mA |
| 97 | [VPmU]*fG*mGmAfAmCmUfGmAmGmCmAmCfUmUfGmUmAmCmAmG*mG*mA |
| 98 | [VPmU]*fG*fGmAmAmCfUmGmAmGmCmAmCfUmUfGmUmAmCmAmG*mG*mA |
| 99 | iAbmC*mU*mGmUmAmCfAmAfGfUfGmCmUmCmAmGmUmUmC*mC*mA*(C6 amino) |
| 100 | mU*mG*mUmAmCmAfAmGfUfGfCmUmCmAmGmUmUmCmC*mA*mA*(C6 amino) |
| 101 | [VPmU]*fU*mGmGmAfAmCmUmGmAmGmCmAfCmUfUmGmUmAmCmA*mG*mG |
| 102 | mG*mU*mGmAmCmUfAmCfCfAfCmUmUmAmUmUmUmCmU*mA*mA*(C6 amino) |
| 103 | [VPmU]*fU*mAmGmAfAmAmUmAmAmGmUmGfGmUfAmGmUmCmAmC*mU*mU |
| 104 | mG*mA*mGmCmAmAfGmUfGfAfCmAmAmAmUmGmUmUmG*mG*mA*(C6 amino) |
| 105 | [VPmU]*fC*mCmAmAfCmAmUmUmUmGmUmCfAmCfUmUmGmCmUmC*mU*mU |
| 106 | mA*mG*mUmGmAmCfUmAfCfCfAmCmUmUmAmUmUmUmC*mU*mA*(C6 amino) |
| 107 | [VPmU]*fA*mGmAmAfAmUmAmAmGmUmGmGfUmAfGmUmCmAmCmU*mU*mA |
| 108 | iAbmA*mG*mUmGmAmCfUmAfCfCfAmCmUmUmAmUmUmUmC*mU*mA*(C6 amino) |
| 109 | 1 GGCGACGACC AGAAGGGGCC CAAGAGAGGG GGCGAGCGAC CGAGCGCCGC GACGCGGAAG |
| | 61 TGAGGTGCGT GCGGGCTGCA GCGCAGACCC CGGCCCGGCC CCTCCGAGAG CGTCCTGGGC |
| | 121 GCTCCCTCAC GCCTTGCCTT CAAGCCTTCT GCCTTTCCAC CCTCGTGAGC GGAGAACTGG |
| | 181 GAGTGGCCAT TCGACGACAG TGTGGTGTAA AGGAATTCAT TAGCCATGGA TGTATTCATG |
| | 241 AAAGGACTTT CAAAGGCCAA GGAGGGAGTT GTGGCTGCTG CTGAGAAAAC CAAACAGGGT |
| | 361 GAGGGAGTGG TGCATGGTGT GGCAACAGTG GCTGAGAAGA CCAAAGAGCA AGTGACAAAT |
| | 301 GTGGCAGAAG CAGCAGGAAA GACAAAAGAG GGTGTTCTCT ATGTAGGCTC CAAAACCAAG |
| | 361 GAGGGAGTGG TGCATGGTGT GGCAACAGTG GCTGAGAAGA CCAAAGAGCA AGTGACAAAT |
| | 421 GTTGGAGGAG CAGTGGTGAC GGGTGTGACA GCAGTAGCCC AGAAGACAGT GGAGGGAGCA |
| | 481 GGGAGCATTG CAGCAGCCAC TGGCTTTGTC AAAAAGGACC AGTTGGGCAA GAATGAAGAA |
| | 541 GGAGCCCCAC AGGAAGGAAT TCTGGAAGAT ATGCCTGTGG ATCCTGACAA TGAGGCTTAT |
| | 601 GAAATGCCTT CTGAGGAAGG GTATCAAGAC TACGAACCTG AAGCCTAAGA AATATCTTTG |
| | 661 CTCCCAGTTT CTTGAGATCT GCTGACAGAT GTTCCATCCT GTACAAGTGC TCAGTTCCAA |
| | 721 TGTGCCCAGT CATGACATTT CTCAAAGTTT TTACAGTGTA TCTCGAAGTC TTCCATCAGC |
| | 781 AGTGATTGAA GTATCTGTAC CTGCCCCCAC TCAGCATTTC GGTGCTTCCC TTTCACTGAA |
| | 841 GTGAATACAT GGTAGCAGGG TCTTTGTGTG CTGTGGATTT TGTGGCTTCA ATCTACGATG |
| | 901 TTAAAACAAA TTAAAAACAC CTAAGTGACT ACCACTTATT TCTAAATCCT CACTATTTTT |
| | 961 TTGTTGCTGT TGTTCAGAAG TTGTTAGTGA TTTGCTATCA TATATTATAA GATTTTTAGG |
| | 1021 TGTCTTTTAA TGATACTGTC TAAGAATAAT GACGTATTGT GAAATTTGTT AATATATATA |
| | 1081 ATACTTAAAA ATATGTGAGC ATGAAACTAT GCACCTATAA ATACTAAATA TGAAATTTTA |
| | 1141 CCATTTTGCG ATGTGTTTTA TTCACTTGTG TTTGTATATA AATGGTGAGA ATTAAAATAA |
| | 1201 AACGTTATCT CATTGCAAAA ATATTTTATT TTTATCCCAT CTCACTTTAA TAATAAAAAT |
| | 1261 CATGCTTATA AGCAACATGA ATTAAGAACT GACACAAAGG ACAAAAATAT AAAGTTATTA |
| | 1321 ATAGCCATTT GAAGAAGGAG GAATTTTAGA AGAGGTAGAG AAAATGGAAC ATTAACCCTA |
| | 1381 CACTCGGAAT TCCCTGAAGC AACACTGCCA GAAGTGTGTT TTGGTATGCA CTGGTTCCTT |
| | 1441 AAGTGGCTGT GATTAATTAT TGAAAGTGGG GTGTTGAAGA CCCCAACTAC TATTGTAGAG |
| | 1501 TGGTCTATTT CTCCCTTCAA TCCTGTCAAT GTTTGCTTTA CGTATTTTGG GGAACTGTTG |
| | 1561 TTTGATGTGT ATGTGTTTAT AATTGTTATA CATTTTTAAT TGAGCCTTTT ATTAACATAT |
| | 1621 ATTGTTATTT TTGTCTCGAA ATAATTTTTT AGTTAAAATC TATTTTGTCT GATATTGGTG |
| | 1681 TGAATGCTGT ACCTTTCTGA CAATAAATAA TATTCGACCA TGAATAAAAA AAAAAAAAAA |
| | 1741 GTGGGTTCCC GGGAACTAAG CAGTGTAGAA GATGATTTTG ACTACACCCT CCTTAGAGAG |
| | 1801 CCATAAGACA CATTAGCACA TATTAGCACA TTCAAGGCTC TGAGAGAATG TGGTTAACTT |
| | 1861 TGTTTAACTC AGCATTCCTC ACTTTTTTTT TTTAATCATC AGAAATTCTC TCTCTCTCTC |
| | 1921 TCTCTTTTTC TCTCGCTCTC TTTTTTTTTT TTTTTTTACA GGAAATGCCT TTAAACATCG |
| | 1981 TTGGAACTAC CAGAGTCACC TTAAAGGAGA TCAATTCTCT AGACTGATAA AAATTTCATG |
| | 2041 GCCTCCTTTA AATGTTGCCA AATATATGAA TTCTAGGATT TTTCCTTAGG AAAGGTTTTT |
| | 2101 CTCTTTCAGG GAAGATCTAT TAACTCCCCA TGGGTGCTGA AAATAAACTT GATGGTGAAA |
| | 2161 AACTCTGTAT AAATTAATTT AAAAATTATT TGGTTTCTCT TTTTAATTAT TCTGGGGCAT |
| | 2221 AGTCATTTCT AAAAGTCACT AGTAGAAAGT ATAATTTCAA GACAGAATAT TCTAGACATG |
| | 2281 CTAGCAGTTT ATATGTATTC ATGAGTAATG TGATATATAT TGGGCGCTGG TGAGGAAGGA |
| | 2341 AGGAGGAATG AGTGACTATA AGGATGGTTA CCATAGAAAC TTCCTTTTTT ACCTAATTGA |
| | 2401 AGAGAGACTA CTACAGAGTG CTAAGCTGCA TGTGTCATCT TACACTAGAG AGAAATGGTA |
| | 2461 AGTTTCTTGT TTTATTTAAG TTATGTTTAA GCAAGGAAAG GATTTGTTAT TGAACAGTAT |
| | 2521 ATTTCAGGAA GGTTAGAAAG TGGCGGTTAG GATATATTTT AAATCTACCT AAAGCAGCAT |
| | 2581 ATTTTAAAAA TTTAAAAGTA TTGGTATTAA ATTAAGAAAT AGAGGACAGA ACTAGACTGA |
| | 2641 TAGCAGTGAC CTAGAACAAT TTGAGATTAG GAAAGTTGTG ACCATGAATT TAAGGATTTA |
| | 2701 TGTGGATACA AATTCTCCTT TAAAGTGTTT CTTCCCTTAA TATTTATCTG ACGGTAATTT |
| | 2761 TTGAGCAGTG AATTACTTTA TATATCTTAA TAGTTTATTT GGGACCAAAC ACTTAAACAA |
| | 2821 AAAGTTCTTT AAGTCATATA AGCCTTTTCA GGAAGCTTGT CTCATATTCA CTCCCGAGAC |
| | 2881 ATTCACCTGC CAAGTGGCCT GAGGATCAAT CCAGTCCTAG GTTTATTTTG CAGACTTACA |
| | 2941 TTCTCCCAAG TTATTCAGCC TCATATGACT CCACGGTCGG CTTTACCAAA ACAGTTCAGA |
| | 3001 GTGCACTTTG GCACACAATT GGGAACAGAA CAATCTAATG TGTGGTTTGG TATTCCAAGT |
| | 3061 GGGGTCTTTT TCAGAATCTC TGCACTAGTG TGAGATGCAA ACATGTTTCC TCATCTTTCT |
| | 3121 GGCTTATCCA GTATGTAGCT ATTTGTGACA TAATAAATAT ATACATATAT GAAAATA |
| 110 | 1 MDVFMKGLSK AKEGVVAAAE KTKQGVAEAA GKTKEGVLYV GSKTKEGVVH GVATVAEKTK |
| | 61 EQVTNVGGAV VTGVTAVAQK TVEGAGSIAA ATGFVKKDQL GKNEEGAPQE GILEDMPVDP |
| | 121 DNEAYEMPSE EGYQDYEPEA |
| 111 | 1 MMDQARSAFS NLFGGEPLSY TRFSLARQVD GDNSHVEMKL AVDEEENADN NTKANVTKPK |
| | 61 RCSGSICYGT IAVIVFFLIG FMIGYLGYCK GVEPKTECER LAGTESPVRE EPGEDFPAAR |
| | 121 RLYWDDLKRK LSEKLDSTDF TGTIKLLNEN SYVPREAGSQ KDENLALYVE NQFREFKLSK |
| | 181 VWRDQHFVKI QVKDSAQNSV IIVDKNGRLV YLVENPGGYV AYSKAATVTG KLVHANFGTK |
| | 241 KDFEDLYTPV NGSIVIVRAG KITFAEKVAN AESLNAIGVL IYMDQTKFPI VNAELSFFGH |
| | 301 AHLGTGDPYT PGFPSFNHTQ FPPSRSSGLP NIPVQTISRA AAEKLFGNME GDCPSDWKTD |
| | 361 STCRMVTSES KNVKLTVSNV LKEIKILNIF GVIKGFVEPD HYVVVGAQRD AWGPGAAKSG |
| | 421 VGTALLLKLA QMFSDMVLKD GFQPSRSIIF ASWSAGDFGS VGATEWLEGY LSSLHLKAFT |
| | 481 YINLDKAVLG TSNFKVSASP LLYTLIEKTM QNVKHPVTGQ FLYQDSNWAS KVEKLTLDNA |
| | 541 AFPFLAYSGI PAVSFCFCED TDYPYLGTTM DTYKELIERI PELNKVARAA AEVAGQFVIK |
| | 601 LTHDVELNLD YERYNSQLLS FVRDLNQYRA DIKEMGLSLQ WLYSARGDFF RATSRLTTDF |
| | 661 GNAEKTDRFV MKKLNDRVMR VEYHFLSPYV SPKESPFRHV FWGSGSHTLP ALLENLKLRK |
| | 721 QNNGAFNETL FRNQLALATW TIQGAANALS GDVWDIDNEF |
| 112 | 1 MMDQARSAFS NLFGGEPLSY TRFSLARQVD GDNSHVEMKL AADEEENADN NMKASVRKPK |
| | 61 RFNGRLCFAA IALVIFFLIG FMSGYLGYCK RVEQKEECVK LAETEETDKS ETMETEDVPT |
| | 121 SSRLYWADLK TLLSEKLNSI EFADTIKQLS QNTYTPREAG SQKDESLAYY IENQFHEFKF |
| | 181 SKVWRDEHYV KIQVKSSIGQ NMVTIVQSNG NLDPVESPEG YVAFSKPTEV SGKLVHANFG |
| | 241 TKKDFEELSY SVNGSLVIVR AGEITFAEKV ANAQSFNAIG VLIYMDKNKF PVVEADLALF |
| | 361 IDSSCKLELS QNQNVKLIVK NVLKERRILN IFGVIKGYEE PDRYVVVGAQ RDALGAGVAA |
| | 301 GHAHLGTGDP YTPGFPSFNH TQFPPSQSSG LPNIPVQTIS RAAAEKLFGK MEGSCPARWN |
| | 361 IDSSCKLELS QNQNVKLIVK NVLKERRILN IFGVIKGYEE PDRYVVVGAQ RDALGAGVAA |
| | 421 KSSVGTGLLL KLAQVFSDMI SKDGFRPSRS IIFASWTAGD FGAVGATEWL EGYLSSLHLK |
| | 481 AFTYINLDKV VLGTSNFKVS ASPLLYTLMG KIMQDVKHPV DGKSLYRDSN WISKVEKLSF |
| | 541 DNAAYPFLAY SGIPAVSFCF CEDADYPYLG TRLDTYEALT QKVPQLNQMV RTAAEVAGQL |
| | 601 IIKLTHDVEL NLDYEMYNSK LLSFMKDLNQ FKTDIRDMGL SLOWLYSARG DYFRATSRLT |
| | 661 TDFHNAEKTN RFVMREINDR IMKVEYHFLS PYVSPRESPF RHIFWGSGSH TLSALVENLK |
| | 721 LRQKNITAFN ETLFRNQLAL ATWTIQGVAN ALSGDIWNID NEF |
| 113 | HHHHHHCKRVEQKEECVKLAETEETDKSETMETEDVPTSSRLYWADLKTLLSEKLNSIE |
| | FADTIKQLSQNTYTPREAGSQKDESLAYYIENQFHEFKFSKVWRDEHYVKIQVKSSIGQ |
| | NMVTIVQSNGNLDPVESPEGYVAFSKPTEVSGKLVHANFGTKKDFEELSYSVNGSLVIV |
| | RAGEITFAEKVANAQSFNAIGVLIYMDKNKFPVVEADLALFGHAHLGTGDPYTPGFPSF |
| | NHTQFPPSQSSGLPNIPVQTISRAAAEKLFGKMEGSCPARWNIDSSCKLELSQNQNVKL |
| | IVKNVLKERRILNIFGVIKGYEEPDRYVVVGAQRDALGAGVAAKSSVGTGLLLKLAQVF |
| | SDMISKDGFRPSRSIIFASWTAGDFGAVGATEWLEGYLSSLHLKAFTYINLDKVVLGTS |
| | NFKVSASPLLYTLMGKIMQDVKHPVDGKSLYRDSNWISKVEKLSFDNAAYPFLAYSGIP |
| | AVSFCFCEDADYPYLGTRLDTYEALTQKVPQLNQMVRTAAEVAGQLIIKLTHDVELNLD |
| | YEMYNSKLLSFMKDLNQFKTDIRDMGLSLOWLYSARGDYFRATSRLTTDFHNAEKTNRF |
| | VMREINDRIMKVEYHFLSPYVSPRESPFRHIFWGSGSHTLSALVENLKLRQKNITAFNE |
| | TLFRNQLALATWTIQGVANALSGDIWNIDNEF |
| 114 | HHHHHHCKGVEPKTECERLAGTESPVREEPGEDFPAARRLYWDDLKRKLSEKLDSTDFT |
| | GTIKLLNENSYVPREAGSQKDENLALYVENQFREFKLSKVWRDQHFVKIQVKDSAQNSV |
| | IIVDKNGRLVYLVENPGGYVAYSKAATVTGKLVHANFGTKKDFEDLYTPVNGSIVIVRA |
| | GKITFAEKVANAESLNAIGVLIYMDQTKFPIVNAELSFFGHAHLGTGDPYTPGFPSFNH |
| | TQFPPSRSSGLPNIPVQTISRAAAEKLFGNMEGDCPSDWKTDSTCRMVTSESKNVKLTV |
| | SNVLKEIKILNIFGVIKGFVEPDHYVVVGAQRDAWGPGAAKSGVGTALLLKLAQMFSDM |
| | VLKDGFQPSRSIIFASWSAGDFGSVGATEWLEGYLSSLHLKAFTYINLDKAVLGTSNFK |
| | VSASPLLYTLIEKTMQNVKHPVTGQFLYQDSNWASKVEKLTLDNAAFPFLAYSGIPAVS |
| | FCFCEDTDYPYLGTTMDTYKELIERIPELNKVARAAAEVAGQFVIKLTHDVELNLDYER |
| | YNSQLLSFVRDLNQYRADIKEMGLSLOWLYSARGDFFRATSRLTTDFGNAEKTDRFVMK |
| | KLNDRVMRVEYHFLSPYVSPKESPFRHVFWGSGSHTLPALLENLKLRKQNNGAFNETLF |
| | RNQLALATWTIQGAANALSGDVWDIDNEF |
| 115 | HHHHHHHHGKPIPNPLLGLDSTGGGGSDSAQNSVIIVDKNGRLVYLVENPGGYVAYSKA |
| | ATVTGKLVHANFGTKKDFEDLYTPVNGSIVIVRAGKITFAEKVANAESLNAIGVLIYMD |
| | QTKFPIVNAELSFFGHAHLGGGGGGLPNIPVQTISRAAAEKLFGNMEGDCPSDWKTDST |
| | CRMVTSESKNVKLTVS |
| 116 | CUGUACAAGnGCUCAGUUCCA, wherein n is an abasic moiety. |
| 117 | mC*mU*mGmUmAmCmAmAfGnfGmCmUmCmAmGmUmUmC*mC*mA*(C6 amino), |
| | wherein n is the abasic moiety in Table 10. |
| 118 | mC*mU*mGmUmAmCfAmAfGnfGmCmUmCmAmGmUmUmC*mC*mA*(C6 amino), |
| | wherein n is the abasic moiety in Table 10. |
| 119 | FGNMEGDCPSDWKTDSTCR |
| 120 | GUGGAAGUAAAAUCUGAGAAA |
| 121 | UUUCUCAGAUUUUACUUCCACCU |
| 122 | CCAAGUGUGGCUCAUUAGGCA |
| 123 | UGCCUAAUGAGCCACACUUGGAG |
| 124 | UGCAAAUAGUCUACAAACCAA |
| 125 | UUGGUUUGUAGACUAUUUGCACC |
| 126 | mG*mU*mGmGmAmAfGmUfAfAfAmAmUmCmUmGmAmGmA*mA*mA* (C6 amino) |
| 127 | [VPmU]*fU*mUmCmUfCmAmGmAmUmUmUmUfAmCfUmUmCmCmAmC*mC*mU |
| 128 | mC*mC*mAmAmGmUfGmUfGfGfCmUmCmAmUmUmAmGmG*mC*mA* (C6 amino) |
| 129 | [VPmU]*fG*mCmCmUfAmAmUmGmAmGmCmCfAmCfAmCmUmUmGmG*mA*mG |
| 130 | mU*mG*mCmAmAmAfUmAfGfUfCmUmAmCmAmAmAmCmC*mA*mA* (C6 amino) |
| 131 | [VPmU]*fU*mGmGmUfUmUmGmUmAmGmAmCfUmAfUmUmUmGmCmA*mC*mC |
| 132 | mG*mU*mGmGmAmAmGmUfAfAfAmAmUmCmUmGmAmGmA*mA*mA*(C6 amino) |
| 133 | [VPmU]*fU*mUmCfUmCmAfGmAmUmUmUmUfAmCfUmUmCmCmAmC*mC*mU |
| 134 | mC*mC*mAmAmGmUmGmUfGfGfCmUmCmAmUmUmAmGmG*mC*mA*(C6 amino) |
| 135 | [VPmU]*fG*mCmCfUmAmAfUmGmAmGmCmCfAmCfAmCmUmUmGmG*mA*mG |
| 136 | mU*mG*mCmAmAmAmUmAfGfUfCmUmAmCmAmAmAmCmC*mA*mA*(C6 amino) |
| 137 | [VPmU]*fU*mGmGfUmUmUfGmUmAmGmAmCfUmAfUmUmUmGmCmA*mC*mC |
| 138 | EVQLVESGGGLVKPGGSLRLSCVASGFTFSSYSMNWVRQAPGKGLEWVSSISSSSSYIY |
| | YADSVKGRFTISRDNAKNSLYLQMNSLRAEDTAVYYCARRHGYSNSDAFDNWGQGTLVT |
| | VSSASTKGPCVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEAA |
| | GGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVSTLPP |
| | SQEEMTKNQVSLMCLVYGFYPSDIAVEWESNGQPENNYKTTPPVLDSDGSFFLYSVLTV |
| | DKSRWQEGNVFSCSVMHEALHNHYTQKSLSLSLG |
| 139 | ESKYGPPCPPCPAPEAAGGPSVFLFPPKPKDTLMISRTPEVTCVVVDVSQEDPEVQFNW |
| | YVDGVEVHNAKTKPREEQFNSTYRVVSVLTVLHQDWLNGKEYKCKVSNKGLPSSIEKTI |
| | SKAKGQPREPQVYTLPPSQGDMTKNQVQLTCLVKGFYPSDIAVEWESNGQPENNYKTTP |
| | PVLDSDGSFFLASRLTVDKSRWQEGNVFSCSVMHEALHNHYTQKSLSLSLG |
| 140 | mC*mU*mGmUmAmCfAmAfGfUfGmCmUmCmAmGmUmUmC*mC*mA |
| 141 | mC*mU*mGmUmAmCmAmAfGfUfGmCmUmCmAmGmUmUmC*mC*mA |
| 142 | iAbmC*mU*mGmUmAmCfAmAfGfUfGmCmUmCmAmGmUmUmC*mC*mA |
| 143 | mU*mG*mUmAmCmAfAmGfUfGfCmUmCmAmGmUmUmCmC*mA*mA |
| 144 | mG*mU*mGmAmCmUfAmCfCfAfCmUmUmAmUmUmUmCmU*mA*mA |
| 145 | mG*mA*mGmCmAmAfGmUfGfAfCmAmAmAmUmGmUmUmG*mG*mA |
| 146 | mA*mG*mUmGmAmCfUmAfCfCfAmCmUmUmAmUmUmUmC*mU*mA |
| 147 | iAbmA*mG*mUmGmAmCfUmAfCfCfAmCmUmUmAmUmUmUmC*mU*mA |
| 148 | mC*mU*mGmUmAmCmAmAfGnfGmCmUmCmAmGmUmUmC*mC*mA |
| 149 | mC*mU*mGmUmAmCfAmAfGnfGmCmUmCmAmGmUmUmC*mC*mA |
| 150 | mG*mU*mGmGmAmAfGmUfAfAfAmAmUmCmUmGmAmGmA*mA*mA |
| 151 | mC*mC*mAmAmGmUfGmUfGfGfCmUmCmAmUmUmAmGmG*mC*mA |
| 152 | mU*mG*mCmAmAmAfUmAfGfUfCmUmAmCmAmAmAmCmC*mA*mA |
| 153 | mG*mU*mGmGmAmAmGmUfAfAfAmAmUmCmUmGmAmGmA*mA*mA |
| 154 | mC*mC*mAmAmGmUmGmUfGfGfCmUmCmAmUmUmAmGmG*mC*mA |
| 155 | mU*mG*mCmAmAmAmUmAfGfUfCmUmAmCmAmAmAmCmC*mA*mA |
| 156 | GCAGTCACCGCCACCCACCAGCTCCGGCACCAACAGCAGCGCCGCTGCCACCGCCCACC |
| | TTCTGCCGCCGCCACCACAGCCACCTTCTCCTCCTCCGCTGTCCTCTCCCGTCCTCGCC |
| | TCTGTCGACTATCAGGTGAACTTTGAACCAGGATGGCTGAGCCCCGCCAGGAGTTCGAA |
| | GTGATGGAAGATCACGCTGGGACGTACGGGTTGGGGGACAGGAAAGATCAGGGGGGCTA |
| | CACCATGCACCAAGACCAAGAGGGTGACACGGACGCTGGCCTGAAAGAATCTCCCCTGC |
| | AGACCCCCACTGAGGACGGATCTGAGGAACCGGGCTCTGAAACCTCTGATGCTAAGAGC |
| | ACTCCAACAGCGGAAGATGTGACAGCACCCTTAGTGGATGAGGGAGCTCCCGGCAAGCA |
| | GGCTGCCGCGCAGCCCCACACGGAGATCCCAGAAGGAACCACAGCTGAAGAAGCAGGCA |
| | TTGGAGACACCCCCAGCCTGGAAGACGAAGCTGCTGGTCACGTGACCCAAGAGCCTGAA |
| | AGTGGTAAGGTGGTCCAGGAAGGCTTCCTCCGAGAGCCAGGCCCCCCAGGTCTGAGCCA |
| | CCAGCTCATGTCCGGCATGCCTGGGGCTCCCCTCCTGCCTGAGGGCCCCAGAGAGGCCA |
| | CACGCCAACCTTCGGGGACAGGACCTGAGGACACAGAGGGCGGCCGCCACGCCCCTGAG |
| | CTGCTCAAGCACCAGCTTCTAGGAGACCTGCACCAGGAGGGGCCGCCGCTGAAGGGGGC |
| | AGGGGGCAAAGAGAGGCCGGGGAGCAAGGAGGAGGTGGATGAAGACCGCGACGTCGATG |
| | AGTCCTCCCCCCAAGACTCCCCTCCCTCCAAGGCCTCCCCAGCCCAAGATGGGCGGCCT |
| | CCCCAGACAGCCGCCAGAGAAGCCACCAGCATCCCAGGCTTCCCAGCGGAGGGTGCCAT |
| | CCCCCTCCCTGTGGATTTCCTCTCCAAAGTTTCCACAGAGATCCCAGCCTCAGAGCCCG |
| | ACGGGCCCAGTGTAGGGCGGGCCAAAGGGCAGGATGCCCCCCTGGAGTTCACGTTTCAC |
| | GTGGAAATCACACCCAACGTGCAGAAGGAGCAGGCGCACTCGGAGGAGCATTTGGGAAG |
| | GGCTGCATTTCCAGGGGCCCCTGGAGAGGGGCCAGAGGCCCGGGGCCCCTCTTTGGGAG |
| | AGGACACAAAAGAGGCTGACCTTCCAGAGCCCTCTGAAAAGCAGCCTGCTGCTGCTCCG |
| | CGGGGGAAGCCCGTCAGCCGGGTCCCTCAACTCAAAGCTCGCATGGTCAGTAAAAGCAA |
| | AGACGGGACTGGAAGCGATGACAAAAAAGCCAAGACATCCACACGTTCCTCTGCTAAAA |
| | CCTTGAAAAATAGGCCTTGCCTTAGCCCCAAACACCCCACTCCTGGTAGCTCAGACCCT |
| | CTGATCCAACCCTCCAGCCCTGCTGTGTGCCCAGAGCCACCTTCCTCTCCTAAATACGT |
| | CTCTTCTGTCACTTCCCGAACTGGCAGTTCTGGAGCAAAGGAGATGAAACTCAAGGGGG |
| | CTGATGGTAAAACGAAGATCGCCACACCGCGGGGAGCAGCCCCTCCAGGCCAGAAGGGC |
| | CAGGCCAACGCCACCAGGATTCCAGCAAAAACCCCGCCCGCTCCAAAGACACCACCCAG |
| | CTCTGCGACTAAGCAAGTCCAGAGAAGACCACCCCCTGCAGGGCCCAGATCTGAGAGAG |
| | GTGAACCTCCAAAATCAGGGGATCGCAGCGGCTACAGCAGCCCCGGCTCCCCAGGCACT |
| | CCCGGCAGCCGCTCCCGCACCCCGTCCCTTCCAACCCCACCCACCCGGGAGCCCAAGAA |
| | GGTGGCAGTGGTCCGTACTCCACCCAAGTCGCCGTCTTCCGCCAAGAGCCGCCTGCAGA |
| | CAGCCCCCGTGCCCATGCCAGACCTGAAGAATGTCAAGTCCAAGATCGGCTCCACTGAG |
| | AACCTGAAGCACCAGCCGGGAGGCGGGAAGGTGCAGATAATTAATAAGAAGCTGGATCT |
| | TAGCAACGTCCAGTCCAAGTGTGGCTCAAAGGATAATATCAAACACGTCCCGGGAGGCG |
| | GCAGTGTGCAAATAGTCTACAAACCAGTTGACCTGAGCAAGGTGACCTCCAAGTGTGGC |
| | TCATTAGGCAACATCCATCATAAACCAGGAGGTGGCCAGGTGGAAGTAAAATCTGAGAA |
| | GCTTGACTTCAAGGACAGAGTCCAGTCGAAGATTGGGTCCCTGGACAATATCACCCACG |
| | TCCCTGGCGGAGGAAATAAAAAGATTGAAACCCACAAGCTGACCTTCCGCGAGAACGCC |
| | AAAGCCAAGACAGACCACGGGGCGGAGATCGTGTACAAGTCGCCAGTGGTGTCTGGGGA |
| | CACGTCTCCACGGCATCTCAGCAATGTCTCCTCCACCGGCAGCATCGACATGGTAGACT |
| | CGCCCCAGCTCGCCACGCTAGCTGACGAGGTGTCTGCCTCCCTGGCCAAGCAGGGTTTG |
| | TGATCAGGCCCCTGGGGCGGTCAATAATTGTGGAGAGGAGAGAATGAGAGAGTGTGGAA |
| | AAAAAAAGAATAATGACCCGGCCCCCGCCCTCTGCCCCCAGCTGCTCCTCGCAGTTCGG |
| | TTAATTGGTTAATCACTTAACCTGCTTTTGTCACTCGGCTTTGGCTCGGGACTTCAAAA |
| | TCAGTGATGGGAGTAAGAGCAAATTTCATCTTTCCAAATTGATGGGTGGGCTAGTAATA |
| | AAATATTTAAAAAAAAACATTCAAAAACATGGCCACATCCAACATTTCCTCAGGCAATT |
| | CCTTTTGATTCTTTTTTCTTCCCCCTCCATGTAGAAGAGGGAGAAGGAGAGGCTCTGAA |
| | AGCTGCTTCTGGGGGATTTCAAGGGACTGGGGGTGCCAACCACCTCTGGCCCTGTTGTG |
| | GGGGTGTCACAGAGGCAGTGGCAGCAACAAAGGATTTGAAACTTGGTGTGTTCGTGGAG |
| | CCACAGGCAGACGATGTCAACCTTGTGTGAGTGTGACGGGGGTTGGGGTGGGGCGGGAG |
| | GCCACGGGGGAGGCCGAGGCAGGGGCTGGGCAGAGGGGAGAGGAAGCACAAGAAGTGGG |
| | AGTGGGAGAGGAAGCCACGTGCTGGAGAGTAGACATCCCCCTCCTTGCCGCTGGGAGAG |
| | CCAAGGCCTATGCCACCTGCAGCGTCTGAGCGGCCGCCTGTCCTTGGTGGCCGGGGGTG |
| | GGGGCCTGCTGTGGGTCAGTGTGCCACCCTCTGCAGGGCAGCCTGTGGGAGAAGGGACA |
| | GCGGGTAAAAAGAGAAGGCAAGCTGGCAGGAGGGTGGCACTTCGTGGATGACCTCCTTA |
| | GAAAAGACTGACCTTGATGTCTTGAGAGCGCTGGCCTCTTCCTCCCTCCCTGCAGGGTA |
| | GGGGGCCTGAGTTGAGGGGCTTCCCTCTGCTCCACAGAAACCCTGTTTTATTGAGTTCT |
| | GAAGGTTGGAACTGCTGCCATGATTTTGGCCACTTTGCAGACCTGGGACTTTAGGGCTA |
| | ACCAGTTCTCTTTGTAAGGACTTGTGCCTCTTGGGAGACGTCCACCCGTTTCCAAGCCT |
| | GGGCCACTGGCATCTCTGGAGTGTGTGGGGGTCTGGGAGGCAGGTCCCGAGCCCCCTGT |
| | CCTTCCCACGGCCACTGCAGTCACCCCGTCTGCGCCGCTGTGCTGTTGTCTGCCGTGAG |
| | AGCCCAATCACTGCCTATACCCCTCATCACACGTCACAATGTCCCGAATTCCCAGCCTC |
| | ACCACCCCTTCTCAGTAATGACCCTGGTTGGTTGCAGGAGGTACCTACTCCATACTGAG |
| | GGTGAAATTAAGGGAAGGCAAAGTCCAGGCACAAGAGTGGGACCCCAGCCTCTCACTCT |
| | CAGTTCCACTCATCCAACTGGGACCCTCACCACGAATCTCATGATCTGATTCGGTTCCC |
| | TGTCTCCTCCTCCCGTCACAGATGTGAGCCAGGGCACTGCTCAGCTGTGACCCTAGGTG |
| | TTTCTGCCTTGTTGACATGGAGAGAGCCCTTTCCCCTGAGAAGGCCTGGCCCCTTCCTG |
| | TGCTGAGCCCACAGCAGCAGGCTGGGTGTCTTGGTTGTCAGTGGTGGCACCAGGATGGA |
| | AGGGCAAGGCACCCAGGGCAGGCCCACAGTCCCGCTGTCCCCCACTTGCACCCTAGCTT |
| | GTAGCTGCCAACCTCCCAGACAGCCCAGCCCGCTGCTCAGCTCCACATGCATAGTATCA |
| | GCCCTCCACACCCGACAAAGGGGAACACACCCCCTTGGAAATGGTTCTTTTCCCCCAGT |
| | CCCAGCTGGAAGCCATGCTGTCTGTTCTGCTGGAGCAGCTGAACATATACATAGATGTT |
| | GCCCTGCCCTCCCCATCTGCACCCTGTTGAGTTGTAGTTGGATTTGTCTGTTTATGCTT |
| | GGATTCACCAGAGTGACTATGATAGTGAAAAGAAAAAAAAAAAAAAAAAAGGACGCATG |
| | TATCTTGAAATGCTTGTAAAGAGGTTTCTAACCCACCCTCACGAGGTGTCTCTCACCCC |
| | CACACTGGGACTCGTGTGGCCTGTGTGGTGCCACCCTGCTGGGGCCTCCCAAGTTTTGA |
| | AAGGCTTTCCTCAGCACCTGGGACCCAACAGAGACCAGCTTCTAGCAGCTAAGGAGGCC |
| | GTTCAGCTGTGACGAAGGCCTGAAGCACAGGATTAGGACTGAAGCGATGATGTCCCCTT |
| | CCCTACTTCCCCTTGGGGCTCCCTGTGTCAGGGCACAGACTAGGTCTTGTGGCTGGTCT |
| | GGCTTGCGGCGCGAGGATGGTTCTCTCTGGTCATAGCCCGAAGTCTCATGGCAGTCCCA |
| | AAGGAGGCTTACAACTCCTGCATCACAAGAAAAAGGAAGCCACTGCCAGCTGGGGGGAT |
| | CTGCAGCTCCCAGAAGCTCCGTGAGCCTCAGCCACCCCTCAGACTGGGTTCCTCTCCAA |
| | GCTCGCCCTCTGGAGGGGCAGCGCAGCCTCCCACCAAGGGCCCTGCGACCACAGCAGGG |
| | ATTGGGATGAATTGCCTGTCCTGGATCTGCTCTAGAGGCCCAAGCTGCCTGCCTGAGGA |
| | AGGATGACTTGACAAGTCAGGAGACACTGTTCCCAAAGCCTTGACCAGAGCACCTCAGC |
| | CCGCTGACCTTGCACAAACTCCATCTGCTGCCATGAGAAAAGGGAAGCCGCCTTTGCAA |
| | AACATTGCTGCCTAAAGAAACTCAGCAGCCTCAGGCCCAATTCTGCCACTTCTGGTTTG |
| | GGTACAGTTAAAGGCAACCCTGAGGGACTTGGCAGTAGAAATCCAGGGCCTCCCCTGGG |
| | GCTGGCAGCTTCGTGTGCAGCTAGAGCTTTACCTGAAAGGAAGTCTCTGGGCCCAGAAC |
| | TCTCCACCAAGAGCCTCCCTGCCGTTCGCTGAGTCCCAGCAATTCTCCTAAGTTGAAGG |
| | GATCTGAGAAGGAGAAGGAAATGTGGGGTAGATTTGGTGGTGGTTAGAGATATGCCCCC |
| | CTCATTACTGCCAACAGTTTCGGCTGCATTTCTTCACGCACCTCGGTTCCTCTTCCTGA |
| | AGTTCTTGTGCCCTGCTCTTCAGCACCATGGGCCTTCTTATACGGAAGGCTCTGGGATC |
| | TCCCCCTTGTGGGGCAGGCTCTTGGGGCCAGCCTAAGATCATGGTTTAGGGTGATCAGT |
| | GCTGGCAGATAAATTGAAAAGGCACGCTGGCTTGTGATCTTAAATGAGGACAATCCCCC |
| | CAGGGCTGGGCACTCCTCCCCTCCCCTCACTTCTCCCACCTGCAGAGCCAGTGTCCTTG |
| | GGTGGGCTAGATAGGATATACTGTATGCCGGCTCCTTCAAGCTGCTGACTCACTTTATC |
| | AATAGTTCCATTTAAATTGACTTCAGTGGTGAGACTGTATCCTGTTTGCTATTGCTTGT |
| | TGTGCTATGGGGGGAGGGGGGAGGAATGTGTAAGATAGTTAACATGGGCAAAGGGAGAT |
| | CTTGGGGTGCAGCACTTAAACTGCCTCGTAACCCTTTTCATGATTTCAACCACATTTGC |
| | TAGAGGGAGGGAGCAGCCACGGAGTTAGAGGCCCTTGGGGTTTCTCTTTTCCACTGACA |
| | GGCTTTCCCAGGCAGCTGGCTAGTTCATTCCCTCCCCAGCCAGGTGCAGGCGTAGGAAT |
| | ATGGACATCTGGTTGCTTTGGCCTGCTGCCCTCTTTCAGGGGTCCTAAGCCCACAATCA |
| | TGCCTCCCTAAGACCTTGGCATCCTTCCCTCTAAGCCGTTGGCACCTCTGTGCCACCTC |
| | TCACACTGGCTCCAGACACACAGCCTGTGCTTTTGGAGCTGAGATCACTCGCTTCACCC |
| | TCCTCATCTTTGTTCTCCAAGTAAAGCCACGAGGTCGGGGCGAGGGCAGAGGTGATCAC |
| | CTGCGTGTCCCATCTACAGACCTGCAGCTTCATAAAACTTCTGATTTCTCTTCAGCTTT |
| | GAAAAGGGTTACCCTGGGCACTGGCCTAGAGCCTCACCTCCTAATAGACTTAGCCCCAT |
| | GAGTTTGCCATGTTGAGCAGGACTATTTCTGGCACTTGCAAGTCCCATGATTTCTTCGG |
| | TAATTCTGAGGGTGGGGGGAGGGACATGAAATCATCTTAGCTTAGCTTTCTGTCTGTGA |
| | ATGTCTATATAGTGTATTGTGTGTTTTAACAAATGATTTACACTGACTGTTGCTGTAAA |
| | AGTGAATTTGGAAATAAAGTTATTACTCTGATTAAA |
| 157 | MAEPRQEFEVMEDHAGTYGLGDRKDQGGYTMHQDQEGDTDAGLKESPLQTPTEDGSEEP |
| | GSETSDAKSTPTAEDVTAPLVDEGAPGKQAAAQPHTEIPEGTTAEEAGIGDTPSLEDEA |
| | AGHVTQEPESGKVVQEGFLREPGPPGLSHQLMSGMPGAPLLPEGPREATRQPSGTGPED |
| | TEGGRHAPELLKHQLLGDLHQEGPPLKGAGGKERPGSKEEVDEDRDVDESSPQDSPPSK |
| | ASPAQDGRPPQTAAREATSIPGFPAEGAIPLPVDFLSKVSTEIPASEPDGPSVGRAKGQ |
| | DAPLEFTFHVEITPNVQKEQAHSEEHLGRAAFPGAPGEGPEARGPSLGEDTKEADLPEP |
| | SEKQPAAAPRGKPVSRVPQLKARMVSKSKDGTGSDDKKAKTSTRSSAKTLKNRPCLSPK |
| | HPTPGSSDPLIQPSSPAVCPEPPSSPKYVSSVTSRTGSSGAKEMKLKGADGKTKIATPR |
| | GAAPPGQKGQANATRIPAKTPPAPKTPPSSATKQVQRRPPPAGPRSERGEPPKSGDRSG |
| | YSSPGSPGTPGSRSRTPSLPTPPTREPKKVAVVRTPPKSPSSAKSRLQTAPVPMPDLKN |
| | VKSKIGSTENLKHQPGGGKVQIINKKLDLSNVQSKCGSKDNIKHVPGGGSVQIVYKPVD |
| | LSKVTSKCGSLGNIHHKPGGGQVEVKSEKLDFKDRVQSKIGSLDNITHVPGGGNKKIET |
| | HKLTFRENAKAKTDHGAEIVYKSPVVSGDTSPRHLSNVSSTGSIDMVDSPQLATLADEV |
| | SASLAKQGL |
| 158 | GCAGTCACCGCCACCCACCAGCTCCGGCACCAACAGCAGCGCCGCTGCCACCGCCCACC |
| | TTCTGCCGCCGCCACCACAGCCACCTTCTCCTCCTCCGCTGTCCTCTCCCGTCCTCGCC |
| | TCTGTCGACTATCAGGTGAACTTTGAACCAGGATGGCTGAGCCCCGCCAGGAGTTCGAA |
| | GTGATGGAAGATCACGCTGGGACGTACGGGTTGGGGGACAGGAAAGATCAGGGGGGCTA |
| | CACCATGCACCAAGACCAAGAGGGTGACACGGACGCTGGCCTGAAAGAATCTCCCCTGC |
| | AGACCCCCACTGAGGACGGATCTGAGGAACCGGGCTCTGAAACCTCTGATGCTAAGAGC |
| | ACTCCAACAGCGGAAGCTGAAGAAGCAGGCATTGGAGACACCCCCAGCCTGGAAGACGA |
| | AGCTGCTGGTCACGTGACCCAAGCTCGCATGGTCAGTAAAAGCAAAGACGGGACTGGAA |
| | GCGATGACAAAAAAGCCAAGGGGGCTGATGGTAAAACGAAGATCGCCACACCGCGGGGA |
| | GCAGCCCCTCCAGGCCAGAAGGGCCAGGCCAACGCCACCAGGATTCCAGCAAAAACCCC |
| | GCCCGCTCCAAAGACACCACCCAGCTCTGGTGAACCTCCAAAATCAGGGGATCGCAGCG |
| | GCTACAGCAGCCCCGGCTCCCCAGGCACTCCCGGCAGCCGCTCCCGCACCCCGTCCCTT |
| | CCAACCCCACCCACCCGGGAGCCCAAGAAGGTGGCAGTGGTCCGTACTCCACCCAAGTC |
| | GCCGTCTTCCGCCAAGAGCCGCCTGCAGACAGCCCCCGTGCCCATGCCAGACCTGAAGA |
| | ATGTCAAGTCCAAGATCGGCTCCACTGAGAACCTGAAGCACCAGCCGGGAGGCGGGAAG |
| | GTGCAGATAATTAATAAGAAGCTGGATCTTAGCAACGTCCAGTCCAAGTGTGGCTCAAA |
| | GGATAATATCAAACACGTCCCGGGAGGCGGCAGTGTGCAAATAGTCTACAAACCAGTTG |
| | ACCTGAGCAAGGTGACCTCCAAGTGTGGCTCATTAGGCAACATCCATCATAAACCAGGA |
| | GGTGGCCAGGTGGAAGTAAAATCTGAGAAGCTTGACTTCAAGGACAGAGTCCAGTCGAA |
| | GATTGGGTCCCTGGACAATATCACCCACGTCCCTGGCGGAGGAAATAAAAAGATTGAAA |
| | CCCACAAGCTGACCTTCCGCGAGAACGCCAAAGCCAAGACAGACCACGGGGCGGAGATC |
| | GTGTACAAGTCGCCAGTGGTGTCTGGGGACACGTCTCCACGGCATCTCAGCAATGTCTC |
| | CTCCACCGGCAGCATCGACATGGTAGACTCGCCCCAGCTCGCCACGCTAGCTGACGAGG |
| | TGTCTGCCTCCCTGGCCAAGCAGGGTTTGTGATCAGGCCCCTGGGGCGGTCAATAATTG |
| | TGGAGAGGAGAGAATGAGAGAGTGTGGAAAAAAAAAGAATAATGACCCGGCCCCCGCCC |
| | TCTGCCCCCAGCTGCTCCTCGCAGTTCGGTTAATTGGTTAATCACTTAACCTGCTTTTG |
| | TCACTCGGCTTTGGCTCGGGACTTCAAAATCAGTGATGGGAGTAAGAGCAAATTTCATC |
| | TTTCCAAATTGATGGGTGGGCTAGTAATAAAATATTTAAAAAAAAACATTCAAAAACAT |
| | GGCCACATCCAACATTTCCTCAGGCAATTCCTTTTGATTCTTTTTTCTTCCCCCTCCAT |
| | GTAGAAGAGGGAGAAGGAGAGGCTCTGAAAGCTGCTTCTGGGGGATTTCAAGGGACTGG |
| | GGGTGCCAACCACCTCTGGCCCTGTTGTGGGGGTGTCACAGAGGCAGTGGCAGCAACAA |
| | AGGATTTGAAACTTGGTGTGTTCGTGGAGCCACAGGCAGACGATGTCAACCTTGTGTGA |
| | GTGTGACGGGGGTTGGGGTGGGGGGGGAGGCCACGGGGGAGGCCGAGGCAGGGGCTGGG |
| | CAGAGGGGAGAGGAAGCACAAGAAGTGGGAGTGGGAGAGGAAGCCACGTGCTGGAGAGT |
| | AGACATCCCCCTCCTTGCCGCTGGGAGAGCCAAGGCCTATGCCACCTGCAGCGTCTGAG |
| | CGGCCGCCTGTCCTTGGTGGCCGGGGGTGGGGGCCTGCTGTGGGTCAGTGTGCCACCCT |
| | CTGCAGGGCAGCCTGTGGGAGAAGGGACAGCGGGTAAAAAGAGAAGGCAAGCTGGCAGG |
| | AGGGTGGCACTTCGTGGATGACCTCCTTAGAAAAGACTGACCTTGATGTCTTGAGAGCG |
| | CTGGCCTCTTCCTCCCTCCCTGCAGGGTAGGGGGCCTGAGTTGAGGGGCTTCCCTCTGC |
| | TCCACAGAAACCCTGTTTTATTGAGTTCTGAAGGTTGGAACTGCTGCCATGATTTTGGC |
| | CACTTTGCAGACCTGGGACTTTAGGGCTAACCAGTTCTCTTTGTAAGGACTTGTGCCTC |
| | TTGGGAGACGTCCACCCGTTTCCAAGCCTGGGCCACTGGCATCTCTGGAGTGTGTGGGG |
| | GTCTGGGAGGCAGGTCCCGAGCCCCCTGTCCTTCCCACGGCCACTGCAGTCACCCCGTC |
| | TGCGCCGCTGTGCTGTTGTCTGCCGTGAGAGCCCAATCACTGCCTATACCCCTCATCAC |
| | ACGTCACAATGTCCCGAATTCCCAGCCTCACCACCCCTTCTCAGTAATGACCCTGGTTG |
| | GTTGCAGGAGGTACCTACTCCATACTGAGGGTGAAATTAAGGGAAGGCAAAGTCCAGGC |
| | ACAAGAGTGGGACCCCAGCCTCTCACTCTCAGTTCCACTCATCCAACTGGGACCCTCAC |
| | CACGAATCTCATGATCTGATTCGGTTCCCTGTCTCCTCCTCCCGTCACAGATGTGAGCC |
| | AGGGCACTGCTCAGCTGTGACCCTAGGTGTTTCTGCCTTGTTGACATGGAGAGAGCCCT |
| | TTCCCCTGAGAAGGCCTGGCCCCTTCCTGTGCTGAGCCCACAGCAGCAGGCTGGGTGTC |
| | TTGGTTGTCAGTGGTGGCACCAGGATGGAAGGGCAAGGCACCCAGGGCAGGCCCACAGT |
| | CCCGCTGTCCCCCACTTGCACCCTAGCTTGTAGCTGCCAACCTCCCAGACAGCCCAGCC |
| | CGCTGCTCAGCTCCACATGCATAGTATCAGCCCTCCACACCCGACAAAGGGGAACACAC |
| | CCCCTTGGAAATGGTTCTTTTCCCCCAGTCCCAGCTGGAAGCCATGCTGTCTGTTCTGC |
| | TGGAGCAGCTGAACATATACATAGATGTTGCCCTGCCCTCCCCATCTGCACCCTGTTGA |
| | GTTGTAGTTGGATTTGTCTGTTTATGCTTGGATTCACCAGAGTGACTATGATAGTGAAA |
| | AGAAAAAAAAAAAAAAAAAAGGACGCATGTATCTTGAAATGCTTGTAAAGAGGTTTCTA |
| | ACCCACCCTCACGAGGTGTCTCTCACCCCCACACTGGGACTCGTGTGGCCTGTGTGGTG |
| | CCACCCTGCTGGGGCCTCCCAAGTTTTGAAAGGCTTTCCTCAGCACCTGGGACCCAACA |
| | GAGACCAGCTTCTAGCAGCTAAGGAGGCCGTTCAGCTGTGACGAAGGCCTGAAGCACAG |
| | GATTAGGACTGAAGCGATGATGTCCCCTTCCCTACTTCCCCTTGGGGCTCCCTGTGTCA |
| | GGGCACAGACTAGGTCTTGTGGCTGGTCTGGCTTGCGGCGCGAGGATGGTTCTCTCTGG |
| | TCATAGCCCGAAGTCTCATGGCAGTCCCAAAGGAGGCTTACAACTCCTGCATCACAAGA |
| | AAAAGGAAGCCACTGCCAGCTGGGGGGATCTGCAGCTCCCAGAAGCTCCGTGAGCCTCA |
| | GCCACCCCTCAGACTGGGTTCCTCTCCAAGCTCGCCCTCTGGAGGGGCAGCGCAGCCTC |
| | CCACCAAGGGCCCTGCGACCACAGCAGGGATTGGGATGAATTGCCTGTCCTGGATCTGC |
| | TCTAGAGGCCCAAGCTGCCTGCCTGAGGAAGGATGACTTGACAAGTCAGGAGACACTGT |
| | TCCCAAAGCCTTGACCAGAGCACCTCAGCCCGCTGACCTTGCACAAACTCCATCTGCTG |
| | CCATGAGAAAAGGGAAGCCGCCTTTGCAAAACATTGCTGCCTAAAGAAACTCAGCAGCC |
| | TCAGGCCCAATTCTGCCACTTCTGGTTTGGGTACAGTTAAAGGCAACCCTGAGGGACTT |
| | GGCAGTAGAAATCCAGGGCCTCCCCTGGGGCTGGCAGCTTCGTGTGCAGCTAGAGCTTT |
| | ACCTGAAAGGAAGTCTCTGGGCCCAGAACTCTCCACCAAGAGCCTCCCTGCCGTTCGCT |
| | GAGTCCCAGCAATTCTCCTAAGTTGAAGGGATCTGAGAAGGAGAAGGAAATGTGGGGTA |
| | GATTTGGTGGTGGTTAGAGATATGCCCCCCTCATTACTGCCAACAGTTTCGGCTGCATT |
| | TCTTCACGCACCTCGGTTCCTCTTCCTGAAGTTCTTGTGCCCTGCTCTTCAGCACCATG |
| | GGCCTTCTTATACGGAAGGCTCTGGGATCTCCCCCTTGTGGGGCAGGCTCTTGGGGCCA |
| | GCCTAAGATCATGGTTTAGGGTGATCAGTGCTGGCAGATAAATTGAAAAGGCACGCTGG |
| | CTTGTGATCTTAAATGAGGACAATCCCCCCAGGGCTGGGCACTCCTCCCCTCCCCTCAC |
| | TTCTCCCACCTGCAGAGCCAGTGTCCTTGGGTGGGCTAGATAGGATATACTGTATGCCG |
| | GCTCCTTCAAGCTGCTGACTCACTTTATCAATAGTTCCATTTAAATTGACTTCAGTGGT |
| | GAGACTGTATCCTGTTTGCTATTGCTTGTTGTGCTATGGGGGGAGGGGGGAGGAATGTG |
| | TAAGATAGTTAACATGGGCAAAGGGAGATCTTGGGGTGCAGCACTTAAACTGCCTCGTA |
| | ACCCTTTTCATGATTTCAACCACATTTGCTAGAGGGAGGGAGCAGCCACGGAGTTAGAG |
| | GCCCTTGGGGTTTCTCTTTTCCACTGACAGGCTTTCCCAGGCAGCTGGCTAGTTCATTC |
| | CCTCCCCAGCCAGGTGCAGGCGTAGGAATATGGACATCTGGTTGCTTTGGCCTGCTGCC |
| | CTCTTTCAGGGGTCCTAAGCCCACAATCATGCCTCCCTAAGACCTTGGCATCCTTCCCT |
| | CTAAGCCGTTGGCACCTCTGTGCCACCTCTCACACTGGCTCCAGACACACAGCCTGTGC |
| | TTTTGGAGCTGAGATCACTCGCTTCACCCTCCTCATCTTTGTTCTCCAAGTAAAGCCAC |
| | GAGGTCGGGGCGAGGGCAGAGGTGATCACCTGCGTGTCCCATCTACAGACCTGCAGCTT |
| | CATAAAACTTCTGATTTCTCTTCAGCTTTGAAAAGGGTTACCCTGGGCACTGGCCTAGA |
| | GCCTCACCTCCTAATAGACTTAGCCCCATGAGTTTGCCATGTTGAGCAGGACTATTTCT |
| | GGCACTTGCAAGTCCCATGATTTCTTCGGTAATTCTGAGGGTGGGGGGAGGGACATGAA |
| | ATCATCTTAGCTTAGCTTTCTGTCTGTGAATGTCTATATAGTGTATTGTGTGTTTTAAC |
| | AAATGATTTACACTGACTGTTGCTGTAAAAGTGAATTTGGAAATAAAGTTATTACTCTG |
| | ATTAAA |
| 159 | MAEPRQEFEVMEDHAGTYGLGDRKDQGGYTMHQDQEGDTDAGLKESPLQTPTEDGSEEP |
| | GSETSDAKSTPTAEAEEAGIGDTPSLEDEAAGHVTQARMVSKSKDGTGSDDKKAKGADG |
| | KTKIATPRGAAPPGQKGQANATRIPAKTPPAPKTPPSSGEPPKSGDRSGYSSPGSPGTP |
| | GSRSRTPSLPTPPTREPKKVAVVRTPPKSPSSAKSRLQTAPVPMPDLKNVKSKIGSTEN |
| | LKHQPGGGKVQIINKKLDLSNVQSKCGSKDNIKHVPGGGSVQIVYKPVDLSKVTSKCGS |
| | LGNIHHKPGGGQVEVKSEKLDFKDRVQSKIGSLDNITHVPGGGNKKIETHKLTFRENAK |
| | AKTDHGAEIVYKSPVVSGDTSPRHLSNVSSTGSIDMVDSPQLATLADEVSASLAKQGL |
| 160 | GCAGTCACCGCCACCCACCAGCTCCGGCACCAACAGCAGCGCCGCTGCCACCGCCCACC |
| | TTCTGCCGCCGCCACCACAGCCACCTTCTCCTCCTCCGCTGTCCTCTCCCGTCCTCGCC |
| | TCTGTCGACTATCAGGTGAACTTTGAACCAGGATGGCTGAGCCCCGCCAGGAGTTCGAA |
| | GTGATGGAAGATCACGCTGGGACGTACGGGTTGGGGGACAGGAAAGATCAGGGGGGCTA |
| | CACCATGCACCAAGACCAAGAGGGTGACACGGACGCTGGCCTGAAAGCTGAAGAAGCAG |
| | GCATTGGAGACACCCCCAGCCTGGAAGACGAAGCTGCTGGTCACGTGACCCAAGCTCGC |
| | ATGGTCAGTAAAAGCAAAGACGGGACTGGAAGCGATGACAAAAAAGCCAAGGGGGCTGA |
| | TGGTAAAACGAAGATCGCCACACCGCGGGGAGCAGCCCCTCCAGGCCAGAAGGGCCAGG |
| | CCAACGCCACCAGGATTCCAGCAAAAACCCCGCCCGCTCCAAAGACACCACCCAGCTCT |
| | GGTGAACCTCCAAAATCAGGGGATCGCAGCGGCTACAGCAGCCCCGGCTCCCCAGGCAC |
| | TCCCGGCAGCCGCTCCCGCACCCCGTCCCTTCCAACCCCACCCACCCGGGAGCCCAAGA |
| | AGGTGGCAGTGGTCCGTACTCCACCCAAGTCGCCGTCTTCCGCCAAGAGCCGCCTGCAG |
| | ACAGCCCCCGTGCCCATGCCAGACCTGAAGAATGTCAAGTCCAAGATCGGCTCCACTGA |
| | GAACCTGAAGCACCAGCCGGGAGGCGGGAAGGTGCAAATAGTCTACAAACCAGTTGACC |
| | TGAGCAAGGTGACCTCCAAGTGTGGCTCATTAGGCAACATCCATCATAAACCAGGAGGT |
| | GGCCAGGTGGAAGTAAAATCTGAGAAGCTTGACTTCAAGGACAGAGTCCAGTCGAAGAT |
| | TGGGTCCCTGGACAATATCACCCACGTCCCTGGCGGAGGAAATAAAAAGATTGAAACCC |
| | ACAAGCTGACCTTCCGCGAGAACGCCAAAGCCAAGACAGACCACGGGGCGGAGATCGTG |
| | TACAAGTCGCCAGTGGTGTCTGGGGACACGTCTCCACGGCATCTCAGCAATGTCTCCTC |
| | CACCGGCAGCATCGACATGGTAGACTCGCCCCAGCTCGCCACGCTAGCTGACGAGGTGT |
| | CTGCCTCCCTGGCCAAGCAGGGTTTGTGATCAGGCCCCTGGGGCGGTCAATAATTGTGG |
| | AGAGGAGAGAATGAGAGAGTGTGGAAAAAAAAAGAATAATGACCCGGCCCCCGCCCTCT |
| | GCCCCCAGCTGCTCCTCGCAGTTCGGTTAATTGGTTAATCACTTAACCTGCTTTTGTCA |
| | CTCGGCTTTGGCTCGGGACTTCAAAATCAGTGATGGGAGTAAGAGCAAATTTCATCTTT |
| | CCAAATTGATGGGTGGGCTAGTAATAAAATATTTAAAAAAAAACATTCAAAAACATGGC |
| | CACATCCAACATTTCCTCAGGCAATTCCTTTTGATTCTTTTTTCTTCCCCCTCCATGTA |
| | GAAGAGGGAGAAGGAGAGGCTCTGAAAGCTGCTTCTGGGGGATTTCAAGGGACTGGGGG |
| | TGCCAACCACCTCTGGCCCTGTTGTGGGGGTGTCACAGAGGCAGTGGCAGCAACAAAGG |
| | ATTTGAAACTTGGTGTGTTCGTGGAGCCACAGGCAGACGATGTCAACCTTGTGTGAGTG |
| | TGACGGGGGTTGGGGTGGGGGGGGAGGCCACGGGGGAGGCCGAGGCAGGGGCTGGGCAG |
| | AGGGGAGAGGAAGCACAAGAAGTGGGAGTGGGAGAGGAAGCCACGTGCTGGAGAGTAGA |
| | CATCCCCCTCCTTGCCGCTGGGAGAGCCAAGGCCTATGCCACCTGCAGCGTCTGAGCGG |
| | CCGCCTGTCCTTGGTGGCCGGGGGTGGGGGCCTGCTGTGGGTCAGTGTGCCACCCTCTG |
| | CAGGGCAGCCTGTGGGAGAAGGGACAGCGGGTAAAAAGAGAAGGCAAGCTGGCAGGAGG |
| | GTGGCACTTCGTGGATGACCTCCTTAGAAAAGACTGACCTTGATGTCTTGAGAGCGCTG |
| | GCCTCTTCCTCCCTCCCTGCAGGGTAGGGGGCCTGAGTTGAGGGGCTTCCCTCTGCTCC |
| | ACAGAAACCCTGTTTTATTGAGTTCTGAAGGTTGGAACTGCTGCCATGATTTTGGCCAC |
| | TTTGCAGACCTGGGACTTTAGGGCTAACCAGTTCTCTTTGTAAGGACTTGTGCCTCTTG |
| | GGAGACGTCCACCCGTTTCCAAGCCTGGGCCACTGGCATCTCTGGAGTGTGTGGGGGTC |
| | TGGGAGGCAGGTCCCGAGCCCCCTGTCCTTCCCACGGCCACTGCAGTCACCCCGTCTGC |
| | GCCGCTGTGCTGTTGTCTGCCGTGAGAGCCCAATCACTGCCTATACCCCTCATCACACG |
| | TCACAATGTCCCGAATTCCCAGCCTCACCACCCCTTCTCAGTAATGACCCTGGTTGGTT |
| | GCAGGAGGTACCTACTCCATACTGAGGGTGAAATTAAGGGAAGGCAAAGTCCAGGCACA |
| | AGAGTGGGACCCCAGCCTCTCACTCTCAGTTCCACTCATCCAACTGGGACCCTCACCAC |
| | GAATCTCATGATCTGATTCGGTTCCCTGTCTCCTCCTCCCGTCACAGATGTGAGCCAGG |
| | GCACTGCTCAGCTGTGACCCTAGGTGTTTCTGCCTTGTTGACATGGAGAGAGCCCTTTC |
| | CCCTGAGAAGGCCTGGCCCCTTCCTGTGCTGAGCCCACAGCAGCAGGCTGGGTGTCTTG |
| | GTTGTCAGTGGTGGCACCAGGATGGAAGGGCAAGGCACCCAGGGCAGGCCCACAGTCCC |
| | GCTGTCCCCCACTTGCACCCTAGCTTGTAGCTGCCAACCTCCCAGACAGCCCAGCCCGC |
| | TGCTCAGCTCCACATGCATAGTATCAGCCCTCCACACCCGACAAAGGGGAACACACCCC |
| | CTTGGAAATGGTTCTTTTCCCCCAGTCCCAGCTGGAAGCCATGCTGTCTGTTCTGCTGG |
| | AGCAGCTGAACATATACATAGATGTTGCCCTGCCCTCCCCATCTGCACCCTGTTGAGTT |
| | GTAGTTGGATTTGTCTGTTTATGCTTGGATTCACCAGAGTGACTATGATAGTGAAAAGA |
| | AAAAAAAAAAAAAAAAAGGACGCATGTATCTTGAAATGCTTGTAAAGAGGTTTCTAACC |
| | CACCCTCACGAGGTGTCTCTCACCCCCACACTGGGACTCGTGTGGCCTGTGTGGTGCCA |
| | CCCTGCTGGGGCCTCCCAAGTTTTGAAAGGCTTTCCTCAGCACCTGGGACCCAACAGAG |
| | ACCAGCTTCTAGCAGCTAAGGAGGCCGTTCAGCTGTGACGAAGGCCTGAAGCACAGGAT |
| | TAGGACTGAAGCGATGATGTCCCCTTCCCTACTTCCCCTTGGGGCTCCCTGTGTCAGGG |
| | CACAGACTAGGTCTTGTGGCTGGTCTGGCTTGCGGCGCGAGGATGGTTCTCTCTGGTCA |
| | TAGCCCGAAGTCTCATGGCAGTCCCAAAGGAGGCTTACAACTCCTGCATCACAAGAAAA |
| | AGGAAGCCACTGCCAGCTGGGGGGATCTGCAGCTCCCAGAAGCTCCGTGAGCCTCAGCC |
| | ACCCCTCAGACTGGGTTCCTCTCCAAGCTCGCCCTCTGGAGGGGCAGCGCAGCCTCCCA |
| | CCAAGGGCCCTGCGACCACAGCAGGGATTGGGATGAATTGCCTGTCCTGGATCTGCTCT |
| | AGAGGCCCAAGCTGCCTGCCTGAGGAAGGATGACTTGACAAGTCAGGAGACACTGTTCC |
| | CAAAGCCTTGACCAGAGCACCTCAGCCCGCTGACCTTGCACAAACTCCATCTGCTGCCA |
| | TGAGAAAAGGGAAGCCGCCTTTGCAAAACATTGCTGCCTAAAGAAACTCAGCAGCCTCA |
| | GGCCCAATTCTGCCACTTCTGGTTTGGGTACAGTTAAAGGCAACCCTGAGGGACTTGGC |
| | AGTAGAAATCCAGGGCCTCCCCTGGGGCTGGCAGCTTCGTGTGCAGCTAGAGCTTTACC |
| | TGAAAGGAAGTCTCTGGGCCCAGAACTCTCCACCAAGAGCCTCCCTGCCGTTCGCTGAG |
| | TCCCAGCAATTCTCCTAAGTTGAAGGGATCTGAGAAGGAGAAGGAAATGTGGGGTAGAT |
| | TTGGTGGTGGTTAGAGATATGCCCCCCTCATTACTGCCAACAGTTTCGGCTGCATTTCT |
| | TCACGCACCTCGGTTCCTCTTCCTGAAGTTCTTGTGCCCTGCTCTTCAGCACCATGGGC |
| | CTTCTTATACGGAAGGCTCTGGGATCTCCCCCTTGTGGGGCAGGCTCTTGGGGCCAGCC |
| | TAAGATCATGGTTTAGGGTGATCAGTGCTGGCAGATAAATTGAAAAGGCACGCTGGCTT |
| | GTGATCTTAAATGAGGACAATCCCCCCAGGGCTGGGCACTCCTCCCCTCCCCTCACTTC |
| | TCCCACCTGCAGAGCCAGTGTCCTTGGGTGGGCTAGATAGGATATACTGTATGCCGGCT |
| | CCTTCAAGCTGCTGACTCACTTTATCAATAGTTCCATTTAAATTGACTTCAGTGGTGAG |
| | ACTGTATCCTGTTTGCTATTGCTTGTTGTGCTATGGGGGGAGGGGGGAGGAATGTGTAA |
| | GATAGTTAACATGGGCAAAGGGAGATCTTGGGGTGCAGCACTTAAACTGCCTCGTAACC |
| | CTTTTCATGATTTCAACCACATTTGCTAGAGGGAGGGAGCAGCCACGGAGTTAGAGGCC |
| | CTTGGGGTTTCTCTTTTCCACTGACAGGCTTTCCCAGGCAGCTGGCTAGTTCATTCCCT |
| | CCCCAGCCAGGTGCAGGCGTAGGAATATGGACATCTGGTTGCTTTGGCCTGCTGCCCTC |
| | TTTCAGGGGTCCTAAGCCCACAATCATGCCTCCCTAAGACCTTGGCATCCTTCCCTCTA |
| | AGCCGTTGGCACCTCTGTGCCACCTCTCACACTGGCTCCAGACACACAGCCTGTGCTTT |
| | TGGAGCTGAGATCACTCGCTTCACCCTCCTCATCTTTGTTCTCCAAGTAAAGCCACGAG |
| | GTCGGGGCGAGGGCAGAGGTGATCACCTGCGTGTCCCATCTACAGACCTGCAGCTTCAT |
| | AAAACTTCTGATTTCTCTTCAGCTTTGAAAAGGGTTACCCTGGGCACTGGCCTAGAGCC |
| | TCACCTCCTAATAGACTTAGCCCCATGAGTTTGCCATGTTGAGCAGGACTATTTCTGGC |
| | ACTTGCAAGTCCCATGATTTCTTCGGTAATTCTGAGGGTGGGGGGAGGGACATGAAATC |
| | ATCTTAGCTTAGCTTTCTGTCTGTGAATGTCTATATAGTGTATTGTGTGTTTTAACAAA |
| | TGATTTACACTGACTGTTGCTGTAAAAGTGAATTTGGAAATAAAGTTATTACTCTGATT |
| | AAA |
| 161 | MAEPRQEFEVMEDHAGTYGLGDRKDOGGYTMHQDQEGDTDAGLKAEEAGIGDTPSLEDE |
| | AAGHVTQARMVSKSKDGTGSDDKKAKGADGKTKIATPRGAAPPGQKGQANATRIPAKTP |
| | PAPKTPPSSGEPPKSGDRSGYSSPGSPGTPGSRSRTPSLPTPPTREPKKVAVVRTPPKS |
| | PSSAKSRLQTAPVPMPDLKNVKSKIGSTENLKHQPGGGKVQIVYKPVDLSKVTSKCGSL |
| | GNIHHKPGGGQVEVKSEKLDFKDRVQSKIGSLDNITHVPGGGNKKIETHKLTFRENAKA |
| | KTDHGAEIVYKSPVVSGDTSPRHLSNVSSTGSIDMVDSPQLATLADEVSASLAKQGL |
| 162 | FEDLY |
| 163 | LFGNMEEGDCPSDWKTDSTCR |
| 164 | AGKIT |
| 165 | VEKLTLD |
| 166 | EVQLVESGGGLVKPGGSLRLSCVASGFTFSSYSMNWVRQAPGKGLEWVSSISRSSSYIY |
| | YADSVKGRFTISRDNAKNSLYLQMNSLRAEDTAVYYCARIHGYSNSDAFDKWGQGTLVT |
| | VSSASTKGPCVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEAA |
| | GGPSVFLFPPKPKDTLMISRTPEVTCVVVDVSQEDPEVQFNWYVDGVEVHNAKTKPREE |
| | QFNSTYRVVSVLTVLHQDWLNGKEYKCKVSNKGLPSSIEKTISKAKGQPREPQVYTLPP |
| | SQEEMTKNQVSLTCLVKGFYPSDIAVEWESNGQPENNYKTTPPVLDSDGSFFLYSRLTV |
| | DKSRWQEGNVFSCSVMHEALHNHYTQKSLSLSLG |
| 167 | ESKYGPPCPPCPAPEAAGGPSVFLFPPKPKDTLMISRTPEVTCVVVDVSQEDPEVQFNW |
| | YVDGVEVHNAKTKPREEQFNSTYRVVSVLTVLHQDWLNGKEYKCKVSNKGLPSSIEKTI |
| | SKAKGQPREPQVYTLPPSQEEMTKNQVSLTCLVKGFYPSDIAVEWESNGQPENNYKTTP |
| | PVLDSDGSFLLYSKLTVDKSRWQEGNVFSCSVMHEALHNHYTQKSLSLSLG |

1. A protein comprising one monovalent human transferrin receptor (TfR) binding domain, wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, and wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 21, HCDR3 comprises SEQ ID NO: 22, LCDR1 comprises SEQ ID NO: 23, LCDR2 comprises SEQ ID NO: 5, and LCDR3 comprises SEQ ID NO: 24; or
(b) HCDR1 comprises SEQ ID NO: 13, HCDR2 comprises SEQ ID NO: 25, HCDR3 comprises SEQ ID NO: 26, LCDR1 comprises SEQ ID NO: 16, LCDR2 comprises SEQ ID NO: 17, and LCDR3 comprises SEQ ID NO: 18.

2. The protein of claim 1, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6;
(b) HCDR1 comprises SEQ ID NO: 1, HCDR2 comprises SEQ ID NO: 2, HCDR3 comprises SEQ ID NO: 7, LCDR1 comprises SEQ ID NO: 4, LCDR2 comprises SEQ ID NO: 5, and LCDR3 comprises SEQ ID NO: 6;
(c) HCDR1 comprises SEQ ID NO: 1, HCDR2 comprises SEQ ID NO: 2, HCDR3 comprises SEQ ID NO: 8, LCDR1 comprises SEQ ID NO: 9, LCDR2 comprises SEQ ID NO: 5, and LCDR3 comprises SEQ ID NO: 6;
(d) HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12;
(e) HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 17, and LCDR3 comprises SEQ ID NO: 18;
(f) HCDR1 comprises SEQ ID NO: 13, HCDR2 comprises SEQ ID NO: 19, HCDR3 comprises SEQ ID NO: 15, LCDR1 comprises SEQ ID NO: 16, LCDR2 comprises SEQ ID NO: 17, and LCDR3 comprises SEQ ID NO: 18; or
(g) HCDR1 comprises SEQ ID NO: 13, HCDR2 comprises SEQ ID NO: 19, HCDR3 comprises SEQ ID NO: 20, LCDR1 comprises SEQ ID NO: 16, LCDR2 comprises SEQ ID NO: 17, and LCDR3 comprises SEQ ID NO: 18.

3. The protein of claim 1, wherein the VH and VL comprise the following sequences:
(a) VH comprises SEQ ID NO: 27 and VL comprises SEQ ID NO: 28;
(b) VH comprises SEQ ID NO: 29 and VL comprises SEQ ID NO: 28;
(c) VH comprises SEQ ID NO: 30 and VL comprises SEQ ID NO: 31;
(d) VH comprises SEQ ID NO: 32 and VL comprises SEQ ID NO: 33;
(e) VH comprises SEQ ID NO: 34 and VL comprises SEQ ID NO: 35;
(f) VH comprises SEQ ID NO: 36 and VL comprises SEQ ID NO: 37; or
(g) VH comprises SEQ ID NO: 38 and VL comprises SEQ ID NO: 37.

4. The protein of claim 1, wherein the human TfR binding domain is a Fab, scFv, Fv, or scFab.

5. The protein of claim 1, wherein the human TfR binding domain further comprises a heavy chain constant region comprising cysteine at residue 124 (according to the EU Index numbering).

6. The protein of claim 1, wherein the human TfR binding domain further comprises a light chain constant region comprising cysteine at residue 156 (according to the EU Index numbering).

7. The protein of claim 1 further comprising a half-life extender.

8. The protein of claim 7, wherein the half-life extender is selected from an immunoglobulin Fc region or a VHH that binds human serum albumin (HSA).

9. The protein of claim 8, wherein the half-life extender is an immunoglobulin Fc region.

10. The protein of claim 9, wherein the Fc region is a modified human IgG4 Fc region.

11. The protein of claim 10, wherein the modified human IgG4 Fc region comprises proline at residue 228, and alanine at residues 234 and 235 (all residues are numbered according to the EU Index numbering).

12. The protein of claim 9, wherein the protein comprises an immunoglobulin Fc region comprising cysteine at residue 378 (according to the EU Index numbering).

13. The protein of claim 9, wherein the Fc region comprises:
(a) a first Fc CH3 domain comprising a serine at position 349, a methionine at position 366, a tyrosine at position 370, and a valine at position 409; and a second Fc CH3 domain comprising a glycine at position 356, an aspartic acid at position 357, a glutamine at position 364, and an alanine at position 407 (all residues are numbered according to the EU Index numbering); or
(b) a first Fc CH3 domain comprising leucine at residue 405, and a second Fc CH3 domain comprising arginine at residue 409 (all residues are numbered according to the EU Index numbering).

14. The protein of claim 1, wherein the protein comprises one heavy chain (HC) and one light chain (LC), wherein the HC and LC comprise the following sequences:
(a) HC comprises SEQ ID NO: 53 and LC comprises SEQ ID NO: 54;
(b) HC comprises SEQ ID NO: 55 and LC comprises SEQ ID NO: 54;
(c) HC comprises SEQ ID NO: 56 and LC comprises SEQ ID NO: 57;
(d) HC comprises SEQ ID NO: 58 and LC comprises SEQ ID NO: 59;
(e) HC comprises SEQ ID NO: 60 and LC comprises SEQ ID NO: 61;
(f) HC comprises SEQ ID NO: 62 and LC comprises SEQ ID NO: 63; or
(g) HC comprises SEQ ID NO: 64 and LC comprises SEQ ID NO: 63.

15. The protein of claim 1, wherein the protein comprises two heavy chains HC1 and HC2 and one light chain LC1, wherein HC1 comprises SEQ ID NO: 68, LC1 comprises SEQ ID NO: 59, HC2 comprises SEQ ID NO: 69.

16. The protein of claim 1, wherein the protein comprises two heavy chains HC1 and HC2 and one light chain LC1, wherein HC1 comprises SEQ ID NO: 138, LC1 comprises SEQ ID NO: 59, HC2 comprises SEQ ID NO: 139.

17. The protein of claim 1, wherein the protein comprises two heavy chains HC1 and HC2 and one light chain LC1, wherein HC1 comprises SEQ ID NO: 166, LC1 comprises SEQ ID NO: 54, HC2 comprises SEQ ID NO: 167.

18. The protein of claim 1, wherein the protein comprises one heavy chain (HC) and one light chain (LC), and wherein the HC comprises SEQ ID NO: 65 and the LC comprises SEQ ID NO: 59.

19. The protein of claim 8, wherein the half-life extender is a VHH that binds HSA.

20. The protein of claim 19, wherein the VHH comprises CDR1 comprising SEQ ID NO: 39, CDR2 comprising SEQ ID NO: 40, and CDR3 comprising SEQ ID NO: 41.

21. The protein of claim 19, wherein the VHH comprises SEQ ID NO: 42.

22. The protein of claim 19, wherein the protein comprises one heavy chain (HC) and one light chain (LC), and wherein the HC comprises SEQ ID NO: 66 and the LC comprises SEQ ID NO: 67.

23. The protein of claim 1, wherein the protein is a heterodimeric antibody that comprises a first arm comprising one monovalent human TfR binding domain and a second arm that is a null arm.

24. The protein of claim 23, wherein the second arm comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 43, HCDR2 comprises SEQ ID NO: 44, HCDR3 comprises SEQ ID NO: 45, LCDR1 comprises SEQ ID NO: 46, LCDR2 comprises SEQ ID NO: 47, and LCDR3 comprises SEQ ID NO: 48.

25. The protein of claim 24, wherein the VH comprises SEQ ID NO: 49 and the VL comprises SEQ ID NO: 50.

26. The protein of claim 23, wherein the protein comprises two heavy chains HC1 and HC2 and two light chains LC1 and LC2, wherein HC1, LC1, HC2, and LC2 comprise the following sequences:
(a) HC1 comprises SEQ ID NO: 64, LC1 comprises SEQ ID NO: 63, HC2 comprises SEQ ID NO: 51, and LC2 comprises SEQ ID NO: 52;
(b) HC1 comprises SEQ ID NO: 55, LC1 comprises SEQ ID NO: 54, HC2 comprises SEQ ID NO: 51, and LC2 comprises SEQ ID NO: 52;
(c) HC1 comprises SEQ ID NO: 56, LC1 comprises SEQ ID NO: 57, HC2 comprises SEQ ID NO: 51, and LC2 comprises SEQ ID NO: 52; or
(d) HC1 comprises SEQ ID NO: 58, LC1 comprises SEQ ID NO: 59, HC2 comprises SEQ ID NO: 51, and LC2 comprises SEQ ID NO: 52.

27. A protein comprising one monovalent human transferrin receptor (TfR) binding domain, wherein the human TfR binding domain binds an epitope comprising one or more residues in (a) residues 346-364 FGNMEGDCPSDWKTDSTCR (SEQ ID NO: 119), (b) residues 243-247 FEDLY (SEQ ID NO: 162) and residues 345-364 LFGNMEEGDCPSDWKTDSTCR) (SEQ ID NO: 163), or (c) residues 243-247 FEDLY (SEQ ID NO: 162), residues 259-263 AGKIT (SEQ ID NO: 164), and residues 532-538 (VEKLTLD) (SEQ ID NO: 165), of human TfR.

28. A protein comprising one monovalent mouse transferrin receptor (TfR) binding domain, wherein the mouse TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 71, HCDR2 comprises SEQ ID NO: 72, HCDR3 comprises SEQ ID NO: 73, LCDR1 comprises SEQ ID NO: 74, LCDR2 comprises SEQ ID NO: 75, and LCDR3 comprises SEQ ID NO: 76.

29. The protein of claim 28, wherein the VH comprising SEQ ID NO: 77 and the VL comprising SEQ ID NO: 78.

30. The protein of claim 28, wherein the protein comprises a heavy chain (HC) comprising SEQ ID NO: 79 and a light chain (LC) comprising SEQ ID NO: 80.

31. The protein of claim 28, wherein the protein is a heterodimeric antibody that comprises a first arm comprising one monovalent mouse TfR binding domain and a second arm that is a null arm.

32. The protein of claim 28, wherein the protein comprises two heavy chains HC1 and HC2 and two light chains LC1 and LC2, wherein HC1 comprises SEQ ID NO: 79, LC1 comprises SEQ ID NO: 80, HC2 comprises SEQ ID NO: 51, and LC2 comprises SEQ ID NO: 52.

33. A conjugate comprising the protein of claim 1 and a therapeutic agent.

34. The conjugate of claim 33, wherein the therapeutic agent is selected from a double stranded RNA, oligonucleotide, peptide, small molecule, nanoparticle, lipid nanoparticle, exosome, antibody or antigen binding fragment thereof, or a combination thereof.

35. The conjugate of claim 33, wherein the therapeutic agent is linked to the protein through a linker.

36. The conjugate of claim 33, wherein the therapeutic agent is a double stranded RNA (dsRNA).

37. The conjugate of claim 36, wherein the dsRNA comprises a sense strand and an antisense stand, wherein the antisense strand is complementary to a target mRNA selected from SNCA, MAPT, APP, ATXN2, ATXN3, SARM1, APOE, BACE1, FMR1, LRRK2, HTT, SOD1, SCN10A, SCN9A or CACNA1B mRNA.

38. The conjugate of claim 37, wherein the antisense strand is complementary to SNCA mRNA.

39. The conjugate of claim 37, wherein the antisense strand is complementary to MAPT mRNA.

40. The conjugate of claim 35, wherein the linker is a Mal-Tet-TCO linker, SMCC linker, or GDM linker.

41. The conjugate of claim 33, wherein the therapeutic agent to protein ratio is about 1:1 to 3:1.

42. A conjugate of Formula (I): R-L-P, wherein R is a double stranded RNA (dsRNA) comprising a sense stand and an antisense strand;
wherein P is a protein comprising one monovalent human TfR binding domain; and
wherein L is a linker, or optionally absent,
wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 21, HCDR3 comprises SEQ ID NO: 22, LCDR1 comprises SEQ ID NO: 23, LCDR2 comprises SEQ ID NO: 5, and LCDR3 comprises SEQ ID NO: 24; or
(b) HCDR1 comprises SEQ ID NO: 13, HCDR2 comprises SEQ ID NO: 25, HCDR3 comprises SEQ ID NO: 26, LCDR1 comprises SEQ ID NO: 16, LCDR2 comprises SEQ ID NO: 17, and LCDR3 comprises SEQ ID NO: 18.

43. The conjugate of claim 42, wherein the R to P ratio is about 1:1 to 3:1.

44. A conjugate of Formula (II): (R-L)ₙ-P, wherein R is a double stranded RNA (dsRNA) comprising a sense stand and an antisense strand;
wherein P is a protein comprising one monovalent human TfR binding domain; and
wherein L is a linker, or optionally absent,
wherein the human TfR binding domain comprises a heavy chain variable region (VH) and a light chain variable region (VL), wherein the VH comprises heavy chain complementarity determining regions HCDR1, HCDR2, and HCDR3, and the VL comprises light chain complementarity determining regions LCDR1, LCDR2, and LCDR3, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 21, HCDR3 comprises SEQ ID NO: 22, LCDR1 comprises SEQ ID NO: 23, LCDR2 comprises SEQ ID NO: 5, and LCDR3 comprises SEQ ID NO: 24; or
(b) HCDR1 comprises SEQ ID NO: 13, HCDR2 comprises SEQ ID NO: 25, HCDR3 comprises SEQ ID NO: 26, LCDR1 comprises SEQ ID NO: 16, LCDR2 comprises SEQ ID NO: 17, and LCDR3 comprises SEQ ID NO: 18;
and wherein n is 1 to 3.

45. The conjugate of claim 44, wherein n is 1.

46. The conjugate of claim 44, wherein n is 2.

47. The conjugate of claim 44, wherein the HCDR1, HCDR2, HCDR3, LCDR1, LCDR2, and LCDR3 comprise the following sequences:
(a) HCDR1 comprises SEQ ID NO: 1, HCDR2 comprises SEQ ID NO: 2, HCDR3 comprises SEQ ID NO: 3, LCDR1 comprises SEQ ID NO: 4, LCDR2 comprises SEQ ID NO: 5, and LCDR3 comprises SEQ ID NO: 6;
(b) HCDR1 comprises SEQ ID NO: 1, HCDR2 comprises SEQ ID NO: 2, HCDR3 comprises SEQ ID NO: 7, LCDR1 comprises SEQ ID NO: 4, LCDR2 comprises SEQ ID NO: 5, and LCDR3 comprises SEQ ID NO: 6;
(c) HCDR1 comprises SEQ ID NO: 1, HCDR2 comprises SEQ ID NO: 2, HCDR3 comprises SEQ ID NO: 8, LCDR1 comprises SEQ ID NO: 9, LCDR2 comprises SEQ ID NO: 5, and LCDR3 comprises SEQ ID NO: 6;
(d) HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12;
(e) HCDR1 comprises SEQ ID NO: 13, HCDR2 comprises SEQ ID NO: 14, HCDR3 comprises SEQ ID NO: 15, LCDR1 comprises SEQ ID NO: 16, LCDR2 comprises SEQ ID NO: 17, and LCDR3 comprises SEQ ID NO: 18;
(f) HCDR1 comprises SEQ ID NO: 13, HCDR2 comprises SEQ ID NO: 19, HCDR3 comprises SEQ ID NO: 15, LCDR1 comprises SEQ ID NO: 16, LCDR2 comprises SEQ ID NO: 17, and LCDR3 comprises SEQ ID NO: 18; or
(g) HCDR1 comprises SEQ ID NO: 13, HCDR2 comprises SEQ ID NO: 19, HCDR3 comprises SEQ ID NO: 20, LCDR1 comprises SEQ ID NO: 16, LCDR2 comprises SEQ ID NO: 17, and LCDR3 comprises SEQ ID NO: 18.

48. The conjugate of claim 44, wherein the VH and VL comprise the following sequences:
(a) VH comprises SEQ ID NO: 27 and VL comprises SEQ ID NO: 28;
(b) VH comprises SEQ ID NO: 29 and VL comprises SEQ ID NO: 28;
(c) VH comprises SEQ ID NO: 30 and VL comprises SEQ ID NO: 31;
(d) VH comprises SEQ ID NO: 32 and VL comprises SEQ ID NO: 33;
(e) VH comprises SEQ ID NO: 34 and VL comprises SEQ ID NO: 35;
(f) VH comprises SEQ ID NO: 36 and VL comprises SEQ ID NO: 37; or
(g) VH comprises SEQ ID NO: 38 and VL comprises SEQ ID NO: 37.

49. The conjugate of claim 44, wherein HCDR1 comprises SEQ ID NO: 1, HCDR2 comprises SEQ ID NO: 10, HCDR3 comprises SEQ ID NO: 11, LCDR1 comprises SEQ ID NO: 9, LCDR2 comprises SEQ ID NO: 5, and LCDR3 comprises SEQ ID NO: 12.

50. The conjugate of claim 44, wherein VH comprises SEQ ID NO: 32 and VL comprises SEQ ID NO: 33.

51. The conjugate of claim 44, wherein the human TfR binding domain is a Fab, scFv, Fv, or scFab.

52. The conjugate of claim 44, wherein the human TfR binding domain further comprises a heavy chain constant region comprising cysteine at residue 124 (according to the EU Index numbering).

53. The conjugate of claim 44, wherein the human TfR binding domain further comprises a light chain constant region comprising cysteine at residue 156 (according to the EU Index numbering).

54. The conjugate of claim 44, wherein the protein further comprises a half-life extender.

55. The conjugate of claim 54, wherein the half-life extender is selected from an immunoglobulin Fc region or a VHH that binds human serum albumin (HSA).

56. The conjugate of claim 55, wherein the half-life extender is an immunoglobulin Fc region.

57. The conjugate of claim 56, wherein the immunoglobulin Fc region is a modified human IgG4 Fc region.

58. The conjugate of claim 57, wherein the modified human IgG4 Fc region comprises proline at residue 228, and alanine at residues 234 and 235 (all residues are numbered according to the EU Index numbering).

59. The conjugate of claim 56, wherein the protein comprises an immunoglobulin Fc region comprising cysteine at residue 378 (according to the EU Index numbering).

60. The conjugate of claim 56, wherein the Fc region comprises:
(a) a first Fc CH3 domain comprising a serine at position 349, a methionine at position 366, a tyrosine at position 370, and a valine at position 409; and a second Fc CH3 domain comprising a glycine at position 356, an aspartic acid at position 357, a glutamine at position 364, and an alanine at position 407 (all residues are numbered according to the EU Index numbering); or
(b) a first Fc CH3 domain comprising leucine at residue 405, and a second Fc CH3 domain comprising arginine at residue 409 (all residues are numbered according to the EU Index numbering).

61. The conjugate of claim 44, wherein the protein comprises one heavy chain (HC) and one light chain (LC), wherein the HC and LC comprise the following sequences:
(a) HC comprises SEQ ID NO: 53 and LC comprises SEQ ID NO: 54;
(b) HC comprises SEQ ID NO: 55 and LC comprises SEQ ID NO: 54;
(c) HC comprises SEQ ID NO: 56 and LC comprises SEQ ID NO: 57;
(d) HC comprises SEQ ID NO: 58 and LC comprises SEQ ID NO: 59;
(e) HC comprises SEQ ID NO: 60 and LC comprises SEQ ID NO: 61;
(f) HC comprises SEQ ID NO: 62 and LC comprises SEQ ID NO: 63; or
(g) HC comprises SEQ ID NO: 64 and LC comprises SEQ ID NO: 63.

62. The conjugate of claim 44, wherein the protein comprises two heavy chains HC1 and HC2 and one light chain LC1, wherein HC1 comprises SEQ ID NO: 68, LC1 comprises SEQ ID NO: 59, HC2 comprises SEQ ID NO: 69.

63. The conjugate of claim 44, wherein the protein comprises two heavy chains HC1 and HC2 and one light chain LC1, wherein HC1 comprises SEQ ID NO: 138, LC1 comprises SEQ ID NO: 59, HC2 comprises SEQ ID NO: 139.

64. The conjugate of claim 44, wherein the protein comprises two heavy chains HC1 and HC2 and one light chain LC1, wherein HC1 comprises SEQ ID NO: 166, LC1 comprises SEQ ID NO: 54, HC2 comprises SEQ ID NO: 167.

65. The conjugate of claim 44, wherein the protein comprises one heavy chain (HC) and one light chain (LC), and wherein the HC comprises SEQ ID NO: 65 and the LC comprises SEQ ID NO: 59.

66. The conjugate of claim 54, wherein the half-life extender is a VHH that binds HSA.

67. The conjugate of claim 66, wherein the VHH comprises CDR1 comprising SEQ ID NO:
39, CDR2 comprising SEQ ID NO: 40, and CDR3 comprising SEQ ID NO: 41.

68. The conjugate of claim 66, wherein the VHH comprises SEQ ID NO: 42.

69. The conjugate of claim 66, wherein the protein comprises one heavy chain (HC) and one light chain (LC), and wherein the HC comprises SEQ ID NO: 66 and the LC comprises SEQ ID NO: 67.

70. The conjugate of claim 44, wherein the protein is a heterodimeric antibody that comprises a first arm comprising one monovalent human TfR binding domain and a second arm that is a null arm.

71. The conjugate of claim 70, wherein the second arm comprises a VH and a VL, wherein the VH comprises heavy chain complementarity determining regions HCDR1, HCDR2, and HCDR3, and the VL comprises light chain complementarity determining regions LCDR1, LCDR2, and LCDR3, and wherein HCDR1 comprises SEQ ID NO: 43, HCDR2 comprises SEQ ID NO: 44, HCDR3 comprises SEQ ID NO: 45, LCDR1 comprises SEQ ID NO: 46, LCDR2 comprises SEQ ID NO: 47, and LCDR3 comprises SEQ ID NO: 48.

72. The conjugate of claim 71, wherein the VH comprises SEQ ID NO: 49 and the VL comprises SEQ ID NO: 50.

73. The conjugate of claim 70, wherein the protein comprises two heavy chains HC1 and HC2 and two light chains LC1 and LC2, wherein HC1, LC1, HC2, and LC2 comprise the following sequences:
(a) HC1 comprises SEQ ID NO: 64, LC1 comprises SEQ ID NO: 63, HC2 comprises SEQ ID NO: 51, and LC2 comprises SEQ ID NO: 52;
(b) HC1 comprises SEQ ID NO: 55, LC1 comprises SEQ ID NO: 54, HC2 comprises SEQ ID NO: 51, and LC2 comprises SEQ ID NO: 52;
(c) HC1 comprises SEQ ID NO: 56, LC1 comprises SEQ ID NO: 57, HC2 comprises SEQ ID NO: 51, and LC2 comprises SEQ ID NO: 52; or
(d) HC1 comprises SEQ ID NO: 58, LC1 comprises SEQ ID NO: 59, HC2 comprises SEQ ID NO: 51, and LC2 comprises SEQ ID NO: 52.

74. The conjugate of claim 44, wherein the linker is a Mal-Tet-TCO linker, SMCC linker, or GDM linker.

75. The conjugate of claim 44, wherein the linker is a SMCC linker.

76. The conjugate of claim 44, wherein P is linked to the 3′ end of the sense strand of dsRNA, optionally via the linker.

77. The conjugate of claim 44, wherein the antisense strand is complementary to a target mRNA selected from SNCA, MAPT, APP, ATXN2, ATXN3, SARM1, APOE, BACE1, FMR1, LRRK2, HTT, SOD1, SCN10A, SCN9A or CACNA1B mRNA.

78. The conjugate of claim 77, wherein the antisense strand is complementary to SNCA mRNA.

79. The conjugate of claim 78, wherein the sense strand and the antisense strand comprise a pair of nucleic acid sequences selected from the group consisting of:
(a) the sense strand comprises SEQ ID NO: 81, and the antisense strand comprises SEQ ID NO: 82;
(b) the sense strand comprises SEQ ID NO: 83, and the antisense strand comprises SEQ ID NO: 84;
(c) the sense strand comprises SEQ ID NO: 85, and the antisense strand comprises SEQ ID NO: 86;
(d) the sense strand comprises SEQ ID NO: 87, and the antisense strand comprises SEQ ID NO: 88;
(e) the sense strand comprises SEQ ID NO: 89, and the antisense strand comprises SEQ ID NO: 90;
(f) the sense strand comprises SEQ ID NO: 91, and the antisense strand comprises SEQ ID NO: 92; and
(g) the sense strand comprises SEQ ID NO: 116, and the antisense strand comprises SEQ ID NO: 82,
wherein optionally one or more nucleotides of the sense strand and the antisense strand are independently modified nucleotides, and wherein optionally one or more internucleotide linkages of the sense strand and the antisense strand are modified internucleotide linkages.

80. The conjugate of claim 79, wherein the sense strand comprises SEQ ID NO: 81, and the antisense strand comprises SEQ ID NO: 82.

81. The conjugate of claim 77, wherein the antisense strand is complementary to MAPT mRNA.

82. The conjugate of claim 81, wherein the sense strand and the antisense strand comprise a pair of nucleic acid sequences selected from the group consisting of:
(a) the sense strand comprises SEQ ID NO: 120, and the antisense strand comprises SEQ ID NO: 121;
(b) the sense strand comprises SEQ ID NO: 122, and the antisense strand comprises SEQ ID NO: 123; and
(c) the sense strand comprises SEQ ID NO: 124, and the antisense strand comprises SEQ ID NO: 125,
wherein optionally one or more nucleotides of the sense strand and the antisense strand are independently modified nucleotides, and wherein optionally one or more internucleotide linkages of the sense strand and the antisense strand are modified internucleotide linkages.

83. The conjugate of claim 44, wherein one or more nucleotides of the sense strand are modified nucleotides.

84. The conjugate of claim 83, wherein each nucleotide of the sense strand is a modified nucleotide.

85. The conjugate of claim 44, wherein one or more nucleotides of the antisense strand are modified nucleotides.

86. The conjugate of claim 85, wherein each nucleotide of the antisense strand is a modified nucleotide.

87. The conjugate of claim 83, wherein the modified nucleotide is a 2′-fluoro modified nucleotide, 2′-O-methyl modified nucleotide or 2′-O-alkyl modified nucleotide.

88. The conjugate of claim 85, wherein the modified nucleotide is a 2′-fluoro modified nucleotide, 2′-O-methyl modified nucleotide or 2′-O-alkyl modified nucleotide.

89. The conjugate of claim 87, wherein the sense strand has four 2′-fluoro modified nucleotides at positions 7, 9, 10, and 11 from the 5′ end of the sense strand.

90. The conjugate of claim 89, wherein nucleotides at positions other than positions 7, 9, 10, and 11 of the sense strand are 2′-O-methyl modified nucleotides.

91. The conjugate of claim 89, wherein the antisense strand has four 2′-fluoro modified nucleotides at positions 2, 6, 14, and 16 from the 5′ end of the antisense strand.

92. The conjugate of claim 91, wherein nucleotides at positions other than positions 2, 6, 14 and 16 of the antisense strand are 2′-O-methyl modified nucleotides.

93. The conjugate of claim 87, wherein the sense strand has three 2′-fluoro modified nucleotides at positions 9, 10, and 11 from the 5′ end of the sense strand.

94. The conjugate of claim 93, wherein nucleotides at positions other than positions 9, 10, and 11 of the sense strand are 2′-O-methyl modified nucleotides.

95. The conjugate of claim 93, wherein the antisense strand has five 2′-fluoro modified nucleotides at positions 2, 5, 7, 14, and 16 from the 5′ end of the antisense strand.

96. The conjugate of claim 95, wherein nucleotides at positions other than positions 2, 5, 7, 14, and 16 of the antisense strand are 2′-O-methyl modified nucleotides.

97. The conjugate of claim 93, wherein the antisense strand has five 2′-fluoro modified nucleotides at positions 2, 5, 8, 14, and 16 from the 5′ end of the antisense strand.

98. The conjugate of claim 97, wherein nucleotides at positions other than positions 2, 5, 8, 14, and 16 of the antisense strand are 2′-O-methyl modified nucleotides.

99. The conjugate of claim 93, wherein the antisense strand has five 2′-fluoro modified nucleotides at positions 2, 3, 7, 14, and 16 from the 5′ end of the antisense strand.

100. The conjugate of claim 99, wherein nucleotides at positions other than positions 2, 3, 7, 14, and 16 of the antisense strand are 2′-O-methyl modified nucleotides.

101. The conjugate of claim 44, wherein the sense strand and the antisense strand have one or more modified internucleotide linkages.

102. The conjugate of claim 101, wherein the modified internucleotide linkage is phosphorothioate linkage.

103. The conjugate of claim 101, wherein the sense strand has four or five phosphorothioate linkages.

104. The conjugate of claim 101, wherein the antisense strand has four or five phosphorothioate linkages.

105. The conjugate of claim 44, wherein the antisense strand has a phosphate analog at 5′ end.

106. The conjugate of claim 105, wherein the phosphate analog is 5′-vinylphosphonate.

107. The conjugate of claim 44, wherein the sense strand comprises an abasic moiety or inverted abasic moiety.

108. The conjugate of claim 44, wherein the sense strand comprises an abasic moiety at position 10.

109. The conjugate of claim 78, wherein the sense strand and the antisense strand comprise a pair of nucleic acid sequences selected from the group consisting of:
(a) the sense strand comprises SEQ ID NO: 93 or 140, and the antisense strand comprises SEQ ID NO: 94;
(b) the sense strand comprises SEQ ID NO: 95 or 141, and the antisense strand comprises SEQ ID NO: 96;
(c) the sense strand comprises SEQ ID NO: 95 or 141, and the antisense strand comprises SEQ ID NO: 97;
(d) the sense strand comprises SEQ ID NO: 95 or 141, and the antisense strand comprises SEQ ID NO: 98;
(e) the sense strand comprises SEQ ID NO: 99 or 142, and the antisense strand comprises SEQ ID NO: 94;
(f) the sense strand comprises SEQ ID NO: 100 or 143, and the antisense strand comprises SEQ ID NO: 101;
(g) the sense strand comprises SEQ ID NO: 102 or 144, and the antisense strand comprises SEQ ID NO: 103;
(h) the sense strand comprises SEQ ID NO: 104 or 145, and the antisense strand comprises SEQ ID NO: 105;
(i) the sense strand comprises SEQ ID NO: 106 or 146, and the antisense strand comprises SEQ ID NO: 107;
(j) the sense strand comprises SEQ ID NO: 108 or 147, and the antisense strand comprises SEQ ID NO: 107;
(k) the sense strand comprises SEQ ID NO: 117 or 148, and the antisense strand comprises SEQ ID NO: 97; and
(l) the sense strand comprises SEQ ID NO: 118 or 149, and the antisense strand comprises SEQ ID NO: 97.

110. The conjugate of claim 78, wherein the sense strand and the antisense strand have a pair of nucleic acid sequences selected from the group consisting of:
(a) the sense strand consists of SEQ ID NO: 93 or 140, and the antisense strand consists of SEQ ID NO: 94;
(b) the sense strand consists of SEQ ID NO: 95 or 141, and the antisense strand consists of SEQ ID NO: 96;
(c) the sense strand consists of SEQ ID NO: 95 or 141, and the antisense strand consists of SEQ ID NO: 97;
(d) the sense strand consists of SEQ ID NO: 95 or 141, and the antisense strand consists of SEQ ID NO: 98;
(e) the sense strand consists of SEQ ID NO: 99 or 142, and the antisense strand consists of SEQ ID NO: 94;
(f) the sense strand consists of SEQ ID NO: 100 or 143, and the antisense strand consists of SEQ ID NO: 101;
(g) the sense strand consists of SEQ ID NO: 102 or 144, and the antisense strand consists of SEQ ID NO: 103;
(h) the sense strand consists of SEQ ID NO: 104 or 145, and the antisense strand consists of SEQ ID NO: 105;
(i) the sense strand consists of SEQ ID NO: 106 or 146, and the antisense strand consists of SEQ ID NO: 107;
(j) the sense strand consists of SEQ ID NO: 108 or 147, and the antisense strand consists of SEQ ID NO: 107;
(k) the sense strand consists of SEQ ID NO: 117 or 148, and the antisense strand consists of SEQ ID NO: 97; and
(l) the sense strand consists of SEQ ID NO: 118 or 149, and the antisense strand consists of SEQ ID NO: 97.

111. The conjugate of claim 81, wherein the sense strand and the antisense strand comprise a pair of nucleic acid sequences selected from the group consisting of:
(a) the sense strand comprises SEQ ID NO: 126 or 150, and the antisense strand comprises SEQ ID NO: 127;
(b) the sense strand comprises SEQ ID NO: 128 or 151, and the antisense strand comprises SEQ ID NO: 129;
(c) the sense strand comprises SEQ ID NO: 130 or 152, and the antisense strand comprises SEQ ID NO: 131;
(d) the sense strand comprises SEQ ID NO: 132 or 153, and the antisense strand comprises SEQ ID NO: 133;
(e) the sense strand comprises SEQ ID NO: 134 or 154, and the antisense strand comprises SEQ ID NO: 135; and
(f) the sense strand comprises SEQ ID NO: 136 or 155, and the antisense strand comprises SEQ ID NO: 137.

112. The conjugate of claim 81, wherein the sense strand and the antisense strand comprise a pair of nucleic acid sequences selected from the group consisting of:
(a) the sense strand consists of SEQ ID NO: 126 or 150, and the antisense strand consists of SEQ ID NO: 127;
(b) the sense strand consists of SEQ ID NO: 128 or 151, and the antisense strand consists of SEQ ID NO: 129;
(c) the sense strand consists of SEQ ID NO: 130 or 152, and the antisense strand consists of SEQ ID NO: 131;
(d) the sense strand consists of SEQ ID NO: 132 or 153, and the antisense strand consists of SEQ ID NO: 133;
(e) the sense strand consists of SEQ ID NO: 134 or 154, and the antisense strand consists of SEQ ID NO: 135; and
(f) the sense strand consists of SEQ ID NO: 136 or 155, and the antisense strand consists of SEQ ID NO: 137.

113. A pharmaceutical composition comprising the conjugate of claim 44, and a pharmaceutically acceptable carrier.

114. A method of treating a CNS disease in a patient in need thereof, the method comprising administering to the patient an effective amount of the conjugate of claim 44.

115. A method of treating a neurodegenerative synucleinopathy in a patient in need thereof, the method comprising administering to the patient an effective amount of the conjugate of claim 44.

116. The method of claim 115, wherein the neurodegenerative synucleinopathy is selected from Parkinson's disease, Alzheimer's disease, multiple system atrophy, or Lewy body dementia.

117. A method of treating a tauopathy in a patient in need thereof, the method comprising administering to the patient an effective amount of the conjugate of claim 44.

118. The method of claim 117, wherein the tauopathy is selected from Alzheimer's disease, frontotemporal dementia (FTD), frontotemporal dementia with parkinsonism linked to chromosome 17 (FTDP-17), frontotemporal lobar degeneration (FTLD), behavioral variant frontotemporal dementia (bvFTD), nonfluent variant primary progressive aphasia (nfvPPA), Parkinson's discase, Pick's disease (PiD), primary progressive aphasia-semantic (PPA-S), primary progressive aphasia-logopenic (PPA-L), multiple system tauopathy with presenile dementia (MSTD), neurofibrillary tangle (NFT) dementia, FTD with motor neuron disease, progressive supranuclear palsy (PSP), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, epilepsy, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, postencephalitic parkinsonism, prion protein cerebral amyloid angiopathy, progressive subcortical gliosis, tangle only dementia, tangle-predominant dementia, ganglioglioma, gangliocytoma, subacute sclerosingpan encephalitis, tuberous sclerosis, lipofuscinosis, primary age-related tauopathy (PART), or globular glial tauopathies (GGT).

119. The method of claim 115, wherein the conjugate is administered to the patient intravenously or subcutaneously.

120. The method of claim 117, wherein the conjugate is administered to the patient intravenously or subcutaneously.
